(19) United States
(12) Patent Application Publication
(10) Pub. No.: US 2024/0042063 A1
(43) Pub. Date: Feb. 8, 2024

(54) LINKED AND OTHER pH-TRIGGERED COMPOUNDS

(71) Applicants: UNIVERSITY OF RHODE ISLAND BOARD OF TRUSTEES, Kingston, RI (US); YALE UNIVERSITY, New Haven, CT (US)

(72) Inventors: Yana K. RESHETNYAK, Saunderstown, RI (US); Oleg A. ANDREEV, Saunderstown, RI (US); Donald M. ENGELMAN, New Haven, CT (US)

(21) Appl. No.: 18/331,904

(22) Filed: Jun. 8, 2023

Related U.S. Application Data
(63) Continuation of application No. 16/005,195, filed on Jun. 11, 2018, now abandoned.
(60) Provisional application No. 62/517,830, filed on Jun. 9, 2017.

(51) Int. Cl. A61K 49/00; G01N 33/84; C07K 7/06; A61K 47/65; A61K 47/42; A61B 5/00; A61K 47/64; C07K 14/00

(52) U.S. Cl. CPC A61K 49/0056; G01N 33/84; C07K 7/06; A61K 47/65; A61K 47/42; A61B 5/0071; A61K 47/64; C07K 14/00; G01N 2033/0003

(57) ABSTRACT

Provided herein are, inter alia, pH-triggered compounds and compositions comprising one or more peptides that are capable of inserting into a lipid bilayer below a certain pH. Treatment, imaging, diagnostic, and other uses of such compounds and compositions are also provided.

### CROSS-REFERENCE TO RELATED APPLICATIONS

This application is a continuation of U.S. patent application Ser. No. 16/005,195, filed Jun. 11, 2018, which claims the benefit of priority to U.S. Provisional Application No. 62/517,830 filed Jun. 9, 2017, the entire contents of which are incorporated herein by reference.

### STATEMENT AS TO FEDERALLY SPONSORED RESEARCH

This invention was made with government support under grant number R01 GM073857 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 8, 2023, is named 5095.0070003_SequenceListing_St26 and is 483,744 bytes in size.

### FIELD OF THE INVENTION

The invention generally relates to compositions and methods for the delivery of molecules to cell membranes, cells, and tissues, peptides with increased affinity to membrane lipid bilayers at low pH, as well as peptide insertion into and passage across membrane lipid bilayers.

### BACKGROUND

It has been observed that many diseased tissues and some normal tissues are acidic, and that tumors are especially so. Tumor development, progression, and invasiveness, as well as other pathological states such as ischemia, stroke, inflammation, arthritis, infection, atherosclerosis are associated with the elevation of extracellular acidosis. Extracellular acidity is established at early stages of tumor development, during the avascular phase of carcinoma in situ. As a tumor continues to grow, acidosis increases due to the poor blood perfusion, a switch of cancer cells to glycolytic mechanism of energy production even in the presence of oxygen, and overexpression of carbonic anhydrases (CA). Adaptations to the highly acidic microenvironment are critical steps in the transition from an avascular pre-invasive tumor to a malignant invasive carcinoma (Wojtkowiak et al. (2011) Mol Pharm 8(6):2032-2038; Mahoney et al. (2003) Biochem Pharmacol 66(7):1207-1218; Gatenby R A & Gillies R J (2008) Nat Rev Cancer 8(1):56-61; Lamonte et al. (2013) Cancer Metab 1(1):23).

New compositions and methods for targeting acidic tissues are needed.

### SUMMARY

Provided herein are, inter alia, pH-triggered peptide (pHLIP peptide) compounds that include one pHLIP peptide or multiple pHLIP peptides. Compounds comprising one or more pHLIP peptides may be referred to herein as “pHLIP compounds.” In various embodiments, a pHLIP compound comprises a linker. In some embodiments, a pHLIP compound is conjugated to or comprises a cargo compound. In certain embodiments, a pHLIP compound comprises more than one pHLIP peptide.

In an aspect, provided herein is a pH-triggered compound comprising a pH-triggered peptide (pHLIP peptide) that is covalently attached to at least one other pHLIP peptide via a linker or a covalent bond. In various embodiments, the compound comprises the following structure: A-L-B. In some embodiments, A is a first pHLIP peptide comprising the sequence DDQNPWRAYLDLLFPTDTLLLDLLW (SEQ ID NO: 1), B is a second pHLIP peptide comprising the sequence DDQNPWRAYLDLLFPTDTLLLDLLW (SEQ ID NO: 1), L is a polyethylene glycol linker, and each - is a covalent bond. In certain embodiments, A is a first pHLIP peptide comprising the sequence AEQNPIYWARYADWLFTTPLLLLDLALLVDADEGT (SEQ ID NO: 2), B is a second pHLIP peptide comprising the sequence AEQNPIYWARYADWLFTTPLLLLDLALLVDADEGT (SEQ ID NO: 2), L is a polyethylene glycol linker, and each - is a covalent bond. In some embodiments, A is a first pHLIP peptide comprising the sequence GLAGLAGLLGLEGLLGLPLGLLEGLWLGLELEGN (SEQ ID NO: 3), B is a second pHLIP peptide comprising the sequence GLAGLAGLLGLEGLLGLPLGLLEGLWLGLELEGN (SEQ ID NO: 3), L is a polyethylene glycol linker, and each - is a covalent bond.

In various embodiments, a pHLIP compound comprises at least one pHLIP peptide comprising one or more of the following sequences: AYLDLLFP (SEQ ID NO: 4), YLDLLFPT (SEQ ID NO: 5), LDLLFPTD (SEQ ID NO: 6), DLLFPTDT (SEQ ID NO: 7), LLFPTDT (SEQ ID NO: 8), LFPTDTLL (SEQ ID NO: 9), FPTDTLLL (SEQ ID NO: 10), PTDTLLLD (SEQ ID NO: 11), TDTLLLDL (SEQ ID NO: 12), DTLLLDLL (SEQ ID NO: 13), or TLLLDLLW (SEQ ID NO: 14). In some embodiments, a pHLIP compound comprises at least one pHLIP peptide comprising the sequence DDQNPWRAYLDLLFPTDTLLLDLLW (SEQ ID NO: 1), ACDDQNPWRAYLDLLFPTDTLLLDLLWA (SEQ ID NO: 15), AKDDQNPWRAYLDLLFPTDTLLLDLLWA (SEQ ID NO: 16), ADDQNPWRAYLDLLFPTDTLLLDLLWCA (SEQ ID NO: 17), ADDQNPWRAYLDLLFPTDTLLLDLLWKA (SEQ ID NO: 18), ACDDQNPWRAYLDLLFPTDTLLLDLLWKA (SEQ ID NO: 19), or AKDDQNPWRAYLDLLFPTDTLLLDLLWCA (SEQ ID NO: 20). In certain embodiments, a pHLIP compound comprises at least one pHLIP peptide comprising the sequence

(SEQ ID NO: 1)
DDQNPWRAYLDLLFPTDTLLLDLLW.

In various embodiments, compounds provided herein have increased potency, making them particularly suitable for the delivery of highly toxic molecules (such as amanitin) to acidic tissues such as tumors. In some embodiments, linking multiple pHLIP peptides together increases tumor targeting and/or the delivery of diagnostic (imaging) and/or therapeutic cargo compounds. In certain embodiments, linking two or more pHLIP peptides increases the efficiency of delivery, which increases the translocation of cargo compounds across cell membranes.

A non-limiting example of a general formula for a pHLIP compound is:
[pHLIP peptide]ₖ-Linker,
where the pHLIP peptide is a pH-triggered linear peptide comprising at least 8 amino acids, wherein (i) at least 4 of the 8 amino acids of said peptide are a non-polar amino acids, and (ii) at least one of the at least 8 amino acids of said peptide is protonatable. In various embodiments, the peptide has a higher affinity for a membrane lipid bilayer at pH 5.0 compared to the affinity at pH 8.0. In some embodiments, the linker is a natural polymer or a synthetic polymer. In certain embodiments, k is an integer from 1 to 32. In some embodiments, the peptide comprises one or more non-coded amino acids such as gamma-carboxyglutamic acid (Gla) or alpha-aminoadipic acid (Aad).

In various embodiments, the pHLIP peptide has the sequence: XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYₗXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein, (i) Y is a non-polar amino acid with solvation energy, ΔG_{X}^{cor}>+0.50, or Gly (see, e.g., Table 1), (ii) X is a protonatable amino acid, and (iii) n, m, i, j, l, h, g, f are integers from 1 to 8.

Aspects of the present subject matter relate to “Variant 3” or “Var3” pHLIP peptides. Var3 pHLIP peptides comprise the following sequence: DDQNPWRAYLDLLFPTDTLLLDLLW (SEQ ID NO: 1). Var3 family pHLIP peptides comprise at least 8 consecutive amino acids that are within this sequence, wherein the least 8 consecutive amino acids include at least one protonatable amino acid (i.e., aspartic acid). In various embodiments, a Var3 family pHLIP peptide comprises one or more of the following sequences (protonatable amino acids are underlined):

(SEQ ID NO: 4)
Ala-Tyr-Leu-Asp-Leu-Leu-Phe-Pro
(SEQ ID NO: 5)
Tyr-Leu-Asp-Leu-Leu-Phe-Pro-Thr
(SEQ ID NO: 6)
Leu-Asp-Leu-Leu-Phe-Pro-Thr-Asp
(SEQ ID NO: 7)
Asp-Leu-Leu-Phe-Pro-Thr-Asp-Thr
(SEQ ID NO: 325)
Leu-Leu-Phe-Pro-Thr-Asp-Thr-Leu
(SEQ ID NO: 9)
Leu-Phe-Pro-Thr-Asp-Thr-Leu-Leu
(SEQ ID NO: 10)
Phe-Pro-Thr-Asp-Thr-Leu-Leu-Leu
(SEQ ID NO: 11)
Pro-Thr-Asp-Thr-Leu-Leu-Leu-Asp
(SEQ ID NO: 12)
Thr-Asp-Thr-Leu-Leu-Leu-Asp-Leu
(SEQ ID NO: 13)
Asp-Thr-Leu-Leu-Leu-Asp-Leu-Leu
(SEQ ID NO: 14)
Thr-Leu-Leu-Leu-Asp-Leu-Leu-Trp

In certain embodiments, a Var3 family pHLIP peptide includes a stretch of amino acids in the sequence LFPTDTLL (SEQ ID NO: 9). Non-limiting examples of Var3 family pHLIP peptide sequences include ADDQNPWRAYLDLLFPTDTLLLDLLWG (SEQ ID NO: 21),

(SEQ ID NO: 22)
AKDDQNPWRAYLDLLFPTDTLLLDLLWG,
(SEQ ID NO: 15)
ACDDQNPWRAYLDLLFPTDTLLLDLLWA,
(SEQ ID NO: 23)
ADDQNPWRAYLDLLFPTDTLLLDLLWA,
(SEQ ID NO: 17)
ADDQNPWRAYLDLLFPTDTLLLDLLWCA,
(SEQ ID NO: 18)
ADDQNPWRAYLDLLFPTDTLLLDLLWKA,
(SEQ ID NO: 16)
AKDDQNPWRAYLDLLFPTDTLLLDLLWA,
(SEQ ID NO: 24)
ACDDQNPWRAYLDLLFPTDTLLLDLLWG,
(SEQ ID NO: 25)
ADDQNPWRAYLDLLFPTDTLLLDLLWCG,
(SEQ ID NO: 26)
ADDQNPWRAYLDLLFPTDTLLLDLLWKG,
(SEQ ID NO: 27)
ACDDQNPWRAYLDLLFPTDTLLLDLLWKG,
(SEQ ID NO: 28)
AKDDQNPWRAYLDLLFPTDTLLLDLLWCG,
and
(SEQ ID NO: 29)
ACKDDQNPWRAYLDLLFPTDTLLLDLLWG.

In some embodiments, a Cys and/or Lys is positioned at or near (at an end or within 1, 2, or 3 positions from an end) of the N- or C-terminal end of a pHLIP peptide (such as a Var3 family pHLIP peptide) for conjugation purposes to make a pHLIP bundle. In certain embodiments, such a pHLIP peptide is used with other groups for click chemistry at the Cys and/or Lys position(s). In some examples, the amino terminal residue is acetylated (acetylation is indicated below with the abbreviation “Ac”). Acetylation is used to block the amino moiety (NH₂) of an amino acid; such a block is used in some circumstances to prevent or reduce undesirable conjugation. The term “Free” in the sequences below indicates the absence of a blocking group, e.g, by acetylation. In such peptides, the terminal residue has an NH₂ moiety that is not blocked, e.g, it is accessible to chemical reactions. When conjugation to make bundles is carried out via a Cys residue, blocking of the amino terminal residue is typically absent, e.g., it is not needed to prevent/reduce undesirable conjugation. In some embodiments, a Var3 family pHLIP peptide has the following sequence (Cys and Lys residues are underlined):

(SEQ ID NO: 15)
Free-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Ala-COOH,
(SEQ ID NO: 16)
Free-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Ala-COOH,
(SEQ ID NO: 15)
Ac-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Ala-COOH,
(SEQ ID NO: 16)
Ac-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Ala-COOH,
where ″Ac-″ means ″N-Terminal Acetylation″,
(SEQ ID NO: 17)
Free-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Cys-Ala-COOH,
(SEQ ID NO: 18)
Free-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Lys-Ala-COOH,
(SEQ ID NO: 17)
Ac-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Cys-Ala-COOH,
(SEQ ID NO: 18)
Ac-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Lys-Ala-COOH,
(SEQ ID NO: 20)
Free-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-Ala-COOH,
(SEQ ID NO: 30)
Free-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-Ala-COOH,
(SEQ ID NO: 20)
Ac-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-Ala-COOH,
(SEQ ID NO: 30)
Ac-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-Ala-COOH,
(SEQ ID NO: 31)
Free-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-Ala-COOH,
(SEQ ID NO: 19)
Free-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-Ala-COOH,
(SEQ ID NO: 31)
Ac-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-Ala-COOH,
(SEQ ID NO: 19)
Ac-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-Ala-COOH,
(SEQ ID NO: 326)
Free-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Cys-COOH,
(SEQ ID NO: 32)
Free-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Lys-COOH,
(SEQ ID NO: 326)
Ac-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Cys-COOH,
(SEQ ID NO: 32)
Ac-Ala-Asp-Asp-Gln-Asn-Pro-Trp-Arg-Ala-Tyr-Leu-
Asp-Leu-Leu-Phe-Pro-Thr-Asp-Thr-Leu-Leu-Leu-Asp-
Leu-Leu-Trp-Lys-COOH,
(SEQ ID NO: 33)
Free-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-COOH,
(SEQ ID NO: 34)
Free-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-COOH,
(SEQ ID NO: 33)
Ac-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-COOH,
(SEQ ID NO: 34)
Ac-Ala-Lys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-COOH,
(SEQ ID NO: 35)
Free-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-COOH,
(SEQ ID NO: 36)
Free-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-COOH,
(SEQ ID NO: 35)
Ac-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Cys-COOH,
or
(SEQ ID NO: 36)
Ac-Ala-Cys-Asp-Asp-Gln-Asn-Pro-Trp-Arg-Ala-Tyr-
Leu-Asp-Leu-Leu-Phe-Pro-Thr-Asp-Thr-Leu-Leu-Leu-
Asp-Leu-Leu-Trp-Lys-COOH.

Variants of the pHLIP peptides exemplified or otherwise disclosed herein may be designed using substitution techniques that are well understood in the art. Neither the pHLIP peptides exemplified herein nor the variants discussed below limit the full scope of the subject matter disclosed herein.

In certain embodiments, the pHLIP peptide comprises the sequence:

(SEQ ID NO: 37)
WARYADWLFTTPLLLLDLALLV,
(SEQ ID NO: 38)
WARYAGlaWLFTTPLLLLDLALLV,
(SEQ ID NO: 39)
WARYAGlaWLFTTPLLLLAadLALLV,
(SEQ ID NO: 40)
PWRAYLDLLFPTDTLLLDLLW,
(SEQ ID NO: 41)
PWRAYLGlaLLFPTDTLLLDLLW,
(SEQ ID NO: 42)
PWLGAYLDLLFPLELLGLLELGLWG,
or
(SEQ ID NO: 43)
LLGLEGLLGLPLGLLEGLWLGLEL.

In various embodiments, the pHLIP peptide comprises the sequence: PWRAYLDLLFPTDTLLLDLLW (SEQ ID NO: 40).

In certain embodiments, the pHLIP peptide comprises the sequence:

(SEQ ID NO: 2)
AEQNPIYWARYADWLFTTPLLLLDLALLVDADEGT,
(SEQ ID NO: 44)
AEQNPIYWARYAGlaWLFTTPLLLLDLALLVDADEGT,
(SEQ ID NO: 45)
AEQNPIYWARYAGlaWLFTTPLLLLAadLALLVDADEGT,
(SEQ ID NO: 46)
ADDQNPWRAYLDLLFPTDTLLLDLLW,
(SEQ ID NO: 47)
ADDQNPWRAYLGlaLLFPTDTLLLDLLW,
(SEQ ID NO: 48)
GEEQNPWLGAYLDLLFPLELLGLLELGLWG,
or
(SEQ ID NO: 3)
GLAGLAGLLGLEGLLGLPLGLLEGLWLGLELEGN.

In some embodiments, different amino acid pHLIP peptide sequences are linked together by a linker. In certain embodiments, the pHLIP compound comprises a mixture of different pHLIP peptides for k≥1. In various embodiments, the same amino acid pHLIP peptide sequence is linked together by a linker k times, where 1<k≤32. In certain embodiments, the same amino acid pHLIP peptide sequence is linked together by a linker k times, where 1≤k. In some embodiments, the same amino acid pHLIP peptide sequence is linked together by a linker k times, where k≥32. In certain embodiments, the same amino acid pHLIP peptide sequence is linked together by a linker k times, where k≤32.

In some embodiments, each pHLIP peptide has a net negative charge at a pH of about 7.25, 7.5, or 7.75 in water.

In certain embodiments, each pHLIP peptide has an acid dissociation constant on a base 10 logarithmic scale (pKa) of less than about 4.0, 4.5, 5.0, 5.5, 6.0, 6.5, or 7.0. In various embodiments, each pHLIP peptide has a pKa of at least about 6.5, 6.6, 6.7, 6.8, 6.9, or 7.0. In some embodiments, each pHLIP peptide has a pKa between about 6.5 and about 7.0, e.g., about 6.6 and about 7.0, about 6.7 and about 7.0, about 6.8 and about 7.0, or about 6.9 and about 7.0. In certain embodiments, each pHLIP peptide has a pKa of about 6.5, 6.6, 6.7, 6.8, 6.9, or 7.0.

In various embodiments, each pHLIP peptide comprises 1 protonatable amino acid which is aspartic acid, glutamic acid, alpha-aminoadipic acid, or gamma-carboxyglutamic acid. In some embodiments, each pHLIP peptide comprises at least 2, 3, or 4 protonatable amino acids, wherein the protonatable amino acids comprise one or more of aspartic acid, glutamic acid, alpha-aminoadipic acid, and gamma-carboxyglutamic acid, or any combination thereof.

In certain embodiments, a pHLIP peptide comprises at least 1 non-native protonatable amino acid. In various embodiments, the non-native protonatable amino acid of a pHLIP peptide comprises at least 1, 2, 3 or 4 carboxyl groups.

In some embodiments, a pHLIP peptide comprises at least 1, 2, 3, 4, 5, 6, 7, 8, 9, 10, 11, 12, 13, 14, 15, or 16 carboxyl groups. In some embodiments, a pHLIP peptide comprises between 1, 2, or 3 and 4, 5, 6, 7, 8, 9, or 10 carboxyl groups.

In certain embodiments, a pHLIP peptide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 coded amino acids.

In various embodiments, a pHLIP peptide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 non-coded amino acids.

In some embodiments, every amino acid of a pHLIP peptide is a non-native amino acid.

In certain embodiments, a pHLIP peptide comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 D-amino acids.

In various embodiments, a pHLIP peptide comprises at least 1 non-coded amino acid, wherein the non-coded amino acid is an aspartic acid derivative, or a glutamic acid derivative.

In some embodiments, a pHLIP peptide comprises at least 8 amino acids, wherein, at least 2, 3, or 4 of the 8 amino acids of said peptide are non-polar, and at least 1, 2, 3, or 4 of the at least 8 amino acids of said pHLIP peptide is protonatable.

In certain embodiments, a pHLIP peptide comprises a functional group to which a linker is attached.

In various embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides are linked together by a linker.

In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides are directly linked to a linker by covalent bonds.

In certain embodiments, the pHLIP peptides are attached to a linker by covalent bonds.

In various embodiments, the covalent bond between a pHLIP peptide and the linker compound is a peptide bond.

In some embodiments, the covalent bond between a pHLIP peptide and the linker compound is a disulfide bond, a bond between two selenium atoms, or a bond between a sulfur and a selenium atom.

In certain embodiments, the covalent bond between a pHLIP peptide and the linker compound is a bond that has been formed by a click chemistry reaction.

In various embodiments, the covalent bond between a pHLIP peptide and the linker compound is a bond that has been formed by a reaction between (i) an azide and an alkyne; (ii) an alkyne and a strained difluorooctyne; (iii) a diaryl-strained-cyclooctyne and a 1,3-nitrone; (iv) a cyclooctene, trans-cycloalkene, or oxanorbornadiene and an azide, tetrazine, or tetrazole; (v) an activated alkene or oxanorbornadiene and an azide; (vi) a strained cyclooctene or other activated alkene and a tetrazine; or (vii) a tetrazole that has been activated by ultraviolet light and an alkene.

In some embodiments, the linker comprises a natural polymer or a synthetic polymer.

In certain embodiments, the linker comprises of a peptide bond, a polypeptide, a polylysine, a polyarginine, a polyglutamic acid, a polyaspartic acid, a polycysteine, or a polynucleic acid.

In various embodiments, the linker comprises a polysaccharide, a chitosan, or an alginate.

In some embodiments, the linker comprises a poly(ethylene glycol), a poly(lactic acid), a poly(glycolic acid), a poly(lactic-co-glycolic acid), a poly(malic acid), a polyorthoesters, a poly(vinylalcohol), a poly(vinylpyrrolidone), a poly(methyl rnethacrylate), a poly(acrylic acid), a poly(acrylamide), a poly(methacrylic acid), a poly(amidoamine), a polyanhydrides, or a polycyanoacrylate.

In certain embodiments, the linker comprises a linear polymer or a branched polymer.

In various embodiments, the linker comprises a cell, a particle, a dendrimer, or a nanoparticle.

In some embodiments, the linker comprises a particle, a metallic particle, a polymeric particle, a nanoparticle, a metallic nanoparticle, a lipid-based nanoparticle, a surfactant-based nanoparticle, a polymeric nanoparticle, a peptide-based nanoparticle.

In certain embodiments, a pHLIP peptide comprises a functional group to which a cargo compound may be attached.

In various embodiments, a linker comprises a functional group to which a cargo compound may be attached.

In some embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides are linked to a cargo compound.

In certain embodiments, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides are directly linked to a cargo compound by a covalent bond.

In various embodiments, the covalent bond between a pHLIP peptide and the cargo is an ester bond, a disulfide bond, a bond between two selenium atoms, a bond between a sulfur and a selenium atom, or an acid-liable bond.

In some embodiments, the covalent bond between a pHLIP peptide and the cargo compound is a bond that has been formed by a click chemistry reaction.

In certain embodiments, the click chemistry reaction was a reaction between (i) an azide and an alkyne; (ii) an alkyne and a strained difluorooctyne; (iii) a diaryl-strained-cyclooctyne and a 1,3-nitrone; (iv) a cyclooctene, trans-cycloalkene, or oxanorbornadiene and an azide, tetrazine, or tetrazole; (v) an activated alkene or oxanorbornadiene and an azide; (vi) a strained cyclooctene or other activated alkene and a tetrazine; or (vii) a tetrazole that has been activated by ultraviolet light and an alkene.

In various embodiments, the functional group of a pHLIP peptide is a side chain of an amino acid of the peptide.

In some embodiments, the functional group of a pHLIP peptide is an amino acid side chain to which a cargo compound may be attached via a disulfide bond.

In certain embodiments, the functional group of a pHLIP peptide to which a cargo compound may be attached comprises a free sulfhydryl (SH) or selenohydryl (SeH) group.

In various embodiments, the functional group of a pHLIP peptide comprises a cysteine, homocysteine, selenocysteine, or homoselenocysteine.

In some embodiments, the functional group of a pHLIP peptide comprises a primary amine.

In certain embodiments, the functional group of a pHLIP peptide comprises an azido modified amino acid.

In various embodiments, the functional group of a pHLIP peptide comprises an alkynyl modified amino acid.

In some embodiments, a linker comprises a functional group to which a cargo compound may be attached.

In certain embodiments, the covalent bond between a linker and the cargo is an ester bond, a disulfide bond, a bond between two selenium atoms, a bond between a sulfur and a selenium atom, or an acid-liable bond.

In various embodiments, the covalent bond between a linker and the cargo compound is a bond that has been formed by a click chemistry reaction.

In some embodiments, the click chemistry reaction was a reaction between (i) an azide and an alkyne; (ii) an alkyne and a strained difluorooctyne; (iii) a diaryl-strained-cyclooctyne and a 1,3-nitrone; (iv) a cyclooctene, trans-cycloalkene, or oxanorbornadiene and an azide, tetrazine, or tetrazole; (v) an activated alkene or oxanorbornadiene and an azide; (vi) a strained cyclooctene or other activated alkene and a tetrazine; or (vii) a tetrazole that has been activated by ultraviolet light and an alkene.

In certain embodiments, the functional group of a linker is a side chain of an amino acid.

In various embodiments, the functional group of a linker is an amino acid side chain to which a cargo compound may be attached via a disulfide bond.

In some embodiments, the functional group of a linker to which a cargo compound may be attached comprises a free sulfhydryl (SH) or selenohydryl (SeH) group.

In certain embodiments, the functional group of a linker comprises a cysteine, homocysteine, selenocysteine, or homoselenocysteine.

In various embodiments, the functional group of a linker comprises a primary amine.

In some embodiments, the functional group of a linker comprises an azido modified amino acid.

In certain embodiments, the functional group of a linker comprises an alkynyl modified amino acid.

In various embodiments, the cargo is polar or nonpolar.

In some embodiments, the cargo is a marker.

In certain embodiments, the cargo is a prophylactic, therapeutic, diagnostic, radiation-enhancing, radiation-sensitizing, imaging, gene regulation, immune activation, cytotoxic, apoptotic, or research reagent.

In various embodiments, pHLIP peptides comprising one or more cargo molecules attached to said functional groups is/are used as a therapeutic, diagnostic, imaging, ex vivo imaging, immune activation, gene regulation, cell function regulation, radiation-enhancing, radiation-sensitizing agent, or as a research tool.

In some embodiments, the cargo comprises a dye (e.g., a fluorescent dye), a fluorescence quencher, or a fluorescent protein.

In certain embodiments, the cargo comprises a magnetic resonance agent, a positron emission tomography agent, a single photon emission computed tomography agent, a fluorescent agent, an optoacoustic agent, an ultrasound agent, or an x-ray contrast imaging agent.

In various embodiments, 1 or more of the amino acid side chains of a pHLIP peptide is chemically modified to be radioactive or detectable by probing radiation.

In some embodiments, one or more atoms of a pHLIP peptide is replaced by a radioactive isotope or a stable isotope.

In an aspect, provided herein is a pHLIP compound for use as an agent for ex vivo imaging and/or ex vivo diagnostics.

In certain embodiments, the cargo comprises a peptide, a protein, an enzyme, a polynucleotide, or a polysaccharide.

In various embodiments, the cargo comprises an aptamer, an antigen, a protease, an amylase, a lipase, a Fc receptor, a tissue factor, or a C3 protein.

In some embodiments, the cargo comprises a toxin, an inhibitor, a DNA intercalator, an alkylating agent, an antimetabolite, an anti-microtubule agents, a topoisomerase inhibitor, or an antibiotic.

In certain embodiments, the cargo comprises an amanita toxin, a vinca alkaloid, a taxane, an anthracycline, a bleomycin, a nitrogen mustard, a nitrosourea, a tetrazine, an aziridine, a cisplatin or a derivative thereof, a procarbazine, or a hexamethylmelamine.

In various embodiments, the cargo comprises a DNA, a RNA, or an analog thereof, such as a peptide nucleic acid (PNA), a bis PNA, a gamma PNA, a locked nucleic acid (LNA), or a morpholino.

In some embodiments, the cargo is a chemotherapeutic compound.

In certain embodiments, the cargo is an antimicrobial compound.

In various embodiments, the cargo is a gene-regulation compound. In certain embodiments, the cargo is an antisense oligonucleotide. In some embodiments, the gene-regulation compound is a PNA. Non-limiting descriptions of PNAs are provided in Reshetnyak et al., 2006, PNAS, 103, 6460-6465; Cheng et al., 2015, Nature, 518, 107-110; and Ozes et al., 2017, Sci Reports, 7, 894, 1-11, the entire contents of each of which are incorporated herein by reference.

A non-limiting example of a PNA that targets MDM2 mRNA is TAMRA-o-o-CATAGTATAAGT-o-Cys-NH₂ [TAMRA-o-o-(SEQ ID NO: 331)-o-Cys-NH₂], where TAMRA is a single-isomer 5-carboxytetramethylrhodamine. See, e.g., Reshetnyak et al., 2006, PNAS, 103, 6460-6465.

Non-limiting examples of antimiR PNAs include:
anti155: TAMRA-ooo-ACCCCTATCACAATTAGCATTAA-ooo-Cys [TAMRA-ooo-(SEQ ID NO: 49)-ooo-Cys],
anti21: TAMRA-ooo-TCAACATCAGTCTGATAAGCTA-ooo-Cys [TAMRA-ooo-(SEQ ID NO: 50)-ooo-Cys], and
anti182: TAMRA-ooo-CGGTGTGAGTTCTACCATTGCCAAA-ooo-Cys [TAMRA-ooo-(SEQ ID NO: 51)-ooo-Cys], where TAMRA is a single-isomer 5-carboxytetramethylrhodamine. See, e.g., Cheng et al., 2015, Nature, 518, 107-110.

Non-limiting examples of PNA sequences for suppressing lncRNA HOTAIR (HOX transcript antisense RNA) activity include:
TACTGCAGGC (SEQ ID NO: 52),
GTAACTCTGGG (SEQ ID NO: 53),
TCTGTAACTC (SEQ ID NO: 54), and
CCCTCTCTCC (SEQ ID NO: 55). See, e.g., Ozes et al., 2017, Sci Reports, 7, 894, 1-11. In some embodiments, a PNA comprising these sequences further comprises a cell penetrating peptide comprising the sequence RRRQRRKKR (SEQ ID NO: 56). In certain embodiments, a PNA does not comprise a cell penetrating peptide.

In certain embodiments, pHLIP peptides can solve the challenging problem of delivering PNA into cells, while also targeting the delivery to diseased tissues, enabling a wide range of uses of PNA in the clinic. In various embodiments, a pHLIP compound provided herein is used to treatment cancer, a genetic disease, an infectious disease, arthritis, atherosclerosis, or ischemic myocardium. In some embodiments, antisense offers a platform to regulate targets that have not been druggable such as the KRAS pathway, mdm2 oncogene, or cyclin B1 gene. In certain embodiments, pHLIP compounds are used for targeted disruption of specific pathways for particular tumors, especially resistant tumors, such as Her2 overexpression, EGFR, RAF and many others. In various embodiments, modification of auto-immune responses in immuno-therapy is accomplished using a pHLIP compound provided herein. In some embodiments, a pHLIP compound provided herein is used for gene editing (e.g., by targeting dsDNA associated with a genetic disorder). In certain embodiments, silencing of miRNA or lncRNA is achieved with a pHLIP compound provided herein (e.g., targeting of miRNA or long non-coding RNA is used to treat cancer and other diseases). In various embodiments, silencing of miRNA or lncRNA with a pHLIP compound is used in the treatment of a drug-resistant tumor. In some embodiments, a pHLIP peptide provided herein is used to target a telomeres or a telomerase, e.g., as a monotherapy or in combination with a chemo- or radiation therapy.

In an aspect, provided herein is a pHLIP peptide comprising the sequence: WARYADWLFTTPLLLLDLALLV (SEQ ID NO: 37), WARYAGlaWLFTTPLLLLDLALLV (SEQ ID NO: 38), WARYAGlaWLFTTPLLLLAadLALLV (SEQ ID NO: 39), PWRAYLDLLFPTDTLLLDLLW (SEQ ID NO: 40), PWRAYLGlaLLFPTDTLLLDLLW (SEQ ID NO: 41), PWLGAYLDLLFPLELLGLLELGLWG (SEQ ID NO: 42), LLGLEGLLGLPLGLLEGLWLGLEL (SEQ ID NO: 43), AEQNPIYWARYADWLFTTPLLLLDLALLVDADEGT (SEQ ID NO: 2), AEQNPIYWARYAGlaWLFTTPLLLLDLALLVDADEGT (SEQ ID NO: 44), AEQNPIYWARYAGlaWLFTTPLLLLAadLALLVDADEGT (SEQ ID NO: 45), ADDQNPWRAYLDLLFPTDTLLLDLLW (SEQ ID NO: 46), ADDQNPWRAYLGlaLLFPTDTLLLDLLW (SEQ ID NO: 47), GEEQNPWLGAYLDLLFPLELLGLLELGLWG (SEQ ID NO: 48), or GLAGLAGLLGLEGLLGLPLGLLEGLWLGLELEGN (SEQ ID NO: 3). In various embodiments, the pHLIP peptide compries the sequence: PWRAYLDLLFPTDTLLLDLLW (SEQ ID NO: 40).

In an aspect, provided herein is a pHLIP compound comprising 2-32 pHLIP peptides having the same sequence, wherein the sequence is: WARYADWLFTTPLLLLDLALLV (SEQ ID NO: 37), WARYAGlaWLFTTPLLLLDLALLV (SEQ ID NO: 38), WARYAGlaWLFTTPLLLLAadLALLV (SEQ ID NO: 39), PWRAYLDLLFPTDTLLLDLLW (SEQ ID NO: 40), PWRAYLGlaLLFPTDTLLLDLLW (SEQ ID NO: 41), PWLGAYLDLLFPLELLGLLELGLWG (SEQ ID NO: 42), LLGLEGLLGLPLGLLEGLWLGLEL (SEQ ID NO: 43), AEQNPIYWARYADWLFTTPLLLLDLALLVDADEGT (SEQ ID NO: 2), AEQNPIYWARYAGlaWLFTTPLLLLDLALLVDADEGT (SEQ ID NO: 44), AEQNPIYWARYAGlaWLFTTPLLLLAadLALLVDADEGT (SEQ ID NO: 45), ADDQNPWRAYLDLLFPTDTLLLDLLW (SEQ ID NO: 46), ADDQNPWRAYLGlaLLFPTDTLLLDLLW (SEQ ID NO: 47), GEEQNPWLGAYLDLLFPLELLGLLELGLWG (SEQ ID NO: 48), or GLAGLAGLLGLEGLLGLPLGLLEGLWLGLELEGN (SEQ ID NO: 3). In various embodiments, the sequence is PWRAYLDLLFPTDTLLLDLLW (SEQ ID NO: 40).

In an aspect, included herein is a pHLIP compound for use as an agent to deliver a cargo molecule across cell membranes into cells in a diseased tissue with a naturally acidic extracellular environment or in a tissue with an artificially induced acidic extracellular environment relative to normal physiological pH.

Various implementations provide pHLIP compounds for use an agents to deliver cargo molecules to the surfaces of cells in a diseased tissue with a naturally acidic extracellular environment or in a tissue with an artificially induced acidic extracellular environment relative to normal physiological pH.

In certain embodiments, a pHLIP peptide and a polypeptide linker are expressed genetically at the surfaces of cells.

In an aspect, provided herein is a pHLIP compound for use in coating of a cell, a particle, a nanoparticle, or a surface.

In various embodiments, the nanoparticle is a metallic, a polymeric, a lipid-based, a surfactant-based, or a peptide-based nanoparticle.

In some embodiments, diseased tissue is cancerous tissue, inflamed tissue, ischemic tissue, arthritic tissue, cystic fibrotic tissue, tissue infected with a microorganism, or atherosclerotic tissue.

Also included is a formulation comprising the pHLIP compound for parenteral, local, or systemic administration.

Provided herein is a formulation comprising the pHLIP compound for intravenous, intraarterial, intraperitoneal, intracerebral, intracerebroventricular, intrathecal, intracardiac, intracavernous, intraosseous, intraocular, or intravitreal administration.

In an aspect, included herein is a formulation comprising the pHLIP compound for intramuscular, intradermal, transdermal, transmucosal, intralesional, subcutaneous, topical, epicutaneous, extra-amniotic, intravaginal, intravesical, nasal, or oral administration.

Some implementations provide a formulation comprising the pHLIP compound for an intravesical instillation for treatment of bladder cancer.

Included herein is a formulation comprising the pHLIP compound for systemic administration for treatment of bladder cancer.

In an aspect, included herein is a pHLIP compound comprising a pHLIP peptide, a peptide linker and an amanitin toxic cargo for treatment of superficial and muscle invasive bladder tumors.

Certain implementations include a formulation comprising the pHLIP compound for the ex vivo contacting of biopsy specimens, liquid biopsy specimens, surgically removed tissue, surgically removed liquids, or blood with the pHLIP compound.

In an aspect, provided herein is a pHLIP peptide (as well as compounds comprising at least one such pHLIP peptide) comprising a sequence of at least 8 to 25 consecutive amino acids (e.g., 8, 9, 10, 11, 12, 13, 14, 15, 16, 17, 18, 19, 20, 21, 22, 23, 24, or 25 consecutive amino acids) that is present in any one of the following sequences:

(SEQ ID NO: 57)
WARYADWLFTTPLLLLDLALL,
(SEQ ID NO: 58)
YARYADWLFTTPLLLLDLALL,
(SEQ ID NO: 59)
WARYSDWLFTTPLLLYDLGLL,
(SEQ ID NO: 60)
WARYTDWFTTPLLLYDLALLA,
(SEQ ID NO: 61)
WARYTDWLFTTPLLLYDLGLL,
(SEQ ID NO: 62)
WARYADWLFTTPLLLLDLSLL,
(SEQ ID NO: 63)
LLALDLLLLPTTFLWDAYRAW,
(SEQ ID NO: 64)
LLALDLLLLPTTFLWDAYRAY,
(SEQ ID NO: 65)
LLGLDYLLLPTTFLWDSYRAW,
(SEQ ID NO: 66)
ALLALDYLLLPTTFWDTYRAW,
(SEQ ID NO: 67)
LLGLDYLLLPTTFLWDTYRAW,
(SEQ ID NO: 328)
LLSLDLLLLPTTFLWDAYRAW,
(SEQ ID NO: 68)
GLAGLLGLEGLLGLPLGLLEGLWLGL,
(SEQ ID NO: 69)
LGLWLGELLGLPLGLLGELGLLGALG,
(SEQ ID NO: 70)
WRAYLDLLFPTDTLLLDLLW,
(SEQ ID NO: 71)
WLLDLLLTDTPFLLDLYARW,
(SEQ ID NO: 72)
WARYLEWLFPTETLLLEL,
(SEQ ID NO: 73)
WAQYLELLFPTETLLLEW,
(SEQ ID NO: 74)
LELLLTETPFLWELYRAW,
(SEQ ID NO: 75)
WELLLTETPFLLELYQAW,
(SEQ ID NO: 76)
WLFTTPLLLLNGALLVE,
(SEQ ID NO: 77)
WLFTTPLLLLPGALLVE,
(SEQ ID NO: 78)
WARYADLLFPTTLAW,
(SEQ ID NO: 79)
EVLLAGNLLLLPTTFLW,
(SEQ ID NO: 80)
EVLLAGPLLLLPTTFLW,
(SEQ ID NO: 81)
WALTTPFLLDAYRAW,
(SEQ ID NO: 82)
NLEGFFATLGGEIALWSLVVLAIE,
(SEQ ID NO: 83)
EGFFATLGGEIALWSDVVLAIE,
(SEQ ID NO: 84)
EGFFATLGGEIPLWSDVVLAIE,
(SEQ ID NO: 85)
EIALVVLSWLAIEGGLTAFFGELN,
(SEQ ID NO: 86)
EIALVVDSWLAIEGGLTAFFGE,
(SEQ ID NO: 87)
EIALVVDSWLPIEGGLTAFFGE,
(SEQ ID NO: 88)
ILDLVFGLLFAVTSVDFLVQW,
and
(SEQ ID NO: 89)
WQVLFDVSTVAFLLGFVLDLI.

In an aspect, provided herein is a pHLIP peptide (as well as compounds comprising at least one such pHLIP peptide) comprising the sequence: WARYADWLFTTPLLLLDLALL (SEQ ID NO: 57), YARYADWLFTTPLLLLDLALL (SEQ ID NO: 58), WARYSDWLFTTPLLLYDLGLL (SEQ ID NO: 59), WARYTDWFTTPLLLYDLALLA (SEQ ID NO: 60), WARYTDWLFTTPLLLYDLGLL (SEQ ID NO: 61), WARYADWLFTTPLLLLDLSLL (SEQ ID NO: 62), LLALDLLLLPTTFLWDAYRAW (SEQ ID NO: 63), LLALDLLLLPTTFLWDAYRAY (SEQ ID NO: 64), LLGLDYLLLPTTFLWDSYRAW (SEQ ID NO: 65), ALLALDYLLLPTTFWDTYRAW (SEQ ID NO: 66), LLGLDYLLLPTTFLWDTYRAW (SEQ ID NO: 67), LLSLDLLLLPTTFLWDAYRAW (SEQ ID NO: 328), GLAGLLGLEGLLGLPLGLLEGLWLGL (SEQ ID NO: 68), LGLWLGELLGLPLGLLGELGLLGALG (SEQ ID NO: 69), WRAYLDLLFPTDTLLLDLLW (SEQ ID NO: 70), WLLDLLLTDTPFLLDLYARW (SEQ ID NO: 71), WARYLEWLFPTETLLLEL (SEQ ID NO: 72), WAQYLELLFPTETLLLEW (SEQ ID NO: 73), LELLLTETPFLWELYRAW (SEQ ID NO: 74), WELLLTETPFLLELYQAW (SEQ ID NO: 75), WLFTTPLLLLNGALLVE (SEQ ID NO: 76), WLFTTPLLLLPGALLVE (SEQ ID NO: 77), WARYADLLFPTTLAW (SEQ ID NO: 78), EVLLAGNLLLLPTTFLW (SEQ ID NO: 79), EVLLAGPLLLLPTTFLW (SEQ ID NO: 80), WALTTPFLLDAYRAW (SEQ ID NO: 81), NLEGFFATLGGEIALWSLVVLAIE (SEQ ID NO: 82), EGFFATLGGEIALWSDVVLAIE (SEQ ID NO: 83), EGFFATLGGEIPLWSDVVLAIE (SEQ ID NO: 84), EIALVVLSWLAIEGGLTAFFGELN (SEQ ID NO: 85), EIALVVDSWLAIEGGLTAFFGE (SEQ ID NO: 86), EIALVVDSWLPIEGGLTAFFGE (SEQ ID NO: 87), ILDLVFGLLFAVTSVDFLVQW (SEQ ID NO: 88), or WQVLFDVSTVAFLLGFVLDLI (SEQ ID NO: 89).

In an aspect, provided herein is a pHLIP peptide (as well as compounds comprising at least one such pHLIP peptide) comprising a sequence of at least 8 to 25 consecutive amino acids (e.g., 8, 9, 10, 11, 12, 13, 14, 15, 16, 17, 18, 19, 20, 21, 22, 23, 24, or 25 consecutive amino acids) that is present in any one of the following sequences: WARYAXWLFTTPLLLLXLALL (SEQ ID NO: 90), YARYAXWLFTTPLLLLXLALL (SEQ ID NO: 91), WARYSXWLFTTPLLLYXLGLL (SEQ ID NO: 92), WARYTXWFTTPLLLYXLALLA (SEQ ID NO: 93), WARYTXWLFTTPLLLYXLGLL (SEQ ID NO: 94), WARYAXWLFTTPLLLLXLSLL (SEQ ID NO:95), LLALXLLLLPTTFLWXAYRAW (SEQ ID NO: 96), LLALXLLLLPTTFLWXAYRAY (SEQ ID NO: 97), LLGLXYLLLPTTFLWXSYRAW (SEQ ID NO: 98), ALLALXYLLLPTTFWXTYRAW (SEQ ID NO:99), LLGLXYLLLPTTFLWXTYRAW (SEQ ID NO:100), LLSLXLLLLPTTFLWXAYRAW (SEQ ID NO:101), GLAGLLGLXGLLGLPLGLLXGLWLGL (SEQ ID NO: 102), LGLWLGXLLGLPLGLLGXLGLLGALG (SEQ ID NO: 103), WRAYLXLLFPTXTLLLXLLW (SEQ ID NO: 104), WLLXLLLTXTPFLLXLYARW (SEQ ID NO: 105), WARYLXWLFPTXTLLLXL (SEQ ID NO: 106), WAQYLXLLFPTXTLLLXW (SEQ ID NO: 107), LXLLLTXTPFLWXLYRAW (SEQ ID NO: 108), WXLLLTXTPFLLXLYQAW (SEQ ID NO: 109), WLFTTPLLLLNGALLVX (SEQ ID NO: 110), WLFTTPLLLLPGALLVX (SEQ ID NO: 111), WARYAXLLFPTTLAW (SEQ ID NO: 112), XVLLAGNLLLLPTTFLW (SEQ ID NO: 113), XVLLAGPLLLLPTTFLW (SEQ ID NO: 114), WALTTPFLLXAYRAW (SEQ ID NO: 115), NLXGFFATLGGXIALWSLVVLAIX (SEQ ID NO: 116), XGFFATLGGXIALWSXVVLAIX (SEQ ID NO: 117), XGFFATLGGXIPLWSXVVLAIX (SEQ ID NO: 118), XIALVVLSWLAIXGGLTAFFGXLN (SEQ ID NO: 119), XIALVVXSWLAIXGGLTAFFGX (SEQ ID NO: 120), XIALVVXSWLPIXGGLTAFFGX (SEQ ID NO: 121), ILXLVFGLLFAVTSVXFLVQW (SEQ ID NO: 122), and WQVLFXVSTVAFLLGFVLXLI (SEQ ID NO: 123), wherein each X is, individually, D, E, Gla, or Aad.

In an aspect, provided herein is a pHLIP peptide (as well as compounds comprising at least one such pHLIP peptide) comprising the sequence: WARYAXWLFTTPLLLLXLALL (SEQ ID NO: 90), YARYAXWLFTTPLLLLXLALL (SEQ ID NO: 91), WARYSXWLFTTPLLLYXLGLL (SEQ ID NO: 92), WARYTXWFTTPLLLYXLALLA (SEQ ID NO: 93), WARYTXWLFTTPLLLYXLGLL (SEQ ID NO: 94), WARYAXWLFTTPLLLLXLSLL (SEQ ID NO: 95), LLALXLLLLPTTFLWXAYRAW (SEQ ID NO: 96), LLALXLLLLPTTFLWXAYRAY (SEQ ID NO: 97), LLGLXYLLLPTTFLWXSYRAW (SEQ ID NO: 98), ALLALXYLLLPTTFWXTYRAW (SEQ ID NO: 99), LLGLXYLLLPTTFLWXTYRAW (SEQ ID NO: 100), LLSLXLLLLPTTFLWXAYRAW (SEQ ID NO: 101), GLAGLLGLXGLLGLPLGLLXGLWLGL (SEQ ID NO: 102), LGLWLGXLLGLPLGLLGXLGLLGALG (SEQ ID NO: 103), WRAYLXLLFPTXTLLLXLLW (SEQ ID NO: 104), WLLXLLLTXTPFLLXLYARW (SEQ ID NO: 105), WARYLXWLFPTXTLLLXL (SEQ ID NO: 106), WAQYLXLLFPTXTLLLXW (SEQ ID NO: 107), LXLLLTXTPFLWXLYRAW (SEQ ID NO: 108), WXLLLTXTPFLLXLYQAW (SEQ ID NO: 109), WLFTTPLLLLNGALLVX (SEQ ID NO: 110), WLFTTPLLLLPGALLVX (SEQ ID NO: 111), WARYAXLLFPTTLAW (SEQ ID NO: 112), XVLLAGNLLLLPTTFLW (SEQ ID NO: 113), XVLLAGPLLLLPTTFLW (SEQ ID NO: 114), WALTTPFLLXAYRAW (SEQ ID NO: 115), NLXGFFATLGGXIALWSLVVLAIX (SEQ ID NO: 116), XGFFATLGGXIALWSXVVLAIX (SEQ ID NO: 117), XGFFATLGGXIPLWSXVVLAIX (SEQ ID NO: 118), XIALVVLSWLAIXGGLTAFFGXLN (SEQ ID NO: 119), XIALVVXSWLAIXGGLTAFFGX (SEQ ID NO: 120), XIALVVXSWLPIXGGLTAFFGX (SEQ ID NO: 121), ILXLVFGLLFAVTSVXFLVQW (SEQ ID NO: 122), or WQVLFXVSTVAFLLGFVLXLI (SEQ ID NO: 123), wherein each X is, individually, D, E, Gla, or Aad.

In an aspect, provided herein is a pHLIP peptide (as well as compounds comprising at least one such pHLIP peptide) comprising a sequence of at least 8 to 25 consecutive amino acids (e.g., 8, 9, 10, 11, 12, 13, 14, 15, 16, 17, 18, 19, 20, 21, 22, 23, 24, or 25 consecutive amino acids) that is present in any one of the following sequences: X₂X₂RX₂X₂X₁X₂X₂X₂X₃X₃X₂X₂X₂X₂X₂X₁X₂X₂X₂X₂ (SEQ ID NO: 124), X₂X₂RX₂X₃X₁X₂X₂X₂X₃X₃X₂X₂X₂X₂X₂X₁X₂GX₂X₂ (SEQ ID NO: 125), X₂X₂RX₂X₃X₁X₂X₂X₃X₃X₂X₂X₂X₂X₂X₁X₂X₂X₂X₂X₂ (SEQ ID NO: 126), X₂X₂RX₂X₂X₁X₂X₂X₂X₃X₃X₂X₂X₂X₂X₂X₁X₂X₃X₂X₂ (SEQ ID NO: 127), X₂X₂X₂X₂X₁X₂X₂X₂X₂X₂X₃X₃X₂X₂X₂X₁X₂X₂RX₂X₂ (SEQ ID NO: 128), X₂X₂GX₂X₁X₂X₂X₂X₂X₂X₃X₃X₂X₂X₂X₁X₃X₂RX₂X₂ (SEQ ID NO: 129), X₂X₂X₂X₂X₂X₁X₂X₂X₂X₂X₂X₃X₃X₂X₂X₁X₃X₂RX₂X₂ (SEQ ID NO: 130), X₂X₂X₃X₂X₁X₂X₂X₂X₂X₂X₃X₃X₂X₂X₂X₁X₂X₂RX₂X₂ (SEQ ID NO: 131), GX₂X₂GX₂X₂GX₂X₁GX₂X₂GX₂X₂X₂GX₂X₂X₁GX₂X₂X₂GX₂ (SEQ ID NO: 132), X₂GX₂X₂X₂GX₁X₂X₂GX₂X₂X₂GX₂X₂GX₁X₂GX₂X₂GX₂X₂G (SEQ ID NO: 133), X₂RX₂X₂X₂X₁X₂X₂X₂X₂X₃X₁X₃X₂X₂X₂X₁X₂X₂X₂ (SEQ ID NO: 134), X₂X₂X₂X₁X₂X₂X₂X₃X₁X₃X₂X₂X₂X₂X₁X₂X₂X₂RX₂ (SEQ ID NO: 135), X₂X₂RX₂X₂X₁X₂X₂X₂X₂X₃X₁X₃X₂X₂X₂XX₂ (SEQ ID NO: 136), X₂X₂QX₂X₂X₁X₂X₂X₂X₂X₃X₁X₃X₂X₂X₂X₁X₂ (SEQ ID NO: 137), X₂X₁X₂X₂X₂X₃X₁X₃X₂X₂X₂X₂X₁X₂X₂RX₂X₂ (SEQ ID NO: 138), X₂X₁X₂X₂X₂X₃X₁X₃X₂X₂X₂X₂X₁X₂X₂QX₂X₂ (SEQ ID NO: 139), X₂X₂X₂X₃X₃X₂X₂X₂X₂X₂NGX₂X₂X₂X₂X₁ (SEQ ID NO: 140), X₂X₂X₂X₃X₃X₂X₂X₂X₂X₂X₂GX₂X₂X₂X₂X₁ (SEQ ID NO: 141), X₂X₂RX₂X₂X₁X₂X₂X₂X₂X₃X₃X₂X₂X₂ (SEQ ID NO: 142), X₁X₂X₂X₂X₂GNX₂X₂X₂X₂X₂X₃X₃X₂X₂X₂ (SEQ ID NO: 143), X₁X₂X₂X₂X₂GX₂X₂X₂X₂X₂X₂X₃X₃X₂X₂X₂ (SEQ ID NO: 144), X₂X₂X₂X₃X₃X₂X₂X₂X₂X₁X₂X₂RX₂X₂ (SEQ ID NO: 145), GNX₂X₁GX₂X₂X₂X₃X₂GGX₁X₂X₂X₂X₂X₃X₂X₂X₂X₂X₂X₂X₁ (SEQ ID NO: 146), X₁GX₂X₂X₂X₃X₂GGX₁X₂X₂X₂X₂X₃X₁X₂X₂X₂X₂X₂X₁ (SEQ ID NO: 147), X₁GX₂X₂X₂X₃X₂GGX₁X₂X₂X₂X₂X₃X₁X₂X₂X₂X₂X₂X₁ (SEQ ID NO: 148), X₁X₂X₂X₂X₂X₂X₂X₃X₂X₂X₂X₂X₁GGX₂X₃X₂X₂X₂GX₁X₂NG (SEQ ID NO: 149), X₁X₂X₂X₂X₂X₂X₁X₃X₂X₂X₂X₂X₁GGX₂X₃X₂X₂X₂GX₁ (SEQ ID NO: 150), X₁X₂X₂X₂X₂X₂X₁X₃X₂X₂X₂X₂X₁GGX₂X₃X₂X₂X₂GX₁ (SEQ ID NO: 151), X₂X₂X₁X₂X₂X₂GX₂X₂X₂X₂X₂X₃X₃X₂X₁X₂X₂X₂QX₂ (SEQ ID NO: 152), and X₂QX₂X₂X₂X₁X₂X₃X₃X₂X₂X₂X₂X₂GX₂X₂X₂X₁X₂X₂ (SEQ ID NO: 153), wherein each X₁ is, individually, D, E, Gla, or Aad, each X₂ is, individually, A, I, L, M, F, P, W, Y, V, or G and each X₃ is, individually, S, T, or G.

In an aspect, provided herein is a pHLIP peptide (as well as compounds comprising at least one such pHLIP peptide) comprising the sequence: X₂X₂RX₂X₂X₁X₂X₂X₂X₃X₃X₂X₂X₂X₂X₂X₁X₂X₂X₂X₂ (SEQ ID NO: 124), X₂X₂RX₂X₃X₁X₂X₂X₂X₃X₃X₂X₂X₂X₂X₂X₁X₂GX₂X₂ (SEQ ID NO: 125), X₂X₂RX₂X₃X₁X₂X₂X₃X₃X₂X₂X₂X₂X₂X₁X₂X₂X₂X₂X₂ (SEQ ID NO: 126), X₂X₂RX₂X₂X₁X₂X₂X₂X₃X₃X₂X₂X₂X₂X₂X₁X₂X₃X₂X₂ (SEQ ID NO: 127), X₂X₂X₂X₂X₁X₂X₂X₂X₂X₂X₃X₃X₂X₂X₂X₁X₂X₂RX₂X₂ (SEQ ID NO: 128), X₂X₂GX₂X₁X₂X₂X₂X₂X₂X₃X₃X₂X₂X₂X₁X₃X₂RX₂X₂ (SEQ ID NO: 129), X₂X₂X₂X₂X₂X₁X₂X₂X₂X₂X₂X₃X₃X₂X₂X₁X₃X₂RX₂X₂ (SEQ ID NO: 130), X₂X₂X₃X₂X₁X₂X₂X₂X₂X₂X₃X₃X₂X₂X₂X₁X₂X₂RX₂X₂ (SEQ ID NO: 131), GX₂X₂GX₂X₂GX₂X₁GX₂X₂GX₂X₂X₂GX₂X₂X₁GX₂X₂X₂GX₂ (SEQ ID NO: 132), X₂GX₂X₂X₂GX₁X₂X₂GX₂X₂X₂GX₂X₂GX₁X₂GX₂X₂GX₂X₂G (SEQ ID NO: 133), X₂RX₂X₂X₂X₁X₂X₂X₂X₂X₃X₁X₃X₂X₂X₂X₁X₂X₂X₂ (SEQ ID NO: 134), X₂X₂X₂X₁X₂X₂X₂X₃X₁X₃X₂X₂X₂X₂X₁X₂X₂X₂RX₂ (SEQ ID NO: 135), X₂X₂RX₂X₂X₁X₂X₂X₂X₂X₃X₁X₃X₂X₂X₂XX₂ (SEQ ID NO: 136), X₂X₂QX₂X₂X₁X₂X₂X₂X₂X₃X₁X₃X₂X₂X₂X₁X₂ (SEQ ID NO: 137), X₂X₁X₂X₂X₂X₃X₁X₃X₂X₂X₂X₂X₁X₂X₂RX₂X₂ (SEQ ID NO: 138), X₂X₁X₂X₂X₂X₃X₁X₃X₂X₂X₂X₂X₁X₂X₂QX₂X₂ (SEQ ID NO: 139), X₂X₂X₂X₃X₃X₂X₂X₂X₂X₂NGX₂X₂X₂X₂X₁ (SEQ ID NO: 140), X₂X₂X₂X₃X₃X₂X₂X₂X₂X₂X₂GX₂X₂X₂X₂X₁ (SEQ ID NO: 141), X₂X₂RX₂X₂X₁X₂X₂X₂X₂X₃X₃X₂X₂X₂ (SEQ ID NO: 142), X₁X₂X₂X₂X₂GNX₂X₂X₂X₂X₂X₃X₃X₂X₂X₂ (SEQ ID NO: 143), X₁X₂X₂X₂X₂GX₂X₂X₂X₂X₂X₂X₃X₃X₂X₂X₂ (SEQ ID NO: 144), X₂X₂X₂X₃X₃X₂X₂X₂X₂X₁X₂X₂RX₂X₂ (SEQ ID NO: 145), GNX₂X₁GX₂X₂X₂X₃X₂GGX₁X₂X₂X₂X₂X₃X₂X₂X₂X₂X₂X₂X₁ (SEQ ID NO: 146), X₁GX₂X₂X₂X₃X₂GGX₁X₂X₂X₂X₂X₃X₁X₂X₂X₂X₂X₂X₁ (SEQ ID NO: 147), X₁GX₂X₂X₂X₃X₂GGX₁X₂X₂X₂X₂X₃X₁X₂X₂X₂X₂X₂X₁ (SEQ ID NO: 148), X₁X₂X₂X₂X₂X₂X₂X₃X₂X₂X₂X₂X₁GGX₂X₃X₂X₂X₂GX₁X₂NG (SEQ ID NO: 149), X₁X₂X₂X₂X₂X₂X₁X₃X₂X₂X₂X₂X₁GGX₂X₃X₂X₂X₂GX₁ (SEQ ID NO: 150), X₁X₂X₂X₂X₂X₂X₁X₃X₂X₂X₂X₂X₁GGX₂X₃X₂X₂X₂GX₁ (SEQ ID NO: 151), X₂X₂X₁X₂X₂X₂GX₂X₂X₂X₂X₂X₃X₃X₂X₁X₂X₂X₂QX₂ (SEQ ID NO: 152), and X₂QX₂X₂X₂X₁X₂X₃X₃X₂X₂X₂X₂X₂GX₂X₂X₂X₁X₂X₂ (SEQ ID NO: 153), wherein each X₁ is, individually, D, E, Gla, or Aad, each X₂ is, individually, A, I, L, M, F, P, W, Y, V, or G, and each X₃ is, individually, S, T, or G.

In an aspect, provided herein is a pHLIP peptide (as well as compounds comprising at least one such pHLIP peptide) comprising a sequence of at least 8 to 25 consecutive amino acids (e.g., 8, 9, 10, 11, 12, 13, 14, 15, 16, 17, 18, 19, 20, 21, 22, 23, 24, or 25 consecutive amino acids) that is present in any one of the following sequences:

(SEQ ID NO: 154)
AEQNPIYWARYAEWLFTTPLLLLELALLVEAEET,
(SEQ ID NO: 155)
ADDQNPWRAYLDLLFPDTTDLLLLDLLWDADET,
(SEQ ID NO: 156)
AEEQNPWRAYLELLFPETTELLLLELLWEAEET,
(SEQ ID NO: 157)
AEQNPIYWARYAGlaWLFTTPLLLLGlaLALLVDADET,
(SEQ ID NO: 158)
AEQNPIYWARYAAadWLFTTPLLLLAadLALLVDADET,
(SEQ ID NO: 159)
AEQNPIYWARYAAadWLFTTPLLLLGlaLALLVDADET,
(SEQ ID NO: 160)
CEQNPIYWARYADWHFTTPLLLLDLALLVDADE,
(SEQ ID NO: 161)
ADNNPWIYARYADLTTFPLLLLDLALLVDFDD,
(SEQ ID NO: 162)
ADNNPFIYARYADLTTWPLLLLDLALLVDFDD,
(SEQ ID NO: 163)
ADNNPFIYARYADLTTFPLLLLDLALLVDWDD,
(SEQ ID NO: 164)
ADNNPFPYARYADLTTWILLLLDLALLVDFDD,
(SEQ ID NO: 165)
ADNNPFIYAYRADLTTFPLLLLDLALLVDWDD,
(SEQ ID NO: 166)
ADNNPFIYATYADLRTFPLLLLDLALLVDWDD,
(SEQ ID NO: 167)
ADDQNPWRAYLDLLFPTDTLLLDLLWDADE,
(SEQ ID NO: 47)
ADDQNPWRAYLGlaLLFPTDTLLLDLLW,
(SEQ ID NO: 168)
ADDQNPWRAYLDLLFPTGlaTLLLDLLW,
(SEQ ID NO: 169)
ADDQNPWRAYLDLLFPTDTLLLGlaLLW,
(SEQ ID NO: 170)
ADDQNPWRAYLGlaLLFPTGlaTLLLDLLW,
(SEQ ID NO: 171)
ADDQNPWRAYLGlaLLFPTDTLLLGlaLLW,
(SEQ ID NO: 172)
ADDQNPWRAYLDLLFPTGlaTLLLGlaLLW,
(SEQ ID NO: 173)
ADDQNPWRAYLGlaLLFPTGlaTLLLGlaLLW,
(SEQ ID NO: 174)
ADDQNPWRAYLAadLLFPTDTLLLDLLW,
(SEQ ID NO: 175)
ADDQNPWRAYLDLLFPTAadTLLLDLLW,
(SEQ ID NO: 176)
ADDQNPWRAYLDLLFPTDTLLLAadLLW,
(SEQ ID NO: 177)
ADDQNPWRAYLAadLLFPTAadTLLLDLLW,
(SEQ ID NO: 178)
ADDQNPWRAYLAadLLFPTDTLLLAadLLW,
(SEQ ID NO: 179)
ADDQNPWRAYLDLLFPTAadTLLLAadLLW,
(SEQ ID NO: 180)
ADDQNPWRAYLAadLLFPTAadTLLLAadLLW,
(SEQ ID NO: 181)
ADDQNPWRAYLGlaLLFPTAadTLLLDLLW,
(SEQ ID NO: 182)
ADDQNPWRAYLGlaLLFPTDTLLLAadLLW,
(SEQ ID NO: 183)
ADDQNPWRAYLGlaLLFPTGlaTLLLAadLLW,
(SEQ ID NO: 184)
ADDQNPWRAYLAadLLFPTGlaTLLLDLLW,
(SEQ ID NO: 185)
ADDQNPWRAYLAadLLFPTDTLLLGlaLLW,
(SEQ ID NO: 186)
ADDQNPWRAYLGlaLLFPTAadTLLLGlaLLW,
(SEQ ID NO: 187)
GEEQNPWLGAYLDLLFPLELLGLLELGLW,
(SEQ ID NO: 188)
EQNPIYILDLVFGLLFAVTSVDFLVQWDDAGD,
(SEQ ID NO: 189)
NNEGFFATLGGEIALWSDVVLAIE,
and
(SEQ ID NO: 190)
DNNEGFFATLGGEIPLWSDVVLAIE.

In an aspect, provided herein is a pHLIP peptide (as well as compounds comprising at least one such pHLIP peptide) comprising the sequence:

(SEQ ID NO: 154)
AEQNPIYWARYAEWLFTTPLLLLELALLVEAEET,
(SEQ ID NO: 155)
ADDQNPWRAYLDLLFPDTTDLLLLDLLWDADET,
(SEQ ID NO: 156)
AEEQNPWRAYLELLFPETTELLLLELLWEAEET,
(SEQ ID NO: 157)
AEQNPIYWARYAGlaWLFTTPLLLLGlaLALLVDADET,
(SEQ ID NO: 158)
AEQNPIYWARYAAadWLFTTPLLLLAadLALLVDADET,
(SEQ ID NO: 159)
AEQNPIYWARYAAadWLFTTPLLLLGlaLALLVDADET,
(SEQ ID NO: 160)
CEQNPIYWARYADWHFTTPLLLLDLALLVDADE,
(SEQ ID NO: 161)
ADNNPWIYARYADLTTFPLLLLDLALLVDFDD,
(SEQ ID NO: 162)
ADNNPFIYARYADLTTWPLLLLDLALLVDFDD,
(SEQ ID NO: 163)
ADNNPFIYARYADLTTFPLLLLDLALLVDWDD,
(SEQ ID NO: 164)
ADNNPFPYARYADLTTWILLLLDLALLVDFDD,
(SEQ ID NO: 165)
ADNNPFIYAYRADLTTFPLLLLDLALLVDWDD,
(SEQ ID NO: 166)
ADNNPFIYATYADLRTFPLLLLDLALLVDWDD,
(SEQ ID NO: 167)
ADDQNPWRAYLDLLFPTDTLLLDLLWDADE,
(SEQ ID NO: 47)
ADDQNPWRAYLGlaLLFPTDTLLLDLLW,
(SEQ ID NO: 168)
ADDQNPWRAYLDLLFPTGlaTLLLDLLW,
(SEQ ID NO: 169)
ADDQNPWRAYLDLLFPTDTLLLGlaLLW,
(SEQ ID NO: 170)
ADDQNPWRAYLGlaLLFPTGlaTLLLDLLW,
(SEQ ID NO: 171)
ADDQNPWRAYLGlaLLFPTDTLLLGlaLLW,
(SEQ ID NO: 172)
ADDQNPWRAYLDLLFPTGlaTLLLGlaLLW,
(SEQ ID NO: 173)
ADDQNPWRAYLGlaLLFPTGlaTLLLGlaLLW,
(SEQ ID NO: 174)
ADDQNPWRAYLAadLLFPTDTLLLDLLW,
(SEQ ID NO: 175)
ADDQNPWRAYLDLLFPTAadTLLLDLLW,
(SEQ ID NO: 176)
ADDQNPWRAYLDLLFPTDTLLLAadLLW,
(SEQ ID NO: 177)
ADDQNPWRAYLAadLLFPTAadTLLLDLLW,
(SEQ ID NO: 178)
ADDQNPWRAYLAadLLFPTDTLLLAadLLW,
(SEQ ID NO: 179)
ADDQNPWRAYLDLLFPTAadTLLLAadLLW,
(SEQ ID NO: 180)
ADDQNPWRAYLAadLLFPTAadTLLLAadLLW,
(SEQ ID NO: 181)
ADDQNPWRAYLGlaLLFPTAadTLLLDLLW,
(SEQ ID NO: 182)
ADDQNPWRAYLGlaLLFPTDTLLLAadLLW,
(SEQ ID NO: 183)
ADDQNPWRAYLGlaLLFPTGlaTLLLAadLLW,
(SEQ ID NO: 184)
ADDQNPWRAYLAadLLFPTGlaTLLLDLLW,
(SEQ ID NO: 185)
ADDQNPWRAYLAadLLFPTDTLLLGlaLLW,
(SEQ ID NO: 186)
ADDQNPWRAYLGlaLLFPTAadTLLLGlaLLW,
(SEQ ID NO: 187)
GEEQNPWLGAYLDLLFPLELLGLLELGLW,
(SEQ ID NO: 188)
EQNPIYILDLVFGLLFAVTSVDFLVQWDDAGD,
(SEQ ID NO: 189)
NNEGFFATLGGEIALWSDVVLAIE,
or
(SEQ ID NO: 190)
DNNEGFFATLGGEIPLWSDVVLAIE.

In an aspect, provided herein is a pHLIP peptide (as well as compounds comprising at least one such pHLIP peptide) comprising a sequence of at least 8 to 25 consecutive amino acids (e.g., 8, 9, 10, 11, 12, 13, 14, 15, 16, 17, 18, 19, 20, 21, 22, 23, 24, or 25 consecutive amino acids) that is present in any one of the following sequences: AXQNPIYWARYAXWLFTTPLLLLXLALLVXAXXT (SEQ ID NO: 191), AXXQNPWRAYLXLLFPXTTXLLLLXLLWXAXXT (SEQ ID NO: 192), AXXQNPWRAYLXLLFPXTTXLLLLXLLWXAXXT (SEQ ID NO: 193), AXQNPIYWARYAXWLFTTPLLLLXLALLVXAXXT (SEQ ID NO: 194), AXQNPIYWARYAXWLFTTPLLLLXLALLVXAXXT (SEQ ID NO: 195), AXQNPIYWARYAXWLFTTPLLLLXLALLVXAXXT (SEQ ID NO: 196), CXQNPIYWARYAXWHFTTPLLLLXLALLVXAXX (SEQ ID NO: 197), AXNNPWIYARYAXLTTFPLLLLXLALLVXFXX (SEQ ID NO: 198), AXNNPFIYARYAXLTTWPLLLLXLALLVXFXX (SEQ ID NO: 199), AXNNPFIYARYAXLTTFPLLLLXLALLVXWXX (SEQ ID NO: 200), AXNNPFPYARYAXLTTWILLLLXLALLVXFXX (SEQ ID NO: 201), AXNNPFIYAYRAXLTTFPLLLLXLALLVXWXX (SEQ ID NO: 202), AXNNPFIYATYAXLRTFPLLLLXLALLVXWXX (SEQ ID NO: 203), AXXQNPWRAYLXLLFPTXTLLLXLLWXAXX (SEQ ID NO: 204), AXXQNPWRAYLXLLFPTXTLLLXLLW (SEQ ID NO: 205), XXQNPWRAYLXLLFPTXTLLLXLLW (SEQ ID NO: 206), ANNPFIYATYAXLLFPTXTLLLXLLW (SEQ ID NO: 207), NNPFIYATYAXLLFPTXTLLLXLLW (SEQ ID NO: 208), AXXQNPWRAYLXLRTFPLLLLXLAW (SEQ ID NO: 209), XXQNPWRAYLXLRTFPLLLLXLAW (SEQ ID NO: 210), AXXQNPWRAYLXLRTFPLLLLXLALL (SEQ ID NO: 211), XXQNPWRAYLXLRTFPLLLLXLALL (SEQ ID NO: 212), ANPIYWARYAXLLFPTXTLLLXLLW (SEQ ID NO: 213), NPIYWARYAXLLFPTXTLLLXLLW (SEQ ID NO: 214), AXXQNPWRAYLXWLFTTPLLLLXLALLV (SEQ ID NO: 215), XXQNPWRAYLXWLFTTPLLLLXLALLV (SEQ ID NO: 216), CXQNPIYWARYAXWHFTTPLLLLXLALLV (SEQ ID NO: 217), XQNPIYWARYAXWHFTTPLLLLXLALLV (SEQ ID NO: 218), AXXQNPWRAYLXLLFPTXTLLLXLLV (SEQ ID NO: 219), XXQNPWRAYLXLLFPTXTLLLXLLV (SEQ ID NO: 220), AXQNPIYWARYAXLLFPTXTLLLXLLW (SEQ ID NO: 221), XQNPIYWARYAXLLFPTXTLLLXLLW (SEQ ID NO: 222), AXQNPIYWARYLXLLFPTXTLLLXLLW (SEQ ID NO: 223), XQNPIYWARYLXLLFPTXTLLLXLLW (SEQ ID NO: 224), GXXQNPWLGAYLXLLFPLXLLGLLXLGLW (SEQ ID NO: 225), XQNPIYILXLVFGLLFAVTSVXFLVQWXXAGX (SEQ ID NO: 226), NNXGFFATLGGXIALWSXVVLAIX (SEQ ID NO: 227), and XNNXGFFATLGGXIPLWSXVVLAIX (SEQ ID NO: 228), wherein each X is, individually, D, E, Gla, or Aad.

In an aspect, provided herein is a pHLIP peptide (as well as compounds comprising at least one such pHLIP peptide) comprising the sequence: AXQNPIYWARYAXWLFTTPLLLLXLALLVXAXXT (SEQ ID NO: 191), AXXQNPWRAYLXLLFPXTTXLLLLXLLWXAXXT (SEQ ID NO: 192), AXXQNPWRAYLXLLFPXTTXLLLLXLLWXAXXT (SEQ ID NO: 193), AXQNPIYWARYAXWLFTTPLLLLXLALLVXAXXT (SEQ ID NO: 194), AXQNPIYWARYAXWLFTTPLLLLXLALLVXAXXT (SEQ ID NO: 195), AXQNPIYWARYAXWLFTTPLLLLXLALLVXAXXT (SEQ ID NO: 196), CXQNPIYWARYAXWHFTTPLLLLXLALLVXAXX (SEQ ID NO: 197), AXNNPWIYARYAXLTTFPLLLLXLALLVXFXX (SEQ ID NO: 198), AXNNPFIYARYAXLTTWPLLLLXLALLVXFXX (SEQ ID NO: 199), AXNNPFIYARYAXLTTFPLLLLXLALLVXWXX (SEQ ID NO: 200), AXNNPFPYARYAXLTTWILLLLXLALLVXFXX (SEQ ID NO: 201), AXNNPFIYAYRAXLTTFPLLLLXLALLVXWXX (SEQ ID NO: 202), AXNNPFIYATYAXLRTFPLLLLXLALLVXWXX (SEQ ID NO: 203), AXXQNPWRAYLXLLFPTXTLLLXLLWXAXX (SEQ ID NO: 204), AXXQNPWRAYLXLLFPTXTLLLXLLW (SEQ ID NO: 205), XXQNPWRAYLXLLFPTXTLLLXLLW (SEQ ID NO: 206), ANNPFIYATYAXLLFPTXTLLLXLLW (SEQ ID NO: 207), NNPFIYATYAXLLFPTXTLLLXLLW (SEQ ID NO: 208), AXXQNPWRAYLXLRTFPLLLLXLAW (SEQ ID NO: 209), XXQNPWRAYLXLRTFPLLLLXLAW (SEQ ID NO: 210), AXXQNPWRAYLXLRTFPLLLLXLALL (SEQ ID NO: 211), XXQNPWRAYLXLRTFPLLLLXLALL (SEQ ID NO: 212), ANPIYWARYAXLLFPTXTLLLXLLW (SEQ ID NO: 213), NPIYWARYAXLLFPTXTLLLXLLW (SEQ ID NO: 214), AXXQNPWRAYLXWLFTTPLLLLXLALLV (SEQ ID NO: 215), XXQNPWRAYLXWLFTTPLLLLXLALLV (SEQ ID NO: 216), CXQNPIYWARYAXWHFTTPLLLLXLALLV (SEQ ID NO: 217), XQNPIYWARYAXWHFTTPLLLLXLALLV (SEQ ID NO: 218), AXXQNPWRAYLXLLFPTXTLLLXLLV (SEQ ID NO: 219), XXQNPWRAYLXLLFPTXTLLLXLLV (SEQ ID NO: 220), AXQNPIYWARYAXLLFPTXTLLLXLLW (SEQ ID NO: 221), XQNPIYWARYAXLLFPTXTLLLXLLW (SEQ ID NO: 222), AXQNPIYWARYLXLLFPTXTLLLXLLW (SEQ ID NO: 223), XQNPIYWARYLXLLFPTXTLLLXLLW (SEQ ID NO: 224), GXXQNPWLGAYLXLLFPLXLLGLLXLGLW (SEQ ID NO: 225), XQNPIYILXLVFGLLFAVTSVXFLVQWXXAGX (SEQ ID NO: 226), NNXGFFATLGGXIALWSXVVLAIX (SEQ ID NO: 227), or XNNXGFFATLGGXIPLWSXVVLAIX (SEQ ID NO: 228), wherein each X is, individually, D, E, Gla, or Aad.

In an aspect, provided herein is a pHLIP peptide (as well as compounds comprising at least one such pHLIP peptide) comprising a sequence of at least 8 to 25 consecutive amino acids (e.g., 8, 9, 10, 11, 12, 13, 14, 15, 16, 17, 18, 19, 20, 21, 22, 23, 24, or 25 consecutive amino acids) that is present in any one of the following sequences: X₂X₁QNX₂X₂X₂X₂X₂RX₂X₂X₁X₂X₂X₂X₃X₃X₂X₂X₂X₂X₂X₁X₂X₂X₂X₂X₂X₁X₂X₁X₁X₃ (SEQ ID NO: 229), X₂X₁X₁QNX₂X₂RX₂X₂X₂X₁X₂X₂X₂X₂X₁X₃X₃X₁X₂X₂X₂X₂X₁X₂X₂X₂X₁X₂X₁X₁X₃ (SEQ ID NO: 230), CX₁QNX₂X₂X₂X₂X₂RX₂X₂X₁X₂HX₂X₃X₃X₂X₂X₂X₂X₂X₁X₂X₂X₂X₂X₂X₁X₂X₁X₁ (SEQ ID NO: 231), X₂X₁NNX₂X₂X₂X₂X₂RX₂X₂X₁X₂X₃X₃X₂X₂X₂X₂X₂X₂X₁X₂X₂X₂X₂X₂X₁X₂X₁X₁ (SEQ ID NO: 232), X₂X₁NNX₂X₂X₂X₂X₂X₂RX₂X₁X₂X₃X₃X₂X₂X₂X₂X₂X₂X₁X₂X₂X₂X₂X₂X₁X₂X₁X₁ (SEQ ID NO: 233), X₂X₁NNX₂X₂X₂X₂X₂X₃X₂X₂X₁X₂RX₃X₂X₂X₂X₂X₂X₂X₁X₂X₂X₂X₂X₂X₁X₂X₁X₁ (SEQ ID NO: 234), X₂X₁X₁QNX₂X₂RX₂X₂X₂X₁X₂X₂X₂X₂X₃X₁X₃X₂X₂X₂X₁X₂X₂X₂X₁X₂X₁X₁ (SEQ ID NO: 235), X₂X₁X₁QNX₂X₂RX₂X₂X₂X₁X₂X₂X₂X₂X₃X₁X₃X₂X₂X₂X₁X₂X₂X₂ (SEQ ID NO: 236), X₂X₁X₁QNX₂X₂X₂X₂X₂X₂X₂X₁X₂X₂X₂X₂X₂X₁X₂X₂X₂X₂X₂X₁X₂X₂X₂X₂ (SEQ ID NO: 237), X₁QNX₂X₂X₂X₂X₂X₁X₂X₂X₂X₂X₂X₂X₂X₂X₂X₃X₃X₂X₁X₂X₂X₂QX₂X₁X₁X₂X₂ (SEQ ID NO: 238), NNX₁X₂X₂X₂X₂X₃X₂X₂X₂X₁X₂X₂X₂X₂X₃X₁X₂X₂X₂X₂X₂X₁ (SEQ ID NO: 239), and X₁NNX₁X₂X₂X₂X₂X₃X₂X₂X₂X₁X₂X₂X₂X₂X₃X₁X₂X₂X₂X₂X₂X₁ (SEQ ID NO: 240), wherein each X₁ is, individually, D, E, Gla, or Aad, each X₂ is, individually, A, I, L, M, F, P, W, Y, V, or G and each X₃ is, individually, S, T, or G.

In an aspect, provided herein is a pHLIP peptide (as well as compounds comprising at least one such pHLIP peptide) comprising the sequence: X₂X₁QNX₂X₂X₂X₂X₂RX₂X₂X₁X₂X₂X₂X₃X₃X₂X₂X₂X₂X₂X₁X₂X₂X₂X₂X₂X₁X₂X₁X₁X₃ (SEQ ID NO: 229), X₂X₁X₁QNX₂X₂RX₂X₂X₂X₁X₂X₂X₂X₂X₁X₃X₃X₁X₂X₂X₂X₂X₁X₂X₂X₂X₁X₂X₁X₁X₃ (SEQ ID NO: 230), CX₁QNX₂X₂X₂X₂X₂RX₂X₂X₁X₂HX₂X₃X₃X₂X₂X₂X₂X₂X₁X₂X₂X₂X₂X₂X₁X₂X₁X₁ (SEQ ID NO: 231), X₂X₁NNX₂X₂X₂X₂X₂RX₂X₂X₁X₂X₃X₃X₂X₂X₂X₂X₂X₂X₁X₂X₂X₂X₂X₂X₁X₂X₁X₁ (SEQ ID NO: 232), X₂X₁NNX₂X₂X₂X₂X₂X₂RX₂X₁X₂X₃X₃X₂X₂X₂X₂X₂X₂X₁X₂X₂X₂X₂X₂X₁X₂X₁X₁ (SEQ ID NO: 233), X₂X₁NNX₂X₂X₂X₂X₂X₃X₂X₂X₁X₂RX₃X₂X₂X₂X₂X₂X₂X₁X₂X₂X₂X₂X₂X₁X₂X₁X₁ (SEQ ID NO: 234), X₂X₁X₁QNX₂X₂RX₂X₂X₂X₁X₂X₂X₂X₂X₃X₁X₃X₂X₂X₂X₁X₂X₂X₂X₁X₂X₁X₁ (SEQ ID NO: 235), X₂X₁X₁QNX₂X₂RX₂X₂X₂X₁X₂X₂X₂X₂X₃X₁X₃X₂X₂X₂X₁X₂X₂X₂ (SEQ ID NO: 236), X₂X₁X₁QNX₂X₂X₂X₂X₂X₂X₂X₁X₂X₂X₂X₂X₂X₁X₂X₂X₂X₂X₂X₁X₂X₂X₂X₂ (SEQ ID NO: 237), X₁QNX₂X₂X₂X₂X₂X₁X₂X₂X₂X₂X₂X₂X₂X₂X₂X₃X₃X₂X₁X₂X₂X₂QX₂X₁X₁X₂X₂ (SEQ ID NO: 238), NNX₁X₂X₂X₂X₂X₃X₂X₂X₂X₁X₂X₂X₂X₂X₃X₁X₂X₂X₂X₂X₂X₁ (SEQ ID NO: 239), and X₁NNX₁X₂X₂X₂X₂X₃X₂X₂X₂X₁X₂X₂X₂X₂X₃X₁X₂X₂X₂X₂X₂X₁ (SEQ ID NO: 240), wherein each X₁ is, individually, D, E, Gla, or Aad, each X₂ is, individually, A, I, L, M, F, P, W, Y, V, or G and each X₃ is, individually, S, T, or G.

In various embodiments, a pHLIP compound comprises 2 or more pHLIP peptides, each pHLIP peptide comprising the sequence:

(SEQ ID NO: 21)
ADDQNPWRAYLDLLFPTDTLLLDLLWG,
(SEQ ID NO: 22)
AKDDQNPWRAYLDLLFPTDTLLLDLLWG,
(SEQ ID NO: 15)
ACDDQNPWRAYLDLLFPTDTLLLDLLWA,
(SEQ ID NO: 23)
ADDQNPWRAYLDLLFPTDTLLLDLLWA,
(SEQ ID NO: 17)
ADDQNPWRAYLDLLFPTDTLLLDLLWCA,
(SEQ ID NO: 18)
ADDQNPWRAYLDLLFPTDTLLLDLLWKA,
(SEQ ID NO: 16)
AKDDQNPWRAYLDLLFPTDTLLLDLLWA,
(SEQ ID NO: 24)
ACDDQNPWRAYLDLLFPTDTLLLDLLWG,
(SEQ ID NO: 25)
ADDQNPWRAYLDLLFPTDTLLLDLLWCG,
(SEQ ID NO: 26)
ADDQNPWRAYLDLLFPTDTLLLDLLWKG,
(SEQ ID NO: 27)
ACDDQNPWRAYLDLLFPTDTLLLDLLWKG,
(SEQ ID NO: 28)
AKDDQNPWRAYLDLLFPTDTLLLDLLWCG,
(SEQ ID NO: 29)
ACKDDQNPWRAYLDLLFPTDTLLLDLLWG,
(SEQ ID NO: 241)
ACDDQNPWRAYLDLLFPTDTLLLDLLW,
(SEQ ID NO: 33)
AKDDQNPWRAYLDLLFPTDTLLLDLLWC,
(SEQ ID NO: 242)
ACDDQNPWARYLDWLFPTDTLLLDL,
(SEQ ID NO: 243)
CDNNNPWRAYLDLLFPTDTLLLDW,
(SEQ ID NO: 244)
ACEEQNPWARYLEWLFPTETLLLEL,
(SEQ ID NO: 245)
ACEEQNPWRAYLELLFPTETLLLELLW,
(SEQ ID NO: 246)
CEEQQPWAQYLELLFPTETLLLEW,
(SEQ ID NO: 247)
CEEQQPWRAYLELLFPTETLLLEW,
(SEQ ID NO: 248)
AAEEQNPWARYLEWLFPTETLLLEL,
or
(SEQ ID NO: 321)
AKEEQNPWARYLEWLFPTETLLLEL.

In some embodiments, a pHLIP compound comprises 2 or more pHLIP peptides with an amino acid sequence that is less than 100%, 99%, or 95% identical to an amino acid sequence described herein. In certain embodiments, a pHLIP compound comprises 2 or more pHLIP peptides with an amino acid sequence that is 95-100%, 95-99%, or 90-95% identical to an amino acid sequence described herein.

In an aspect, included herein is a pHLIP peptide (as well as compounds comprising at least one such pHLIP peptide) comprising at least 8 amino acids, wherein (i) at least 4 of the 8 amino acids are non-polar amino acids, (ii) at least 1 of the at least 8 amino acids is protonatable, (iii) the pHLIP peptide has a higher affinity for a membrane lipid bilayer at pH 5.0 compared to the affinity at pH 8.0. In certain embodiments, the at least 8 amino acids are consecutive amino acids. In some embodiments, the at least 8 amino acids are not consecutive amino acids. In various embodiments, the pHLIP peptide has less than 30, 25, or 20 total amino acids. In certain embodiments, the sequence of the pHLIP peptide has 8-20 (e.g., 8, 9, 10, 11, 12, 13, 14, 15, 16, 17, 18, 19, 20, 8-15, 8-20, 10-15, 10-20, or 15-20) consecutive amino acids.

In an aspect, included herein is a pHLIP peptide (as well as compounds comprising at least one such pHLIP peptide) comprising at least 8 consecutive amino acids, wherein (i) at least 4 of the 8 consecutive amino acids are non-polar amino acids, (ii) at least 1 of the at least 8 consecutive amino acids is protonatable, (iii) the pHLIP peptide has a higher affinity for a membrane lipid bilayer at pH 5.0 compared to the affinity at pH 8.0, and (iv) the at least 8 consecutive amino acids comprise 8 consecutive amino acids in a sequence that is identical to a sequence of 8 consecutive amino acids that occurs in a naturally occurring human protein.

In various embodiments, the at least 8 consecutive amino acids comprise a sequence that is at least 85%, 90%, or 95% identical to (e.g., is 100% identical to) (i) a sequence of at least 8 consecutive amino acids that occurs in a naturally occurring human protein; or (ii) the reverse of a sequence of at least 8 consecutive amino acids that occurs in a naturally occurring human protein. In some embodiments, the naturally occurring human protein is a human rhodopsin protein. In certain embodiments, the at least 8 consecutive amino acids that occurs in the human rhodopsin protein are within the following sequence: NLEGFFATLGGEIALWSLVVLAIE (SEQ ID NO: 82). The reverse of this sequence is EIALVVLSWLAIEGGLTAFFGELN (SEQ ID NO: 85).

In various embodiments, the sequence of the pHLIP peptide comprises 8-20 (e.g., 8, 9, 10, 11, 12, 13, 14, 15, 16, 17, 18, 19, 20, 8-15, 8-20, 10-15, 10-20, or 15-20) consecutive amino acids that have a sequence that is at least 85%, 90%, or 95% identical to a sequence of 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein, wherein the sequence of the 8-20 (e.g., 8, 9, 10, 11, 12, 13, 14, 15, 16, 17, 18, 19, 20, 8-15, 8-20, 10-15, 10-20, or 15-20) consecutive amino acids of the pHLIP peptide has 1, 2, or 3 amino acid substitutions compared to the sequence of the 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein. In some embodiments, the sequence has a L to D, L to E, A to P, or C to G substitution compared to the 8-20 (e.g., 8, 9, 10, 11, 12, 13, 14, 15, 16, 17, 18, 19, 20, 8-15, 8-20, 10-15, 10-20, or 15-20) consecutive amino acids that occur in the human rhodopsin protein. In certain embodiments, the sequence of the pHLIP peptide comprises 8-20 consecutive amino acids that have a sequence that is 85%, 90%, or 95% identical to the reverse of a sequence of 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein, wherein the sequence of the 8-20 consecutive amino acids of the pHLIP peptide has 1, 2, or 3 amino acid substitutions compared to the reverse of the sequence of the 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein. In some embodiments, the sequence has a L to D, L to E, A to P, or C to G substitution compared to the reverse of the 8-20 (e.g., 8, 9, 10, 11, 12, 13, 14, 15, 16, 17, 18, 19, 20, 8-15, 8-20, 10-15, 10-20, or 15-20) consecutive amino acids that occur in the human rhodopsin protein.

A non-limiting example of a genomic nucleotide sequence that encodes human rhodopsin is available under National Center for Biotechnology Information (NCBI) Reference Sequence No: NC_000003.12, and all information available under NCBI Reference Sequence No: NC_000003.12 is incorporated herein by reference. The nucleotide sequence that is available from NCBI Reference Sequence No: NC_000003.12 is as follows: AGAGTCATCCAGCTGGAGCCCTGAGTGGCTGAGCTCAGGCCTTCGCAGCATTCTT GGGTGGGAGCAGCCACGGGTCAGCCACAAGGGCCACAGCCATGAATGGCACAG AAGGCCCTAACTTCTACGTGCCCTTCTCCAATGCGACGGGTGTGGTACGCAGCCC CTTCGAGTACCCACAGTACTACCTGGCTGAGCCATGGCAGTTCTCCATGCTGGCC GCCTACATGTTTCTGCTGATCGTGCTGGGCTTCCCCATCAACTTCCTCACGCTCTA CGTCACCGTCCAGCACAAGAAGCTGCGCACGCCTCTCAACTACATCCTGCTCAAC CTAGCCGTGGCTGACCTCTTCATGGTCCTAGGTGGCTTCACCAGCACCCTCTACA CCTCTCTGCATGGATACTTCGTCTTCGGGCCCACAGGATGCAATTTGGAGGGCTT CTTTGCCACCCTGGGCGGTATGAGCCGGGTGTGGGTGGGGTGTGCAGGAGCCCG GGAGCATGGAGGGGTCTGGGAGAGTCCCGGGCTTGGCGGTGGTGGCTGAGAGGC CTTCTCCCTTCTCCTGTCCTGTCAATGTTATCCAAAGCCCTCATATATTCAGTCAA CAAACACCATTCATGGTGATAGCCGGGCTGCTGTTTGTGCAGGGCTGGCACTGAA CACTGCCTTGATCTTATTTGGAGCAATATGCGCTTGTCTAATTTCACAGCAAGAA AACTGAGCTGAGGCTCAAAGAAGTCAAGCGCCCTGCTGGGGCGTCACACAGGGA CGGGTGCAGAGTTGAGTTGGAAGCCCGCATCTATCTCGGGCCATGTTTGCAGCAC CAAGCCTCTGTTTCCCTTGGAGCAGCTGTGCTGAGTCAGACCCAGGCTGGGCACT GAGGGAGAGCTGGGCAAGCCAGACCCCTCCTCTCTGGGGGCCCAAGCTCAGGGT GGGAAGTGGATTTTCCATTCTCCAGTCATTGGGTCTTCCCTGTGCTGGGCAATGG GCTCGGTCCCCTCTGGCATCCTCTGCCTCCCCTCTCAGCCCCTGTCCTCAGGTGCC CCTCCAGCCTCCCTGCCGCGTTCCAAGTCTCCTGGTGTTGAGAACCGCAAGCAGC CGCTCTGAAGCAGTTCCTTTTTGCTTTAGAATAATGTCTTGCATTTAACAGGAAA ACAGATGGGGTGCTGCAGGGATAACAGATCCCACTTAACAGAGAGGAAAACTGA GGCAGGGAGAGGGGAAGAGACTCATTTAGGGATGTGGCCAGGCAGCAACAAGA GCCTAGGTCTCCTGGCTGTGATCCAGGAATATCTCTGCTGAGATGCAGGAGGAGA CGCTAGAAGCAGCCATTGCAAAGCTGGGTGACGGGGAGAGCTTACCGCCAGCCA CAAGCGTCTCTCTGCCAGCCTTGCCCTGTCTCCCCCATGTCCAGGCTGCTGCCTCG GTCCCATTCTCAGGGAATCTCTGGCCATTGTTGGGTGTTTGTTGCATTCAATAATC ACAGATCACTCAGTTCTGGCCAGAAGGTGGGTGTGCCACTTACGGGTGGTTGTTC TCTGCAGGGTCAGTCCCAGTTTACAAATATTGTCCCTTTCACTGTTAGGAATGTCC CAGTTTGGTTGATTAACTATATGGCCACTCTCCCTATGGAACTTCATGGGGTGGT GAGCAGGACAGATGTCTGAATTCCATCATTTCCTTCTTCTTCCTCTGGGCAAAAC ATTGCACATTGCTTCATGGCTCCTAGGAGAGGCCCCCACATGTCCGGGTTATTTC ATTTCCCGAGAAGGGAGAGGGAGGAAGGACTGCCAATTCTGGGTTTCCACCACC TCTGCATTCCTTCCCAACAAGGAACTCTGCCCCACATTAGGATGCATTCTTCTGCT AAACACACACACACACACACACACACACAACACACACACACACACACACACAC ACACACACACAAAACTCCCTACCGGGTTCCCAGTTCAATCCTGACCCCCTGATCT GATTCGTGTCCCTTATGGGCCCAGAGCGCTAAGCAAATAACTTCCCCCATTCCCT GGAATTTCTTTGCCCAGCTCTCCTCAGCGTGTGGTCCCTCTGCCCCTTCCCCCTCC TCCCAGCACCAAGCTCTCTCCTTCCCCAAGGCCTCCTCAAATCCCTCTCCCACTCC TGGTTGCCTTCCTAGCTACCCTCTCCCTGTCTAGGGGGGAGTGCACCCTCCTTAGG CAGTGGGGTCTGTGCTGACCGCCTGCTGACTGCCTTGCAGGTGAAATTGCCCTGT GGTCCTTGGTGGTCCTGGCCATCGAGCGGTACGTGGTGGTGTGTAAGCCCATGAG CAACTTCCGCTTCGGGGAGAACCATGCCATCATGGGCGTTGCCTTCACCTGGGTC ATGGCGCTGGCCTGCGCCGCACCCCCACTCGCCGGCTGGTCCAGGTAATGGCACT GAGCAGAAGGGAAGAAGCTCCGGGGGCTCTTTGTAGGGTCCTCCAGTCAGGACT CAAACCCAGTAGTGTCTGGTTCCAGGCACTGACCTTGTATGTCTCCTGGCCCAAA TGCCCACTCAGGGTAGGGGTGTAGGGCAGAAGAAGAAACAGACTCTAATGTTGC TACAAGGGCTGGTCCCATCTCCTGAGCCCCATGTCAAACAGAATCCAAGACATCC CAACCCTTCACCTTGGCTGTGCCCCTAATCCTCAACTAAGCTAGGCGCAAATTCC AATCCTCTTTGGTCTAGTACCCCGGGGGCAGCCCCCTCTAACCTTGGGCCTCAGC AGCAGGGGAGGCCACACCTTCCTAGTGCAGGTGGCCATATTGTGGCCCCTTGGA ACTGGGTCCCACTCAGCCTCTAGGCGATTGTCTCCTAATGGGGCTGAGATGAGAC ACAGTGGGGACAGTGGTTTGGACAATAGGACTGGTGACTCTGGTCCCCAGAGGC CTCATGTCCCTCTGTCTCCAGAAAATTCCCACTCTCACTTCCCTTTCCTCCTCAGT CTTGCTAGGGTCCATTTCTTACCCCTTGCTGAATTTGAGCCCACCCCCTGGACTTT TTCCCCATCTTCTCCAATCTGGCCTAGTTCTATCCTCTGGAAGCAGAGCCGCTGGA CGCTCTGGGTTTCCTGAGGCCCGTCCACTGTCACCAATATCAGGAACCATTGCCA CGTCCTAATGACGTGCGCTGGAAGCCTCTAGTTTCCAGAAGCTGCACAAAGATCC CTTAGATACTCTGTGTGTCCATCTTTGGCCTGGAAAATACTCTCACCCTGGGGCTA GGAAGACCTCGGTTTGTACAAACTTCCTCAAATGCAGAGCCTGAGGGCTCTCCCC ACCTCCTCACCAACCCTCTGCGTGGCATAGCCCTAGCCTCAGCGGGCAGTGGATG CTGGGGCTGGGCATGCAGGGAGAGGCTGGGTGGTGTCATCTGGTAACGCAGCCA CCAAACAATGAAGCGACACTGATTCCACAAGGTGCATCTGCATCCCCATCTGATC CATTCCATCCTGTCACCCAGCCATGCAGACGTTTATGATCCCCTTTTCCAGGGAG GGAATGTGAAGCCCCAGAAAGGGCCAGCGCTCGGCAGCCACCTTGGCTGTTCCC AAGTCCCTCACAGGCAGGGTCTCCCTACCTGCCTGTCCTCAGGTACATCCCCGAG GGCCTGCAGTGCTCGTGTGGAATCGACTACTACACGCTCAAGCCGGAGGTCAAC AACGAGTCTTTTGTCATCTACATGTTCGTGGTCCACTTCACCATCCCCATGATTAT CATCTTTTTCTGCTATGGGCAGCTCGTCTTCACCGTCAAGGAGGTACGGGCCGGG GGGTGGGCGGCCTCACGGCTCTGAGGGTCCAGCCCCCAGCATGCATCTGCGGCT CCTGCTCCCTGGAGGAGCCATGGTCTGGACCCGGGTCCCGTGTCCTGCAGGCCGC TGCCCAGCAGCAGGAGTCAGCCACCACACAGAAGGCAGAGAAGGAGGTCACCC GCATGGTCATCATCATGGTCATCGCTTTCCTGATCTGCTGGGTGCCCTACGCCAG CGTGGCATTCTACATCTTCACCCACCAGGGCTCCAACTTCGGTCCCATCTTCATG ACCATCCCAGCGTTCTTTGCCAAGAGCGCCGCCATCTACAACCCTGTCATCTATA TCATGATGAACAAGCAGGTGCCTACTGCGGGTGGGAGGGCCCCAGTGCCCCAGG CCACAGGCGCTGCCTGCCAAGGACAAGCTACTTCCCAGGGCAGGGGAGGGGGCT CCATCAGGGTTACTGGCAGCAGTCTTGGGTCAGCAGTCCCAATGGGGAGTGTGTG AGAAATGCAGATTCCTGGCCCCACTCAGAACTGCTGAATCTCAGGGTGGGCCCA GGAACCTGCATTTCCAGCAAGCCCTCCACAGGTGGCTCAGATGCTCACTCAGGTG GGAGAAGCTCCAGTCAGCTAGTTCTGGAAGCCCAATGTCAAAGTCAGAAGGACC CAAGTCGGGAATGGGATGGGCCAGTCTCCATAAAGCTGAATAAGGAGCTAAAAA GTCTTATTCTGAGGGGTAAAGGGGTAAAGGGTTCCTCGGAGAGGTACCTCCGAG GGGTAAACAGTTGGGTAAACAGTCTCTGAAGTCAGCTCTGCCATTTTCTAGCTGT ATGGCCCTGGGCAAGTCAATTTCCTTCTCTGTGCTTTGGTTTCCTCATCCATAGAA AGGTAGAAAGGGCAAAACACCAAACTCTTGGATTACAAGAGATAATTTACAGAA CACCCTTGGCACACAGAGGGCACCATGAAATGTCACGGGTGACACAGCCCCCTT GTGCTCAGTCCCTGGCATCTCTAGGGGTGAGGAGCGTCTGCCTAGCAGGTTCCCT CCAGGAAGCTGGATTTGAGTGGATGGGGCGCTGGAATCGTGAGGGGCAGAAGCA GGCAAAGGGTCGGGGCGAACCTCACTAACGTGCCAGTTCCAAGCACACTGTGGG CAGCCCTGGCCCTGACTCAAGCCTCTTGCCTTCCAGTTCCGGAACTGCATGCTCA CCACCATCTGCTGCGGCAAGAACCCACTGGGTGACGATGAGGCCTCTGCTACCGT GTCCAAGACGGAGACGAGCCAGGTGGCCCCGGCCTAAGACCTGCCTAGGACTCT GTGGCCGACTATAGGCGTCTCCCATCCCCTACACCTTCCCCCAGCCACAGCCATC CCACCAGGAGCAGCGCCTGTGCAGAATGAACGAAGTCACATAGGCTCCTTAATT TTTTTTTTTTTTTTAAGAAATAATTAATGAGGCTCCTCACTCACCTGGGACAGCCT GAGAAGGGACATCCACCAAGACCTACTGATCTGGAGTCCCACGTTCCCCAAGGC CAGCGGGATGTGTGCCCCTCCTCCTCCCAACTCATCTTTCAGGAACACGAGGATT CTTGCTTTCTGGAAAAGTGTCCCAGCTTAGGGATAAGTGTCTAGCACAGAATGGG GCACACAGTAGGTGCTTAATAAATGCTGGATGGATGCAGGAAGGAATGGAGGAA TGAATGGGAAGGGAGAACATATCTATCCTCTCAGACCCTCGCAGCAGCAGCAAC TCATACTTGGCTAATGATATGGAGCAGTTGTTTTTCCCTCCCTGGGCCTCACTTTC TTCTCCTATAAAATGGAAATCCCAGATCCCTGGTCCTGCCGACACGCAGCTACTG AGAAGACCAAAAGAGGTGTGTGTGTGTCTATGTGTGTGTTTCAGCACTTTGTAAA TAGCAAGAAGCTGTACAGATTCTAGTTAATGTTGTGAATAACATCAATTAATGTA ACTAGTTAATTACTATGATTATCACCTCCTGATAGTGAACATTTTGAGATTGGGC ATTCAGATGATGGGGTTTCACCCAACCTTGGGGCAGGTTTTTAAAAATTAGCTAG GCATCAAGGCCAGACCAGGGCTGGGGGTTGGGCTGTAGGCAGGGACAGTCACAG GAATGCAGAATGCAGTCATCAGACCTGAAAAAACAACACTGGGGGAGGGGGAC GGTGAAGGCCAAGTTCCCAATGAGGGTGAGATTGGGCCTGGGGTCTCACCCCTA GTGTGGGGCCCCAGGTCCCGTGCCTCCCCTTCCCAATGTGGCCTATGGAGAGACA GGCCTTTCTCTCAGCCTCTGGAAGCCACCTGCTCTTTTGCTCTAGCACCTGGGTCC CAGCATCTAGAGCATGGAGCCTCTAGAAGCCATGCTCACCCGCCCACATTTAATT AACAGCTGAGTCCCTGATGTCATCCTTATCTCGAAGAGCTTAGAAACAAAGAGTG GGAAATTCCACTGGGCCTACCTTCCTTGGGGATGTTCATGGGCCCCAGTTTCCAG TTTCCCTTGCCAGACAAGCCCATCTTCAGCAGTTGCTAGTCCATTCTCCATTCTGG AGAATCTGCTCCAAAAAGCTGGCCACATCTCTGAGGTGTCAGAATTAAGCTGCCT CAGTAACTGCTCCCCCTTCTCCATATAAGCAAAGCCAGAAGCTCTAGCTTTACCC AGCTCTGCCTGGAGACTAAGGCAAATTGGGCCATTAAAAGCTCAGCTCCTATGTT GGTATTAACGGTGGTGGGTTTTGTTGCTTTCACACTCTATCCACAGGATAGATTG AAACTGCCAGCTTCCACCTGATCCCTGACCCTGGGATGGCTGGATTGAGCAATGA GCAGAGCCAAGCAGCACAGAGTCCCCTGGGGCTAGAGGTGGAGGAGGCAGTCCT GGGAATGGGAAAAACCCCA (SEQ ID NO: 249) A non-limiting example of a human rhodopsin amino acid sequence is available under UniProt Accession No: P08100. All information available under UniProt Accession No: P08100 is incorporated herein by reference. An amino acid sequence that is available from UniProt Accession No: P08100 is as follows:

(SEQ ID NO: 250)
MNGTEGPNFYVPFSNATGVVRSPFEYPQYYLAEPWQFSMLAAYMFLLIVL
GFPINFLTLYVTVQHKKLRTPLNYILLNLAVADLFMVLGGFTSTLYTSLH
GYFVFGPTGCNLEGFFATLGGEIALWSLVVLAIERYVVVCKPMSNFRFGE
NHAIMGVAFTWVMALACAAPPLAGWSRYIPEGLQCSCGIDYYTLKPEVNN
ESFVIYMFVVHFTIPMIIIFFCYGQLVFTVKEAAAQQQESATTQKAEKEV
TRMVIIMVIAFLICWVPYASVAFYIFTHQGSNFGPIFMTIPAFFAKSAAI
YNPVIYIMMNKQFRNCMLTTICCGKNPLGDDEASATVSKTETSQVAPA

A non-limiting example of a cDNA sequence that encodes human rhodopsin is available under NCBI Reference Sequence No: NM_000539.3, and all information available under NCBI Reference Sequence No: NM_000539.3 is incorporated herein by reference. The nucleotide sequence that is available from NCBI Reference Sequence No: NM_000539.3 is as follows (the start and stop codons are underlined):

(SEQ ID NO: 251)
AGAGTCATCCAGCTGGAGCCCTGAGTGGCTGAGCTCAGGCCTTCGCAGCA
TTCTTGGGTGGGAGCAGCCACGGGTCAGCCACAAGGGCCACAGCCATGAA
TGGCACAGAAGGCCCTAACTTCTACGTGCCCTTCTCCAATGCGACGGGTG
TGGTACGCAGCCCCTTCGAGTACCCACAGTACTACCTGGCTGAGCCATGG
CAGTTCTCCATGCTGGCCGCCTACATGTTTCTGCTGATCGTGCTGGGCTT
CCCCATCAACTTCCTCACGCTCTACGTCACCGTCCAGCACAAGAAGCTGC
GCACGCCTCTCAACTACATCCTGCTCAACCTAGCCGTGGCTGACCTCTTC
ATGGTCCTAGGTGGCTTCACCAGCACCCTCTACACCTCTCTGCATGGATA
CTTCGTCTTCGGGCCCACAGGATGCAATTTGGAGGGCTTCTTTGCCACCC
TGGGCGGTGAAATTGCCCTGTGGTCCTTGGTGGTCCTGGCCATCGAGCGG
TACGTGGTGGTGTGTAAGCCCATGAGCAACTTCCGCTTCGGGGAGAACCA
TGCCATCATGGGCGTTGCCTTCACCTGGGTCATGGCGCTGGCCTGCGCCG
CACCCCCACTCGCCGGCTGGTCCAGGTACATCCCCGAGGGCCTGCAGTGC
TCGTGTGGAATCGACTACTACACGCTCAAGCCGGAGGTCAACAACGAGTC
TTTTGTCATCTACATGTTCGTGGTCCACTTCACCATCCCCATGATTATCA
TCTTTTTCTGCTATGGGCAGCTCGTCTTCACCGTCAAGGAGGCCGCTGCC
CAGCAGCAGGAGTCAGCCACCACACAGAAGGCAGAGAAGGAGGTCACCCG
CATGGTCATCATCATGGTCATCGCTTTCCTGATCTGCTGGGTGCCCTACG
CCAGCGTGGCATTCTACATCTTCACCCACCAGGGCTCCAACTTCGGTCCC
ATCTTCATGACCATCCCAGCGTTCTTTGCCAAGAGCGCCGCCATCTACAA
CCCTGTCATCTATATCATGATGAACAAGCAGTTCCGGAACTGCATGCTCA
CCACCATCTGCTGCGGCAAGAACCCACTGGGTGACGATGAGGCCTCTGCT
ACCGTGTCCAAGACGGAGACGAGCCAGGTGGCCCCGGCCTAAGACCTGCC
TAGGACTCTGTGGCCGACTATAGGCGTCTCCCATCCCCTACACCTTCCCC
CAGCCACAGCCATCCCACCAGGAGCAGCGCCTGTGCAGAATGAACGAAGT
CACATAGGCTCCTTAATTTTTTTTTTTTTTTTAAGAAATAATTAATGAGG
CTCCTCACTCACCTGGGACAGCCTGAGAAGGGACATCCACCAAGACCTAC
TGATCTGGAGTCCCACGTTCCCCAAGGCCAGCGGGATGTGTGCCCCTCCT
CCTCCCAACTCATCTTTCAGGAACACGAGGATTCTTGCTTTCTGGAAAAG
TGTCCCAGCTTAGGGATAAGTGTCTAGCACAGAATGGGGCACACAGTAGG
TGCTTAATAAATGCTGGATGGATGCAGGAAGGAATGGAGGAATGAATGGG
AAGGGAGAACATATCTATCCTCTCAGACCCTCGCAGCAGCAGCAACTCAT
ACTTGGCTAATGATATGGAGCAGTTGTTTTTCCCTCCCTGGGCCTCACTT
TCTTCTCCTATAAAATGGAAATCCCAGATCCCTGGTCCTGCCGACACGCA
GCTACTGAGAAGACCAAAAGAGGTGTGTGTGTGTCTATGTGTGTGTTTCA
GCACTTTGTAAATAGCAAGAAGCTGTACAGATTCTAGTTAATGTTGTGAA
TAACATCAATTAATGTAACTAGTTAATTACTATGATTATCACCTCCTGAT
AGTGAACATTTTGAGATTGGGCATTCAGATGATGGGGTTTCACCCAACCT
TGGGGCAGGTTTTTAAAAATTAGCTAGGCATCAAGGCCAGACCAGGGCTG
GGGGTTGGGCTGTAGGCAGGGACAGTCACAGGAATGCAGAATGCAGTCAT
CAGACCTGAAAAAACAACACTGGGGGAGGGGGACGGTGAAGGCCAAGTTC
CCAATGAGGGTGAGATTGGGCCTGGGGTCTCACCCCTAGTGTGGGGCCCC
AGGTCCCGTGCCTCCCCTTCCCAATGTGGCCTATGGAGAGACAGGCCTTT
CTCTCAGCCTCTGGAAGCCACCTGCTCTTTTGCTCTAGCACCTGGGTCCC
AGCATCTAGAGCATGGAGCCTCTAGAAGCCATGCTCACCCGCCCACATTT
AATTAACAGCTGAGTCCCTGATGTCATCCTTATCTCGAAGAGCTTAGAAA
CAAAGAGTGGGAAATTCCACTGGGCCTACCTTCCTTGGGGATGTTCATGG
GCCCCAGTTTCCAGTTTCCCTTGCCAGACAAGCCCATCTTCAGCAGTTGC
TAGTCCATTCTCCATTCTGGAGAATCTGCTCCAAAAAGCTGGCCACATCT
CTGAGGTGTCAGAATTAAGCTGCCTCAGTAACTGCTCCCCCTTCTCCATA
TAAGCAAAGCCAGAAGCTCTAGCTTTACCCAGCTCTGCCTGGAGACTAAG
GCAAATTGGGCCATTAAAAGCTCAGCTCCTATGTTGGTATTAACGGTGGT
GGGTTTTGTTGCTTTCACACTCTATCCACAGGATAGATTGAAACTGCCAG
CTTCCACCTGATCCCTGACCCTGGGATGGCTGGATTGAGCAATGAGCAGA
GCCAAGCAGCACAGAGTCCCCTGGGGCTAGAGGTGGAGGAGGCAGTCCTG
GGAATGGGAAAAACCCCA

In an aspect, provided herein is a pHLIP peptide (as well as compounds comprising at least one such pHLIP peptide) having the sequence: XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein, (i) each Y is, individually, a non-polar amino acid with solvation energy, ΔG_{X}^{cor}>+0.50, or Gly; (ii) each X is, individually, a protonatable amino acid, (iii) n, m, i, j, 1, h, g, f are each, individually, an integer from 1 to 8.

In an aspect, provided herein is a pH-triggered compound comprising a pHLIP peptide that is covalently attached to at least one other pHLIP peptide (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more pHLIP peptides) via a linker or a covalent bond. In some embodiments, the compound has the following structure:

[A]ₖ-linker

wherein k is an integer from 2 to 32, and each A is, individually, a pHLIP peptide comprising at least 8 amino acids. In certain embodiments, (i) at least 4 of the at least 8 amino acids are non-polar amino acids, (ii) at least 1 of the at least 8 amino acids is protonatable, and (iii) the pHLIP peptide has a higher affinity for a membrane lipid bilayer at pH 5.0 compared to the affinity at pH 8.0. In various embodiments, the at least 8 amino acids are consecutive amino acids. In some embodiments, the at least 8 amino acids are not consecutive amino acids. In various embodiments, the pHLIP peptide has less than 30, 25, or 20 total amino acids. In certain embodiments, the sequence of the pHLIP peptide has 8-20 (e.g., 8, 9, 10, 11, 12, 13, 14, 15, 16, 17, 18, 19, 20, 8-15, 8-20, 10-15, 10-20, or 15-20) consecutive amino acids.

In certain embodiments, included herein is a pH-triggered compound comprising a pHLIP peptide that is covalently attached to at least one other pHLIP peptide (e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32 or more pHLIP peptides) via a linker or a covalent bond. In some embodiments, the compound has the following structure:

[A]ₖ-linker

wherein k is an integer from 2 to 32, and each A is, individually, a pHLIP peptide comprising at least 8 consecutive amino acids. In certain embodiments, (i) at least 4 of the at least 8 consecutive amino acids are non-polar amino acids, (ii) at least 1 of the at least 8 consecutive amino acids is protonatable, and (iii) the pHLIP peptide has a higher affinity for a membrane lipid bilayer at pH 5.0 compared to the affinity at pH 8.0.

In various embodiments, the linker comprises a polymer that occurs in nature or an artificial polymer.

In some embodiments, each pHLIP peptide, individually, has the sequence: XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein, (i) each Y is, individually, a non-polar amino acid with solvation energy, j>+0.50, or Gly; (ii) each X is, individually, a protonatable amino acid, and (iii) n, m, i, j, 1, h, g, f are each, individually, an integer from 1 to 8.

In certain embodiments, a pH-triggered compound comprises at least two pHLIP peptides with different amino acid sequences.

In various embodiments, each pHLIP peptide of a pH-triggered compound comprises the same amino acid sequence.

In some embodiments, each pHLIP peptide of a pH-triggered compound is attached to the linker via a separate covalent bond.

In certain embodiments, a pH-triggered compound comprises a first pHLIP peptide that is covalently attached to a second pHLIP peptide via a linker or a covalent bond. In various embodiments, the compound comprises the following structure:

A-L-B

wherein A is the first pHLIP peptide, B is the second pHLIP peptide, L is the linker, and each - is a covalent bond.

In some embodiments, a pH-triggered compound comprises a first pHLIP peptide that is covalently attached to a second pHLIP peptide and a third pHLIP peptide via a linker or a covalent bond. In certain embodiments, the compound comprises the following structure:

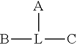

wherein A is the first pHLIP peptide, B is the second pHLIP peptide, C is the third pHLIP peptide, L is the linker, and each - is a covalent bond.

In various embodiments, a pH-triggered compound comprises a first pHLIP peptide that is covalently attached to a second pHLIP peptide, a third pHLIP peptide, and a fourth pHLIP peptide via a linker or a covalent bond. In some embodiments, the compound comprises the following structure:

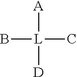

wherein A is the first pHLIP peptide, B is the second pHLIP peptide, C is the third pHLIP peptide, D is the fourth pHLIP peptide, L is the linker, and each - is a covalent bond.

In certain embodiments, a pH-triggered compound comprises k pHLIP peptides, wherein each pHLIP peptide has a unique amino acid sequence compared to each of the other pHLIP peptides in the compound, wherein k≥2.

In various embodiments, a pH-triggered compound comprises k pHLIP peptides, wherein each of the k pHLIP peptides has an identical amino acid sequence, wherein each of the k pHLIP peptides is connected to each of the other k pHLIP peptides by a linker, wherein 1<k≤32.

In certain embodiments, each pHLIP peptide in a pH-triggered compound has a net negative charge at a pH of about 7.25, 7.5, or 7.75 in water.

In various embodiments, each pHLIP peptide in a pH-triggered compound has an acid dissociation constant on a base 10 logarithmic scale (pKa) of less than about 4.0, 4.5, 5.0, 5.5, 6.0, 6.5, or 7.0.

In some embodiments, at least one of the pHLIP peptides in a pH-triggered compound comprises (a) 1 protonatable amino acid which is aspartic acid, glutamic acid, alpha-aminoadipic acid, or gamma-carboxyglutamic acid; or (b) at least 2, 3, or 4 protonatable amino acids, wherein the protonatable amino acids comprise aspartic acid, glutamic acid, alpha-aminoadipic acid, gamma-carboxyglutamic acid, or any combination thereof. In some embodiments, each pHLIP peptide in a pH-triggered compound comprises (a) 1 protonatable amino acid which is aspartic acid, glutamic acid, alpha-aminoadipic acid, or gamma-carboxyglutamic acid; or (b) at least 2, 3, or 4 protonatable amino acids, wherein the protonatable amino acids comprise aspartic acid, glutamic acid, alpha-aminoadipic acid, gamma-carboxyglutamic acid, or any combination thereof.

In certain embodiments, at least one of the pHLIP peptides in a pH-triggered compound comprises at least 1 non-native protonatable amino acid. In certain embodiments, each pHLIP peptide in a pH-triggered compound comprises at least 1 non-native protonatable amino acid. In various embodiments, the non-native protonatable amino acid comprises at least 1, 2, 3, or 4 carboxyl groups. In some embodiments, at least one of the pHLIP peptides comprises at least 1, 2, 3, 4, 5, 6, 7, 8, 9, 10, 11, 12, 13, 14, 15, or 16 carboxyl groups. In some embodiments, each of the pHLIP peptides comprises at least 1, 2, 3, 4, 5, 6, 7, 8, 9, 10, 11, 12, 13, 14, 15, or 16 carboxyl groups. In certain embodiments, at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 coded amino acids. In certain embodiments, each of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 coded amino acids.

In various embodiments, at least one of the pHLIP peptides in a pH-triggered compound comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 non-coded amino acids. In some embodiments, the amino acids of at least one of the pHLIP peptides are non-native amino acids. In certain embodiments, at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 D-amino acids. In various embodiments, each of the pHLIP peptides in a pH-triggered compound comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 non-coded amino acids. In some embodiments, the amino acids of the pHLIP peptides are non-native amino acids. In certain embodiments, each of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 D-amino acids. In various embodiments, at least one of the pHLIP peptides in a pH-triggered compound comprises at least 1 non-coded amino acid, wherein the non-coded amino acid is an aspartic acid derivative, or a glutamic acid derivative. A “coded” amino acid is an amino acid for which there is at least one three-nucleotide human mRNA codon. A non-coded amino acid is any other amino acid, including naturally occurring amino acids that are produced by post-translational modification of an amino acid sequence. Non-limiting examples of coded and non-coded amino acids are listed in Table 2. In certain embodiments, a coded amino acid is an L-amino acid that is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In some embodiments, a naturally occurring amino acid is an L-amino acid that is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, selenocysteine, or pyrrolysine. In certain embodiments, a non-coded amino acid is any amino acid other than alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine. In various embodiments, a non-coded amino acid is a D-amino acid. In certain embodiments, a non-coded amino acid is non-naturally occurring amino acid. In some embodiments, a non-coded amino acid is selenocysteine, selenomethionine, pyrrolysine, alpha-aminoadipic acid, amino-caprylic acid, aminoethyl-cysteine, aminophenyl acetate, gamma-aminobutyric acid, aminoisobutyric acid, alloisoleucine, allylglycine, amino-butyric acid, amino-phenylalanine, bromo-phenylalanine, cyclo-hexylalanine, citrulline, chloroalanine, cycloleucine, chlorophenylalanine, cysteic acid, diaminobutyric acid, diaminopropionic acid, diaminopimelic acid, dehydro-proline, 3,4-dihydroxyphenylalanine, fluorophenylalanine, glucosaminic acid, gamma-carboxyglutamic acid, homoarginine, hydroxylysine, hydroxynorvaline, homoglutamine, homophenylalanine, homoserine, homocysteine, hydroxyproline, iodo-phenylalanine, isoserine, methyl-leucine, methionine-methylsulfonium chloride, naphthyl-alanine, norleucine, N-methyl-alanine, norvaline, O-benzyl-serine, O-benzyl-tyrosine, O-ethyl-tyrosine, O-methyl-serine, O-methy-threonine, O-methyl-tyrosine, ornithine, penicillamine, pyroglutamic acid, pipecolic acid, sarcosine, trifluoro-alanine, hydroxy-Dopa, vinylglycine, amino-aminoethylsulfanylpropanoic acid, amino-hydroxy-dioxanonanolic acid, amino-hydroxy-oxahexanoic acid, amino-hydroxyethylsulfanylpropanoic acid, methoxyphenyl-methylpropanyl oxycarbonylamino propanoic acid, methyl-tryptophan, phosphorylated tyrosine, phosphorylated serine, phosphorylated threonine, biotin-lysine, hydroproline, phenylglycine, cyclohexyl-alanine, cyclohexylglycine, naphthylalanine, pyridyl-alanine, propargylglycine, pentenoic acid, penicillamine, methionine sulfoxide, pyroglutamic acid, acetylated lysine. In various embodiments, each of the pHLIP peptides in a pH-triggered compound comprises at least 1 non-coded amino acid, wherein the non-coded amino acid is an aspartic acid derivative, or a glutamic acid derivative.

In some embodiments, at least one of the pHLIP peptides in a pH-triggered compound comprises at least 8 consecutive amino acids, wherein, at least 2, 3, or 4 of the at least 8 consecutive amino acids are non-polar, and at least 1, 2, 3, or 4 of the at least 8 consecutive amino acids is protonatable. In some embodiments, each of the pHLIP peptides in a pH-triggered compound comprises at least 8 consecutive amino acids, wherein, at least 2, 3, or 4 of the at least 8 consecutive amino acids are non-polar, and at least 1, 2, 3, or 4 of the at least 8 consecutive amino acids is protonatable.

In certain embodiments, a pH-triggered compound comprises at least one pHLIP peptide that comprises a functional group to which the linker is attached.

In various embodiments, a pH-triggered compound comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are linked together by the linker.

In some embodiments, a pH-triggered compound comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each directly linked to the linker by a covalent bond.

In certain embodiments, the pHLIP peptides in a pH-triggered compound are attached to the linker by covalent bonds. In some embodiments pHLIP peptides are not connected to each other or to a linker by a peptide bond.

In various embodiments, multiple (e.g., 2-32, or 2, 3, 4, 5, 6, 7, 8, 9, 10 or more) pHLIP peptides are repeated in a continuous amino acid sequence. In some embodiments, the continuous amino acid sequence comprises an amino acid linker between the pHLIP peptides.

In certain embodiments, a compound comprises multiple (e.g., 2-32, or 2, 3, 4, 5, 6, 7, 8, 9, 10 or more) units, wherein each unit comprises a pHLIP peptide that is connected (e.g., linked by a covalent bond) to a cargo compound. In some embodiments, the cargo compound comprises a fluorophore. In certain embodiments, the fluorophore is ICG.

In various embodiments, a pH-triggered compound comprises at least one pHLIP peptide that is attached to the linker by a covalent bond. In some embodiments, the covalent bond is a peptide bond. In certain embodiments, the covalent bond is a disulfide bond, a bond between two selenium atoms, or a bond between a sulfur and a selenium atom. In various embodiments, the covalent bond is a bond that has been formed by a click chemistry reaction. In some embodiments, the covalent bond is a bond that has been formed by a reaction between (i) an azide and an alkyne; (ii) an alkyne and a strained difluorooctyne; (iii) a diaryl-strained-cyclooctyne and a 1,3-nitrone; (vi) a cyclooctene, trans-cycloalkene, or oxanorbornadiene and an azide, tetrazine, or tetrazole; (v) an activated alkene or oxanorbornadiene and an azide; (vi) a strained cyclooctene or other activated alkene and a tetrazine; or (vii) a tetrazole that has been activated by ultraviolet light and an alkene. In certain embodiments, the covalent bond is a peptide bond. In various embodiments, the covalent bond is not a peptide bond.

In some embodiments, the linker comprises an artificial polymer or a synthetically produced polymer that has the structure of a polymer that exists in nature. In certain embodiments, the linker comprises a polypeptide, a polylysine, a polyarginine, a polyglutamic acid, a polyaspartic acid, a polycysteine, or a polynucleic acid. In various embodiments, the linker does not comprise an amino acid. In some embodiments, the linker comprises a polysaccharide, a chitosan, or an alginate. In certain embodiments, the linker comprises a poly(ethylene glycol), a poly(lactic acid), a poly(glycolic acid), a poly(lactic-co-glycolic acid), a poly(malic acid), a polyorthoester, a poly(vinylalcohol), a poly(vinylpyrrolidone), a poly(methyl methacrylate), a poly(acrylic acid), a poly(acrylamide), a poly(methacrylic acid), a poly(amidoamine), a polyanhydrides, or a polycyanoacrylate. In various embodiments, the linker comprises poly(ethylene glycol). In some embodiments, the linker comprises more than one poly(ethylene glycol) structures (e.g., 2, 3, 4 or more) that are linked together. In certain embodiments, the poly(ethylene glycol) has a molecular weight of 60 to 100000 Daltons, e.g., at least about 60, 100, 200, 300, 400, 500, 600, 700, 800, 900, 1000, 5000, 10000, 15000, 20000, 25000) Daltons, but less than about 100000, 90000, 70000, 60000, 50000, 40000, or 30000 Daltons. In various embodiments, the linker comprises a linear polymer or a branched polymer. In some embodiments, the linker comprises an organic compound structure. In certain embodiments, the organic compound structure has a molecular weight less than about 10, 9, 8, 7, 6, 5, 4, 3, 2, 1, or 0.5 kDa.

In various embodiments, linker comprises a cell, a particle, a dendrimer, or a nanoparticle. In some embodiments, the linker comprises a cell, and at least 2 pHLIP peptides are covalently attached to the cell.

In embodiments, the linker does not comprise a lipid bilayer. In some embodiments, the linker is not a liposome. In various embodiments, each of the pHLIP peptides of a compound is directly covalently attached via a bond, or covalently attached via a linker, to each of the other pHLIP peptides of the compound.

In certain embodiments, the linker comprises a particle, a metallic particle, a polymeric particle, a nanoparticle, a metallic nanoparticle, a lipid-based nanoparticle, a surfactant-based nanoparticle, a polymeric nanoparticle, or a peptide-based nanoparticle. In various embodiments, a pHLIP peptide with a SH group directly interacts with gold nanoparticles (SH forms a bond with gold). In some embodiments, a pHLIP peptide is covalently linked to PEG or any other polymer, which is used for coating of particle or nanoparticle. In certain embodiments, a pHLIP peptide is covalently linked to a lipid, which is used to form a lipid-based nanoparticle. In various embodiments, a pHLIP peptide could be covalently linked to a surfactant, which is used to form surfactant-based nanoparticle. In some embodiments, a pHLIP peptide is covalently linked to a polymer, which is used to form a polymeric nanoparticle. In certain embodiments, a pHLIP peptide is covalently linked to another peptide or peptides, which is/are used to form a peptide-based nanoparticle.

In various embodiments, a pH-triggered compound comprises at least one pHLIP peptide that comprises a functional group for cargo compound attachment. In some embodiments, the compound comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each individually attached to a cargo compound via a linker. In certain embodiments, the compound comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each individually directly attached to a cargo compound by a covalent bond. In various embodiments, at least one of the pHLIP peptides is attached to a cargo compound by a covalent bond, wherein the covalent bond is an ester bond, a disulfide bond, a bond between two selenium atoms, a bond between a sulfur and a selenium atom, or an acid-liable bond. In some embodiments, at least one of the pHLIP peptides is attached to a cargo compound by a covalent bond, wherein the covalent bond is a bond that has been formed by a click chemistry reaction. In certain embodiments, the covalent bond is a bond that has been formed by a reaction between (i) an azide and an alkyne; (ii) an alkyne and a strained difluorooctyne; (iii) a diaryl-strained-cyclooctyne and a 1,3-nitrone; (iv) a cyclooctene, trans-cycloalkene, or oxanorbornadiene and an azide, tetrazine, or tetrazole; (v) an activated alkene or oxanorbornadiene and an azide; (vi) a strained cyclooctene or other activated alkene and a tetrazine; or (vii) a tetrazole that has been activated by ultraviolet light and an alkene. In various embodiments, the functional group is a side chain of an amino acid of a pHLIP peptide. In some embodiments, the side chain is a side chain to which a cargo compound may be attached via a disulfide bond. In certain embodiments, the functional group comprises a free sulfhydryl (SH) or selenohydryl (SeH) group. In various embodiments, the functional group comprises a cysteine, homocysteine, selenocysteine, or homoselenocysteine. In some embodiments, the functional group comprises a primary amine. In certain embodiments, the functional group comprises an azido modified amino acid. In various embodiments, the functional group comprises an alkynyl modified amino acid.

In some embodiments, the linker is attached to a cargo compound via a covalent bond. In certain embodiments, the covalent bond is an ester bond, a disulfide bond, a bond between two selenium atoms, a bond between a sulfur and a selenium atom, or an acid-liable bond. In various embodiments, the covalent bond is a bond that has been formed by a click chemistry reaction. In some embodiments, the covalent bond is a bond that has been formed by a reaction between (i) an azide and an alkyne; (ii) an alkyne and a strained difluorooctyne; (iii) a diaryl-strained-cyclooctyne and a 1,3-nitrone; (vi) a cyclooctene, trans-cycloalkene, or oxanorbornadiene and an azide, tetrazine, or tetrazole; (v) an activated alkene or oxanorbornadiene and an azide; (vi) a strained cyclooctene or other activated alkene and a tetrazine; or (vii) a tetrazole that has been activated by ultraviolet light and an alkene. In certain embodiments, the linker comprises a functional group for cargo compound attachment. In various embodiments, the functional group is an amino acid side chain. In some embodiments, the amino acid side chain is a side chain to which a cargo compound may be attached via a disulfide bond. In certain embodiments, the functional group comprises a free sulfhydryl (SH) or selenohydryl (SeH) group. In various embodiments, the functional group comprises a cysteine, homocysteine, selenocysteine, or homoselenocysteine. In some embodiments, the functional group comprises a primary amine. In certain embodiments, the functional group comprises an azido modified amino acid. In various embodiments, the functional group comprises an alkynyl modified amino acid.

In certain embodiments, a pHLIP peptide comprises a functional group to which a linker or cargo may be attached. Depending on context, a “functional group” may optionally be referred to as an “attachment group.” In various embodiments, a functional group is chemically reactive. In some embodiments, a functional group on a pHLIP peptide reacts with a functional group on a linker or cargo to leave a covalent bond that connects the pHLIP peptide to the linker or cargo. Non-limiting examples of functional groups include amino acid side chains (such as the —SH side chain of cysteine or a —NH2 side chain of lysine); thiols (e.g., moieties comprising, consisting essentially of, or consisting of —SH); esters such as maleimide esters; moieties comprising -she; and moieties that may be involved in click reactions (such as azides, alkynes, strained difluorooctynes, diaryl-strained-cyclooctynes, 1,3-nitrones, cyclooctenes, trans-cycloalkenes, oxanorbornadienes, tetrazines, tetrazoles, activated alkenes, and oxanorbornadienes. In some embodiments, a pHLIP peptide comprises a functional group, and a cargo or linker is non-covalently attached (e.g., via non-covalent binding such as an electrostatic interaction) to the functional group.

In some embodiments, a pH-triggered compound further comprises (e.g., is covalently bound to) a cargo compound. In certain embodiments, the cargo compound is polar. In various embodiments, the cargo compound is nonpolar. In some embodiments, the cargo compound comprises a marker. In certain embodiments, the cargo compound comprises a prophylactic, therapeutic, diagnostic, radiation-enhancing, radiation-sensitizing, imaging, gene regulation, immune activation, cytotoxic, apoptotic, or research agent. In various embodiments, the cargo compound comprises a dye (e.g., a fluorescent dye), a fluorescence quencher, or a fluorescent protein. In some embodiments, the cargo compound comprises a magnetic resonance, positron emission tomography, single photon emission computed tomography, fluorescent, optoacoustic, ultrasound, or X-ray contrast imaging agent. In certain embodiments, the cargo compound comprises a peptide, a protein, an enzyme, a polynucleotide, or a polysaccharide. In variois embodiments, the cargo compound comprises an aptamer, an antigen, a protease, an amylase, a lipase, a Fc receptor, a tissue factor, or a complement component 3 (C3) protein. In some embodiments, the cargo compound comprises a toxin, an inhibitor, a DNA intercalator, an alkylating agent, an antimetabolite, an anti-microtubule agents, a topoisomerase inhibitor, or an antibiotic compound. In certain embodiments, the cargo compound comprises an amanita toxin, a vinca alkaloid, a taxane, an anthracycline, a bleomycin, a nitrogen mustard, a nitrosourea, a tetrazine, an aziridine, a platinum-containing chemotherapeutic agent, cisplatin or a cisplatin derivative, a procarbazine, or a hexamethylmelamine. In various embodiments, the cargo compound comprises a DNA, a DNA analog, a RNA, or a RNA analog. In some embodiments, the cargo compound comprises a peptide nucleic acid (PNA), a bis PNA, a gamma PNA, a locked nucleic acid (LNA), or a morpholino. In certain embodiments, the cargo compound comprises a chemotherapeutic compound. In various embodiments, the cargo compound comprises an antimicrobial compound. In some embodiments, the cargo compound comprises a gene-regulation compound.

In certain embodiments, at least one of the pHLIP peptides in a pH-triggered compound comprises an amino acid side chain that is radioactive or detectable by probing radiation. In various embodiments, one or more atoms of the compound is a radioactive isotope. In some embodiments, one or more atoms of an amino acid of the compound has been replaced with a stable isotope.

In certain embodiments, a pH-triggered compound provided herein is used as an agent for ex vivo imaging or in an ex vivo diagnostic method.

In various embodiments, a pH-triggered compound included herein is used as a therapeutic agent, a diagnostic agent, an imaging agent, an ex vivo imaging agent, an immune activation agent, a gene regulation agent, a cell function regulation agent, a radiation-enhancing agent, a radiation-sensitizing agent, or a research tool.

In some embodiments, a pH-triggered compound provided herein is used as an agent to deliver a cargo compound across a cell membrane into a cell in a diseased tissue with a naturally acidic extracellular environment, or in a tissue with an artificially induced acidic extracellular environment, relative to normal physiological pH.

In certain embodiments, a pH-triggered compound provided herein is used as an agent to deliver a cargo molecule to the surface of a cell in a diseased tissue with a naturally acidic extracellular environment, or in a tissue with an artificially induced acidic extracellular environment, relative to normal physiological pH.

In various embodiments, a pH-triggered compound (i) comprises a pHLIP peptide that is attached to at least one other pHLIP peptide via a peptide linker, (ii) is present on the exterior surface of a cell, and (iii) is expressed by the cell, wherein if the cell is a human cell, then the cell is not within a human being.

In an aspect, provided herein is a cell (a non-ocular cell, e.g., a mammalian cell such as a T-cell, B-cell, neutrophil, eosinophil, basophil, lymphocyte, monocyte, dendritic cell, natural killer cell, macrophage, etc.) comprising a nucleic acid sequence that encodes a pHLIP peptide comprising at least 8 consecutive amino acids with a sequence that is identical to (i) a sequence of at least 8 consecutive amino acids that occurs in a naturally occurring human protein; or (ii) the reverse of a sequence of at least 8 consecutive amino acids that occurs in a naturally occurring human protein. For example, the pHLIP polypeptides described herein are present on the surface of a viable cell, such as a mammalian cell. In some embodiments, the cell is a non-ocular mammalian cell. In various embodiments, the composition does not comprise liposomes. In some embodiments, a purified or isolated population of pHLIP-expressing cells comprises a viable mammalian cell, e.g., an immune cell.

In some embodiments, the pHLIP peptide is expressed on the exterior surface of the cell (e.g., the at least 8 consecutive amino acids are outside the cell). In certain embodiments, the pHLIP peptide is tethered to the outside of the cell and the at least 8 consecutive amino acids are not in contact with the hydrophobic tails of phospholipids in the cell membrane lipid bilayer. In various embodiments, the pHLIP peptide or a fusion protein comprising the pHLIP peptide is trafficked to the outside of the cell where it is presented on the cell membrane (e.g., the outside of the cell is decorated with pHLIP peptides that extend from the cell membrane such that the at least 8 consecutive amino acids do not enter into the cell membrane, e.g., the at least 8 consecutive amino acids are outside of the lipid bilayer of the cell membrane). In some embodiments, at least about 60%, 65%, 70%, 75%, 80%, 85%, 90%, 95%, 96%, 97%, 98%, or 99% of the expressed pHLIP peptide is located on the exterior of the cell. In some embodiments, the naturally occurring human protein is a human rhodopsin protein. In certain embodiments, the at least 8 consecutive amino acids are less than the length of the human rhodopsin protein, e.g., the at least 8 consecutive amino acids are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 8-20, 8-30, 8-40, 8-50, 20-30, 20-40, or 20-50 consecutive amino acids. In certain embodiments, the 8 consecutive amino acids that occur in the human rhodopsin protein are within the following sequence: NLEGFFATLGGEIALWSLVVLAIE (SEQ ID NO: 82) or the reverse thereof. The reverse of NLEGFFATLGGEIALWSLVVLAIE (SEQ ID NO: 82) is EIALVVLSWLAIEGGLTAFFGELN (SEQ ID NO: 85). In some embodiments, the 8 consecutive amino acids comprise LGGEIALW (SEQ ID NO: 322). In various embodiments, the sequence of the pHLIP peptide comprises 8-20 (e.g., 8, 9, 10, 11, 12, 13, 14, 15, 16, 17, 18, 19, 20, 8-15, 8-20, 10-15, 10-20, or 15-20) consecutive amino acids that have a sequence that is 85%, 90%, or 95% identical to a sequence of 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein, wherein the sequence of the 8-20 (e.g., 8, 9, 10, 11, 12, 13, 14, 15, 16, 17, 18, 19, 20, 8-15, 8-20, 10-15, 10-20, or 15-20) consecutive amino acids of the pHLIP peptide has 1, 2, or 3 amino acid substitutions compared to the sequence of the 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein. In some embodiments, the sequence has a L to D, L to E, A to P, or C to G substitution compared to the 8-20 (e.g., 8, 9, 10, 11, 12, 13, 14, 15, 16, 17, 18, 19, 20, 8-15, 8-20, 10-15, 10-20, or 15-20) consecutive amino acids that occur in the human rhodopsin protein. In certain embodiments, the sequence of the pHLIP peptide comprises 8-20 (e.g., 8, 9, 10, 11, 12, 13, 14, 15, 16, 17, 18, 19, 20, 8-15, 8-20, 10-15, 10-20, or 15-20) consecutive amino acids that have a sequence that is 85%, 90%, or 95% identical to the reverse of a sequence of 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein, wherein the sequence of the 8-20 (e.g., 8, 9, 10, 11, 12, 13, 14, 15, 16, 17, 18, 19, 20, 8-15, 8-20, 10-15, 10-20, or 15-20) consecutive amino acids of the pHLIP peptide has 1, 2, or 3 amino acid substitutions compared to the reverse of the sequence of the 8-20 (e.g., 8, 9, 10, 11, 12, 13, 14, 15, 16, 17, 18, 19, 20, 8-15, 8-20, 10-15, 10-20, or 15-20) consecutive amino acids that occurs in a human rhodopsin protein. In some embodiments, the sequence has a L to D, L to E, A to P, or C to G substitution compared to the reverse of the 8-20 (e.g., 8, 9, 10, 11, 12, 13, 14, 15, 16, 17, 18, 19, 20, 8-15, 8-20, 10-15, 10-20, or 15-20) consecutive amino acids that occur in the human rhodopsin protein.

In certain embodiments, the cell comprises an exogenous polynucleotide that encodes the pHLIP peptide. In various embodiments, the cell is a non-ocular cell. In certain embodiments, the cell is a mammalian cell. In some embodiments, the cell is an immune cell. In various embodiments, the cell is a T-cell, B-cell, neutrophil, eosinophil, basophil, lymphocyte, monocyte, dendritic cell, natural killer cell, or macrophage. The exogenous polynucleotide may, e.g., comprise one or more regulatory elements such as a promoter (e.g., that promotes the expression of the pHLIP peptide), and a sequence that encodes the pHLIP peptide. In various embodiments, the exogenous polynucleotide comprises a viral vector or a plasmid. In some embodiments, the exogenous polynucleotide is integrated into the genome of the cell. In certain embodiments, the exogenous polynucleotide is not integrated into the genome of the cell. Any nucleotide sequence that encodes a pHLIP peptide disclosed herein may be used. With respect to pHLIP peptides that are derived from an amino acid sequence within a human rhodopsin protein, the sequence may comprise, e.g., a sequence that is at least about 90%, 91%, 92%, 93%, 94%, 95%, 96%, 97%, 9%8, or 99% identical, or is 100% identical to 24-60 (e.g., at least 24, 27, 30, 33, 36, 39, 42, 45, 48, 51, 54, 57, or 60) consecutive nucleotides in the following sequence:

(SEQ ID NO: 252)
AAYYTNGARGGNTTYTTYGCNACNYTNGGNGGNGARATHGCNYTNTGGWS
NYTNGTNGTNYTNGCNATHGARTRR,

wherein:
each N is, individually, A, C, G, or T;
each Y is, individually, C or T;
each R is, individually, A or G;
each H is, individually, A or C or T;
each W is, individually, A or T; and
each S is, individually, G or C.

In various embodiments, the sequence may comprise, e.g., a sequence that is at least about 90%, 91%, 92%, 93%, 94%, 95%, 96%, 97%, 9%8, or 99% identical, or is 100% identical to 24-60 (e.g., at least 24, 27, 30, 33, 36, 39, 42, 45, 48, 51, 54, 57, or 60) consecutive nucleotides in the following sequence:

(SEQ ID NO: 253)
AATTTGGAGGGCTTCTTTGCCACCCTGGGCGGTGAAATTGCCCTGTGGT
CCTTGGTGGTCCTGGCCATCGAG.

With respect to pHLIP peptides that are derived from an amino acid that is the reverse of a sequence within a human rhodopsin protein, the sequence may comprise, e.g., a sequence that is at least about 90%, 91%, 92%, 93%, 94%, 95%, 96%, 97%, 9%8, or 99% identical, or is 100% identical to 24-60 (e.g., at least 24, 27, 30, 33, 36, 39, 42, 45, 48, 51, 54, 57, or 60) consecutive nucleotides in the following sequence:

(SEQ ID NO: 254)
GARATHGCNYTNGTNGTNYTNWSNTGGYTNGCNATHGARGGNGGNYTNA
CNGCNTTYTTYGGNGARYTNAAYTRR,

wherein:
each N is, individually, A, C, G, or T;
each Y is, individually, C or T;
each R is, individually, A or G;
each H is, individually, A or C or T;
each W is, individually, A or T; and
each S is, individually, G or C.

In various embodiments, the sequence may comprise, e.g., a sequence that is at least about 90%, 91%, 92%, 93%, 94%, 95%, 96%, 97%, 9% 8, or 99% identical, or is 100% identical to 24-60 (e.g., at least 24, 27, 30, 33, 36, 39, 42, 45, 48, 51, 54, 57, or 60) consecutive nucleotides in the following sequence:

(SEQ ID NO: 255)
GAGATCGCCCTGGTCGTGTTGTCCTGGCTGGCCATTGAAGGTGGCCTGA
CCGCCTTTTTCGGCGAGTTGAAT.

Included herein is a cell comprising a pH-triggered compound comprising multiple pHLIP peptides as disclosed herein on the exterior surface thereof, wherein the pHLIP peptides of the compound are outside the hydrophobic tail region of the cell membrane of the cell when the cell is in an environment with a pH of less than 7.0.

Also provided herein is a particle comprising a pH-triggered compound comprising multiple pHLIP peptides as disclosed. In some embodiments, the particle is a nanoparticle.

In certain embodiments, a pH-triggered compound included herein is used to coat a cell, a particle, a nanoparticle, or a surface. In various embodiments, the nanoparticle is a metallic nanoparticle, a polymeric nanoparticle, a lipid-based nanoparticle, a surfactant-based nanoparticle, or a peptide-based nanoparticle. Non-limiting examples include: i) decorating a magnetic particle with pHLIP polypeptides to catch circulating cancer cells and the use these magnetic particles to collect/extract cells, which are associated with (e.g. gathered by) pHLIP peptides; ii) coating a surface (e.g., of a glass slide) to catch circulating cancer cells; iii) using a pHLIP polypeptide with a targeting moiety to decorate immune cells. For example, a pHLIP peptide may be expressed on the surface of T-cells. Alternatively or in addition, a pHLIP-t.m. may be used, where the t.m. is a targeting moiety for a T-cell receptor or a NK-cell receptor, such that immune cells are collected from a patient (e.g., from a biological sample obtained from the patient, such as blood), decorated with pHLIP, and injected back to the patient. In certain embodiments, such an approach decorates immune cells more quickly compared to the expression of pHLIP peptides on their surfaces.

In some embodiments, diseased tissue comprises cancerous tissue, inflamed tissue, ischemic tissue, arthritic tissue, cystic fibrotic tissue, tissue infected with a microorganism, or atherosclerotic tissue.

Certain implementations comprise a formulation for parenteral, a local, or systemic administration comprising a pH-triggered compound as disclosed herein.

Formulations comprising a pH-triggered compound for intravenous, intraarterial, intraperitoneal, intracerebral, intracerebroventricular, intrathecal, intracardiac, intracavernous, intraosseous, intraocular, or intravitreal administration are also provided.

In an aspect, provided herein is a formulation comprising a pH-triggered compound for intramuscular, intradermal, transdermal, transmucosal, intralesional, subcutaneous, topical, epicutaneous, extra-amniotic, intravaginal, intravesical, nasal, or oral administration.

The present subject matter also includes a formulation for intravesical instillation comprising a pH-triggered compound as disclosed herein. In some embodiments, the formulation is used for the treatment of bladder cancer.

Also provided herein is a formulation comprising a pH-triggered compound that comprises multiple pHLIP peptides for systemic administration. In certain embodiments, the formulation is used for the treatment of bladder cancer.

In an aspect, provided herein is a pH-triggered compound for the treatment of a superficial or muscle invasive bladder tumor comprising (i) a pHLIP peptide that is attached to at least one other pHLIP peptide via a peptide linker, and (ii) an amanitin toxic cargo.

In various embodiments, the cargo is amanitin. In some embodiments, two or more pHLIP peptides that are covalently attached to amanitin are linked. In certain embodiments, a compound with the structure

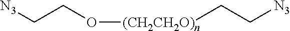

is used to covalently attach one pHLIP peptide that is covalently attached to amanitin to another pHLIP peptide that is covalently attached to amanitin.

In an aspect, included herein is a formulation comprising a compound as disclosed herein for the ex vivo contact or treatment (e.g., for a detection or diagnostic assay) of a biopsy specimen, a liquid biopsy specimen, surgically removed tissue, a surgically removed liquid, or blood.

In an aspect, provided herein is a method of treating cancer in a subject, comprising administering to the subject an effective amount of a pH-triggered compound, wherein the compound comprises an anti-cancer cargo compound. Non-limiting examples of cancer include colon cancer, prostate cancer, breast cancer, bladder cancer, lung cancer, skin cancer, liver cancer, bone cancer, ovarian cancer, stomach cancer, pancreatic cancer, testicular cancer, head and neck cancer, and brain cancer. In some embodiments, the cancer is bladder cancer.

Also included herein are methods for detecting and/or imaging diseased tissue (such as cancer tissue, ischemic tissue, or infected tissue) in a subject or in a biological sample obtained from the subject, comprising administering to the subject or contacting the biological sample with a pH-triggered compound, wherein the compound comprises a detectable cargo compound. In various embodiments, the biological sample comprises cells or tissue such as a biopsy (e.g., a tumor biopsy). In certain embodiments, the biological sample comprises a bodily fluid. Non-limiting examples of bodily fluids comprise, blood, serum, plasma, sweat, sputum, mucus, saliva, sweat, tears, and urine.

In an aspect, provided herein is a method of treating an infection in a subject, comprising administering to the subject an effective amount of a pH-triggered compound, wherein the compound comprises an antimicrobial compound. In various embodiments, the infection is a viral, bacterial, protozoan, or fungal infection.

Included herein are pharmaceutical compositions comprising a pH-triggered compound and a pharmaceutically acceptable carrier.

In various embodiments, compounds, compositions, and methods provided herein are useful for detecting cancerous or precancerous tissue in many bodily organs and tissues. In some embodiments, the bodily organ is a kidney or a urinary bladder. Non-limiting examples of tissues in which cancerous or precancerous tissue may be detected include bone, joint, ligament, muscle, tendon, salivary gland, tooth, gum, parotid gland, submandibular gland, sublingual gland, pharynx, esophagus, stomach, small intestine (e.g., duodenum, jejunum, and/or ileum), large intestine, liver, gallbladder, pancreas, nasal cavity, pharynx, larynx, trachea, bronchi, lung, diaphragm, kidney, ureter, bladder, urethra, ovary, uterus, fallopian tube, uterus, cervix, vagina, teste, epididymis, vas deferens, seminal vesicle, prostate, bulbourethral gland, pituitary gland, pineal gland, thyroid gland, parathyroid gland, adrenal gland, heart, artery, vein, capillary, lymphatic, lymph node, bone marrow, thymus, spleen, brain, cerebral hemisphere, diencephalon, brainstem, midbrain, pons, medulla oblongata, cerebellum, spinal cord, ventricular, choroid plexus, nerve, eye, ear, olfactory, breast, and skin tissue. In some embodiments, the diseased cancer tissue detected is sarcoma or carcinoma tissue. Non-limiting types of cancer that may be detected using compounds, compositions, and methods disclosed herein include bladder cancer, lung cancer, brain cancer, melanoma, breast cancer, cervical cancer, ovarian cancer, adrenal cancer, esophageal cancer, upper gastrointestinal cancer, anal cancer, bile duct cancer, bladder cancer, bone cancer, Castleman Disease, colon/rectum cancer, endometrial cancer, esophagus cancer, eye cancer, gallbladder cancer, gastrointestinal carcinoid tumors, gastrointestinal stromal tumors (GISTs), gestational trophoblastic disease, Kaposi sarcoma, kidney cancer, laryngeal cancer, hypopharyngeal cancer, liver cancer, malignant mesothelioma, nasal cavity cancer, paranasal sinus cancer, nasopharyngeal cancer, neuroblastoma, oral cavity cancer, oropharyngeal cancer, osteosarcoma, pancreatic cancer, penile cancer, pituitary tumors, prostate cancer, retinoblastoma, rhabdomyosarcoma, salivary gland cancer, sarcoma, small intestine cancer, stomach cancer, testicular cancer, thymus cancer, thyroid cancer, uterine sarcoma, vaginal cancer, vulbar cancer, and Wilms tumors. In various embodiments, the cancer comprises a solid tumor.

In some embodiments, the cancerous or precancerous tissue is in the bladder, the upper urinary tract, a lymph node, a breast, a prostate, a head, a neck, a brain, a pancreas, a lung, a liver, or a kidney.

In certain embodiments, compounds, compositions, and methods provided herein are also useful for detecting cancer cells (such as metastatic cancer cells) in tissue such as a lymph node. In some embodiments, the lymph node is in a subject who has cancer. In various embodiments, the lymph node is in a subject with bladder cancer, upper urinary tract cancer, breast cancer, prostate cancer, head and neck cancer, brain cancer, pancreatic cancer, lung cancer, liver cancer, or kidney cancer.

Diseased tissue (e.g., precancerous or cancer tissue) may be detected in tissue samples or biopsies obtained, removed, or provided from a subject. In various embodiments, the tissue comprises a tissue biopsy. Alternatively or in addition, the presence of diseased tissue is detected on a biological surface in vivo or in situ, e.g., the skin surface, the surface of a mucosal membrane, or an internal site (e.g., the internal surface of a bladder, the surface of a colon, the surface of an esophagus, or the surface of a surgical site within the subject). For example, the tissue to be interrogated comprises a lumen, e.g., a duct (such as a kidney duct), a ureter, an intestinal tissue (large or small intestine), an esophagus, or an airway lumen such as a tracheobronchial tube or alveolar tube. In some embodiments, a compound provided herein is used to detect the presence of melanoma tissue. In some embodiments, the bodily organ or tissue is present in a subject.

Optionally, methods disclosed herein may include steps such as washing steps to remove excess unbound or unattached compound, i.e. compound that is not attached to a low pH tissue via insertion of a pH-triggered polypeptide into a cell membrane. For example, an organ sample or tissue biopsy may be washed or perfused before ICG fluorescence is detected (e.g., imaged). In non-limiting examples in which a body cavity or surface has been contacted with a compound (e.g., in liquid or spray form), the cavity or surface may be flushed or washed to remove excess ICG before detection/imaging. In some embodiments, flushing/washing is performed using, e.g., an aqueous solution such as saline or water. In some embodiments, flushing/washing is performed with the carrier that was used to deliver the ICG-pH-triggered compound.

In some embodiments, contacting a bodily organ, tissue, or fluid (such as blood) with a compound provided herein comprises administering the compound to a subject. For example, the compound is detected in vivo. In certain embodiments, the compound is administered to the subject via intravessical instillation, intravenous injection, intraperitoneal injection, topical administration, mucosal administration, or oral administration. For example, the compound may be administered to a site within the subject (e.g., sprayed, applied onto, delivered as a liquid) via tube that is inserted into the subject. The site may be, e.g., an existing, former, or suspected tumor site, and/or normal tissue that is being assessed for the presence of cancerous or precancerous tissue. In some embodiments, a tube or other device (e.g., a catheter, needle, aspirator, inhaler, endoscope, cystoscope, atomizer, spray nozzle, probe, syringe, pipette, or nebulizer) is used to deliver the compound to, e.g., the esophagus, bladder, or colon. In certain embodiments, fluorescence of the compound is detected (e.g., imaged) using an endoscope or a cystoscope. For example, the endoscope or cystoscope may be configured to (i) emit electromagnetic radiation comprising an excitation wavelength of ICG and/or (ii) detect electromagnetic radiation emitted from the compound (i.e., the ICG component of the compound). In some embodiments, the compound is administered by applying a liquid, powder, or spray comprising the compound to a surface of the subject. In some embodiments, the surface comprises a site within the body of the subject that is accessed and/or exposed via surgery. In some embodiments, the surgery comprises endoscopic surgery or cystoscopic surgery. In certain embodiments, the compound is administered to an oral cavity of the subject.

In various embodiments, electromagnetic radiation emitted from the compound is detected ex vivo. In some embodiments, a tissue sample (e.g., a biopsy or an organ) from a subject is perfused, soaked, sprayed, incubated, and/or injected with a composition comprising a compound herein, followed by washing, and then imaging for ICG fluorescence.

Aspects of the present subject matter relate to methods comprising surgically removing cancerous tissue or precancerous tissue, e.g., cancer tissue or precancerous tissue detected with a compound, composition, or method disclosed herein. For example, the fluorescence of the compounds provided herein may be used to guide surgery such that all cancerous and/or precancerous tissue is removed, i.e., clean (non-cancer containing) margins of the surgical site are achieved.

The present subject matter provides methods for identifying precancerous and cancer/tumor tissue faster than existing pathological methods. For example, tissue removed during surgery can be contacted with ICG-pH-triggered compounds, washed, and then rapidly imaged to determine, e.g., whether all of the tissue removed was precancerous or cancerous and/or whether precancerous or cancerous tissue remains in a subject. Alternatively or in addition, the surgical site may be contacted with a compound (e.g., by local or systemic administration) to determine whether any diseased tissue remains at the site. The methods provided herein do not require, e.g., time consuming immunohistological staining or evaluation by a trained pathologist. The speed (e.g., 30 minutes or less) at which the methods provided herein may be performed enable clinicians to test for the presence or absence of precancerous or cancerous tissue (e.g., within a subject or a sample from the subject) during ongoing surgery, e.g., to determine whether and where surgery should continue (e.g., to remove more tissue).

The development, reoccurrence, and treatment of cancer can also be detected and monitored. For example, a subject who has had cancer surgically removed or treated (e.g., with chemotherapy or radiation) may be tested for cancer using compounds and methods disclosed herein. For example, the inside of a bladder, colon, esophagus, or oral cavity, and/or a mucosal membrane/skin surface may be contacted with a compound provided herein and then detected to determine whether precancerous and/or cancerous tissue is developing or has developed. In instances where, e.g., chemotherapy or radiation therapy efficacy is assessed, the amount of cancer tissue may be monitored. Thus, ICG-pH-triggered compounds provided herein can be used to assist decisions regarding whether cancer treatment should be initiated or continued, and/or whether a different treatment regimen should be attempted (e.g., if a previously administered dose/regimen has not reduced the amount of cancer tissue as desired).

Many different types of subjects with various stages of cancer can be assessed and/or treated using the compounds, compositions, and methods provided herein. However, various embodiments relate to the detection and treatment of cancer before the removal of a large amount of tissue (e.g., an organ such as a bladder or kidney, or, e.g. a portion of an organ such as a colon) is warranted or advisable. In various embodiments, the subject does not comprise invasive or metastatic cancer. In certain embodiments, relating to subjects with urothelial carcinoma, the subject does not comprise high grade urothelial carcinoma. In some embodiments, the subject does not comprise invasive high grade urothelial carcinoma.

As used herein, “effective” when referring to an amount of a compound refers to the quantity of the compound that is sufficient to yield a desired response (e.g., therapeutic outcome or imaging signal strength) without undue adverse side effects (such as toxicity, irritation, or allergic response) commensurate with a reasonable benefit/risk ratio when used in the manner of this disclosure.

In some embodiments, a subject is a mammal. In certain embodiments, the mammal is a rodent (e.g., a mouse or a rat), a primate (e.g., a chimpanzee, a gorilla, a monkey, a gibbon, a baboon, or a human), a cow, a camel, a dog, a cat, a horse, a llama, a sheep, a goat, a chicken, a turkey, a goose, or a duck. In certain embodiments, the subject is a human.

As used herein and depending on context, an “isolated” or “purified” compound, nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) is free of the genes or sequences that flank it in its naturally-occurring state. Purified also defines a degree of sterility that is safe for administration to a human subject, e.g., lacking infectious or toxic agents.

Similarly, by “substantially pure” is meant a compound that has been separated from the components that naturally accompany it. Typically, and depending on context, the compound is substantially pure when it is at least 60%, 70%, 80%, 90%, 95%, or even 99%, by weight, free from the proteins and naturally-occurring organic molecules with it is naturally associated.

As used herein, the term “purified” or “isolated” with reference to a cell, refers to a cell that is in an environment different from that in which the cell naturally occurs. For example, when the cell naturally occurs in a multicellular organism, and the cell is removed from the multicellular organism, the cell is “isolated.” In various embodiments, an isolated or purified cell is a cultured cell.

Methods of isolating and purifying immune cells (such as T-cells) from, e.g., blood, are known in the art. Non-limiting examples of such methods include labeling different immune cells according to cell-surface markers (e.g., with an antibody conjugated to a fluorescent marker) such as cluster of differentiation 8 (CD8), cluster of differentiation 4 (CD4), C-X-C Motif Chemokine Receptor 1 (CXCR1), Differentiation Antigen CD1-Alpha-3 (CDIc), cluster of differentiation 3 (CD3), Interleukin-2 Receptor alpha-Chain (CD25), L-selectin (CD62L), Integrin alpha M (CD11b), cluster of differentiation 14 (CD14), and/or forkhead box P3 (Foxp3), and sorting/separating the cells with flow cytometry (e.g., fluorescence-activated cell sorting in flow cytometry). In some embodiments, isolating cells from a bodily fluid comprises centrifugation. In various embodiments, a substrate (such as a bead, such as a microbead) comprising an antibody or antigen to which an immune cell binds is used in a process of isolating the immune cell.

Non-limiting examples of pHLIP peptides and features thereof, as well as pHLIP design considerations, are provided in Wyatt et al. (2018) Peptides of pHLIP family for targeted intracellular and extracellular delivery of cargo molecules to tumors, Proc Natl Acad Sci USA 115(12):E2811-E2818, the entire contents of which are incorporated herein by reference.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the P practice or testing of the present invention, suitable methods and materials are described below.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A and B are schematic representations of exemplary pHLIP compounds (compounds comprising multiple pHLIP peptides, which may also be referred to herein as “pHLIP bundles”): (A) PEG-2WT with 2 kDa 2-arm PEG and 2 WT pHLIPs, and (B) PEG-4WT with 2 kDa 4-arm PEG and 4 WT pHLIPs.

A 2-Arm and 4-Arm PEG-Azide were used for (A) and (B) respectively (each available from Creative PEGWorks, Chapel Hill, NC, USA):

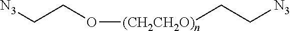

(Azide-PEG-Azide; Bifunctional PEG azide, N3-PEG-N3; Creative PEGWorks Cat No. PSB-325)

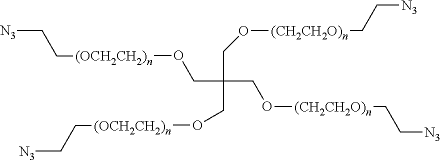

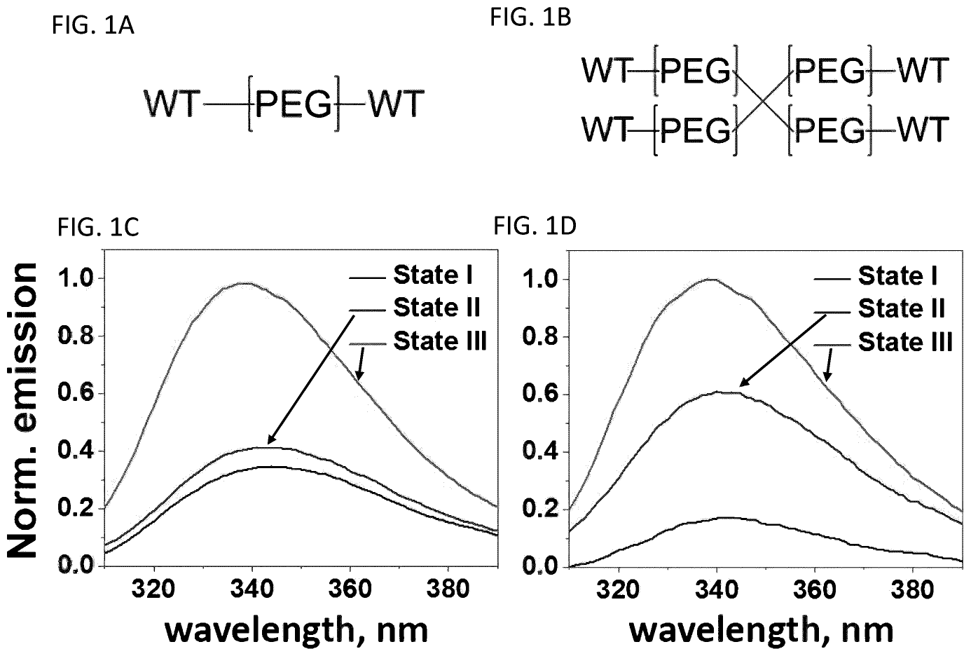

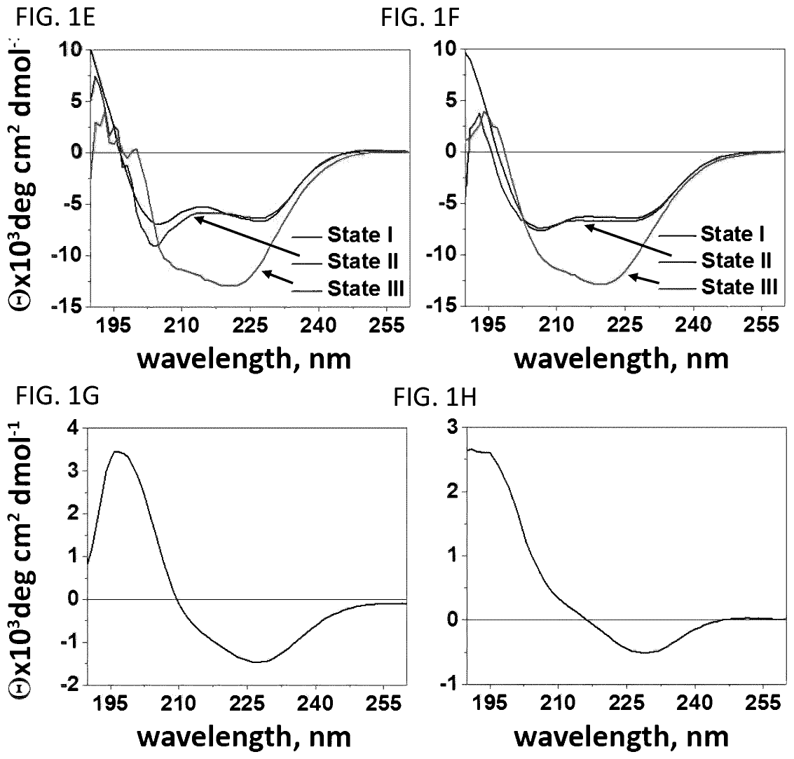

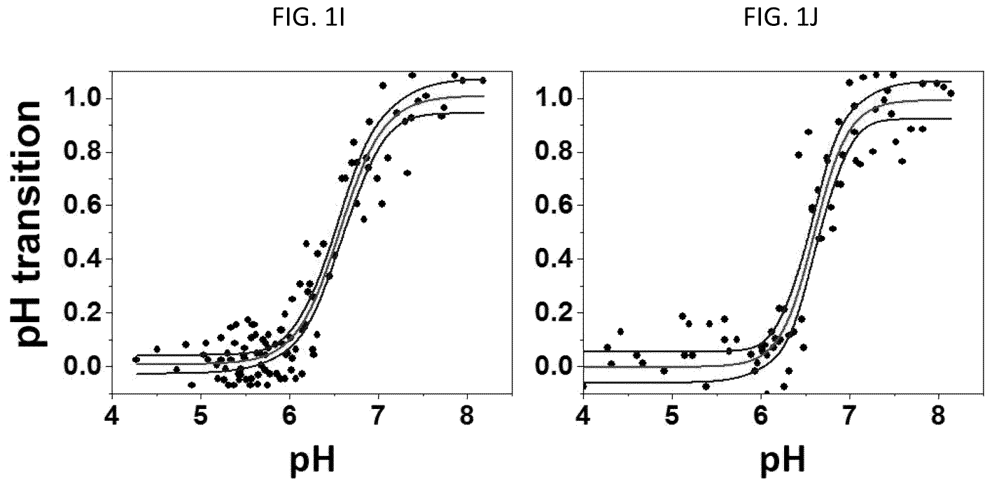

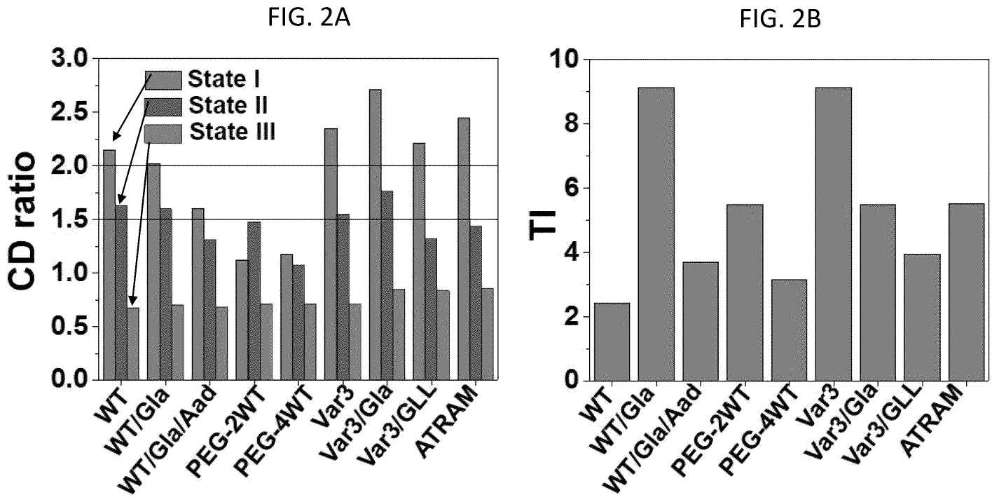

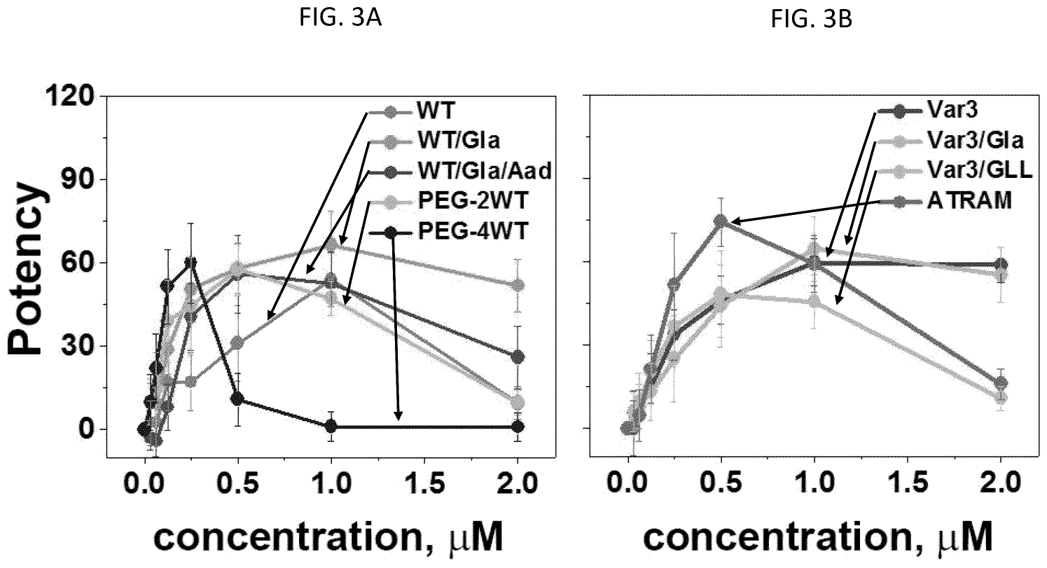

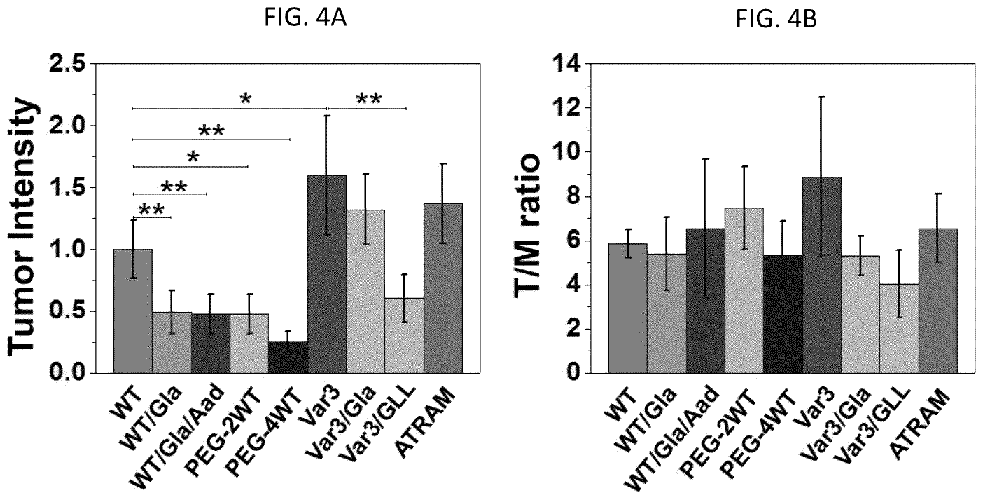

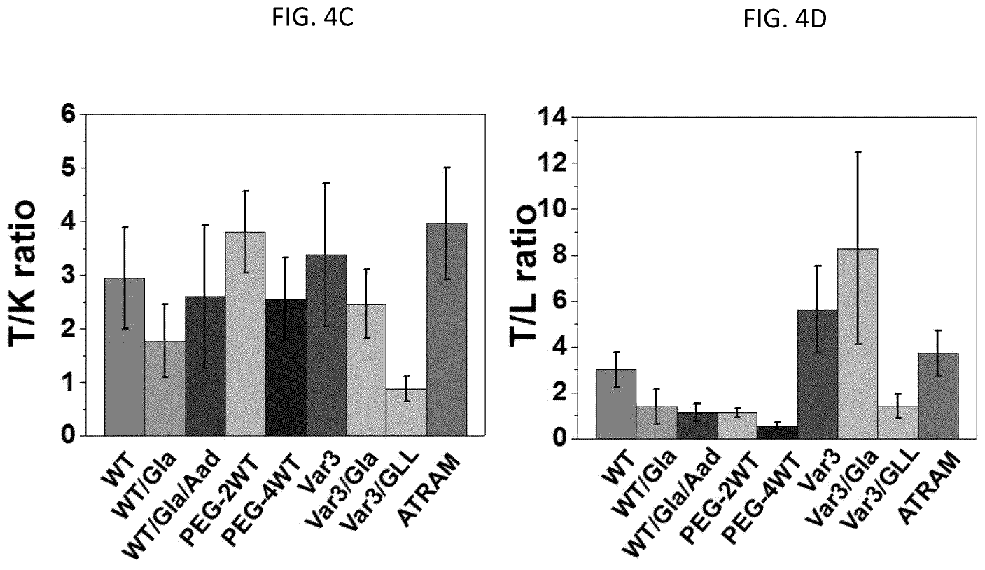

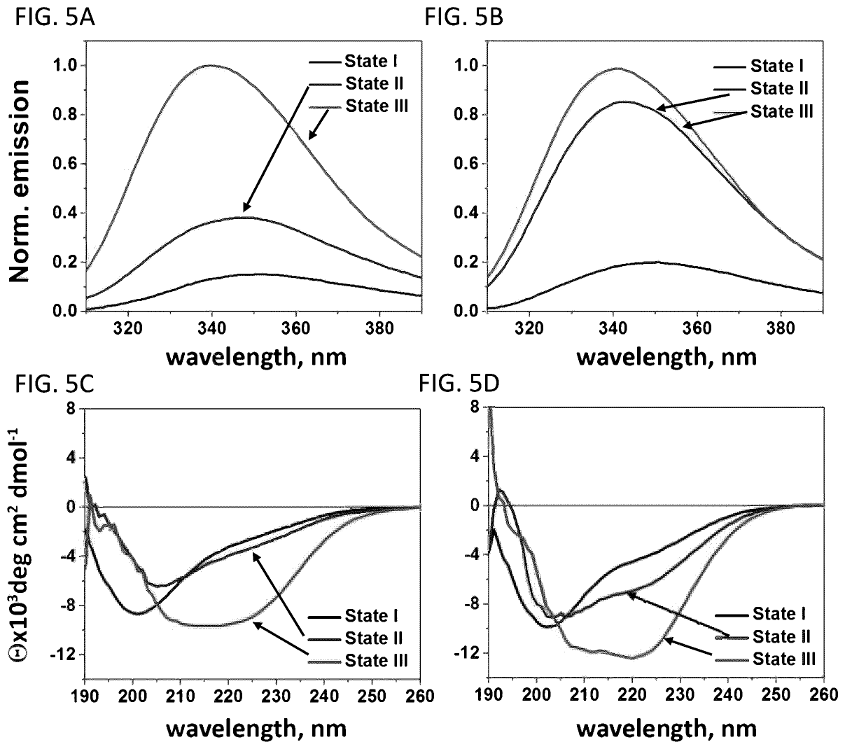

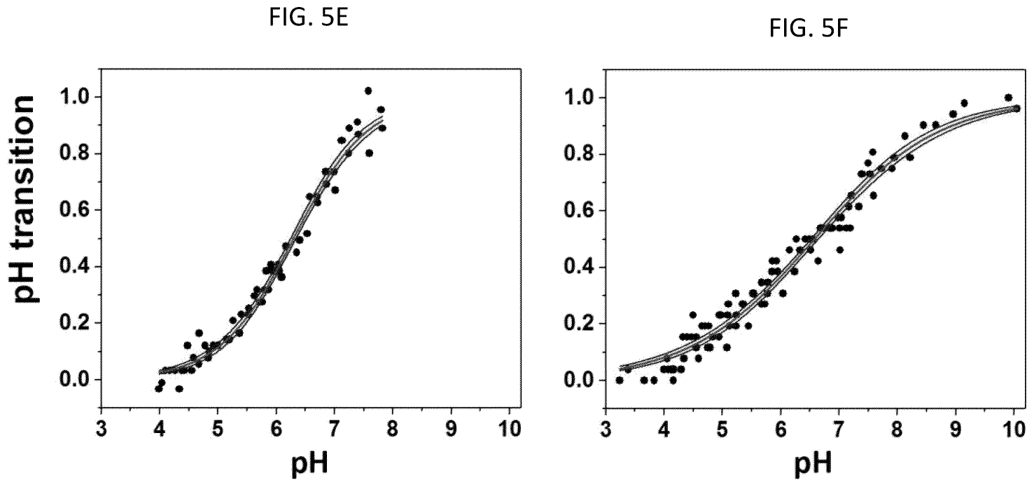

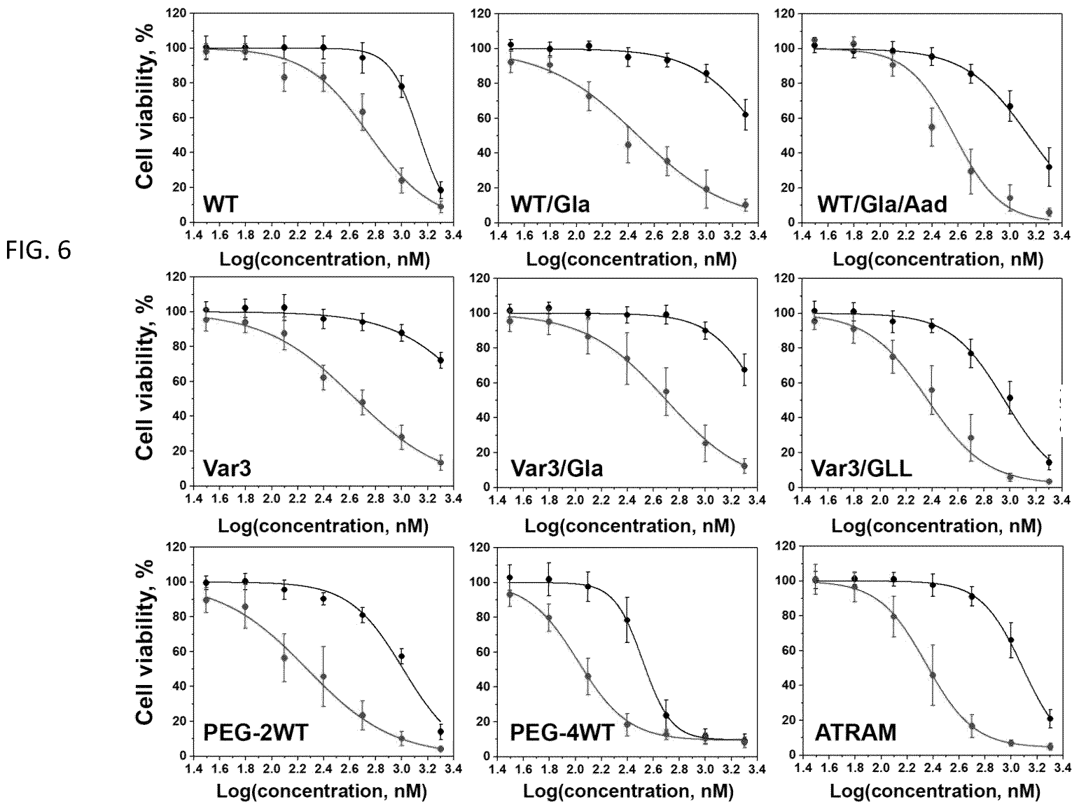

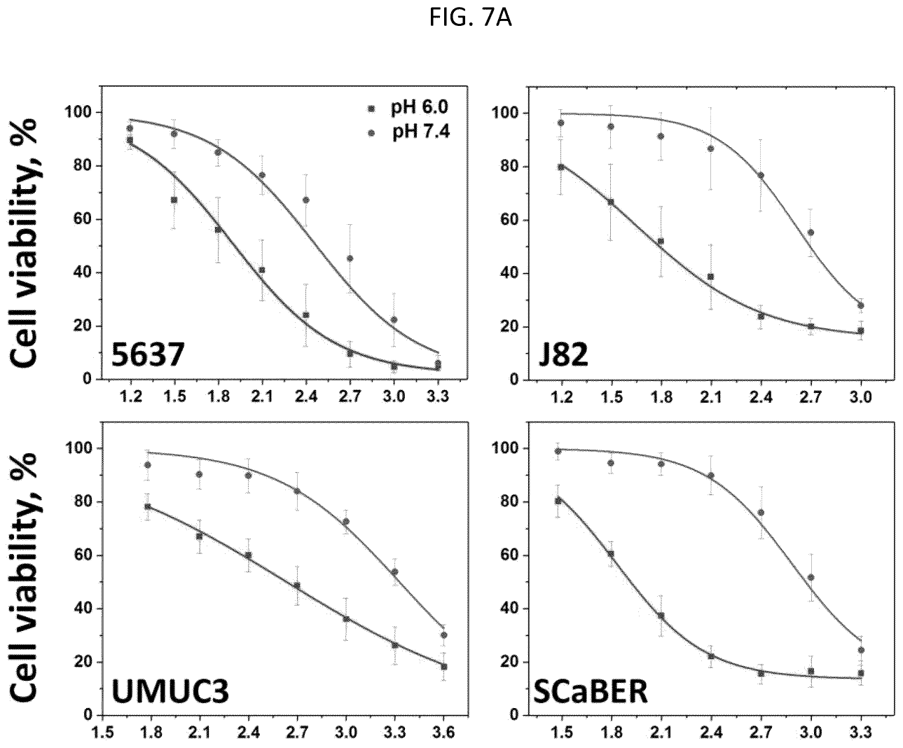

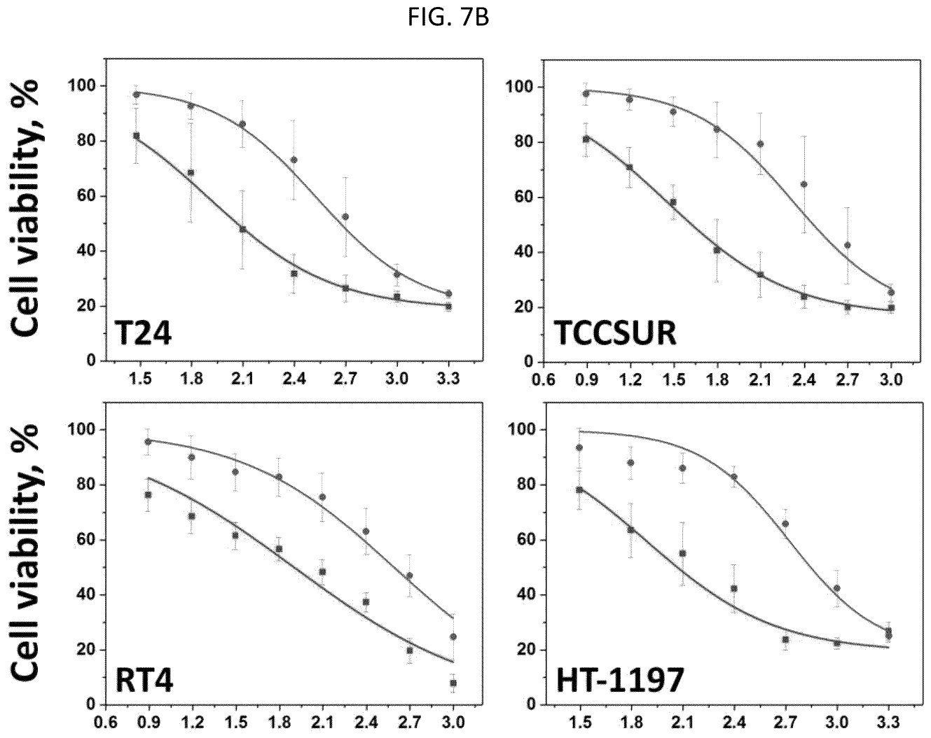

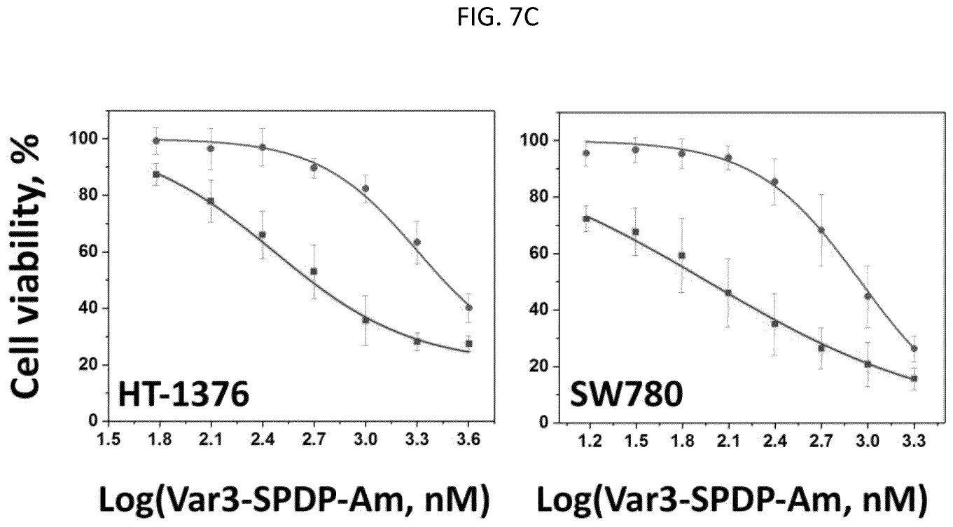

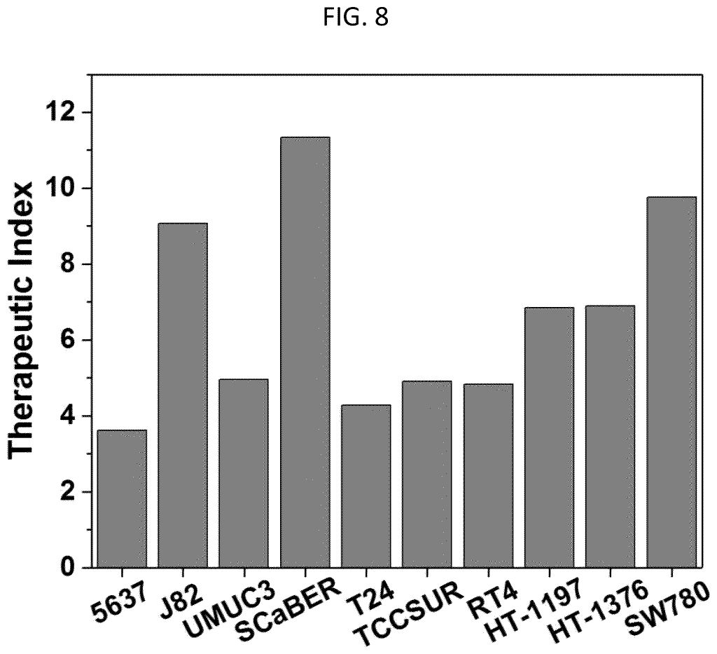

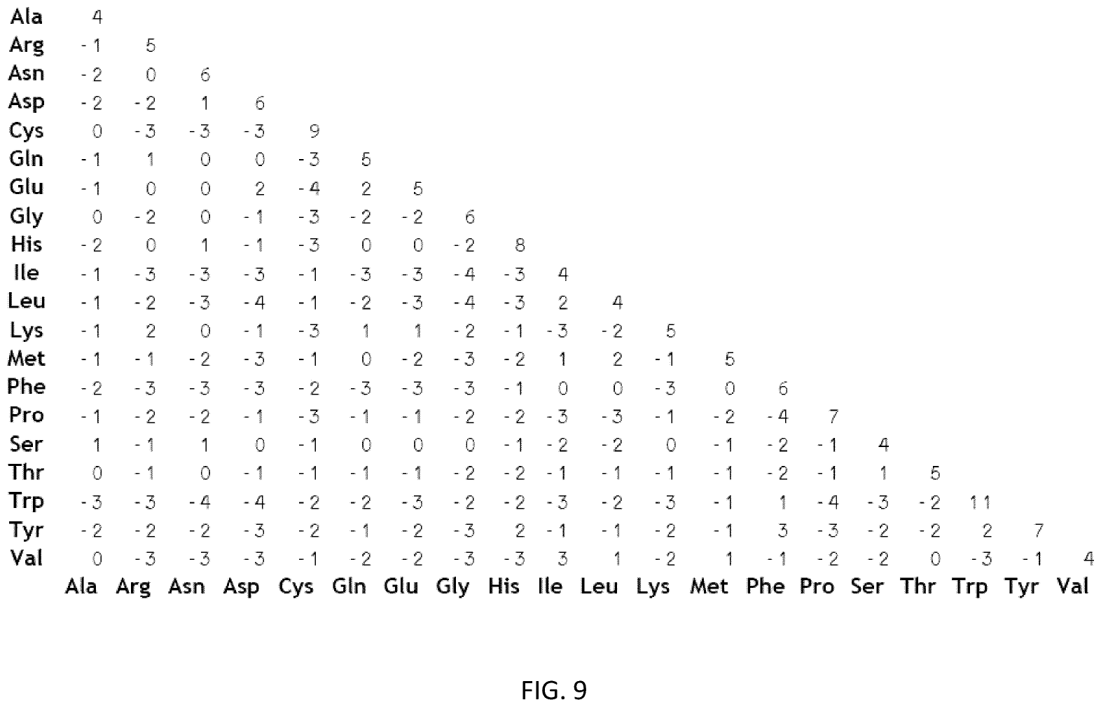

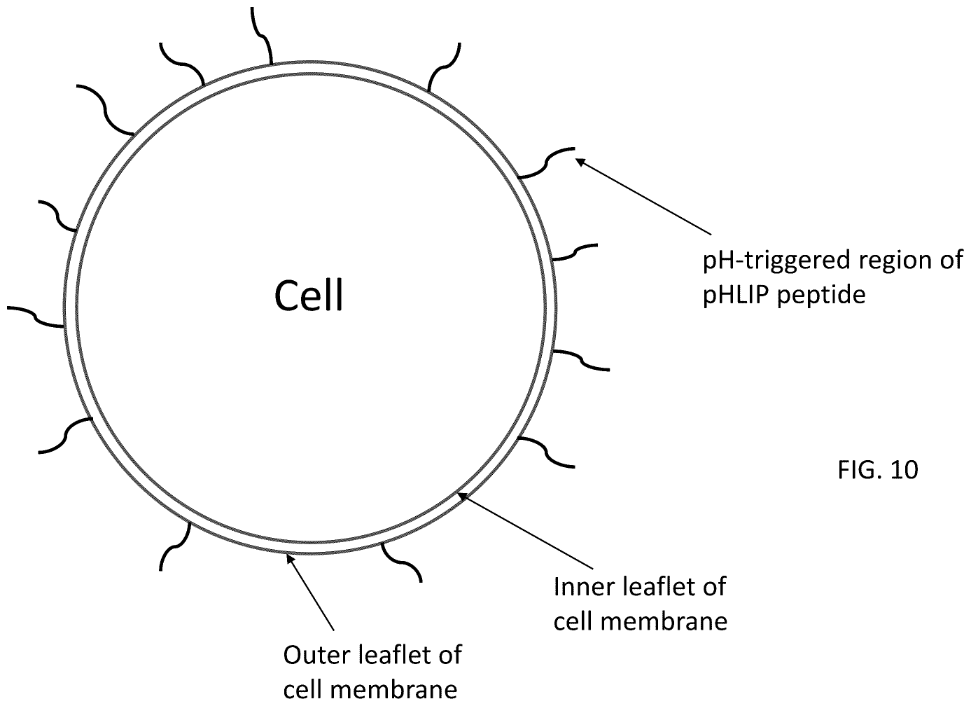

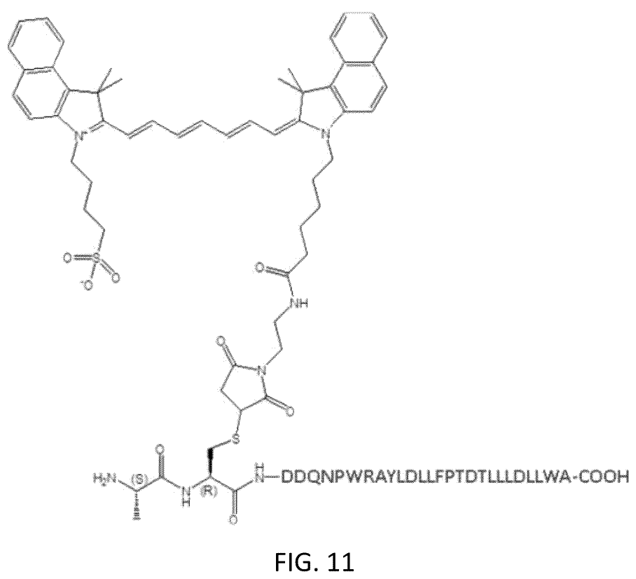

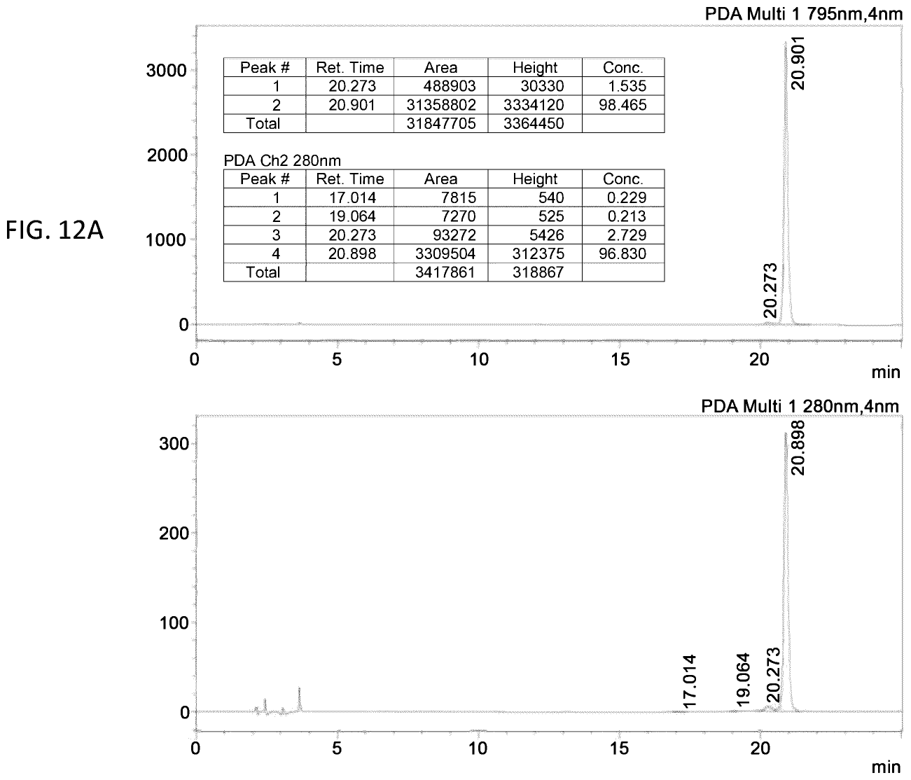

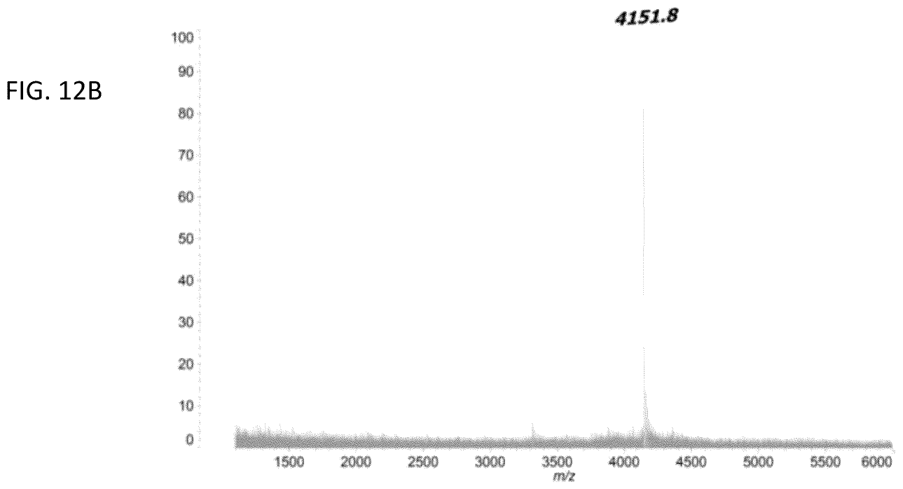

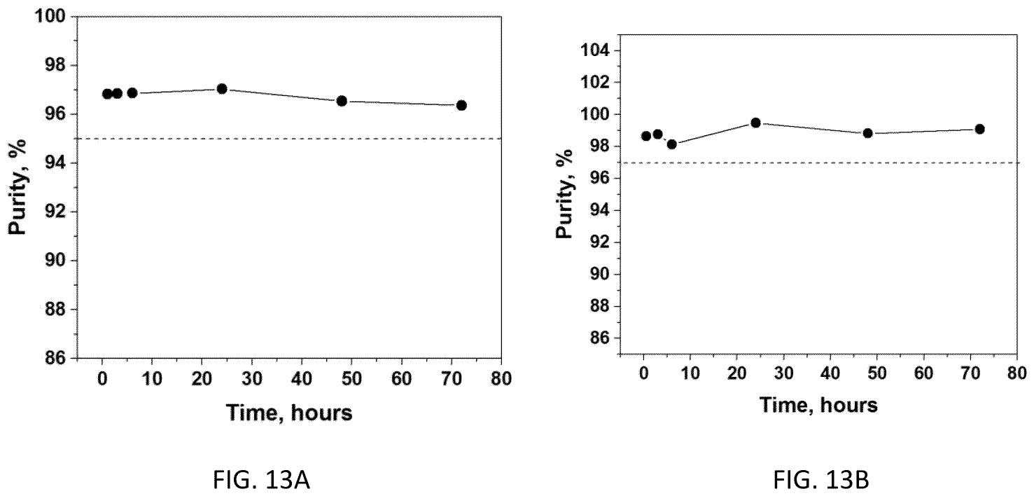

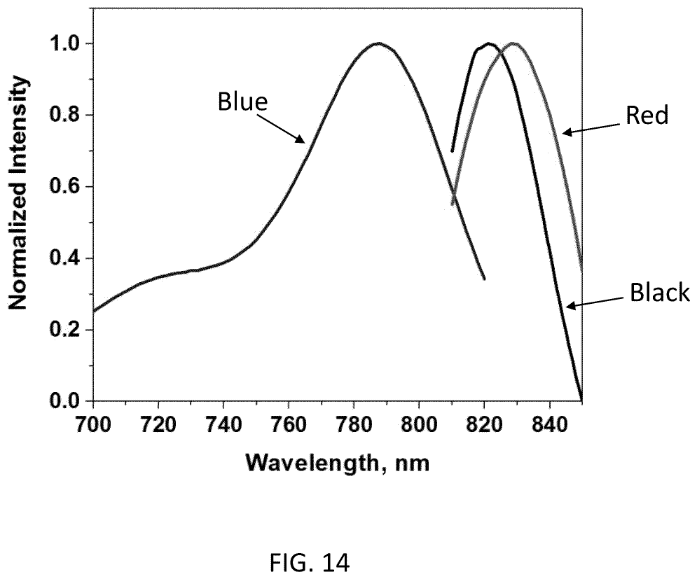

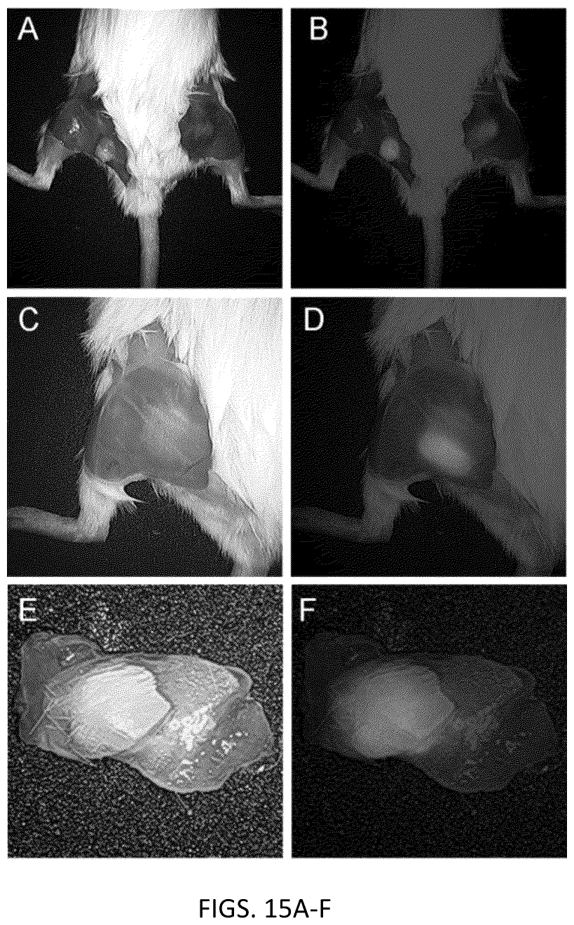

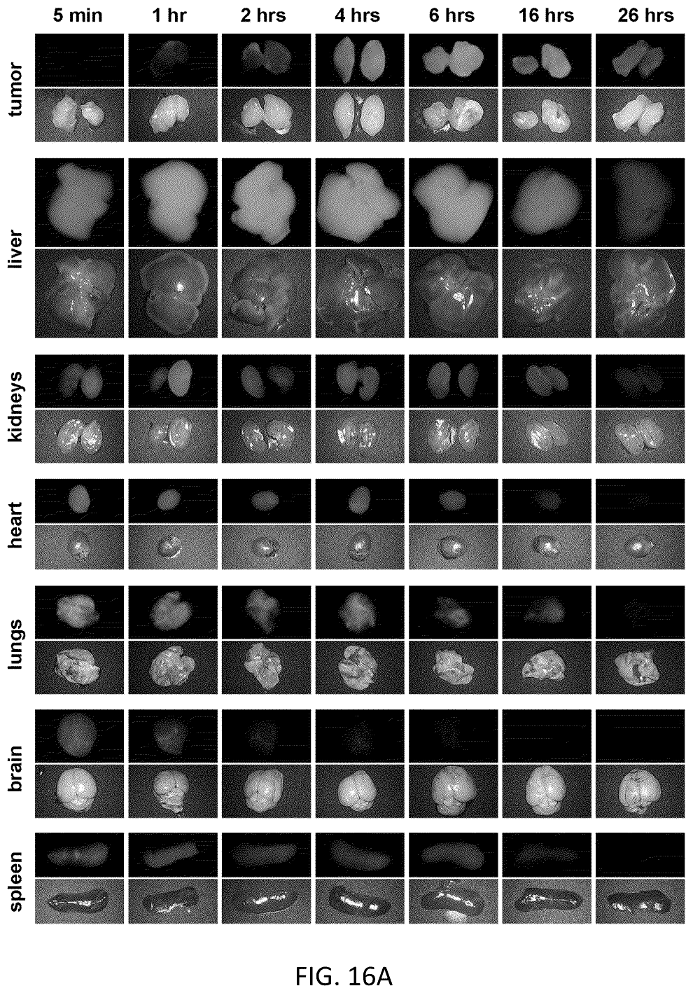

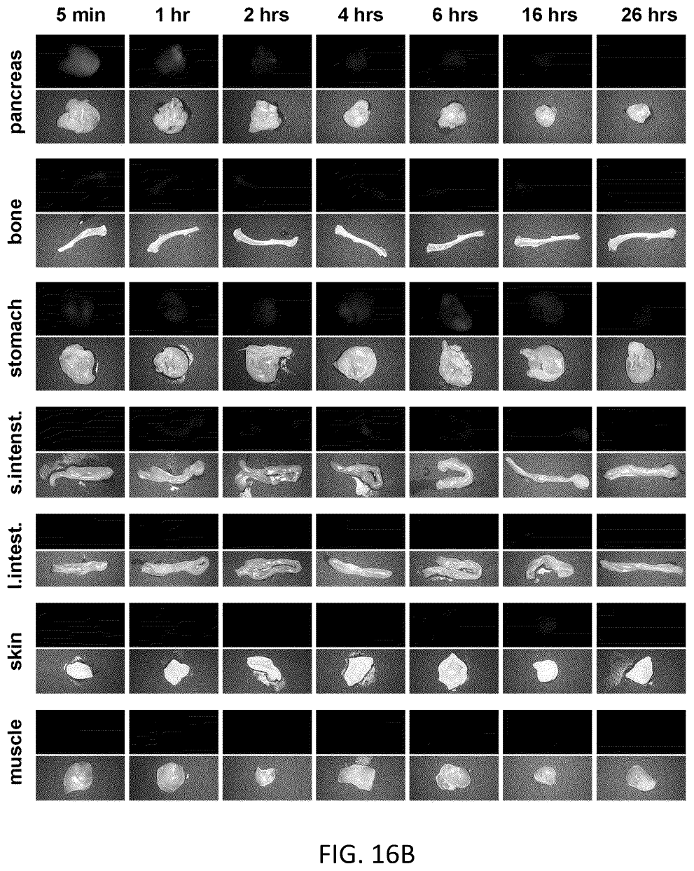

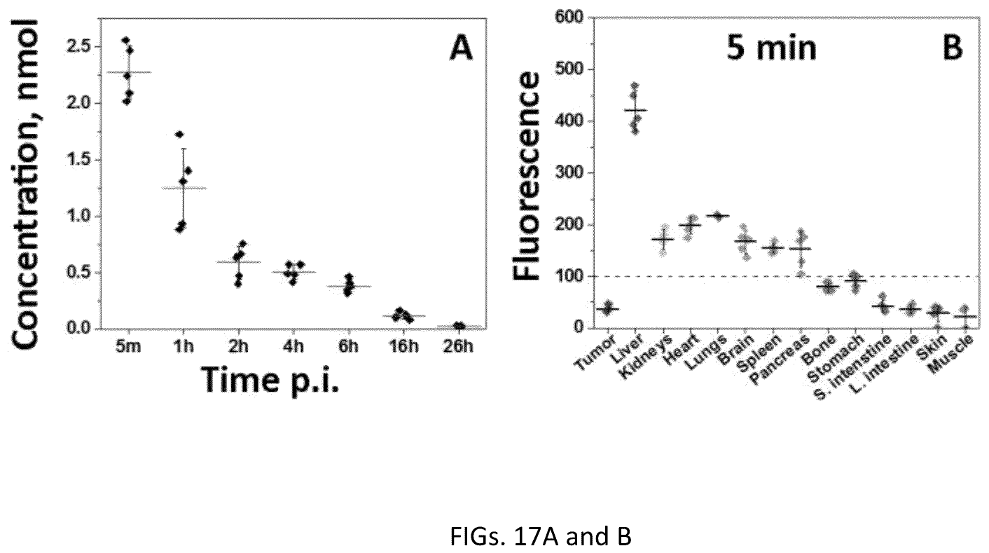

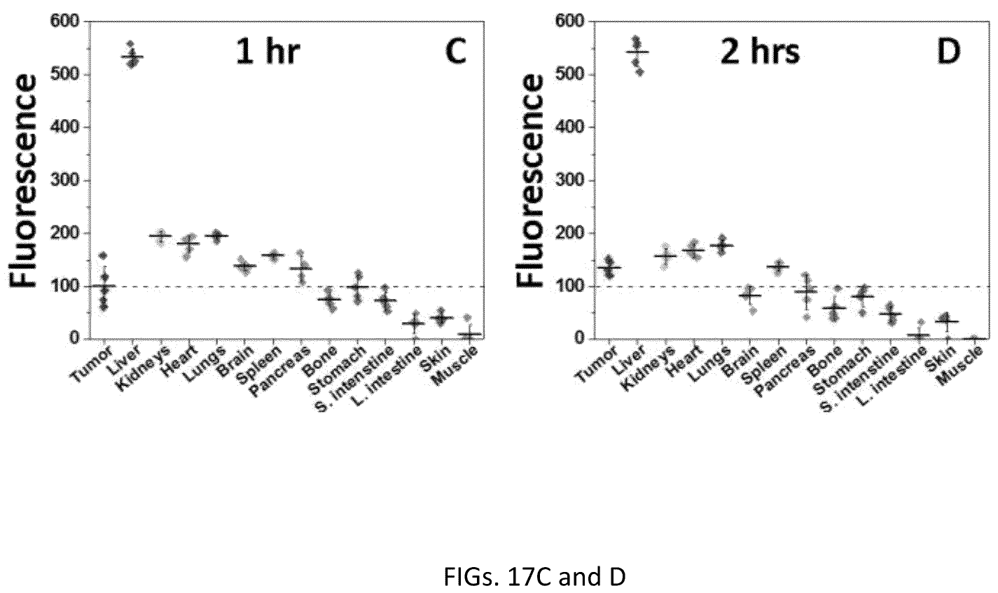

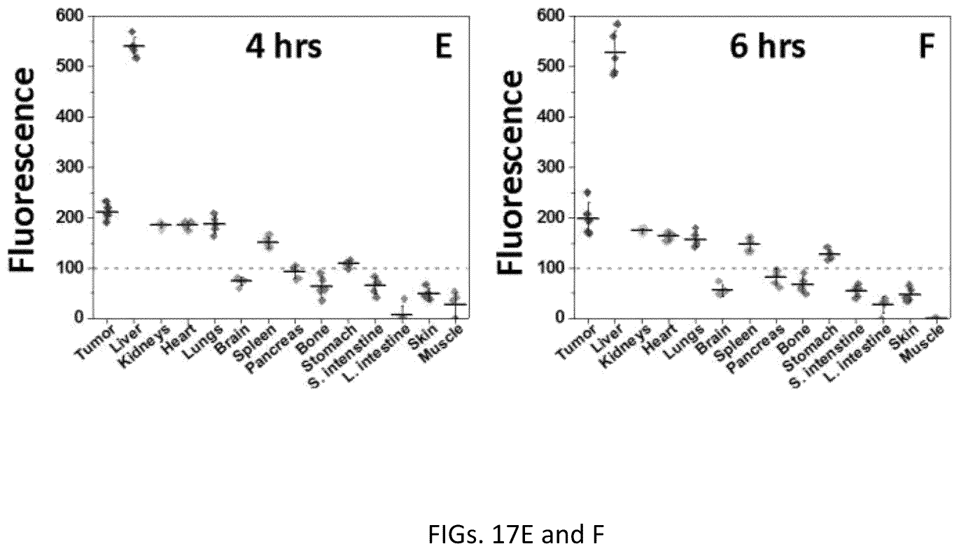

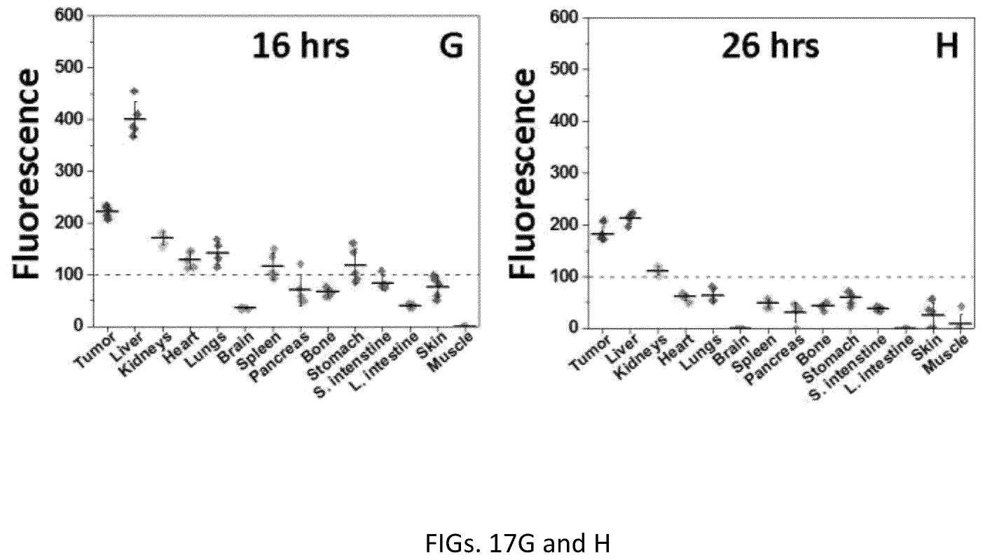

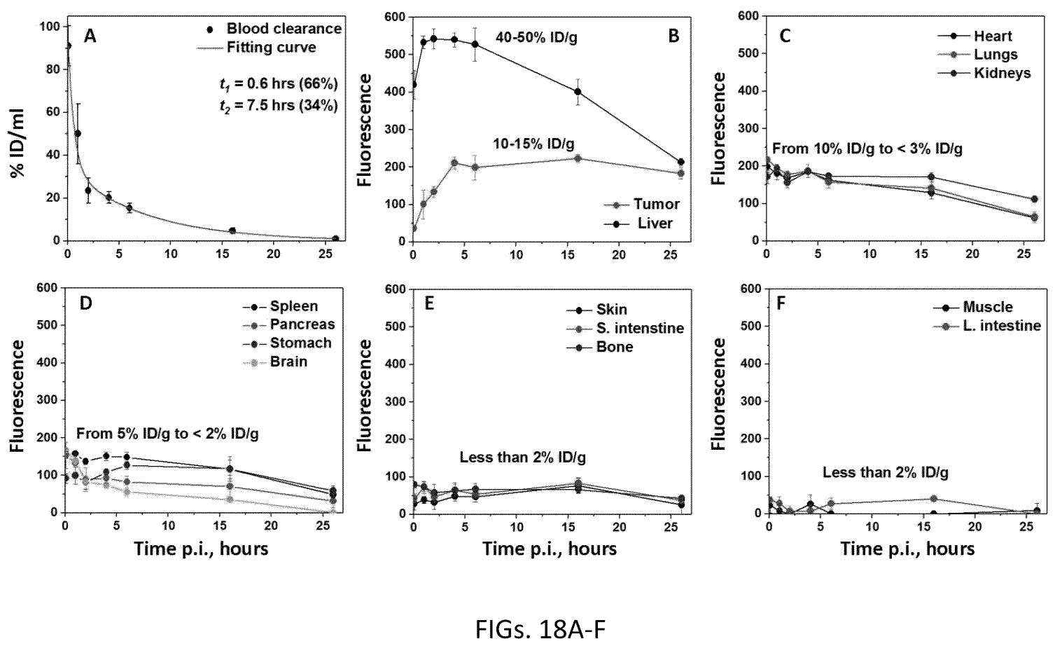

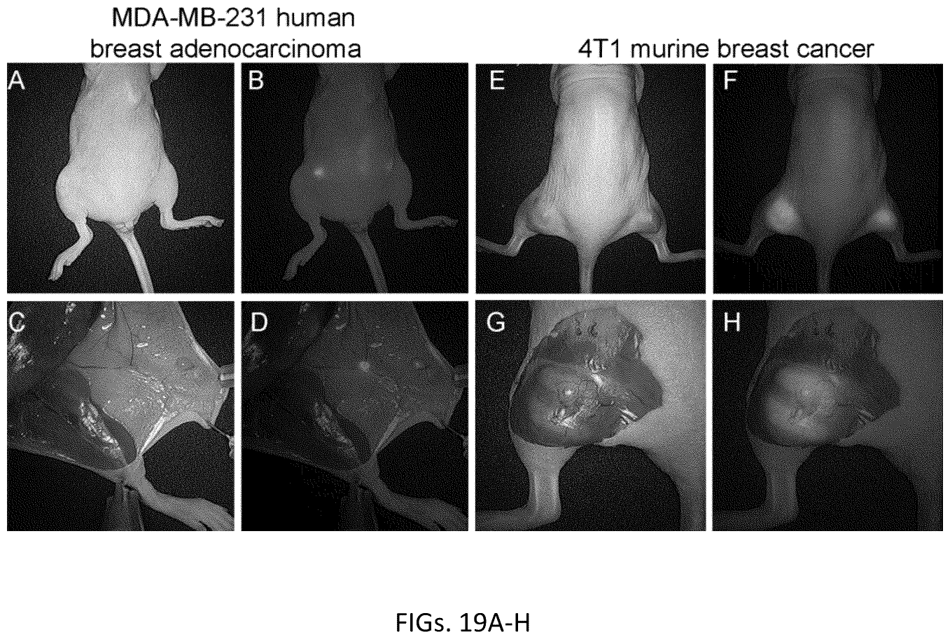

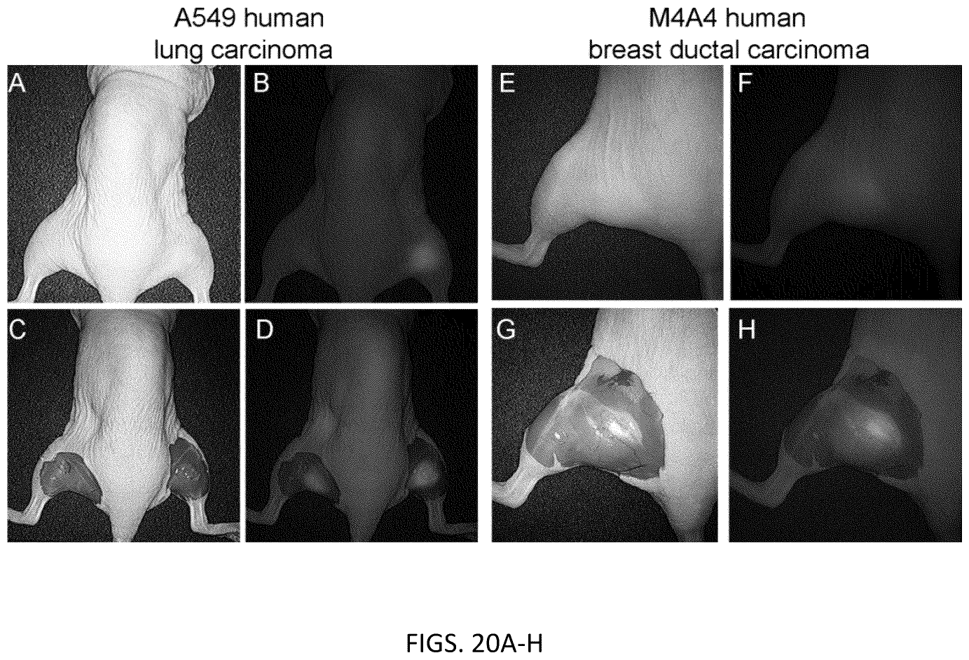

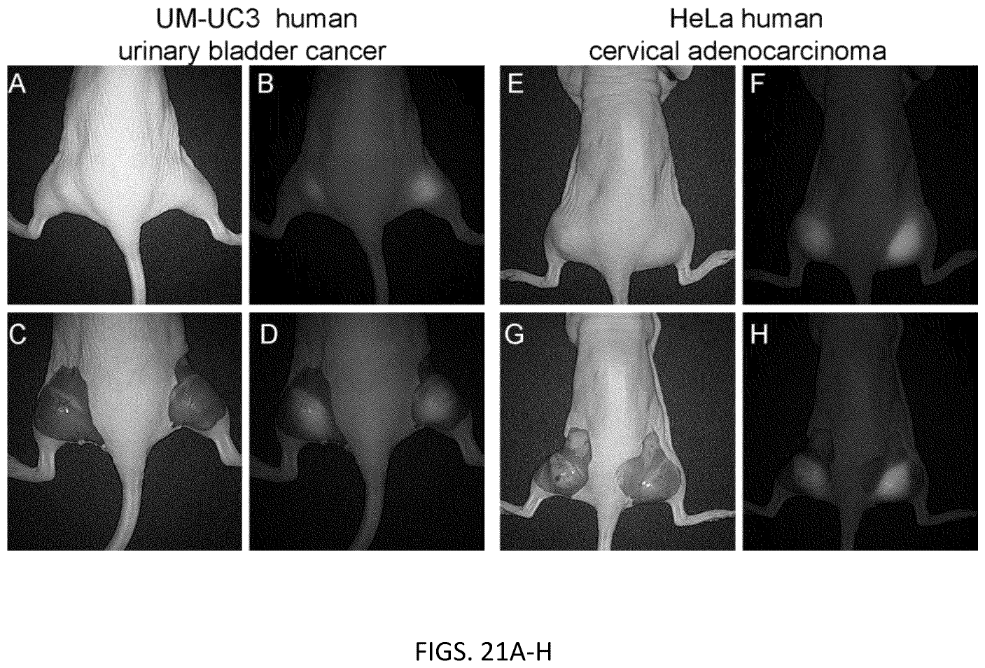

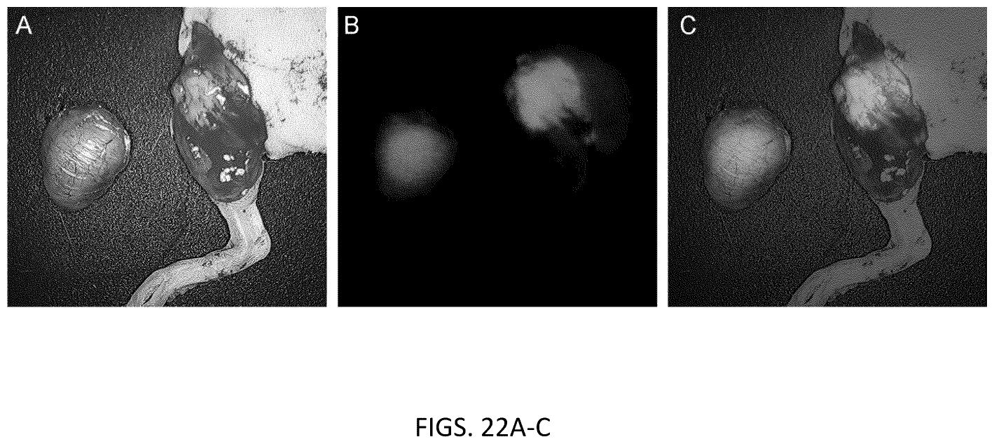

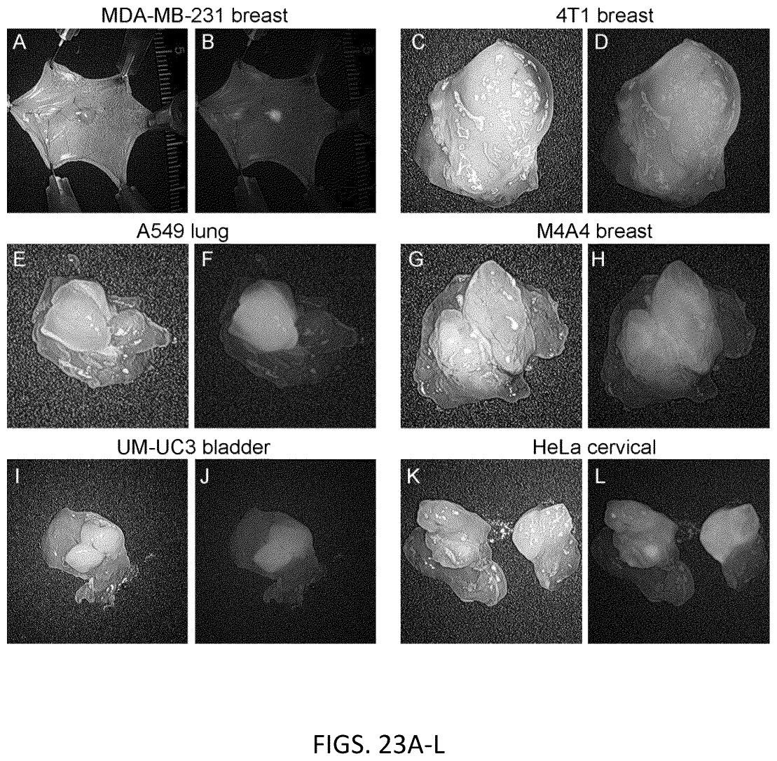

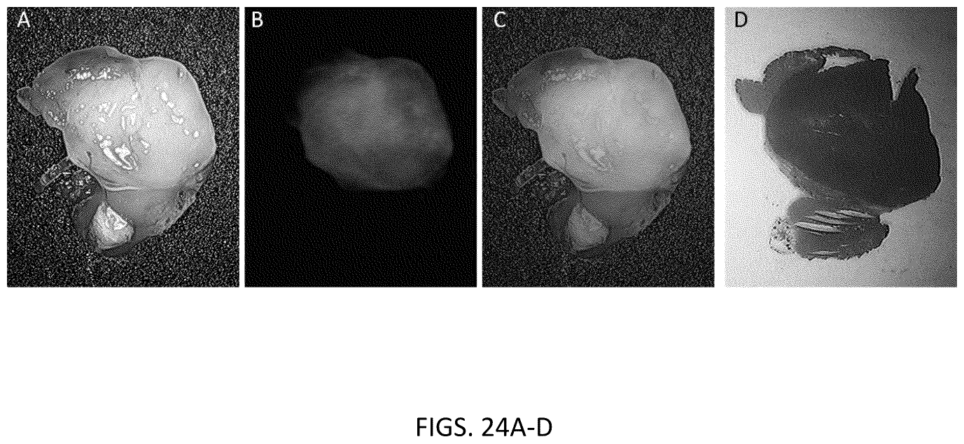

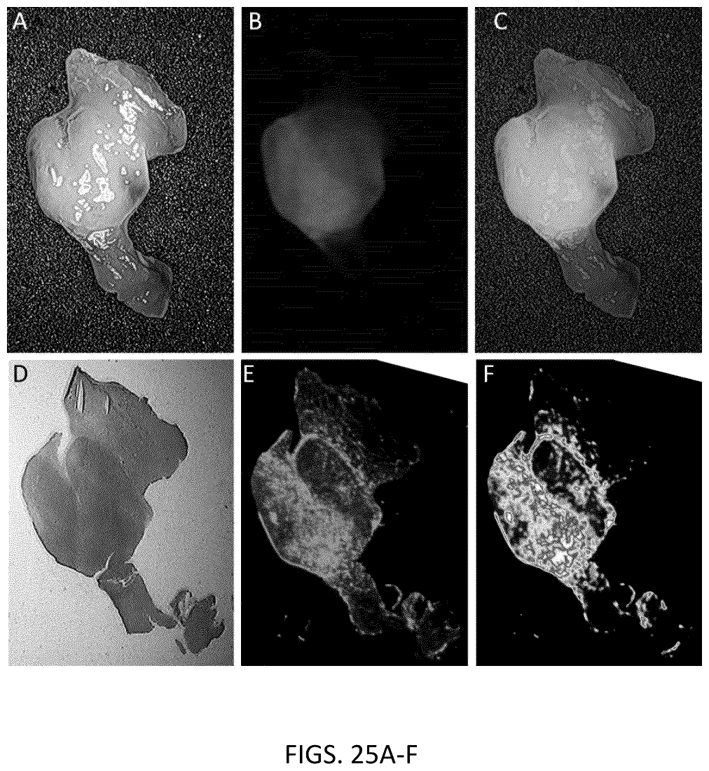

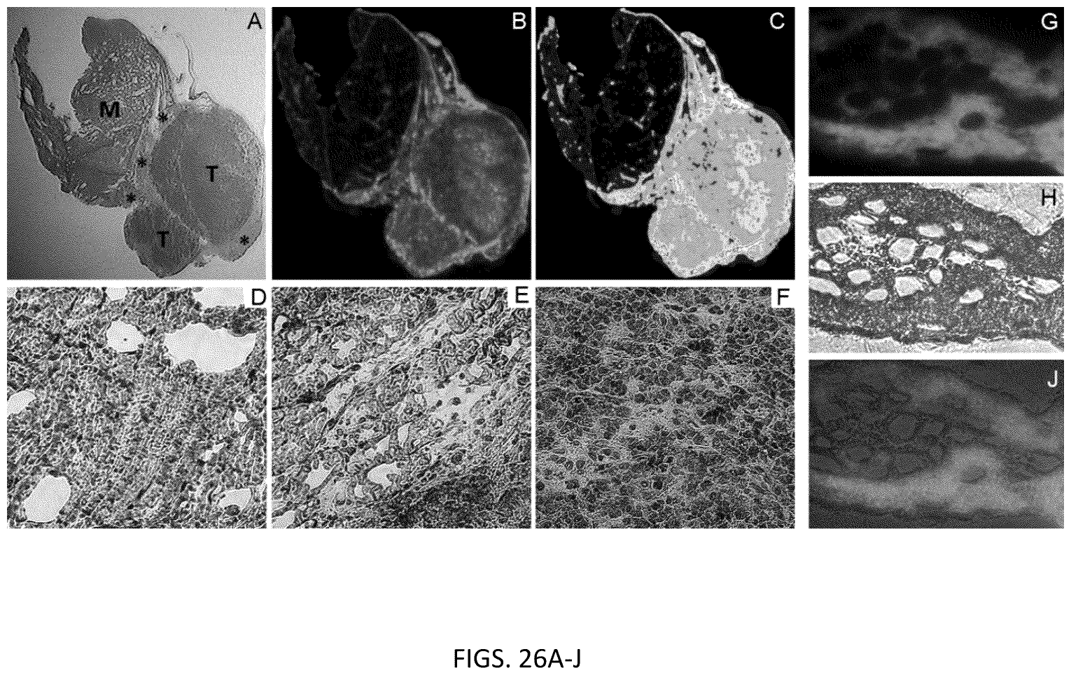

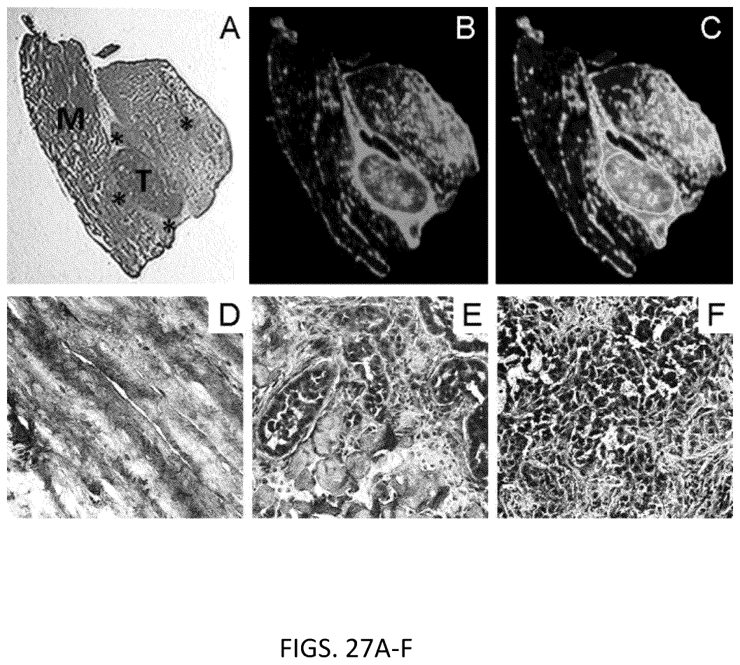

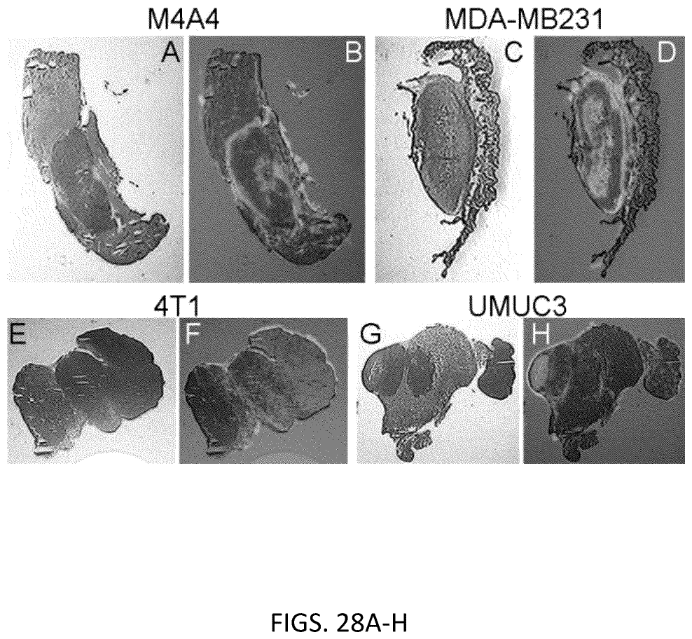

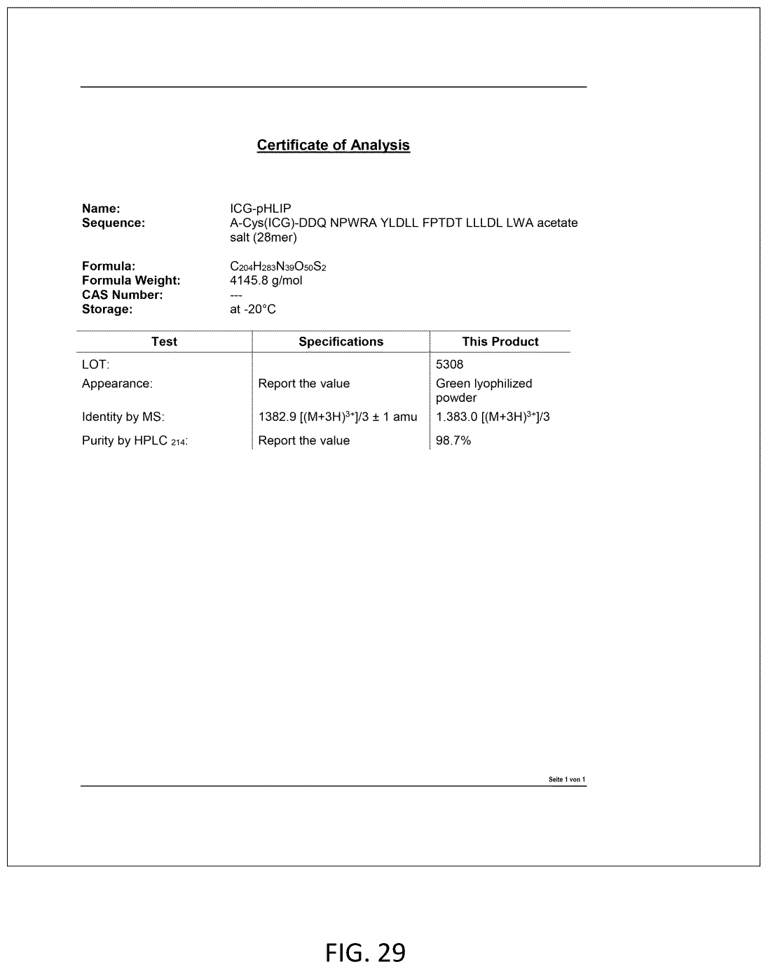

(4-Arm PEG-Azide; Four arm PEG for azido alkyne click chemistry; Creative PEGWorks Cat. No. PSB-491). FIGS. 1C-H are graphs showing transitions between the three states of PEG-2WT and PEG-4WT in phosphate buffer at pH 8 (State I), in the presence of POPC liposomes at pH 8 (State II), and in the presence of liposomes at pH 4 (State III) as monitored by changes of tryptophan fluorescence (C and D), circular dichroism (E and F), and oriented circular dichroism (OCD) (G and H) signals. FIGS. I and J are graphs showing normalized pH-dependent steady-state transitions from State II to State III as examined by analyzing the shift in position of fluorescence spectrum maximum of PEG-2WT (I) and PEG-4WT (J) in the presence of physiological concentrations of calcium and magnesium ions. The data were fitted using the Henderson-Hasselbalch equation; the fitting curves and 95% confidence interval are shown by red and blue lines, respectively.

FIG. 2A is a graph showing ellipticity ratios of CD signals at 205 nm to 222 nm for pHLIP variants in State I, II, and III. The values of ellipticity ratios are given in Table 11 (A). FIG. 2B is a graph showing the therapeutic index (TI) calculated for different pHLIP-amanitin constructs as a ratio of EC₅₀ at pH7.4 to EC₅₀ at pH6.0 (B).

FIGS. 3A and B are graphs showing potency. The pH-dependent potency was defined as the difference between cancer cell viability when cells were incubated at pH 7.4 and pH 6.0 at varying concentrations of different pHLIP-amanitin constructs. The WT-like group is shown in (A), and Var3-like group and ATRAM are shown in (B).

FIGS. 4A-D are graphs showing normalized tumor fluorescence intensities of the AF546-pHLIP constructs; the signals were normalized by the tumor intensity of AF546-WT (A). Tumor-to-muscle, T/M (B), tumor-to-kidney, T/K (C) and tumor-to-liver, T/L (D) fluorescence intensity ratios are provided. Statistically significant differences were determined by two-tailed unpaired Student's t-test, where * means p-level≤0.05 and ** means p-level≤0.005.

FIGS. 5A-F are graphs relating to transitions between the three states of Var3/Gla and Var3/GLL. Transitions between the three states of Var3/Gla (A, C, E) and Var3/GLL (B, D, F) in phosphate buffer at pH 8 (State I), in the presence of POPC liposomes at pH 8 (State II), and in the presence of liposomes at pH 4 (State III) were monitored by changes of tryptophan fluorescence (A and B) and circular dichroism (C and D) signals. Normalized pH-dependent steady-state transitions from State II to State III were examined by analyzing the shift in position of fluorescence spectrum maximum of Var3/Gla (E) and Var3/GLL (F) in the presence of physiological concentrations of calcium and magnesium ions. The data were fitted using the Henderson-Hasselbach equation; the fitting curves and 95% confidence interval are shown by inner line and outer lines, respectively.

FIG. 6 is a set of graphs showing normalized cell viability data. Normalized cell viability data (circles) presented as the logarithm of concentration (in nM) of pHLIP-amanitin constructs were fitted by the dose response function (curves) to calculate the EC₂₀, EC₅₀, EC₈₀ values presented in Table 8. Cell viability data were obtained after treatment of HeLa cells with pHLIP-amanitin constructs for 2 hours at pH 7.4 and pH 6.0, followed by removal of the constructs, transferal of cells to normal cell culture media, and assessment of cell death at 48 hours by MTS assay.

FIGS. 7A-C are sets of graphs representing normalized cell viability data vs the logarithm of concentration of pHLIP-SPDP-amanitin composition (Var3-SPDP-Am) fitted by the dose response function (curves) to calculate the EC₂₀, EC₅₀, EC₈₀ values, which are presented in Table 8.

FIG. 8 is a graph demonstrating therapeutic index (TI). The toxic effect was higher at low pH compared to normal pH in the case of all bladder cancer cell lines. The therapeutic index varied in the range from 3.6 to 11.3 with mean at 6.7±2.6. The pHLIP-SPDP-Amanitin composition could be used for the treatment of bladder cancer by intravesical instillation.

FIG. 9 is the BLOSUM62 matrix.

FIG. 10 is a diagram showing a non-limiting example of a cell that expresses pHLIP peptides on the exterior surface thereof. As shown in the diagram, pHLIP peptides (e.g., the portion of pHLIP peptides that insert into a membrane at low pH) may be presented on the outer leaflet of the cell membrane, and are not within the hydrophobic region of the cell membrane lipid bilayer.

FIG. 11 shows the structure of an exemplary ICG-pHLIP imaging agent. The exemplary Var3 pHLIP shown is a 28-mer peptide (SEQ ID NO: 15) with a free N and C terminus. The chemical structure of the first residue, Ala, and the second residues, Cys, are shown, while all other amino acids are indicated by letters. ICG (structure is shown) is linked to the Cys residue at the second position. In various embodiments, the multiple (e.g., 2-32, or 2, 3, 4, 5, 6, 7, 8, 9, 10 or more) Var3 pHLIP peptides are repeated or linked, e.g., to an ICG compound. In some embodiments, multiple (e.g., 2-32, or 2, 3, 4, 5, 6, 7, 8, 9, 10 or more) compounds as shown in this figure are linked together.

FIGS. 12A and B are HPLC chromatogram and Mass Spectrum graphs obtained for for the ICG pHLIP of FIG. 11. FIG. 12A: Analytical HPLC chromatograms recorded at 795 nm and 280 nm obtained on Zorbax SB-C18 column (4.6×250 mm, 5 μm) with the gradient of binary solvent system using water and acetonitrile with 0.05% TFA for 15-85% over 25 min. The report is inserted. FIG. 12B: Mas spectrum indicates presence of single product with expected mass (4145) plus about 6 Da of mass.

FIGS. 13A and B are graphs showing the purity of an ICG-pHLIP formulation in PBS/5% DMSO (FIG. 13A) and an ICG-pHLIP formulation in PBS/5% Ethanol (FIG. 13B) was accessed by analytical HPLC at 280 nm using Zorbax SB-C18 column (4.6×250 mm, 5 μm) with the gradient of binary solvent system using water and acetonitrile with 0.05% TFA for 15-85% over 25 min. The ICG pHLIP of FIG. 11 as used

FIG. 14 is a graph showing the absorption and fluorescence of the ICG pHLIP of FIG. 11. Normalized absorption (blue line) and fluorescence (black and red lines) are presented. Emission of ICG-pHLIP was recorded in DMSO (black line) and in PBS in presence of model POPC liposomes (red line) at excitation of 805 nm. The emission of ICG-pHLIP in PBS in absence of liposomes is negligible.

FIGS. 15A-F. are images showing the targeting murine 4T1 breast cancer. FIGS. 15A, 15C, and 15E are white light images. FIGS. 15B, 15D, and 15F are an overlay of white light and ICG-pHLIP near infrared fluorescence (NIRF) images.

FIGS. 16A and B are representative images of organs. Ex vivo imaging of organs was performed using Stryker 1588 AIM imaging system. The white light and NIRF images are shown.

FIGS. 17A-H are graphs showing blood clearance and biodistribution. FIG. 17A: Concentration of ICG-pHLIP (nmol) in blood at different time points after single IV administration of 2.5 nmol of ICG-pHLIP. FIGS. 17B-H: Mean tissue/organ fluorescence at different time points p.i. calculated from NIRF images obtained on Stryker 1588 AIM imaging system (representative images are shown on FIGS. 16A and B). The graphs represent data obtained on all 5 animals per individual time points, and open boxes represent mean and standard deviation. The values are given in Tables 16 and 17.

FIGS. 18A-F are graphs showing blood clearance and signal kinetics. FIG. 18A: blood clearance. FIGS. 18B-F: Changes of fluorescence signal in tissues/organs with time after injection. The estimation of ICG-pHLIP % ID/g was done based on the analysis of homogenized tissue and organs mixed with known concentrations of ICG-pHLIP.

FIGS. 19A-H are images showing the targeting of human breast adenocarcinoma and muring breast cancer. FIGS. 19A, 19C, 19E, and 19G: White light images. FIGS. 19B, 19D, 19F, and 19H: Overlay of white light and ICG-pHLIP NIRF images.

FIGS. 20A-H are images showing the targeting of human lung carcinoma and human breast ductal carcinoma. FIGS. 20A, 20C, 20E, and 20G: White light images. FIGS. 20B, 20D, 20F, and 20H: Overlay of white light and ICG-pHLIP NIRF images.

FIGS. 21A-H are images showing the targeting of human urinary bladder cancer and human cervical adenocarcinoma. FIGS. 21A, 21C, 21E, and 21G: White light images. FIGS. 21B, 21D, 21F, and 21H: Overlay of white light and ICG.

FIGS. 22A-C are images showing incomplete surgery. White light image (FIG. 22A), ICG-pHLIP NIRF image (FIG. 22B) and overlay of white light and ICG-pHLIP NIRF images (FIG. 22C).

FIGS. 23A-L are images showing the ex vivo imaging of tumors excised with surrounding muscle. FIGS. 23A, 23C, 23E, 23G, 23I, and 23K: White light images. FIGS. 23B, 23D, 23F, 23H, 23J, and 23L: Overlay of white light and ICG-pHLIP NIRF images.

FIGS. 24A-D are images showing the correlation of ICG-Var3 NIRF signal with H&E histopathology. White light (FIG. 24A), ICG-Var3 NIRF (FIG. 24B) and overlay of white light and ICG-pHLIP NIRF (FIG. 24C) images obtained using Stryker imaging system are shown together with tumor sections stained with H&E (FIG. 24D).

FIGS. 25A-F are images showing the correlation of ICG-Var3 NIRF signal with H&E histopathology. White light (FIG. 25A), ICG-pHLIP NIRF (FIG. 25B) and overlay of white light and ICG-pHLIP NIRF (FIG. 25C) images of 4T1 breast tumor obtained using Stryker imaging system are shown together with tumor sections stained with H&E (FIG. 25D) and adjacent tumor section presented in black/green (FIG. 25E) & 16-color scheme (from blue to red and white as the highest intensity) (FIG. 25F) obtained on Li-Cor scanner.

FIGS. 26A-J are images of tumor sections. H&E image of HeLa tumor section (FIG. 26A) and ICG-pHLIP NIRF images of adjacent HeLa tumor section presented in black/green (FIG. 26B) & 16-color scheme (from blue to red and white as the highest intensity) (FIG. 26C) obtained on Li-Cor are shown. Magnified view of different parts of H&E section: muscle, M (FIG. 26D); tissue surrounding tumor marked by stars (*) on panel A (FIG. 26E); and main tumor mass, T (FIG. 26F). The magnified ICG-pHLIP NIRF image (FIG. 26G), bright field image (FIG. 26H) and overlay of fluorescence and bright field images (FIG. 26J) were obtained under a fluorescent inverted microscope with objective 40×.

FIGS. 27A-F are images of tumor sections. H&E image of A549 tumor section (FIG. 27A) and ICG pHLIP® NIRF images of adjacent A549 tumor section presented in black/green (FIG. 27B) & 16-color scheme (from blue to red and white as the highest intensity) (FIG. 27C) obtained on Li-Cor are shown. Magnified view of different parts of H&E section: muscle, M (FIG. 27D); tissue surrounding tumor marked by stars (*) on panel A (FIG. 27E); and main tumor mass, T (FIG. 27F).

FIGS. 28A-H are images of tumor sections. H&E images (FIGS. 28A, 28C, 28E, and 28G) and overlay of H&E and ICG-pHLIP NIRF images of adjacent tumors section obtained on Li-Cor scanner are shown.

FIG. 29 is a certificate of analysis for ICG-pHLIP (SEQ ID NO: 15).

### DETAILED DESCRIPTION

The present subject matter provides, inter alia, pH-triggered compounds and compositions comprising one or more peptides that are capable of inserting into a lipid bilayer below a certain pH (e.g., one or more pH-triggered polypeptides). A pH-triggered polypeptide (pHLIP peptides, also known as “pH-triggered pH (Low) Insertion Peptides”) is a water-soluble membrane peptide that interacts weakly with a cell membrane at neutral pH, without insertion into the lipid bilayer, but inserts into the cell membrane and forms a stable transmembrane alpha-helix at acidic pH (e.g., at a pH of less than about 7.0, 6.75, 6.5, 6.25, 6, 5.75, 5.5, 5.25, 5.0, 4.75, 4.5, 4.25, 4.0, 3.75, 3.5, 3.25, or 3.0). Treatment, imaging, diagnostic, and other uses of such compounds and compositions are also provided.

A compound is pH-triggered if it has, e.g., a higher affinity to a membrane lipid bilayer at pH 5.0 compared to at pH 8.0. In some embodiments, a pH-triggered compound is or includes a peptide, which may optionally be attached to a cargo compound. In certain embodiments, a pH-triggered compound comprises multiple peptides and, e.g., a linker and/or one or more cargo compounds.

Included herein are improved pHLIP peptides, as well as compounds comprising multiple pHLIP peptides (e.g., linked pHLIPs and pHLIP bundles). As used herein, a pHLIP bundle is a compound comprising at least two pHLIP peptides. For example, a pHLIP bundle includes 2, 3, 4, 5, 6, or more individual pHLIP peptides covalently linked to one another. In various embodiments, the pHLIP peptides are covalently linked directly (e.g., via a covalent bond) or indirectly (e.g., via a linker moiety to which each of the pHLIP peptides are covalently bound). In certain embodiments, the pHLIP peptides are not within the same stretch of amino acids. In some embodiments, assembling pHLIP peptides into bundles (e.g., by linking them together) alters the pH-dependent intracellular delivery of molecules (i.e., cargo compounds) and targeting of acidic diseased tissue, such as cancer. Non-limiting examples of pH-triggered compounds include pHLIP peptides containing standard and/or non-standard amino acids, as well as conjugates (bundles) thereof comprising 2, 3, 4, or more pHLIP peptides linked together by, e.g., polyethelyne glycol. Non-limiting data provided herein correlates the biophysical properties of the membrane interactions of different pHLIP peptides and their bundles to the ability of these constructs to move polar cargo (e.g., cyclic cell-impermeable peptide, mushroom toxin, amanitin) across the cell membrane and to target acidic tumors. pHLIP peptides assembled into the bundles demonstrated surprising new properties of pH-dependent interactions with lipid bilayer of membrane, which led to the enhancement of intracellular delivery of molecules into cancer cells.

In various embodiments, a pH-triggered compound (e.g., a peptide such as a pHLIP peptide, or a compound comprising multiple pHLIP peptides) has a net neutral charge at a low pH and a net negative charge at a neutral or high pH. In some embodiments, a pH-triggered compound has a net neutral charge at a pH of less than about 7, 6.5, 6.0, 5.5, 5.0, 4.5, or 4.0 and a net negative charge at a pH of about 7, 7.25, 7.5, or 7.75 in water, e.g., distilled water. In certain embodiments, a pH-triggered compound has a net neutral charge at a pH of less than about 7 and a net negative charge at a pH of about 7 in water. In some embodiments, a pH-triggered compound has a net neutral charge at a pH of less than about 6.5 and a net negative charge at a pH of about 7 in water. In various embodiments, a pH-triggered compound has a net neutral charge at a pH of less than about 6.0 and a net negative charge at a pH of about 7. In some embodiments, a pH-triggered compound has a net neutral charge at a pH of less than about 5.5 and a net negative charge at a pH of about 7 in water. In certain embodiments, a pH-triggered compound has a net neutral charge at a pH of less than about 5.0 and a net negative charge at a pH of about 7 in water. In various embodiments, a pH-triggered compound has a net neutral charge at a pH of less than about 4.5 and a net negative charge at a pH of about 7 in water. In some embodiments, a pH-triggered compound has a net neutral charge at a pH of less than about 4.0 and a net negative charge at a pH of about 7 in water.

In various embodiments, a pH-triggered compound that comprises multiple pHLIP peptides may comprise any pHLIP peptide (or any combination thereof) disclosed herein.

In some embodiments, a pHLIP peptide monomer or a compound comprising multiple pHLIP peptides has a net negative charge at a pH of about 7, 7.25, 7.5, or 7.75 in water. Alternatively or in addition, the pHLIP peptide or compound comprising multiple pHLIP peptides may have an acid dissociation constant at logarithmic scale (pKa) of less than about 4.0, 4.5, 5.0, 5.5, 6.0, 6.5, or 7.

In various embodiments, a protonatable amino acid is an amino acid with a pKa of less than about 4.0, 4.5, 5.0, 5.5, 6.0, 6.5, or 7. In certain embodiments, a protonatable amino acid is an amino acid with a pKa of less than about 6.5. In some embodiments, a protonatable amino acid is an amino acid with a pKa of less than about 5.5. In certain embodiments, a protonatable amino acid is an amino acid with a pKa of less than about 4.5. In various embodiments, a protonatable amino acid is an amino acid with a pKa of less than about 4.0. In some embodiments, a protonatable amino acid comprises a carboxyl group.

Aspects of the present subject matter relate to pHLIP peptides of various sizes. For example, a pHLIP peptide may have 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 45, 46, 47, 48, 49, 50 or more amino acids; 8 to 15 amino acids; 8 to 50 amino acids; 8 to 40 amino acids; 8 to 30 amino acids; 8 to 20 amino acids; 8 to 10 amino acids; less than about 20 amino acids; less than 9, 10, 11, 12, 13, 14, or 15 amino acids; 10 amino acids; 9 amino acids, or 8 amino acids. In some embodiments, less than 1, 2, 3, 4, or 5 of the amino acids in the pHLIP peptide have a net positive charge at a pH of 7, 7.25, 7.5, or 7.75 in water. In certain embodiments, the pHLIP peptide comprises 0 amino acids having a net positive charge at a pH of about 7, 7.25, 7.5, or 7.75 in water.

In various implementations of the present subject matter, a pH-triggered compound has a functional group (e.g., 1 or more functional groups) to which a cargo compound may be attached. In a non-limiting example, the functional group is a side chain of an amino acid of the pH-triggered compound. In certain embodiments, the functional group is an amino acid side chain to which a cargo compound may be attached via a disulfide bond. In some embodiments, the functional group to which a cargo compound may be attached comprises a free sulfhydryl (SH) or selenohydryl (SeH) group. For example, a functional group may be present within a sidechain of a cysteine, homocysteine, selenocysteine, or homoselenocysteine, or a derivative thereof having at least one, e.g., 1, 2, 3, 4, 5, or more, free SH and/or SeH groups. In various embodiments, the functional group comprises a primary amine. For example, a functional group may be present within a sidechain of a lysine or a derivative thereof having at least one, e.g., 1, 2, 3, 4, 5, or more, primary amines.

In certain embodiments, a pHLIP peptide has about 1, 2, 3, 4, 5, 6, 7, 8, 9, 10 or more aromatic amino acids. For example, the aromatic amino acids may be one or more of a tryptophan, a tyrosine, a phenylalanine, and an artificial aromatic amino acid.

pHLIP peptides of the present subject matter have at least 1 protonatable amino acid. For example, a pHLIP peptide may comprise 1 protonatable amino acid which is aspartic acid, glutamic acid, or gamma-carboxyglutamic acid; or at least 2, 3, 4, 5, 6, 7, 8, 9, 10 or more protonatable amino acids, wherein the protonatable amino acids comprise one or more of aspartic acid, glutamic acid, and gamma-carboxyglutamic acid. In some embodiments, the protonatable amino acid is an artificial amino acid. In a non-limiting example, a pHLIP peptide has at least 2, 3, 4, 5, 6, 7, 8, 9, 10 or more protonatable amino acids, wherein the protonatable amino acids comprise aspartic acid, glutamic acid, gamma-carboxyglutamic acid, or any combination thereof.

Aspects of the present subject matter provide pHLIP peptides having artificial amino acids, such as at least 1 artificial protonatable amino acid. In various embodiments, the artificial protonatable amino acid comprises at least 1, 2, 3, 4 or 5 carboxyl groups and/or the pHLIP peptide may have at least 1, 2, 3, 4, 5, 6, 7, 8, 9, 10, 11, 12, 13, 14, or 15 carboxyl groups. In some embodiments, a pHLIP peptide has at least 1, 2, 3, 4, 5, 6, 7, 8, 9, 10, 11, 12, 13, 14, 15, 16, 17, 18, 19, or 20 artificial amino acids. In a non-limiting example, every amino acid of the pHLIP peptide is an artificial amino acid. In certain embodiments, a pHLIP peptide may include at least 1, 2, 3, 4, 5, 6, 7, 8, 9, 10, 11, 12, 13, 14, 15, 16, 17, 18, 19, or 20 D-amino acids.

Various implementations of the present subject matter relate to pHLIP peptides having at least one artificial amino acid which is a cysteine derivative, an aspartic acid derivative, a glutamic acid derivative, a phenylalanine derivative, a tyrosine derivative, or a tryptophan derivative. For example, a pHLIP peptide may contain a cysteine derivative selected from the group consisting of D-Ethionine, Seleno-L-cystine, S-(2-Thiazolyl)-L-cysteine, and S-(4-Tolyl)-L-cysteine; an aspartic acid derivative which is a N-phenyl(benzyl)amino derivative of aspartic acid; a glutamic acid derivative selected from the group consisting of γ-Carboxy-DL-glutamic acid, 4-Fluoro-DL-glutamic acid, and (4S)-4-(4-Trifluoromethyl-benzyl)-L-glutamic acid; a phenylalanine derivative selected from the group consisting of (S)—N-acetyl-4-bromophenylalanine, N-Acetyl-2-fluoro-DL-phenylalanine, N-Acetyl-4-fluoro-DL-phenylalanine, 4-Chloro-L-phenylalanine, DL-2,3-Difluorophenylalanine, DL-3,5-Difluorophenylalanine, 3,4-Dihydroxy-L-phenylalanine, 3-(3,4-Dimethoxyphenyl)-L-alanine, 4-(Hydroxymethyl)-D-phenylalanine, N-(3-Indolylacetyl)-L-phenylalanine, p-Iodo-D-phenylalanine, α-Methyl-DL-phenylalanine, 4-Nitro-DL-phenylalanine, and 4-(Trifluoromethyl)-D-phenylalanine; a tyrosine derivative selected from the group consisting of α-Methyl-DL-tyrosine, 3-Chloro-L-tyrosine, 3-Nitro-L-tyrosine, and DL-o-Tyrosine; and/or a tryptophan derivative selected from the group consisting of 5-Fluoro-L-tryptophan, 5-Fluoro-DL-tryptophan, 5-Hydroxy-L-tryptophan, 5-Methoxy-DL-tryptophan, or 5-Methyl-DL-tryptophan.

In various embodiments, a pHLIP peptide has at least 8 consecutive amino acids, wherein, at least 2, 3, 4, 5, or 6 of the 8 consecutive amino acids of the pHLIP peptide are non-polar, and at least 1 or 2 of the at least 8 consecutive amino acids of the pHLIP peptide is protonatable. For example, the pHLIP peptide may have 8-10 consecutive amino acids, including at least 2, 3, 4, 5, or 6 amino acids that are non-polar, and at least 1 or 2 amino acids that are protonatable.

Aspects of the present disclosure provide pHLIP peptides that are linked together and/or to a cargo compound. In various implementations, the pHLIP peptide is directly linked to a linker compound, another pHLIP peptide, and/or a cargo compound by a covalent bond. In some non-limiting examples, the covalent bond is an ester bond, a disulfide bond, a bond between two selenium atoms, a bond between a sulfur and a selenium atom, or an acid-liable bond.

In some embodiments, the covalent bond between the pHLIP peptide, a linker compound, another pHLIP peptide, and/or and the cargo compound is a bond that has been formed by a click reaction. Non-limiting examples of click reactions include reactions between an azide and an alkyne; an alkyne and a strained difluorooctyne; a diaryl-strained-cyclooctyne and a 1,3-nitrone; a cyclooctene, trans-cycloalkene, or oxanorbornadiene and an azide, tetrazine, or tetrazole; an activated alkene or oxanorbornadiene and an azide; a strained cyclooctene or other activated alkene and a tetrazine; or a tetrazole that has been activated by ultraviolet light and an alkene.

Some implementations provide a pHLIP peptide that is attached to a linker compound by a covalent bond, wherein the linker compound is attached to the cargo compound or another pHLIP peptide (or, e.g., each of 2 or more pHLIP peptides) by a covalent bond. In non-limiting examples, the covalent bond between a pHLIP peptide and a linker compound and/or the covalent bond between a linker compound and a cargo compound is a disulfide bond, a bond between two selenium atoms, a bond between a sulfur and a selenium atom, or a bond that has been formed by a click reaction.

In various embodiments, the cargo has a weight of (a) at least about 0.5, 1, 1.5, 2, 2.5, 5, 6, 7, 8, 9, or 10 kilodaltons (kDa); or (b) less than about 0.5, 1, 1.5, 2, 2.5, 5, 6, 7, 8, 9, or 10 kDa. In a non-limiting example, a pHLIP peptide is linked to a cargo compound having a weight of at least about 15 kDa. In another non-limiting example, a pHLIP peptide is linked to a cargo compound having a weight of less than about 15 kDa. The cargo may be, e.g., polar or nonpolar.

In certain embodiments, the cargo is a marker and/or a therapeutic, diagnostic, radiation-enhancing, radiation-sensitizing, imaging, gene regulation, cytotoxic, apoptotic, or research reagent. In some embodiments, a pHLIP peptide or linker is linked to one or more cargo molecules used as a therapeutic, diagnostic, imaging, immune activation, gene regulation or cell function regulation agent, radiation-enhancing agent, radiation-sensitizing agent, or as a research tool. In various non-limiting examples, the cargo comprises a dye, a fluorescent dye, a fluorescent protein, a nanoparticle, or a radioactive isotope. For example, the cargo may include, e.g., phalloidin, phallo toxin, amanitin toxin, a DNA intercalator, or a peptide nucleic acid. In some embodiments, the cargo comprises a magnetic resonance agent, positron emission tomography agent, X-ray contrast agent, single photon emission computed tomography agent, or fluorescence imaging agent.

In some implementations of the present subject matter, 1 or more of the amino acid side chains of the pHLIP peptide are chemically modified to be radioactive or detectable by probing radiation. In various embodiments one or more atoms of a pHLIP peptide are replaced by a radioactive isotope or a stable isotope.

Aspects of the present subject matter relate to the use of a pH-triggered compound as an agent to deliver a cargo molecule across a cell membrane to a cell in a diseased tissue with a naturally acidic extracellular environment or in a tissue with an artificially induced acidic extracellular environment relative to normal physiological pH. In a non-limiting example, the diseased tissue is selected from the group consisting of inflamed tissue, ischemic tissue, arthritic tissue, tissue infected with a microorganism, and atherosclerotic tissue.

In various embodiments, artificially inducing an acidic extracellular environment relative to normal physiological pH comprises administering glucose or an acidic solution to the subject. For example, glucose or an acidic solution (e.g., comprising lactic acid) may be administered to the skin or a tissue (e.g., tumor) site.

Alternatively or in addition, a pH-triggered compound may be used as an agent to facilitate the attachment of a cargo molecule to the surface of skin. For example, a pH-triggered compound may be linked to a cargo molecule that is an antibiotic compound.

In some embodiments, the cargo is a chemotherapeutic agent.

Various implementations of the present subject matter relate to a diagnostic conjugate comprising a pH-triggered compound and a pharmaceutically acceptable detectable marker linked thereto. In some embodiments, the detectable marker comprises a dye or a nanoparticle.

In various embodiments, the compound has a higher affinity for a membrane lipid bilayer at low pH compared to that at normal pH. In some embodiments, the affinity is at least 5 times higher at pH 5.0 than at pH 8.0. In some embodiments, the affinity is at least 10 times higher at pH 5.0 than at pH 8.0. In some embodiments, the binding/association/partitioning of a pH-triggered compound with a membrane lipid bilayer is stronger at low pH (e.g., pH<6.5 or 7.0) compared to a higher pH (e.g., pH>6.5 or 7.0).

In some embodiments, the non-polar amino acid or amino acids comprise alanine, valine, isoleucine, leucine, methionine, phenylalanine, tyrosine, tryptophan, or any combination thereof. In some embodiments, a polar amino acid or amino acids comprise serine, threonine, asparagine, glutamine, or any combination thereof. In some embodiments, the non-polar amino acid is an artificial amino acid such as 1-methyl-tryptophan.

In various embodiments, a non-polar amino acid is defined as one having a side-chain solvation energy≥0.5 kcal/mol. The values of solvation energy (ΔG_{X}^{corr}) for the 20 common natural amino acids are known, e.g., as determined by Wimley W C, Creamer T P & White S H (1996) Biochemistry 35, 5109-5124 or by Moon and Fleming, (2011) Proc. Nat. Acad. Sci. USA 101:10174-10177 (hereinafter Wimley et al. 2011), the entire content of which is incorporated herein by reference. The table below provides exemplary side chain solvation energies for naturally occurring amino acids.

TABLE 1
Solvation Free Energies of the Side Chains (X) of the 20 Natural Amino
Acids in AcWL-X-LL (SEQ ID NO: 329). Non-polar residues are shown
in bold and defined as residues with ΔG_{X}^{cor} > +0.50. Gly was used as
a reference, its energy ΔG_{X}^{cor} was set as zero.

| Residue | Charge | ΔG_{X}^{cor} |
|---|---|---|
| Ala | 0 | +0.65 |
| Arg | +1 | −0.66 |
| Asn | 0 | +0.30 |
| Asp | −1 | −2.49 |
| Cys | 0 | +1.17 |
| Gln | 0 | +0.38 |
| Glu | −1 | −2.48 |
| Gly | 0 | 0 |
| His | +1 | −1.18 |
| Ile | 0 | +2.27 |
| Leu | 0 | +2.40 |
| Lys | +1 | −1.65 |
| Met | 0 | +1.82 |
| Phe | 0 | +2.86 |
| Pro | 0 | +1.01 |
| Ser | 0 | +0.69 |
| Thr | 0 | +0.90 |
| Trp | 0 | +3.24 |
| Tyr | 0 | +1.86 |
| Val | 0 | +1.61 |

Residue solvation free energies of the 20 natural amino acids relative to glycine calculated from the data in Table 1 of Wimley et al. 2011. Free energies were corrected for the occlusion of neighboring residue areas (see text of Wimley et al. 2011) and for the anomalous properties of glycine (see text of Wimley et al. 2011). Residue solvation free energies calculated with mole-fraction units. Residue solvation free energies for the X residue in the context of a AcWL-X-LL (SEQ ID NO: 329) peptide calculated from the free energies in Table 1 or Wimley et al. 2011 using the virtual glycine (Gly) as the reference (see text of Wimley et al. 2011) (SEQ ID NOS 329-330, 329, and 329 are disclosed below, respectively, in order of appearance).

ΔG_{X}^{cor}=ΔG_{WLXLL}−ΔG_{WLG*LL}+ΔσₙₚΔAₕₒₛₜ,

Aₕₒₛₜ(X)=A_{Tnp}(WLXLL)−A_{Xnp}(WLXLL)

These “corrected” values account for X-dependent changes in the nonpolar ASA of the host peptide. Values for Arg and Lys were calculated from experimental free energies measured at pH 1 where the ionic interaction between the side chain and carboxyl group does not occur. ΔG_{X}^{cor} is the best estimate of the solvation energy of residues occluded by neighboring residues of moderate size.

Coded amino acids and exemplary non-coded amino acids are listed below in Table 2.

In some embodiments, a pHLIP peptide (e.g., a monomer or within a compound that comprises multiple pHLIP peptides) comprises one or more cysteine residues. The cysteine residue(s) may serve as a point of conjugation of cargo, e.g., using thiol linkage. Other means of linking cargo to a pHLIP peptide include esters and/or acid-liable linkages. Ester linkages are particularly useful in humans, the cells of which contain esterases in the cytoplasm to liberate the cargo inside the cells. In certain embodiments, this system is less useful in the mouse or other rodents, which species are characterized by a high level of esterases in the blood (thereby leading to premature release of cargo molecules). Non-cleavable covalent chemical linkages may also be made to secure a cargo permanently to a pHLIP peptide.

pH-triggered compounds provided herein are useful for topical, dermatological and internal medical applications, e.g., as therapeutic, diagnostic, prophylactic, imaging, gene regulation, or as research reagents/tools, e.g., to evaluate cell function regulation, apoptosis, or other cell activities. For such applications, the composition further comprises a moiety attached to a functional group. Exemplary moieties include imaging agents, dyes, or other detectable labels; and prophylactic, therapeutic and cytotoxic agents. For example, in some implementations, pH-triggered compounds translocate cell permeable and/or cell impermeable cargo molecules (such as nanoparticles, organic dyes, peptides, peptide nucleic acids and toxins) across the membrane. In certain embodiments, the pH-triggered compounds target cargo (e.g., an imaging agent such as a dye or another detectable label) to cell surfaces in tissues such as acidic tissues. For example, a pH-triggered compound linked to an imaging cargo such as a dye or stain can be used during a chromoendoscopy procedure (such as during a colonoscopy) to enhance tissue differentiation or characterization. In various embodiments, the pH-triggered compound itself is non-toxic, especially when an effective amount of the pH-triggered compound is used. Non-limiting examples of cargo molecules include magnetic resonance (MR) agents, positron emission tomography (PET) agents, single photon emission computed tomography (SPECT) agents, x-ray contrast agents, fluorescence imaging agents, natural toxins, deoxyribonucleic acid (DNA) intercalators, peptide nucleic acids (PNA), morpholinos (e.g., morpholino oligomers), peptides, and naturally-occurring or synthetic drug molecules. Other examples of therapeutic or diagnostic moieties or cargo compounds include radiation-enhancing or radiation-sensitizing compounds such as nanogold particles to enhance imaging or cell destruction, e.g., tumor cell killing, by radiation or boron-containing compounds such as Disodium mercapto-closo-dodecaborate (BSH) for boron neutron capture therapy (BNCT) that kills labeled target cells while sparing unlabeled non-target (non-diseased) cells. For imaging or other applications for which detection is desired, one or more atoms are optionally replaced by radioactive isotopes. For example, one or more of the amino acid side chains may be chemically modified to render them radioactive or detectable by probing radiation.

In various embodiments, the moiety or cargo molecule comprises a marker. As used herein, a “marker” may be any compound that provides an identifiable signal. Non-limiting examples of markers include fluorescent dyes, phosphorescent dyes, and quantum dots.

In some embodiments, the marker is a fluorophore. In various embodiments, 1, 2, 3, 4, 5 or more fluorophores are attached to a pHLIP compound provided herein.

Non-limiting examples of fluorophores include but are not limited to fluorescent dyes, phosphorescent dyes, quantum dots, xanthene derivatives, cyanine derivatives, naphthalene derivatives, coumarin derivatives, oxadiaxol derivatives, pyrene derivatives, acridine derivatives, arylmethine derivatives, and tetrapyrrole derivatives. Xanthene derivatives include but are not limited to fluorescein, rhodamine, Oregon green, eosin, Texas red, and Cal Fluor dyes. Cyanine derivatives include but are not limited to cyanine, indocarbocyanine, indocyanine green (ICG), oxacarbocyanine, thiacarbocyanine, merocyanine, and Quasar dyes. Naphthalene derivatives include but are not limited to dansyl and prodan derivatives. Oxadiazole derivatives include but are not limited to pyridyloxazol, nitrobenzoxadiazole, and benzoxadiazole. A non-limiting example of a pyrene derivative is cascade blue. Oxadine derivatives include but are not limited to Nile red, Nile blue, cresyl violet, and oxazine 170. Acridine derivatives include but are not limited to proflavin, acridine orange, and acridine yellow. Arylmethine derivatives include but are not limited to auramine, crystal violet, and malachite green. Tetrapyrrole derivatives include but are not limited to porphin, phtalocyanine, and bilirubin.

In various embodiments, the moiety is covalently attached to the pH-triggered compound via a linkage such as a thiol linkage or ester linkage or acid-liable linkage. Other types of linkages, chemical bonds, or binding associations may also be used. Exemplary linkages or associations are mediated by a disulfide, and/or a peptide with a protein binding motif, and/or a protein kinase consensus sequence, and/or a protein phosphatase consensus sequence, and/or a protease-reactive sequence, and/or a peptidase-reactive sequence, and/or a transferase-reactive sequence, and/or a hydrolase-reactive sequence, and/or an isomerase-reactive sequence, and/or a ligase-reactive sequence, and/or an extracellular metalloprotease-reactive sequence, and/or a lysosomal protease-reactive sequence, and/or a beta-lactamase-reactive sequence, and/or an oxidoreductase-reactive sequence, and/or an esterase-reactive sequence, and/or a glycosidase-reactive sequence, and/or a nuclease-reactive sequence.

In certain embodiments, the moiety or cargo compound is covalently attached to the pH-triggered compound via a non-cleavable linkage. In various embodiments, a non-cleavable linkage is a covalent bond that is not cleaved by an enzyme expressed by a mammalian cell, and/or not cleaved by glutathione and/or not cleaved at conditions of low pH. Non-limiting examples of non-cleavable linkages include maleimide linkages, linkages resulting from the reaction of a N-hydroxysuccinimide ester with a primary amine (e.g., a primary amine of a lysine side-chain), linkages resulting from a click reaction, thioether linkages, or linkages resulting from the reaction of a primary amine (—NH₂) or thio (—SH) functional group with succinimidyl-4-(N-maleimidomethyl)cyclohexane-1-carboxylate (SMCC). Exemplary non-cleavable linkages include a maleimide alkane linker,

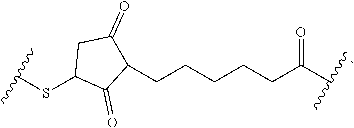 [SMILES: *SC1CC(=O)C(CCCCCC(*)=O)C1=O]

and a maleimide cyclohexane linker,

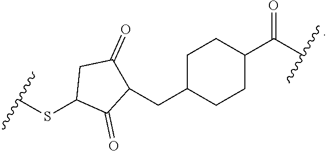 [SMILES: *SC1CC(=O)C(CC2CCC(C(*)=O)CC2)C1=O]

In some embodiments, a linker comprises one or more linear or branched poly(ethylene glycol) (PEG) and/or maleimide structures. In certain embodiments, the PEG has two arms. In various embodiments, the PET has four arms. In some embodiments, each of the PEG arms of a linker comprises a maleimide structure. In certain embodiments, a linker having one of the following structures is used to covalently attach a pHLIP peptide to at least one other pHLIP peptide and/or at least one cargo compound:

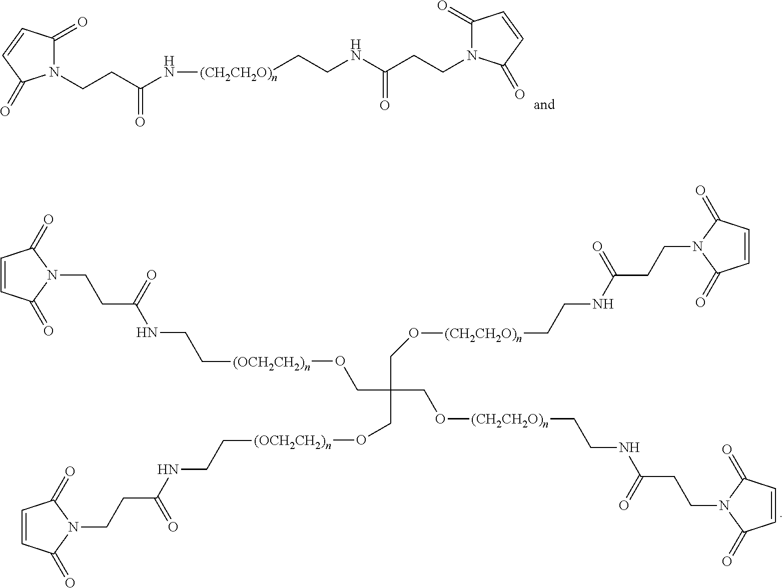

Exemplary uses of the environmentally-sensitive compositions is to tether molecules to a membrane and/or shuttle molecules across a membrane. For example, in some embodiments, a pHLIP compound is used as an agent to deliver a functional moiety (diagnostic or therapeutic) to or across a cell membrane to a cell in a tissue with a naturally acidic extracellular environment or in a tissue with an artificially or disease induced acidic extracellular environment relative to normal physiological pH. Many diseased tissues and normal skin are characterized by an acidic microenvironment. However, acidity in tumors or non-tumor target tissues is optionally induced by co-injection of glucose or a diluted solution of acid at the tissue site at which therapy using the compositions is desired. For example, an acidifying composition (e.g., glucose or dilute acid) may be administered, e.g., injected subcutaneously, before delivery of the pH sensitive compositions (e.g., about 30 s, 1 min, 5 min, 10 min, 30 min, 1 hr, 2 hrs, 6 hrs, 12 hrs, 24 hrs, 48 hrs, or more prior to administration of the environmentally sensitive composition to the target tissue site). Alternatively or in addition, the tissue acidifying agent and the pH-triggered compound composition are co-administered. In some embodiments, the diseased tissue is selected from the group consisting of cancer, inflammation/inflamed tissue, ischemia/ischemic tissue, tissue affected by stroke, arthritis, infection with a microorganism (e.g., a bacteria, virus, or fungus), or atherosclerotic plaques. Compositions provided herein are also useful to deliver a functional moiety to cell surfaces in a diseased tissue with a naturally acidic extracellular environment or in a tissue with an artificially induced acidic extracellular environment relative to normal physiological pH. In certain embodiments, administration of a neutralizing agent to an acidic site, e.g., a bicarbonate solution, is used to reduce pH-triggered compound binding/insertion and pH-triggered compound labeling or targeting of cells at that site. Compounds and compositions provided herein are also useful to tether and deliver a therapeutic compound to the surface of skin with a naturally acidic environment or to a skin with an artificially induced acidic environment.

As is described herein, the compositions may be used in a clinical setting for diagnostic and therapeutic applications in humans as well as animals (e.g., companion animals such as dogs and cats as well as livestock such as horses, cattle, goats, sheep, llamas). In various embodiments, a diagnostic conjugate comprises an environmentally (e.g., pH sensitive) pHLIP compound and a pharmaceutically-acceptable detectable marker linked thereto. Exemplary detectable markers include fluorescent dyes, as well as MR, PET, SPECT, optoacoustic, X-ray, CT and other imaging agents. Such conjugates are used in a variety of clinical diagnostic methods, including real-time image-guided therapeutic interventions. For example, a method of guiding surgical tumor excision is carried out by administering a pHLIP compound disclosed herein to an anatomical site comprising a tumor, removing a primary tumor from the site, and detecting residual tumor cells by virtue of binding of the compound to residual tumor cells.

Included herein are compositions that are administered to the body for diagnostic and therapeutic use, e.g., using administration methods known in the art. For example, in some embodiments the methods are carried out by infusing into a vascular lumen, e.g., intravenously, via a jugular vein, peripheral vein or the perivascular space. In some embodiments, the composition is infused into the lungs of said mammal, e.g., as an aerosol or lavage. In certain embodiments, the composition is administered by injection, e.g., into an anatomical region of interest such as a tumor site or site of another pathological condition or suspected pathological condition. In various embodiments, the composition is administered by intravesical instillation into a human or animal bladder, oral cavity, intestinal cavity, esophagus, or trachea. In some embodiments, the injection can be into the peritoneal cavity of the mammal, subdermally, or subcutaneously. The compositions can also be administered transdermally. Solutions containing the imaging conjugates or therapeutic conjugates are administered intravenously, by lavage of the area (e.g., peritoneal tissue or lung tissue), topically, transdermally, by inhalation, or by injection (e.g., directly into a tumor or tumor border area). In certain embodiments, 1-50 mg in 100 mL is used for lavage and 0.1-100 mg/kg is used for other routes of administration.

Targeting of acidity provides a predictive marker for tumor invasiveness and disease development. In addition to image-guided therapies, compounds and compositions provided herein are useful to diagnose or measure the severity of a pathological condition. In various embodiments, a method of determining the aggressiveness of a primary tumor is carried out by contacting the tumor with the environmentally-sensitive composition (e.g., comprising a pHLIP compound disclosed herein), wherein an increased level of binding of the composition compared to a control level of binding indicates an increased risk of metastasis from the primary tumor. Thus, a compositions included herein aid the physician in determining a prognosis for disease progression and appropriately tailoring therapy based on the severity or aggressiveness of the disease.

A method of preferentially inhibiting proliferation of tumor cells is carried out by administering to a subject suffering from or at risk of developing a tumor the therapeutic conjugate compositions described above to the subject. Tumor cells are preferentially inhibited compared to normal non-tumor cells. The pH-triggered compound delivery system, e.g., exemplified by the therapeutic conjugates, are therefore used in a method of manufacturing a pharmaceutical composition or medicament for treatment of tissues characterized by disease or an acid microenvironment.

### Non-Limiting Variants of Non-Limiting Exemplified Peptides

pH-triggered compounds provided herein may contain one or more pHLIP peptides, e.g. any one of, or (in the case of compounds having more than one pHLIP peptide) any combination of the non-limiting examples pHLIP peptides provided herein or variants thereof. Variants of the membrane insertion peptides exemplified or otherwise disclosed herein may be designed using substitution techniques that are well understood in the art. Neither the membrane insertion peptides exemplified herein nor the variants discussed below limit the full scope of the subject matter disclosed herein. Non-limiting examples of variants of the specific membrane insertion disclosed herein include peptides having the reverse amino acid sequence of the specific membrane insertion peptides disclosed. For example, a disclosure of a membrane insertion peptide comprising the sequence WARYADWL (SEQ ID NO: 256) also provides the disclosure of a pHLIP peptide comprising the sequence LWDAYRAW (SEQ ID NO: 257).

Aspects of the present subject matter relate to pHLIP peptides that result from 1, 2, 3, 4, 5, 6, 7, 8, 9, 10 or more conservative amino acid substitutions compared to a pHLIP peptide exemplified herein. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a residue in a pH-triggered peptide sequence (e.g., corresponding to a location relative to a SEQ ID NO disclosed herein) may be replaced with another amino acid residue from the same side chain family. In certain embodiments, conservative amino acid substitutions may be made using a natural amino acid or a non-natural amino acid.

TABLE 2
Coded and exemplary non-coded amino acids including
L-isomers, D- isomers, alpha-isomers, beta-isomers,
glycol-, and methyl- modifications.

| No. | Abbrev | Name |
|---|---|---|
| 1 | Ala | Alanine |
| 2 | Arg | Arginine |
| 3 | Asn | Asparagine |
| 4 | Asp | Aspartic acid |
| 5 | Cys | Cysteine |
| 6 | Gln | Glutamine |
| 7 | Glu | Glutamic acid |
| 8 | Gly | Glycine |
| 9 | His | Histidine |
| 10 | Ile | Isoleucine |
| 11 | Leu | Leucine |
| 12 | Lys | Lysine |
| 13 | Met | Methionine |
| 14 | Phe | Phenylalanine |
| 15 | Pro | Proline |
| 16 | Ser | Serine |
| 17 | Thr | Threonine |
| 18 | Trp | Tryptophan |
| 19 | Tyr | Tyrosine |
| 20 | Val | Valine |
| 21 | Sec | Selenocysteine |
| 22 | Sem | Selenomethionine |
| 23 | Pyl | Pyrrolysine |
| 24 | Aad | Alpha-aminoadipic acid |
| 25 | Acpa | Amino-caprylic acid |
| 26 | Aecys | Aminoethyl cysteine |
| 27 | Afa | Aminophenyl acetate |
| 28 | Gaba | Gamma-aminobutyric acid |
| 29 | Aiba | Aminoisobutyric acid |
| 30 | Aile | Alloisoleucine |
| 31 | AIg | Allylglycine |
| 32 | Aba | Amino-butyric acid |
| 33 | Aphe | Amino-phenylalanine |
| 34 | Brphe | Bromo-phenylalanine |
| 35 | Cha | Cyclo-hexylalanine |
| 36 | Cit | Citrulline |
| 37 | Clala | Chloroalanine |
| 38 | Cie | Cycloleucine |
| 39 | Clphe | Fenclonine (or chlorophenylalanine) |
| 40 | Cya | Cysteic acid |
| 41 | Dab | Diaminobutyric acid |
| 42 | Dap | Diaminopropionic acid |
| 43 | Dap | Diaminopimelic acid |
| 44 | Dhp | Dehydro-proline |
| 45 | Dhphe | DOPA (or 3,4-dihydroxyphenylalanine) |
| 46 | Fphe | Fluorophenylalanine |
| 47 | Gaa | Glucosaminic acid |
| 48 | Gla | Gamma-carboxyglutamic acid |
| 49 | Hag | Homoarginine |
| 50 | Hlys | Hydroxylysine |
| 51 | Hnvl | Hydroxynorvaline |
| 52 | Hog | Homoglutamine |
| 53 | Hoph | Homophenylalanine |
| 54 | Has | Homoserine |
| 55 | Hse | Homocysteine |
| 56 | Hpr | Hydroxyproline |
| 57 | Iphe | Iodo-phenylalanine |
| 58 | Ise | Isoserine |
| 59 | Mle | Methyl-leucine |
| 60 | Msmet | Methionine-methylsulfonium chloride |
| 61 | Nala | Naphthyl-alanine |
| 62 | Nle | Norleucine (or 2-aminohexanoic acid) |
| 63 | Nmala | N-methyl-alanine |
| 64 | Nva | Norvaline (or 2-aminopentanoic acid) |
| 65 | Obser | O-benzyl-serine |
| 66 | Obtyr | O-benzyl-tyrosine |
| 67 | Oetyr | O-ethyl-tyrosine |
| 68 | Omser | O-methyl-serine |
| 69 | Omthr | O-methy-threonine |
| 70 | Omtyr | O-methyl-tyrosine |
| 71 | Orn | Ornithine |
| 72 | Pen | Penicillamine |
| 73 | Pga | Pyroglutamic acid |
| 74 | Pip | Pipecolic acid |
| 75 | Sar | Sarcosine |
| 76 | Tfa | Trifluoro-alanine |
| 77 | Thphe | Hydroxy-Dopa |
| 78 | Vig | Vinylglycine |
| 79 | Aaspa | Amino-aminoethylsulfanylpropanoic acid |
| 80 | Ahdna | Amino-hydroxy-dioxanonanolic acid |
| 81 | Ahoha | Amino-hydroxy-oxahexanoic acid |
| 82 | Ahsopa | Amino-hydroxyethylsulfanylpropanoic acid |
| 83 | Tyr(Me) | Methoxyphenyl-methylpropanyl |
| | | oxycarbonylamino propanoic acid |
| 84 | MTrp | Methyl-tryptophan |
| 85 | pTyr | Phosphorylated Tyr |
| 86 | pSer | Phosphorylated Ser |
| 87 | pThr | Phosphorylated Thr |
| 88 | BLys | BiotinLys |
| 89 | Hyp | Hydroproline |
| 90 | Phg | Phenylglycine |
| 91 | Cha | Cyclohexyl-alanine |
| 92 | Chg | Cyclohexylglycine |
| 93 | Nal | Naphthylalanine |
| 94 | Pal | Pyridyl-alanine |
| 95 | Pra | Propargylglycine |
| 96 | Gly(allyl) | Pentenoic acid |
| 97 | Pen | Penicillamine |
| 98 | MetO | Methionine sulfoxide |
| 99 | Pca | Pyroglutamic acid |
| 100 | Ac-Lys | Acetylation of Lys |

TABLE 3
Non-limiting examples of protonatable residues
and their substitutions including L- isomers,
D- isomers, alpha-isomers, and beta-isomers.

| Original | |
|---|---|
| Residue | Exemplary amino acids substitution |
| Asp (D) | Glu (E); Gla (Gla); Aad (Aad) |
| Glu (E) | Asp (D); Gla (Gla); Aad (Aad) |

TABLE 4
Examples of coded amino acid substitutions

| Original | |
|---|---|
| Residue | Substitution |
| Ala (A) | Gly; Ile; Leu; Met; Phe; Pro; Trp; Tyr; Val |
| Arg (R) | Lys |
| Asn (N) | Gln; His |
| Asp (D) | Glu |
| Cys (C) | Ser; Met |
| Gln (Q) | Asn; His |
| Glu (E) | Asp |
| Gly (G) | Ala; Ile; Leu; Met; Phe; Pro; Trp; Tyr; Val |
| His (H) | Asn; Gln |
| Ile (I) | Ala; Gly; Leu; Met; Phe; Pro; Trp; Tyr; Val |
| Leu (L) | Ala; Gly; Ile; Met; Phe; Pro; Trp; Tyr; Val |
| Lys (K) | Arg |
| Met (M) | Ala; Gly; Leu; Ile; Phe; Pro; Trp; Tyr; Val |
| Phe (F) | Ala; Gly; Leu; Ile; Met; Pro; Trp; Tyr; Val |
| Pro (P) | Ala; Gly; Leu; Ile; Met; Phe; Trp; Tyr; Val |
| Ser (S) | Thr |
| Thr (T) | Ser |
| Trp (W) | Ala; Gly; Leu; Ile; Met; Pro; Phe; Tyr; Val |
| Tyr (Y) | Ala; Gly; Leu; Ile; Met; Pro; Phe; Trp; Val |
| Val (V) | Ala; Gly; Leu; Ile; Met; Pro; Phe; Trp; Tyr |

TABLE 5
Non-limiting examples of membrane-inserting sequences belonging to
different groups of pHLIP peptides. Each protonatable residue (shown
in bold) could be replaced by its substitution from Table 3. Each
non-polar residue could be replaced by its coded amino acid substi-
tution from Table 4, and/or non-coded amino acid substitutions from
Table 2.

| Groups | Sequences |
|---|---|
| WT-BRC | WARYADWLFTTPLLLLDLALL (SEQ ID NO: 57) |
| | YARYADWLFTTPLLLLDLALL (SEQ ID NO: 58) |
| | WARYSDWLFTTPLLLYDLGLL (SEQ ID NO: 59) |
| | WARYTDWFTTPLLLYDLALLA (SEQ ID NO: 60) |
| | WARYTDWLFTTPLLLYDLGLL (SEQ ID NO: 61) |
| | WARYADWLFTTPLLLLDLSLL (SEQ ID NO: 62) |
| WT-BRC Reverse | LLALDLLLLPTTFLWDAYRAW (SEQ ID NO: 63) |
| | LLALDLLLLPTTFLWDAYRAY (SEQ ID NO: 64) |
| | LLGLDYLLLPTTFLWDSYRAW (SEQ ID NO: 65) |
| | ALLALDYLLLPTTFWDTYRAW (SEQ ID NO: 66) |
| | LLGLDYLLLPTTFLWDTYRAW (SEQ ID NO: 67) |
| | LLSLDLLLLPTTFLWDAYRAW (SEQ ID NO: 328) |
| ATRAM | GLAGLLGLEGLLGLPLGLLEGLWLGL (SEQ ID NO: 68) |
| ATRAM Reverse | LGLWLGELLGLPLGLLGELGLLGALG (SEQ ID NO: 69) |
| Var3 | WRAYLDLLFPTDTLLLDLLW (SEQ ID NO: 70) |
| Var3 Reverse | WLLDLLLTDTPFLLDLYARW (SEQ ID NO: 71) |
| Var7 | WARYLEWLFPTETLLLEL (SEQ ID NO: 72) |
| | WAQYLELLFPTETLLLEW (SEQ ID NO: 73) |
| Var7 Reverse | LELLLTETPFLWELYRAW (SEQ ID NO: 74) |
| | WELLLTETPFLLELYQAW (SEQ ID NO: 75) |
| Single D/E | WLFTTPLLLLNGALLVE (SEQ ID NO: 76) |
| | WLFTTPLLLLPGALLVE (SEQ ID NO: 77) |
| | WARYADLLFPTTLAW (SEQ ID NO: 78) |
| Single D/E Reverse | EVLLAGNLLLLPTTFLW (SEQ ID NO: 79) |
| | EVLLAGPLLLLPTTFLW (SEQ ID NO: 80) |
| | WALTTPFLLDAYRAW (SEQ ID NO: 81) |
| pHLIP-Rho | NLEGFFATLGGEIALWSLVVLAIE (SEQ ID NO: 82) |
| | EGFFATLGGEIALWSDVVLAIE (SEQ ID NO: 83) |
| | EGFFATLGGEIPLWSDVVLAIE (SEQ ID NO: 84) |
| pHLIP-Rho Reverse | EIALVVLSWLAIEGGLTAFFGELN (SEQ ID NO: 85) |
| | EIALVVDSWLAIEGGLTAFFGE (SEQ ID NO: 86) |
| | EIALVVDSWLPIEGGLTAFFGE (SEQ ID NO: 87) |
| pHLIP-CA9 | ILDLVFGLLFAVTSVDFLVQW (SEQ ID NO: 88) |
| pHLIP-CA9 Reverse | WQVLFDVSTVAFLLGFVLDLI (SEQ ID NO: 89) |

TABLE 6
Non-limiting examples of linkers and components thereof

| ID | Name |
|---|---|
| 1 | Peptide bond, (—CO—NH—) |
| 2 | Polypeptide |
| 3 | Polylysine |
| 4 | Polyarginine |
| 5 | Polyglutamic acid |
| 6 | Polyaspartic acid |
| 7 | Polycysteine |
| 8 | Collagen |
| 9 | Fibrinogen |
| 10 | Avidin |
| 11 | Streptavidin |
| 12 | Albumin |
| 13 | Antibody |
| 14 | Protein with 1 or more Lys, Arg, Cys, |
| 15 | Asp, Glu |
| 16 | Polynucleotide |
| 17 | Polysaccharide |
| 18 | Alginate |
| 19 | Chitosan |
| 20 | Poly(ethylene glycol) (PEG) |
| 21 | Poly(lactic acid) (PLA) |
| 22 | Poly(glycolic acid) (PGA) |
| 23 | Poly(lactic-co-glycolic acid) (PLGA) |
| 24 | Poly(malic acid) (PMA) |
| 25 | Polyorthoesters (POE) |
| 26 | Poly(vinylalcohol) (PVOH, PVA, |
| 27 | or PVAl) |
| 28 | Poly(vinylpyrrolidone) (PVP) |
| 29 | Poly(methyl methacrylate) (PMMA) |
| 30 | Poly(acrylic acid) (PAA) |
| 31 | Poly(acrylamide) (PAM) |
| 32 | Poly(methacrylic acid) (PMAA) |
| 33 | Poly(amidoamine) (PAMAM) |
| 34 | Polyanhydrides |
| 35 | Polycyanoacrylate |
| 36 | Particle |
| 37 | Metallic particle |
| 38 | Polymeric particle |
| 39 | Virus-like particle |
| 40 | Nanoparticle |
| 41 | Metallic nanoparticle |
| 42 | Lipid-based nanoparticle |
| 43 | Surfactant-based nanoparticle |
| 44 | Polymeric nanoparticle |
| 45 | Peptide-based nanoparticle |

TABLE 7
Non-limiting examples of pHLIP sequences. A cysteine, a lysine, an
azido-modified amino acid, or an alkynyl modified amino acid can be
incorporated at the N-terminal (first 6 residues) or C-terminal (last
6 residues) parts of the peptides for conjugation with a cargo, and
a linker.

| SEQ ID NO | Name | Sequence |
|---|---|---|
| SEQ ID NO: 258 | WT-2D | AEQNPIYWARYADWLFTTPLLLLDLALLVDADET |
| SEQ ID NO: 154 | WT-6E | AEQNPIYWARYAEWLFTTPLLLLELALLVEAEET |
| SEQ ID NO: 155 | WT-3D | ADDQNPWRAYLDLLFPDTTDLLLLDLLWDADET |
| SEQ ID NO: 156 | WT-9E | AEEQNPWRAYLELLFPETTELLLLELLWEAEET |
| SEQ ID NO: 259 | WT-GlaD | AEQNPIYWARYAGlaWLFTTPLLLLDLALLVDADET |
| SEQ ID NO: 260 | WT-DGla | AEQNPIYWARYADWLFTTPLLLLGlaLALLVDADET |
| SEQ ID NO: 157 | WT-2Gla | AEQNPIYWARYAGlaWLFTTPLLLLGlaLALLVDADET |
| SEQ ID NO: 261 | WT-AadD | AEQNPIYWARYAAadWLFTTPLLLLDLALLVDADET |
| SEQ ID NO: 262 | WT-DAad | AEQNPIYWARYADWLFTTPLLLLAadLALLVDADET |
| SEQ ID NO: 158 | WT-2Aad | AEQNPIYWARYAAadWLFTTPLLLLAadLALLVDADET |
| SEQ ID NO: 263 | WT-GlaAad | AEQNPIYWARYAGlaWLFTTPLLLLAadLALLVDADET |
| SEQ ID NO: 159 | WT-AadGla | AEQNPIYWARYAAadWLFTTPLLLLGlaLALLVDADET |
| SEQ ID NO: 264 | WT-1 | GGEQNPIYWARYADWLFTTPLLLLDLALLVDADEGT |
| SEQ ID NO: 265 | WT-2 | GGEQNPIYWARYADWLFTTPLLLLDLALLVDADEGT |
| SEQ ID NO: 266 | WT-3 | AAEQNPIYWARYADWLFTTPLLLLDLALLVDADEGT |
| SEQ ID NO: 267 | WT-4 | AEQNPIYWARYADWLFTTPLLLLDLALLVDADEGT |
| SEQ ID NO: 323 | WT-2N | AEQNPIYWARYANWLFTTPLLLLNLALLVDADEGT |
| SEQ ID NO: 268 | WT-2K | AEQNPIYWARYAKWLFTTPLLLLKLALLVDADEGT |
| SEQ ID NO: 269 | WT-2DNANQ | GGEQNPIYWARYADWLFTTPLLLLDLALLVNANQGT |
| SEQ ID NO: 270 | WT-D25A | AAEQNPIYWARYADWLFTTPLLLLALALLVDADEGT |
| SEQ ID NO: 271 | WT-D14A | AAEQNPIYWARYAAWLFTTPLLLLDLALLVDADEGT |
| SEQ ID NO: 272 | WT-P20A | AAEQNPIYWARYADWLFTTALLLLDLALLVDADEGT |
| SEQ ID NO: 273 | WT-D25E | AAEQNPIYWARYADWLFTTPLLLLELALLVDADEGT |
| SEQ ID NO: 274 | WT-D14E | AAEQNPIYWARYAEWLFTTPLLLLDLALLVDADEGT |
| SEQ ID NO: 275 | WT-3D-2 | AAEQNPIIYWARYADWLFTDLPLLLLDLLALLVDADEGT |
| SEQ ID NO: 276 | WT-R11Q | GEQNPIYWAQYADWLFTTPLLLLDLALLVDADEG |
| SEQ ID NO: 277 | WT-D25Up | GGEQNPIYWARYADWLFTTPLLLDLLALLVDADEG |
| SEQ ID NO: 278 | WT-D25Down | GGEQNPIYWARYADWLFTTPLLLLLDALLVDADEG |
| SEQ ID NO: 279 | WT-D14Up | GGEQNPIYWARYDAWLFTTPLLLLDLALLVDADEGT |
| SEQ ID NO: 280 | WT-D14Down | GGEQNPIYWARYAWDLFTTPLLLLDLALLVDADEG |
| SEQ ID NO: 281 | WT-P20G | AAEQNPIYWARYADWLFTTGLLLLDLALLVDADEGT |
| SEQ ID NO: 282 | WT-DH | DDDEDNPIYWARYADWLFTTPLLLLHGALLVDAD |
| SEQ ID NO: 283 | WT-2H | DDDEDNPIYWARYAHWLFTTPLLLLHGALLVDADE |
| SEQ ID NO: 160 | WT-L16H | CEQNPIYWARYADWHFTTPLLLLDLALLVDADE |
| SEQ ID NO: 284 | WT-1Wa | AEQNPIYWARYADFLFTTPLLLLDLALLVDADET |
| SEQ ID NO: 285 | WT-1Wb | AEQNPIYFARYADWLFTTPLLLLDLALLVDADE |
| SEQ ID NO: 286 | WT-1Wc | AEQNPIYFARYADFLFTTPLLLLDLALLWDADET |
| SEQ ID NO: 161 | WT-W6 | ADNNPWIYARYADLTTFPLLLLDLALLVDFDD |
| SEQ ID NO: 162 | WT-W17 | ADNNPFIYARYADLTTWPLLLLDLALLVDFDD |
| SEQ ID NO: 163 | WT-W30 | ADNNPFIYARYADLTTFPLLLLDLALLVDWDD |
| SEQ ID NO: 164 | WT-W17-P7 | ADNNPFPYARYADLTTWILLLLDLALLVDFDD |
| SEQ ID NO: 165 | WT-W39-R11 | ADNNPFIYAYRADLTTFPLLLLDLALLVDWDD |
| SEQ ID NO: 166 | WT-W30-R15 | ADNNPFIYATYADLRTFPLLLLDLALLVDWDD |
| SEQ ID NO: 287 | WT-Rev | Ac-TEDADVLLALDLLLLPTTFLWDAYRAWYPNQEA-Am |
| SEQ ID NO: 288 | Van-3D | AEDQNPYWARYADWLFTTPLLLLDLALLVD |
| SEQ ID NO: 289 | Var1-1D2E | AEDQNPYWARYADWLFTTPLLLLELALLVE |
| SEQ ID NO: 290 | Var2-3D | AEDQNPYWRAYADLFTPLTLLDLLALWD |
| SEQ ID NO: 46 | Var3-3D | ADDQNPWRAYLDLLFPTDTLLLDLLW |
| SEQ ID NO: 167 | Var3-WT | ADDQNPWRAYLDLLFPTDTLLLDLLWDADE |
| SEQ ID NO: 47 | Var3-Gla2D | ADDQNPWRAYLGlaLLFPTDTLLLDLLW |
| SEQ ID NO: 168 | Var3-DGlaD | ADDQNPWRAYLDLLFPTGlaTLLLDLLW |
| SEQ ID NO: 169 | Var3-2DGla | ADDQNPWRAYLDLLFPTDTLLLGlaLLW |
| SEQ ID NO: 170 | Var3-2GlaD | ADDQNPWRAYLGlaLLFPTGlaTLLLDLLW |
| SEQ ID NO: 171 | Var3-GlaDGla | ADDQNPWRAYLGlaLLFPTDTLLLGlaLLW |
| SEQ ID NO: 172 | Var3-D2Gla | ADDQNPWRAYLDLLFPTGlaTLLLGlaLLW |
| SEQ ID NO: 173 | Var3-3Gla | ADDQNPWRAYLGlaLLFPTGlaTLLLGlaLLW |
| SEQ ID NO: 174 | Var3-Aad2D | ADDQNPWRAYLAadLLFPTDTLLLDLLW |
| SEQ ID NO: 175 | Var3-DAadD | ADDQNPWRAYLDLLFPTAadTLLLDLLW |
| SEQ ID NO: 176 | Var3-2DAad | ADDQNPWRAYLDLLFPTDTLLLAadLLW |
| SEQ ID NO: 177 | Var3-2AadD | ADDQNPWRAYLAadLLFPTAadTLLLDLLW |
| SEQ ID NO: 178 | Var3-AadDAad | ADDQNPWRAYLAadLLFPTDTLLLAadLLW |
| SEQ ID NO: 179 | Var3-D2Aad | ADDQNPWRAYLDLLFPTAadTLLLAadLLW |
| SEQ ID NO: 180 | Var3-3Aad | ADDQNPWRAYLAadLLFPTAadTLLLAadLLW |
| SEQ ID NO: 181 | Var3-GlaAadD | ADDQNPWRAYLGlaLLFPTAadTLLLDLLW |
| SEQ ID NO: 182 | Var3-GlaDAad | ADDQNPWRAYLGlaLLFPTDTLLLAadLLW |
| SEQ ID NO: 183 | Var3-2GlaAad | ADDQNPWRAYLGlaLLFPTGlaTLLLAadLLW |
| SEQ ID NO: 184 | Var3-AadGlaD | ADDQNPWRAYLAadLLFPTGlaTLLLDLLW |
| SEQ ID NO: 185 | Var3-AadDGla | ADDQNPWRAYLAadLLFPTDTLLLGlaLLW |
| SEQ ID NO: 186 | Var3-GlaAadGla | ADDQNPWRAYLGlaLLFPTAadTLLLGlaLLW |
| SEQ ID NO: 187 | Var3-GLL | GEEQNPWLGAYLDLLFPLELLGLLELGLW |
| SEQ ID NO: 291 | Var3-M | ADDDDDDPWQAYLDLLFPTDTLLLDLLW |
| SEQ ID NO: 292 | Var4-3E | AEEQNPWRAYLELLFPTETLLLELLW |
| SEQ ID NO: 293 | Var5-3Da | ADDQNPWARYLDWLFPTDTLLLDL |
| SEQ ID NO: 294 | Var6-3Db | DNNNPWRAYLDLLFPTDTLLLDW |
| SEQ ID NO: 295 | Var7-3E | AEEQNPWARYLEWLFPTETLLLEL |
| SEQ ID NO: 296 | Var7-M | DDDDDDPWQAYLDLFPTDTLALDLW |
| SEQ ID NO: 297 | Var8-3E | EEQQPWAQYLELLFPTETLLLEW |
| SEQ ID NO: 298 | Var9-3E | EEQQPWRAYLELLFPTETLLLEW |
| SEQ ID NO: 299 | Var10-2D | AEDQNPWARYADWLFPTTLLLLD |
| SEQ ID NO: 300 | Var11-2E | AEEQNPWARYAEWLFPTTLLLLE |
| SEQ ID NO: 301 | Var12-1D | AEDQNPWARYADLLFPTTLAW |
| SEQ ID NO: 302 | Var13-1E | AEEQNPWARYAELLFPTTLAW |
| SEQ ID NO: 303 | Var15-2N | DDDDDNPNYWARYANWLFTTPLLLLNGALLVEAEET |
| SEQ ID NO: 304 | Var16-2P | DDDDDNPNYWARYAPWLFTTPLLLLPGALLVEAEET |
| SEQ ID NO: 305 | Var17 | AEQNPIYFARYADFLFTTPLLLLDLALLWDADET |
| SEQ ID NO: 306 | Var18 | AEQNPIYWARYADFLFTTPLLLLDLALLVDADET |
| SEQ ID NO: 307 | Var19a | AEQNPIYWARYADWLFTTPL |
| SEQ ID NO: 308 | Var20 | AEQNPIYFARYADLLFPTTLAW |
| SEQ ID NO: 309 | Var21 | AEQNPIYWARYADLLFPTTLAF |
| SEQ ID NO: 310 | Var22 | AEQNPIYWARYADLLFPTTLAW |
| SEQ ID NO: 311 | Var23 | AEQNPIYFARYADWLFTTPL |
| SEQ ID NO: 312 | Var24 | EDQNPWARYADLLFPTTLAW |
| SEQ ID NO: 3 | ATRAM | GLAGLAGLLGLEGLLGLPLGLLEGLWLGLELEGN |
| SEQ ID NO: 188 | pHLIP-CA9 | EQNPIYILDLVFGLLFAVTSVDFLVQWDDAGD |
| SEQ ID NO: 82 | pHLIP-Rho | NLEGFFATLGGEIALWSLVVLAIE |
| SEQ ID NO: 189 | pHLIP-RhoM1 | NNEGFFATLGGEIALWSDVVLAIE |
| SEQ ID NO: 190 | pHLIP-RhoM2 | DNNEGFFATLGGEIPLWSDVVLAIE |

Substitutions with natural amino acids may alternatively or additionally be characterized using a BLOcks SUbstitution Matrix (a BLOSUM matrix). An example of a BLOSUM matrix is the BLOSUM62 matrix, which is described in Styczynski et al. (2008) “BLOSUM62 miscalculations improve search performance” Nat Biotech 26 (3): 274-275, the entire content of which is incorporated herein by reference. The BLOSUM62 matrix is shown in FIG. 9.

Substitutions scoring at least 4 on the BLOSUM62 matrix are referred to herein as “Class I substitutions”; substitutions scoring 3 on the BLOSUM62 matrix are referred to herein as “Class II substitutions”; substitutions scoring 2 or 1 on the BLOSUM62 matrix are referred to herein as “Class III substitutions”; substitutions scoring 0 or −1 on the BLOSUM62 matrix are referred to herein as “Class IV substitutions”; substitutions scoring −2, −3, or −4 on the BLOSUM62 matrix are referred to herein as “Class V substitutions.”

Various embodiments of the subject application include pH-triggered peptides that have 1, 2, 3, 4, 5, 6, 7, 8, 9, 10 or more Class I, II, III, IV, or V substitutions compared to a pH-triggered peptide exemplified herein, or 1, 2, 3, 4, 5, 6, 7, 8, 9, 10 or more of any combination of Class I, II, III, IV, and/or V substitutions compared to a pH-triggered peptide exemplified herein.

Aspects of the present subject matter also relate to pHLIP peptides having 1, 2, 3, 4, 5, or more amino acid insertions or deletions compared to pHLIP peptides exemplified herein. Also provided are pHLIP peptide variants having no insertions or deletions compared to a pHLIP peptide exemplified herein.

### D-Amino Acids

Of the standard α-amino acids, all but glycine can exist in either of two optical isomers, called L or D amino acids, which are mirror images of each other. While L-amino acids represent all of the amino acids found in proteins during translation in the ribosome, D-amino acids are found in some proteins produced by enzyme posttranslational modifications after translation and translocation to the endoplasmic reticulum. D amino acids are abundant components of the peptidoglycan cell walls of bacteria, and D-serine acts as a neurotransmitter in the brain. The L and D convention for amino acid configuration refers not to the optical activity of the amino acid itself, but rather to the optical activity of the isomer of glyceraldehyde from which that amino acid can be synthesized (D-glyceraldehyde is dextrorotary; L-glyceraldehyde is levorotary).

pHLIP peptides either fully or partially built of D-amino acids possess advantages over L-pHLIP peptides. For example, D-pHLIP peptides are biodegraded slower than their levorotary counterparts leading to enhanced activity and longer biological half lives (Sela and Zisman, 1997 FASEB J, 11: 449-456, incorporated herein by reference). Thus, D-pHLIP peptides may be used in the methods disclosed herein. Included herein are pHLIP peptides that comprise solely L-amino acids or solely D-amino acids, or a combination of both D-amino acids and L-amino acids.

### Isotopes

pHLIP peptides and/or cargo compounds optionally contain radioactive elements or stable isotopes, or a combination of both. Stable isotopes are chemical isotopes that may or may not be radioactive, but if radioactive, have half-lives too long to be measured. Different isotopes of the same element (whether stable or unstable) have nearly the same chemical characteristics and therefore behave almost identically in biology (a notable exception is the isotopes of hydrogen). The mass differences, due to a difference in the number of neutrons, will result in partial separation of the light isotopes from the heavy isotopes during chemical reactions and during physical processes such as diffusion and vaporization. This process is called isotope fractionation. Examples of stable isotopes include oxygen, carbon, nitrogen, hydrogen and sulfur. Heavier stable isotopes include iron, copper, zinc, and molybdenum.

Gamma cameras are used in e.g. scintigraphy, SPECT and PET to detect regions of biologic activity that may be associated with disease. In various embodiments, a relatively short lived isotope, such as ¹²³I is administered to the patient.

Scintigraphy (“scint”) is a form of diagnostic test wherein radioisotopes are taken internally, for example intravenously or orally. Then, gamma cameras capture and form two-dimensional images from the radiation emitted by the radiopharmaceuticals.

Single-photon emission computed tomography (SPECT) is a 3D tomographic technique that uses gamma camera data from many projections and can be reconstructed in different planes. A dual detector head gamma camera combined with a CT scanner, which provides localization of functional SPECT data, is termed a SPECT/CT camera, and has shown utility in advancing the field of molecular imaging. In SPECT imaging, the patient is injected with a radioisotope, most commonly Thallium ²⁰¹TI, Technetium ^{99m}TC, Iodine ¹²³I and Gallium ⁶⁷Ga.

Positron emission tomography (PET) uses coincidence detection to image functional processes. Short-lived positron emitting isotope, such as ¹⁸F, is incorporated with an organic substance such as glucose, creating F18-fluorodeoxyglucose, which can be used as a marker of metabolic utilization. Images of activity distribution throughout the body can show rapidly growing tissue, like tumor, metastasis, or infection. PET images can be viewed in comparison to computed tomography scans to determine an anatomic correlate. Other radioisotopes used in nuclear medicine thallium-201, tellurium-123, cadmium-113, cobalt-60, and strontium-82.

### Chemotherapeutic Agents

Various chemotherapeutic agents may serve as pH-triggered compound cargo compounds. Non-limiting examples include alkylating agents (such as nitrogen mustards, notrisoureas, alkyl sulfonates, triazines, ethylenimines, and platinum-based compounds); antimetabolites (such as 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®)); topoisomerase inhibitors (e.g., topotecan, irinotecan, etoposide, and teniposide); taxanes (such as paclitaxel and docetaxel); platinum-based chemotherapeutics (such as cisplatin and carboplatin); anthracyclines (such as daunorubicin, doxorubicin (Adriamycin®), epirubicin, and idarubicin); epothilones (e.g., ixabepilone); vinca alkaloids (e.g., vinblastine (Velban®), vincristine (Oncovin®), and vinorelbine (Navelbine®)); estramustine; actinomycin-D; bleomycin; mitomycin-C; mitoxantrone; imatinib; lenalidomide; pemetrexed; bortezomib; leuprorelin; and abiraterone.

### Antimicrobial Cargo Compounds

Various antimicrobial agents may serve as pH-triggered compound cargo compounds. For example, the antimicrobial agent may be an antibacterial agent, an antifungal agent, or an antiprotozoal agent. In some embodiments, an antibacterial agent is also effective at killing fungi and/or protozoans, or slowing the growth thereof. In some embodiments, a composition comprising a pH-triggered compound linked to an antimicrobial cargo is applied to the skin or a mucous membrane to prevent or control a microbial infection. In various embodiments, the infection is a bacterial or a fungal infection. In certain embodiments, the infection is a protozoan infection, such as leishmaniasis.

Non-limiting examples of microbial infections include diaper rashes, vaginal yeast infections, opportunistic skin infections, tineal fungal infections, superficial skin infections, acne, athlete's foot, thrush (candidiasis), and the like. In various embodiments, a cargo compound inhibits the growth of one or more microbe species selected from the group consisting of Staphylococcus species, Streptococcus species, Pseudomonas species, Escherichia coli, Gardnerella vaginalis, Propionibacterium acnes, Blastomyces species, Pneumocystis carinii, Aeromonas hydrophilia, Trichosporon species, Aspergillus species, Proteus species, Acremonium species, Cryptococcus neoformans, Microsporum species, Aerobacter species, Clostridium species, Klebsiella species, Candida species and Trichophyton species.

Non-limiting examples of antibacterial agents include penicillins (e.g., methicillin, nafcillin, oxacillin, cloxacillin, ampicillin, amoxicillin, pivampicillin, hetacillin, bacampicillin, metampicillin, talampicillin, epicillin, dicloxacillin, carbenicillin, ticarcillin, mezlocillin, piperacillin, penicillin G, and penicillin V); cephalosporins (e.g., cefaclor, cefonicid, cefprozil, cefuroxime, cefuzonam, cefmetazole, cefotetan, cefoxitin, cefacetrile, cefadroxil, cephalexin, cefaloglycin, cefalonium, cefaloridine, cefalotin, cefapirin, cefatrizine, cefazaflur, cefazedone, cefazolin, cefradine, cefroxadine, ceftezole, cefcapene, cefdaloxime, cefdinir, cefditoren, cefetamet, cefixime, cefmenoxime, cefodizime, cefotaxime, cefovecin, cefpimizole, cefpodoxime, cefteram, ceftamere, ceftibuten, ceftiofur, ceftiolene, ceftizoxime, ceftriaxone, cefclidine, cefepime, cefluprenam, cefoselis, cefozopran, cefpirome, cefquinome ceftobiprole, ceftaroline, ceftolozane, cefaloram, cefaparole, cefcanel, cefedrolor, cefempidone, cefetrizole, cefivitril, cefmatilen, cefmepidium, cefoxazole, cefrotil, cefsumide, ceftioxide, cefuracetime, and nitrocefin); carbapenems (e.g., meropenem, ertapenem, doripenem, biapenem, panipenem, betamipron); rifamycins (e.g., rifamycin B, rifamycin SV, rifampicin, rifabutin, rifapentine, and rifaximin); lipiarmycins (e.g., lipiarmycin B, fidaxomicin); quinolones (e.g., cinoxacin, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin balofloxacin, grepafloxacin, levofloxacin, pazufloxacin, sparfloxacin, temafloxacin, tosufloxacin, clinafloxacin, gatifloxacin, gemifloxacin, moxifloxacin, sitafloxacin, trovafloxacin, nemonoxacin, delafloxacin, and prulifloxacin); sulfonamides (e.g., sulfacetamide, sulfadiazine, sulfadimidine, sulfafurazole, sulfisomidine, sulfadoxine, sulfamethoxazole, sulfamoxole, sulfanitran, sulfadimethoxine, sulfamethoxypyridazine, sulfametoxydiazine, sulfadoxine, and sulfametopyrazine); macrolides (e.g., azithromycin, clarithromycin, erythromycin, fidaxomicin, telithromycin, carbomycin A, josamycin, kitasamycin, midecamycin, midecamycin acetate, oleandomycin, solithromycin, spiramycin, troleandomycin, tylosin, tylocine, and roxithromycin); lincosamides (e.g., lincomycin and clindamycin); tetracyclines (e.g., tetracycline); aminoglycosides (e.g., streptomycin, kanamycin, amikacin, dibekacin, sisomicin, netilmicin, tobramycin, gentamicin, and neomycin); cyclic lipopeptides (such as daptomycin); glycylcyclines (such as tigecycline); oxazolidinones (such as linezolid); and lipiarmycins (such as fidaxomicin); arsphenamine; prontosil; trimethoprim (TMP); sulfamethoxazole (SMX); co-trimoxaxole (a combination of TMP and SMX); meclocycline; neomycin B, C, or E; poymyxin B; bacitracin; tazobactam; a combination of ceftolozane and tazobactam; ceftazidime; avibactam; a combination of ceftazidime and avibactam; ceftaroline; andavibactam; a combination of ceftaroline and andavibactam; imipenem; plazomicin; eravacycline; and brilacidin. In some embodiments, two or more pH-triggered compounds, each comprising a different antibiotic, are combined to deliver a combination of antibiotics to a site.

Non-limiting examples of antifungal agents include polyene antifungals (e.g., amphotericin B, candicidin, filipin, hamycin, natamycin, nystatin, and rimocidin); imidazoles (bifonazole, butoconazole, clotrimazole, econazole, fenticonazole, isoconazole, ketoconazole, luliconazole, miconazole, omoconazole, oxiconazole, sertaconazole, sulconazole, and tioconazole); triazoles (albaconazole, efinaconazole, epoxiconazole, fluconazole, isavuconazole, itraconazole, posaconazole, propiconazole, ravuconazole, terconazole, voriconazole); thiazoles (e.g., abafungin); allylamines (e.g., amorolfin, butenafine, naftifine, and terbinafine); echinocandins (e.g., anidulafungin, caspofungin, and micafungin); ciclopirox; 5-fluorocytosine; griseofulvin; haloprogin; tolnaftate, undecylenic acid, Crystal violet, and balsam of Peru.

Non-limiting examples of antiprotozoal agents include metronidazole, co-trimoxaxole, eflornithine, furazolidone, melarsoprol, metronidazole, ornidazole, paromomycin sulfate, pentamidine, pyrimethamine, tinidazole, and nifursemizone.

An antimicrobial composition can be formulated to be suitable for application in a variety of ways, for example in a cream for skin (e.g., ringworm or athlete's foot), in a wash for the mouth (e.g., oral thrush), in a douche for vaginal application (e.g., vaginitis), in a powder for chaffing (e.g., dermatitis), in a liquid for toe nails (e.g., tinea pedis), in a bath salt or bath powder for treating genital, foot or other tissue infections in a bath, and the like.

Antimicrobial compositions can be formulated to be suitable for application in a variety of ways, for example in a cream for skin (e.g., ringworm or athlete's foot), in a wash for the mouth (e.g., oral thrush), in a douche for vaginal application (e.g., vaginitis), in a powder for chaffing (e.g., dermatitis), in a liquid for toe nails (e.g., tinea pedis), in a bath salt or bath powder for treating genital, foot or other tissue infections in a bath, and the like. In various embodiments of the invention, there is provided a method of inhibiting growth of or a pathogenic microbe, including applying a pH-triggered compound or a composition comprising a pH-triggered compound to a solid surface, contacting the solid surface with the applied pH-triggered compound thereon to skin or a mucous membrane of a mammal, and allowing the solid surface to contact the skin or mucous membrane for sufficient time to allow the pH-triggered compound to inhibit growth the pathogenic microbe adjacent to or on the skin or mucous membrane. In some embodiments, the applying step includes applying the composition to a diaper, pliable material for wiping skin or a mucous membrane, dermal patch, adhesive tape, absorbent pad, tampon or article of clothing. In another embodiment, the applying step includes impregnating the composition into a fibrous or non-fibrous solid matrix.

The term “topical” is broadly utilized herein to include both epidermal and/or skin surfaces, as well as mucosal surfaces of the body.

Fluorescent pH-Triggered Compounds

Included herein are pH-triggered compounds comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more pHLIP peptides and 1 or more fluorophores. As used herein, the term “fluorophore” includes any compound that emits energy. The energy may be in the form of, e.g., acoustic energy (such as sound waves), heat, or electromagnetic radiation. In various embodiments, the electromagnetic radiation may be visible or non-visible to the human eye. In some embodiments, the electromagnetic radiation is infrared or near-infrared. Non-limiting examples of fluorophores include luminescent compounds, fluorescent compounds, phosphorescent compounds, chemiluminescent compounds, optoacoustic compounds, and quencher compounds (e.g., fluorescent quencher compounds). Fluorophores may comprise, e.g., small molecule compounds (e.g., organic compounds having a molecular weight of less than about 2000, 1000, or 500 daltons), proteins, or chelated metals (e.g., a chelator attached to a metal via covalent or non-covalent coordination bonds, wherein the combination of the chelator and the metal is fluorescent). In some embodiments, a chelated metal is within a “cage” formed by a chelator, and the combination of the chelator and the metal is fluorescent. In certain embodiments, the emission of energy (e.g., electromagnetic radiation such as luminescence, acoustic energy such as sound waves, or heat) does not involve the absorption and then emission of energy. In some embodiments, the emission of energy involves the absorbance and then the emission of energy.

As used herein, a compound that transfers greater than 50% the energy of absorbed light into the heat is called a “quencher.” In some embodiments, a quencher transfers all of the energy of absorbed light into heat. In various embodiments, a quencher can emit some amount of light, but most of the absorbed energy (e.g., at least about 55%, 60%, 65%, 70%, 75%, 80%, 85%, 90%, 95%, 96%, 97%, 98%, 99% of the absorbed energy) is transferred into the heat. Non-limiting examples of quenchers include: i) Dabsyl (dimethylaminoazobenzenesulfonic acid); ii) Black Hole Quenchers (which can quench in wide range of practically the entire visible spectrum); and iii) IRDye QC-1 [which can quench in the range for visible to NIR (500-900 nm)]. A main principle of optoacoustic imaging is the following: Absorption of light by a fluorophore or quencher, and the transfer of energy into heat, which leads to thermal expansion and the generation of acoustic waves, which are detected. In general, fluorophores transfer some, e.g., a minimal amount, of energy to heat; however most of the energy of a fluorophore is emitted in a form of light. In certain preferred embodiments relating to luminescent fluorophores (e.g., fluorophores that emit electromagnetic radiation such as light), a fluorophore emits more energy in the form of electromagnetic radiation (e.g., light), and less energy is transferred to heat. In certain preferred embodiments relating to quenchers, a quencher emits less energy in the form of electromagnetic radiation (e.g., light), and more energy is transferred to heat. Therefore, ICG can be used as a fluorophore in fluorescent imaging, as well as in optoacoustic imaging, due its property of transferring some energy to the heat.

In embodiments, the pHLIP compound is attached to one or more fluorophores (e.g., a fluorophore, a quencher such as a fluorophore quencher, or a combination comprising a fluorophore-quencher pair) to form a pH-triggered compound that is used as a diagnostic, imaging, ex vivo imaging agent, or as a research tool. In various embodiments, the pH-triggered compound comprises one or more fluorophores attached to a functional group used as a diagnostic, imaging, ex vivo imaging agent, or as a research tool.

In some embodiments, the fluorophore comprises a fluorescent dye, or a fluorescent quencher, or a combination of both.

In some embodiments, a fluorophore-quencher system used in fluorescence-guided imaging. For non-limiting descriptions of such systems, see, e.g., www.bachem.com/service-support/newsletter/peptide-trends-july-2016/. A non-limiting example of the use of a fluorophore-quencher system is described in Karabadzhak et al. (2014) ACS Chem Biol. 9(11):2545-53, the entire content of which is incorporated herein by reference. In certain embodiments, when the distance between a fluorophore and a quencher increases [e.g., because of a conformational change or due to the breakage of a bond (such as a peptide or other bond) connecting the fluorophore and the quencher], then the intensity of emission of fluorophore increases. In certain embodiments, the efficiency of fluorescence increases when the distance between the fluorophore and the quencher increases, which results in increased of fluorescent intensity.

In some embodiments, a pH-triggered compound comprising a fluorophore or a quencher (e.g., a pHLIP-quencher) is used for optoacoustic imaging. In various embodiments, optoacoustic imaging comprises a compound or moiety that absorbs light and transfers it to heat (e.g., with a optoacoustic imaging agent), which is measured by ultrasound, as opposed to fluorescence. In embodiments, fluorescence comprises a compound of moiety that absorbs light and emits it in the form of fluorescence or phosphorescence. In some embodiments, a fluorophore (e.g., a fluorophore that emits more energy in the form of light than heat) is used for optoacoustic imaging. In certain embodiments, an ICG-pH-triggered compound is used for optoacoustic imaging. Anon-limiting example of the use of a compound comprising a pH-triggered compound and a fluorescent dye as a multispectral optoacoustic tomography (MSOT) imaging agent is described in Kimbrough et al. (2015) Clin Cancer Res. 21(20):4576-85, the entire content of which is incorporated herein by reference.

In certain embodiments, the fluorophore comprises a near-infrared (NIR) fluorescent dye, e.g., indocyanine green (ICG), which operates in (e.g., has a peak emission wavelength within) NIR wavelengths. Infrared radiation extends from the nominal red edge of the visible spectrum at 700 nanometers (nm) to 1 mm. NIR radiation comprises a wavelength of 750 nm to 1.4 μm. In some embodiments, the ICG has a peak emission wavelength between 810 nm and 880 nm (e.g., in the context of a pH-triggered compound). In certain embodiments, the ICG has a peak emission wavelength between 810 nm and 860 nm. In various embodiments, the ICG has a peak emission wavelength of about 800, 805, 810, 815, 820, 825, 830, 835, 840, 845, 850, 855, 860, 865, 870, or 880 nm. In some embodiments, a 805 nm laser is used for ICG excitation. In certain embodiments, a 801, 802, 803, 804, 804, 805, 806, 807, 808, 809, 810, 800-805, 804-806, or 802-807 nm laser is used for ICG excitation.

Non-limiting examples of NIR imaging systems (which may be useful in, e.g., clinical and diagnostic applications) include INFRARED 800™, available from Carl Zeiss Meditec AG; Artemis®, available from Quest Medical Imaging BV; HyperEye Medical System®, available from Mizuho Medical Co. Ltd.; Near infrared fluorescence imager PDE® C9830, available from Hamamatsu Photonics K.K.; SPECTROPATH® Image-Guided Surgery System, available from Spectropath Inc.; the following from NOVADAQ Technologies Inc.: SPY Elite® (imaging for open surgery), PINPOINT® (endoscopic fluorescence imaging), LUNA® (Fluorescence Angiography for Wound Care); Firefly® Fluorescence imaging for the da Vinci Si System, available from Intuitive Surgical Inc.; NIR Leica® FL800, available from Leica Microsystems; Fluobeam®, available from Fluoptics Minatec-BHT; KG, Storz Karl Storz-Endoskope® (Near-Infrared/Indocyanine Green), available from Karl Storz GmbH & Co.; and InfraVision™ Imaging System, available from Stryker Corporation.

In various embodiments, the fluorophore comprises an agent that operates at a wavelength (e.g., has a peak emission wavelength within) of from about 670 nm to about 750 nm, e.g., methylene blue.

In certain embodiments, the fluorophore comprises a cyanine dye. In embodiments, a cyanine dye operates at a wavelength (e.g., has a peak emission wavelength within) of 550-620 nm, 590-700 nm, 650-730 nm, 680-770 nm, 750-820 nm, or 770-850 nm. Non-limiting examples of cyanine dyes include Cy2, Cy3, Cy3B, Cy3.5, Cy5, Cy5.5, Cy7, and Cy7.5. In some embodiments, the cyanine dye is Cy3, Cy3.5, Cy5, Cy5.5, Cy7, or Cy7.5. In certain embodiments, the Cy3 has a peak emission wavelength between 550 and 620 nm (e.g., in the context of a pH-triggered compound). In various embodiments, the Cy3.5 has a peak emission wavelength between 590 and 700 nm (e.g., in the context of a pH-triggered compound). In some embodiments, the Cy5 has a peak emission wavelength between 650 and 730 nm (e.g., in the context of a pH-triggered compound). In certain embodiments, the Cy5.5 has a peak emission wavelength between 680 and 770 nm (e.g., in the context of a pH-triggered compound). In various embodiments, the Cy7 has a peak emission wavelength between 750 and 820 nm (e.g., in the context of a pH-triggered compound). In certain embodiments, the Cy7.5 has a peak emission wavelength between 770 and 850 nm (e.g., in the context of a pH-triggered compound).

In some embodiments, the peak emission wavelength of a fluorophore may vary (e.g., by about 5, 6, 7, 8, 9, or 10%) based on the environment and/or solvent around the fluorophore.

In some embodiments, the fluorophore comprises a fluorescent, or an optoacoustic contrast imaging agent. In certain embodiments, an optoacoustic imaging agent is fluorescent. In various embodiments, an optoacoustic imaging agent is not fluorescent. In certain embodiments, an optoacoustic imaging agent absorbs light, and transfers most of the light's energy (e.g., at least about 55%, 60%, 65%, 70%, 75%, 80%, 85%, 90%, 95%, 96%, 97%, 98%, 99% of the light's energy) into heat. In various embodiments, the heat is detected by ultrasound. In some embodiments, a quencher is be a fluorophore with a very low quantum yield, such that most of the energy absorbed by the quencher is transferred to heat rather than electromagnetic radiation (such as light).

Non-limiting examples of optoacoustic contrast imaging agents include ICG (which can be used for fluorescent imaging as well as for optoacoustic imaging), Alexa Fluor 750, Evans blue, BHQ3 (Black Hole Quencher®-3; commercially available from, e.g., Biosearch Technologies, California, United States), QXL@680 (commercially available from, e.g., Cambridge Bioscience, Cambridge, United Kingdom), IRDye@800CW (commercially available from, e.g., LI-COR, Nebraska, United States), MMPSense™750 FAST (commercially available from, e.g., PerkinElmer Inc., Texas, United States), diketopyrrolopyrrole cyanine, cypate-C18, Au nanoparticles (such as Au nanospheres, Au nanoshells, Au nanorods, Au nanocages, Au nanoclusters, Au nanostars, and Au nanobeacons), nanoparticles comprising a gold core covered with the Raman molecular tag trans-1,2-bis(4-pyridyl)-ethylene, Ag nanoplates, Ag nanosystems, quantum dots, nanodiamonds, polypyrrole nanoparticles, copper sulfide, graphene nanosheets, iron oxide-gold core-shells, Gd203, single-walled carbon nanotubules, dye-loaded perfluorocarbon-based nanoparticles, AuMBs, triggered nanodroplets, cobalt nanowontons, nanoroses, goldsilica core shell nanorods, superparamagnetic iron oxide, and methylene blue. Non-limiting examples and descriptions of optoacoustic contrast imaging agents are described in Wu et al. (2014) Int. J. Mol. Sci., 15, 23616-23639 (see, e.g., Table 1), the entire contents of which are incorporated herein by reference.

A pH-triggered compound comprising a fluorophore may optionally be referred to herein as a fluorescent pH-triggered compound.

In various embodiments, a fluorescent pH-triggered compound provided herein is for use as an agent in preoperative, intraoperative and postoperative settings.

In some embodiments, a fluorescent pH-triggered compound provided herein is for use as an agent for ex vivo imaging, and ex vivo diagnostics.

In various embodiments, a fluorescent pH-triggered compound provided herein is used to detect or image diseased tissue. Non-limiting examples of diseased tissue include cancerous tissue, inflamed tissue, ischemic tissue, arthritic tissue, cystic fibrotic tissue, tissue infected with a microorganism, and atherosclerotic tissue.

In some embodiments, a fluorescent pH-triggered compound provided herein is for use as an agent in fluorescence angiography. Fluorescence angiography is a procedure in which a fluorescent compound (such as a fluorescent pH-triggered compound disclosed herein) is injected into the bloodstream. The fluorescent pH-triggered compound highlights the blood vessels. In various embodiments, the vessels are in the back of the eye. In some embodiments the vessels are imaged or photographed. In non-limiting examples, fluorescence angiography is used to identify, detect image, or manage an eye disorder. In certain embodiments relating to ophthalmology, fluorescence angiography may be used to look at blood flow in, e.g., the retina and choroid.

In various embodiments, fluorescence angiography provides real-time imaging of blood vessels to follow changes during surgical procedures. Some non-limiting examples include the use of fluorescence in ophthalmology to evaluate the chorioretinal vasculature; in cardiothoracic surgery to assess the effectiveness of a coronary artery bypass; in neurovascular surgery to assess the effect of a superficial temporal artery-middle cerebral artery bypass graft in cerebral revascularization procedure; in hepatobiliary surgery to identify the haptic segment and subsegment for anatomical hepatic resection; in reconstructive surgeries; and in cholecystectomy and colorectal resection. In non-limiting examples of diagnostic applications, fluorescence angiography is used for imaging of hemodynamics in the brain; circulatory features of rheumatoid arthritis; muscle perfusion; burns and to assess various other effects of trauma.

In certain embodiments, a fluorescent pH-triggered compound provided herein is for visualization of blood circulation in ophthalmology, cardiothoracic surgery, bypass coronary surgery, neurosurgery, hepatobiliary surgery, reconstructive surgery, cholecystectomy, colorectal resection, brain surgery, muscle perfusion, wound and trauma surgery, and laparoscopic surgery.

In various embodiments, a fluorescent pH-triggered compound provided herein is for visualization of lymph nodes.

In some embodiments, a fluorescent pH-triggered compound provided herein is for visualization or detection of pre-cancerous tissue or cancerous lesions.

In certain embodiments, a fluorescent pH-triggered compound provided herein is for visualization or detection of pre-cancerous tissue or cancerous lesions in bladder, upper urinary tract, kidney, prostate, breast, head and neck, oral, pancreatic, lungs, liver, cervical, ovarian, or brain tumors.

In various embodiments, a fluorescent pH-triggered compound provided herein for real-time assessment of blood flow and tissue perfusion during intraoperative procedures.

In an aspect, provided herein is a composition for parenteral, local, or systemic administration comprising a fluorescent pH-triggered compound.

In an aspect, included herein is a composition for intravenous, intraarterial, intraperitoneal, intracerebral, intracerebroventricular, intrathecal, intracardiac, intracavernous, intraosseous, intraocular, intravitreal administration of a fluorescent pH-triggered compound.

In an aspect, provided herein is composition for intramuscular, intradermal, transdermal, transmucosal, intralesional, subcutaneous, topical, epicutaneous, extra-amniotic, intravaginal, intravesical, nasal, or oral administration of a fluorescent pH-triggered compound.

In an aspect, included herein is a composition for an ex vivo treatment of biopsy specimens, liquid biopsy specimens, surgically removed tissue, surgically removed liquids, or blood comprising a fluorescent pH-triggered compound.

In an aspect, a subject's blood is contacted with the fluorescent pH-triggered compound (e.g., in vivo or ex vivo).

In various embodiments, a lower dose of a fluorophore (such as ICG) is effective when the fluorophore is part of a fluorescent pH-triggered compound, e.g., conjugate, compared to the effective dose (e.g., for imaging or detection) of the free fluorophore, e.g., the non-conjugated fluorophore. In some embodiments, administration of a lower effective dose of the fluorophore as part of a fluorescent pH-triggered compound results in lower side effects. In certain embodiments, a fluorophore may make a subject more sensitive to solar radiation after administration such that the subject develops a greater degree of sunburn following exposure to solar radiation compared to a subject to which a fluorophore such as ICG has not been administered. In various embodiments, a fluorophore is delivered as part of a fluorescent pH-triggered compound to subject in a lower dose than would be necessary if the fluorophore was administered in free form, thereby reducing or minimizing phototoxicity (e.g., toxicity to the skin/sunburn) from exposure to solar radiation than if the free form of the fluorophore was administered.

In some embodiments, the fluorescent pH-triggered compound comprises a pHLIP compound and ICG (e.g., an ICG-pHLIP peptide such as ICG-Var3). In certain embodiments, the fluorescent pH-triggered compound is administered at a dose of about 0.01-0.5 mg/kg of a subject. In various embodiments, the fluorescent pH-triggered compound is administered at a dose of about 0.02-0.2 mg/kg of a subject. In some embodiments, the fluorescent pH-triggered compound is administered at a dose of about 0.01, 0.02, 0.03, 0.04, 0.05, 0.06, 0.07, 0.08, 0.09, 0.1, 0.125, 0.15, 0.175, 0.2, 0.25, or 0.5 mg/kg of a subject. In certain embodiments, the fluorescent pH-triggered compound is administered at a dose of at least about 0.01, 0.02, 0.03, 0.04, 0.05, 0.06, 0.07, 0.08, 0.09, 0.1, 0.125, 0.15, 0.175, or 0.2 mg/kg, but less than about 0.25, 0.5, 1, 2, 3, 4, or 5 mg/kg. In various embodiments, 1-10 mg of the fluorescent pH-triggered compound is administered to a subject. In some embodiments, about 0.5 1, 2, 3, 4, 5, 6, 7, 8, 9, 10, or 15 mg of the fluorescent pH-triggered compound is administered to a subject. In certain embodiments, at least 0.5, 1, 2, or 3 mg, but less than 10 or 1 mg, of the fluorescent pH-triggered compound is administered to the subject. In various embodiments, about 0.3-3 μmol of the fluorescent pH-triggered compound is administered to the subject. In some embodiments, about 0.1, 0.5, 1, 1.5, 2, 2.5, 3, 3.5, 4, 4.5, or 5 μmol of the fluorescent pH-triggered compound is administered to the subject. In certain embodiments, at least about 0.1, 0.5, or 1 μmol, but less than 3, 4, or 5 μmol, of the fluorescent pH-triggered compound is administered to the subject. In various embodiments, the fluorescent pH-triggered compound is administered by intravenous injection for about 1, 2, 3, 4, 5, 6, 7, 8, 9, 10, 11, 12, 13, 14, 15, 16, 17, 18, 19, 20, 1-10, 1-15, 5-10, 5-15, 5-20, 10-15, 10-20, or 15-20 minutes.

In certain embodiments, about 0.5 1, 2, 3, 4, 5, 6, 7, 8, 9, 10, or 15 mg of the fluorescent pH-triggered compound is instilled into an organ or tissue (e.g. a bladder). In certain embodiments, at least 0.5, 1, 2, or 3 mg, but less than 10 or 1 mg, of the fluorescent pH-triggered compound is instilled into an organ or tissue (e.g. a bladder). In various embodiments, about 0.3-3 μmol of the fluorescent pH-triggered compound is instilled into an organ or tissue (e.g. a bladder). In some embodiments, about 0.1, 0.5, 1, 1.5, 2, 2.5, 3, 3.5, 4, 4.5, or 5 μmol of the fluorescent pH-triggered compound is instilled into an organ or tissue (e.g. a bladder). In certain embodiments, at least about 0.1, 0.5, or 1 μmol, but less than 3, 4, or 5 μmol, of the fluorescent pH-triggered compound is instilled into an organ or tissue (e.g. a bladder). In various embodiments, the fluorescent pH-triggered compound is instilled into an organ or tissue (e.g. a bladder) for about 1, 2, 3, 4, 5, 6, 7, 8, 9, 10, 11, 12, 13, 14, 15, 16, 17, 18, 19, 20, 1-10, 1-15, 5-10, 5-15, 5-20, 10-15, 10-20, or 15-20 minutes.

In certain embodiments, the fluorescent pH-triggered compound further comprises polyethylene glycol. In some embodiments, the fluorescent pH-triggered compound further comprises one or more polyethylene glycol subunits (e.g., 3, 4, 5, 6, 7, 8, 9, 0, 11, 12, 13, 14, 15, 16, 17, 18, 19, 20, 3-10, 10-20, or 3-20 subunits).

Included herein is a method for detecting (e.g., imaging) blood flow in a subject, comprising (a) administering a fluorescent pH-triggered compound comprising a fluorophore (such as ICG) disclosed herein to the subject; (b) contacting the subject (e.g., an area, cell, tissue, or organ of the subject, such as an area or tissue that may comprise a portion of the administered fluorescent pH-triggered compound) with electromagnetic radiation comprising an excitation wavelength of the fluorophore; and (c) detecting electromagnetic radiation emitted from the fluorescent pH-triggered compound in the subject. In embodiments, detection of the radiation indicates the presence (e.g., the location or amount at a location) of blood in the subject. In embodiments, an image of the blood in the subject is produced.

Also provided is a method for detecting (e.g., imaging) a fluorescent pH-triggered compound in a subject, comprising (a) administering a fluorescent pH-triggered compound comprising a fluorophore (such as ICG) disclosed herein to the subject; (b) contacting the subject (e.g., an area or tissue of the subject, such as an area, cell, tissue, or organ that may comprise a portion of the administered fluorescent pH-triggered compound) with electromagnetic radiation comprising an excitation wavelength of the fluorophore; and (c) detecting electromagnetic radiation emitted from the fluorescent pH-triggered compound in the subject. In embodiments, detection of the radiation indicates the presence (e.g., the location or amount at a location) of a bodily fluid such as blood in the subject. In embodiments, an image of the blood in the subject is produced.

Included herein is a method for optoacoustic detection or imaging of blood flow in a subject, comprising (a) administering a fluorescent pH-triggered compound, wherein the fluorophore is an optoacoustic imaging agents such as a luminescent fluorophore or a quencher; (b) contacting the subject (e.g., an area, cell, tissue, or organ of the subject, such as an area or tissue that may comprise a portion of the administered fluorescent pH-triggered compound) with electromagnetic radiation comprising an excitation wavelength of the fluorophore; and (c) detecting energy such as acoustic energy (e.g., sound waves). In embodiments, detection of the energy indicates the presence (e.g., the location or amount at a location) of blood in the subject. In various embodiments, an image of the blood in the subject is produced. In some embodiments, the presence of acoustic energy is detected by ultrasound (e.g., heat is released and creates expansion, generating sound waves, which is detected).

The present subject matter also provides a method for detecting (e.g., imaging) a fluorescent pH-triggered compound in a subject, wherein the fluorophore is an optoacoustic imaging agents such as a luminescent fluorophore or a quencher, the method comprising (a) administering the fluorescent pH-triggered compound to the subject; (b) contacting the subject (e.g., an area or tissue of the subject, such as an area, cell, tissue, or organ that may comprise a portion of the administered fluorescent pH-triggered compound) with electromagnetic radiation comprising an excitation wavelength of the fluorophore; and (c) detecting energy such as acoustic energy (e.g., sound waves). In embodiments, detection of the energy indicates the presence (e.g., the location or amount at a location) of a bodily fluid such as blood in the subject. In embodiments, an image of the blood in the subject is produced. In embodiments, the presence of acoustic energy is detected by ultrasound.

Depending on context, “excitation wavelength” may be used synonymously with “absorption wavelength.”

In various embodiments, the method comprises a fluorescence-guided imaging procedure performed during surgery or during a doctor's visit. In some embodiments, the method comprises fluorescence angiography. In certain embodiments, the method comprises the assessment of the perfusion of tissues and organs. In various embodiments, the method comprises the assessment of hepatic function. In some embodiments, the fluorescence-guided imaging procedure comprises targeting, marking, detecting, or visualization of pre-cancerous tissue, cancerous tissue, inflamed tissue, ischemic tissue, arthritic tissue, tissue infected with a microorganism, and/or atherosclerotic tissue. In certain embodiments, the method comprises assessing patency of a coronary artery bypass during cardiothoracic surgery. In some embodiments, the method comprises assessing the effect of a superficial temporal artery-middle cerebral artery bypass graft during or after neurovascular surgery, e.g., in a cerebral revascularization procedure. In certain embodiments, the method comprises identify the haptic segment and subsegment for anatomical hepatic resection during hepatobiliary surgery. In some embodiments, the method comprises imaging tissue or blood during a reconstructive surgery. In certain embodiments, the method comprises imaging tissue or blood during cholecystectomy or colorectal resection. In some embodiments, the method comprises intraoperatively identifying brain tumors such as malignant gliomas.

In various embodiments, the method comprises a diagnostic imaging procedure. In some embodiments, the method comprises retinal angiography. In certain embodiments, the method comprises detecting or imaging chorioretinal vasculature.

In some embodiments, the method comprises mapping and visualization of lymph nodes. In certain embodiments, the method comprises targeting and marking (e.g., visualizing or detecting) pre-cancerous tissue, cancerous lesions and/or assessment of tumor margins.

In various embodiments, the fluorescent pH-triggered compound is administered by parenteral, local, or systemic administration. In certain embodiments, a fluorescent pH-triggered compound is administered by intravenous, intraarterial, intraperitoneal, intracerebral, intracerebroventricular, intrathecal, intracardiac, intracavernous, intraosseous, intraocular, or intravitreal administration. In various embodiments, fluorescent pH-triggered compound is administered by intramuscular, intradermal, transdermal, transmucosal, intralesional, subcutaneous, topical, epicutaneous, extra-amniotic, intravaginal, intravesical, nasal, or oral administration.

In an aspect, provided herein is a method for the ex vivo staining of human specimens and ex vivo diagnostics, comprising (a) contacting a biological sample from a subject with a fluorescent pH-triggered compound comprising a fluorophore (such as ICG) disclosed herein; (b) contacting the biological sample with electromagnetic radiation comprising an excitation wavelength of the fluorophore; and (c) detecting electromagnetic radiation emitted from the fluorescent pH-triggered compound. In embodiments, the biological sample comprises a biopsy specimen, a liquid biopsy specimen, surgically removed tissue, a surgically removed liquid, or blood.

In certain embodiments, a compound comprises multiple (e.g., 2-32, or 2, 3, 4, 5, 6, 7, 8, 9, 10 or more) units, wherein each unit comprises a pHLIP peptide that is connected (e.g., linked by a covalent bond) to a cargo compound. In some embodiments, the cargo compound comprises a fluorophore. In certain embodiments, the fluorophore is ICG.

In various embodiments, a fluorescent pH-triggered compound comprises two or more of the following compound linked (e.g., covalently) together (SEQ ID NO: 15 is disclosed below):

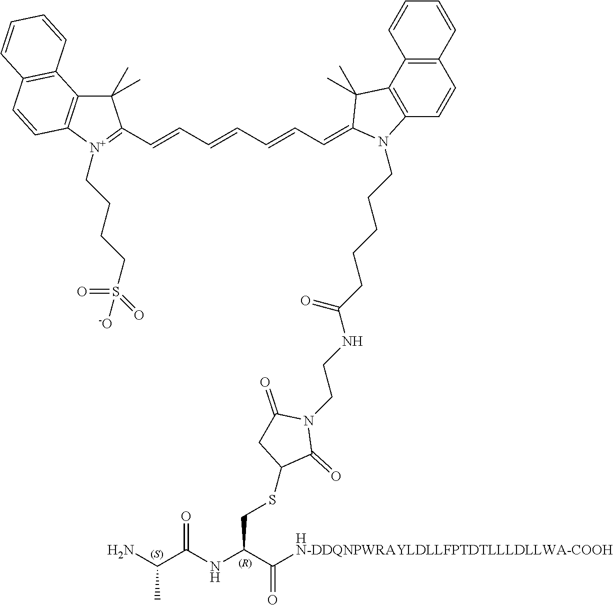

In the sequence above, the pHLIP peptide sequence is NH₂-ACDDQNPWRAYLDLLFPTDTLLLDLLWA-COOH (SEQ ID NO: 15), however the structures of the alanine and the cysteine at the N-terminal end of the peptide are shown.

In various embodiments, a fluorescent pH-triggered compound comprises two or more of one of or any combination of the following compounds linked (e.g., covalently) together:

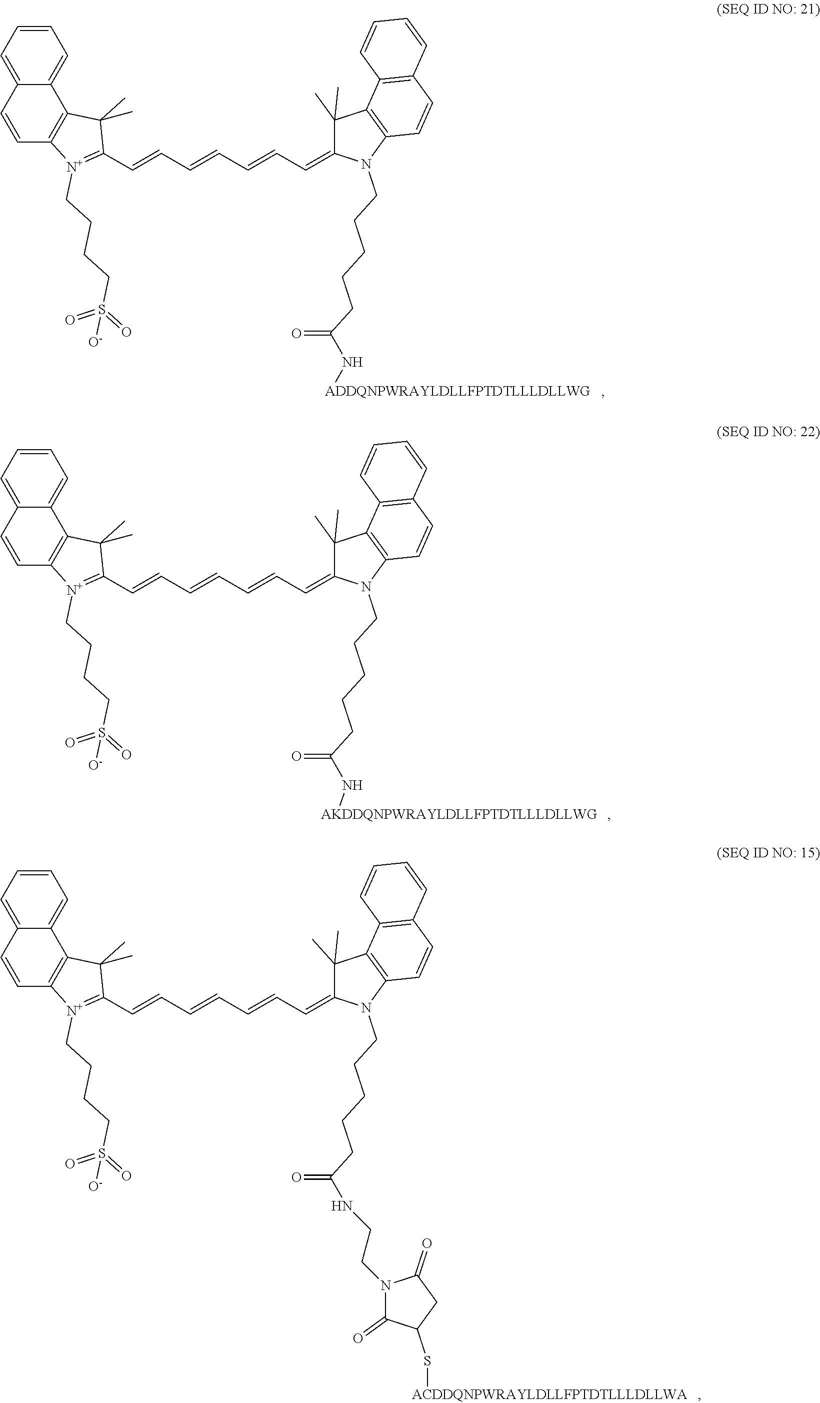

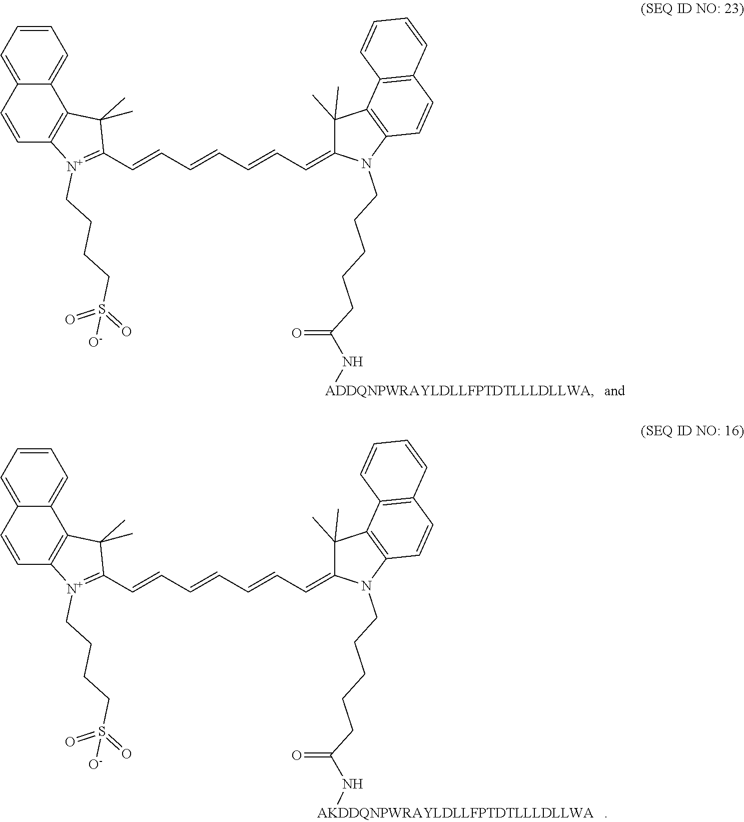

### Indocyanine Green

The non-invasive near-infrared (NIR) fluorescence imaging dye ICG is approved by the United States Food and Drug administration (FDA) for ophthalmologic angiography to determine cardiac output and liver blood flow and function. This dye is also used in cancer patients for the detection of solid tumors, localization of lymph nodes, and for angiography during reconstructive surgery, visualization of retinal and choroidal vasculature, and photodynamic therapy. In cancer diagnostics and therapeutics, ICG could be used as both an imaging dye and a hyperthermia agent.

ICG is a tricarbocyanine-type dye with NIR-absorbing properties (peak absorption around 800 nm) and little absorption in the visible range thus exhibit low autofluorescence, tissue absorbance, and scatter at NIR wavelengths (700-900 nm).

Unconjugated ICG may comprise the following structure:

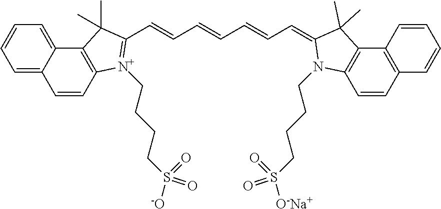

A CAS Registry Number for ICG is 3599-32-4.

ICG may be modified to, e.g., facilitate attachment the attachment thereof to peptides, such as pHLIPs disclosed herein. Non-limiting examples of commercially available (e.g., from Intrace Medical SA, Lausanne, Switzerland) modified ICG compounds include ICG N-succinimidyl ester (ICG-NHS ester), ICG-CBT, ICG-maleimide, ICG-azide, ICG-alkyne, and ICG-PEG-NHS ester.

The succinimidyl esters (NHS) of the ICG dye offer the opportunity to develop optimal conjugates. Succinimidyl ester active groups provide an efficient and convenient way to selectively link ICG dyes to primary amines (R—NH₂) on various substrates (antibodies, peptides, proteins, nucleic-acid, small molecule drugs, etc.). Succinimidyl esters have very low reactivity with aromatic amines, alcohols, and phenols, including tyrosine and histidine. An example of ICG-NHS ester comprises the following features:
Excitation Class: Near infrared, NIR
Excitation/Emission maximum (nm): 790/830
Molecular Weight: 828.04 g·mol⁻¹
Formula: C₄₉H₅₃N₃O₇S

Structure:

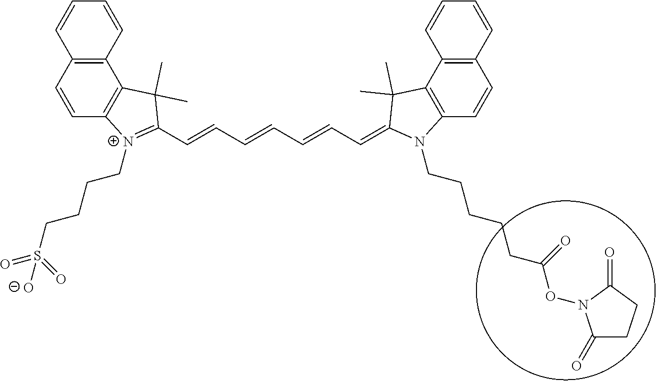 [SMILES: CC1(C)C(/C=C/C=C/C=C/C=C2/N(CCCCCC(=O)ON3C(=O)CCC3=O)c3ccc4ccccc4c3C2(C)C)=[N+](CCCCS(=O)(=O)[O-])c2ccc3ccccc3c21]

The circled portion of the structure above indicates the linker moiety.

A maleimide active group provides an efficient and convenient way to selectively link ICG dye to sulfhydryl groups (free thiol, R—SH) on various substrates (antibodies, peptides, proteins, oligonucleotides, small molecule drugs, etc.) at neutral (physiological) pH without any activation. Maleimides have very low reactivity with amines, alcohols, and phenols (such as tyrosine and histidine) and do not react with histidine and methionine, providing a very high labeling selectivity. An example of ICG-maleimide comprises the following features:
Excitation Class: Near infrared, NIR
Excitation/Emission maximum (nm): 790/830
Molecular Weight: 853.09 g·mol⁻¹
Formula: C₅₁H₅₆N₄O₆S

Structure:

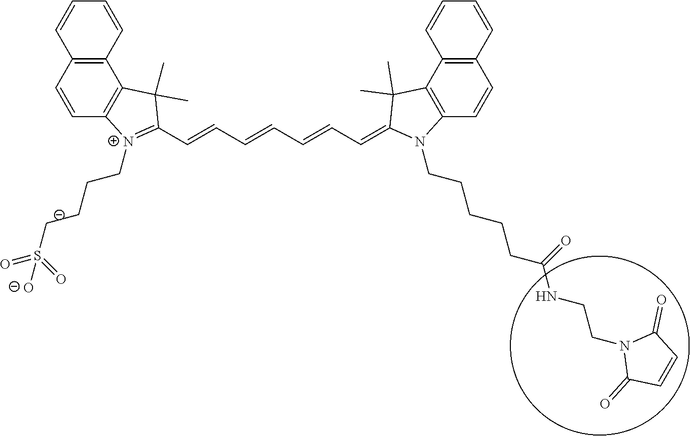

The circled portion of the structure above indicates the linker moiety.

The 2-cyanobenzothiazole labeling procedure is based on the biocompatible click-reaction between 2-cyanobenzothiazole moiety and any 1, 2- or 1, 3-aminothiols (e.g. free or N-terminal cysteine). This click reaction is 3 orders of magnitude faster than commonly used Staudinger ligation and can provide useful conjugates. Cyanobenzothiazole (CBT) active groups provide an efficient and convenient way to site-selectively link ICG dyes to 1,2- or 1,3-aminothiols on various substrates (antibodies, peptides, proteins, nucleic-acid, small molecule drugs, etc.) without any additional activation. The labeling reaction with aminothiols is selective over reaction with simple thiols. The CBT click chemistry can be used together with all other biocompatible click reactions (like azide, alkyne, triphenylphosphine, tetrazine etc.), as it is very selective. In addition in ICG-CBT labeling procedure no side product is formed as here is no leaving group (unlike NHS esters). An example of an ICG-CBT comprises the following features:
Excitation Class: Near infrared, NIR
Excitation/Emission maximum (nm): 790/830
Molecular Weight: 931.38 g·mol⁻¹
Formula: C₅₅H₅₇N₅O₅S₂

Structure:

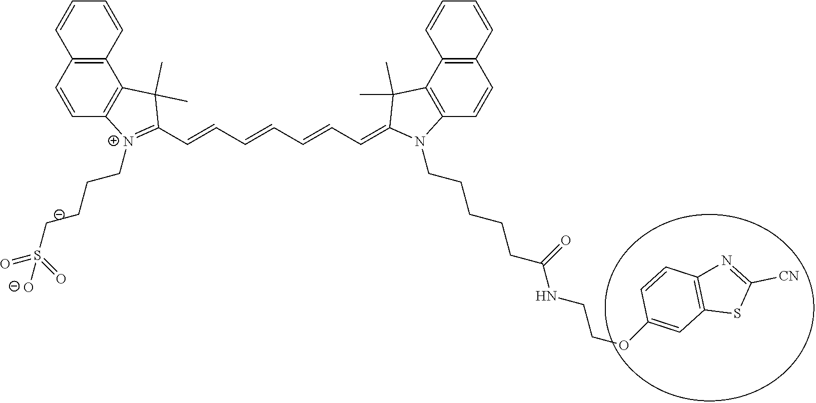

The circled portion of the structure above indicates the linker moiety.

ICG-azide can be used to label alkyne-tagged biomolecules (like proteins, lipids, nucleic acids, sugars) chemoselectively via click-chemistry. An example of ICG-azide comprises the following features:
Excitation Class: Near infrared, NIR
Excitation/Emission maximum (nm): 790/830
Molecular Weight: 931.21 g·mol⁻¹
Formula: C₅₃H₆₆N₆O₇S

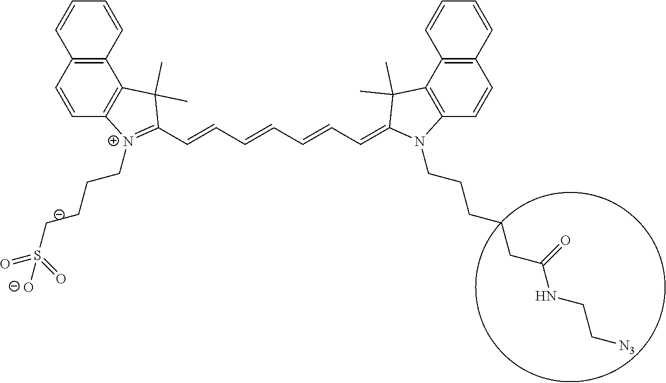

The circled portion of the structure above indicates the linker moiety.

ICG-alkyne can be used to label azide-tagged molecules via Cu(II)-catalyzed click reaction. The reaction is chemoselective and biocompatible. An example of ICG-alkyne comprises the following features:
Excitation Class: Near infrared, NIR
Excitation/Emission maximum (nm): 790/830
Solubility: DMSO, DMF, Acetonitrile, Methanol
Molecular Weight: 767.38 g·mol⁻¹
Formula: C₄₈H₅₃N₃O₄S

### Cyanine Fluorophores

Cyanine fluorophores may optionally be referred to herein as “cyanine dyes.” Cyanine dyes are molecules containing polymethine bridge between two nitrogen atoms with a delocalized charge:

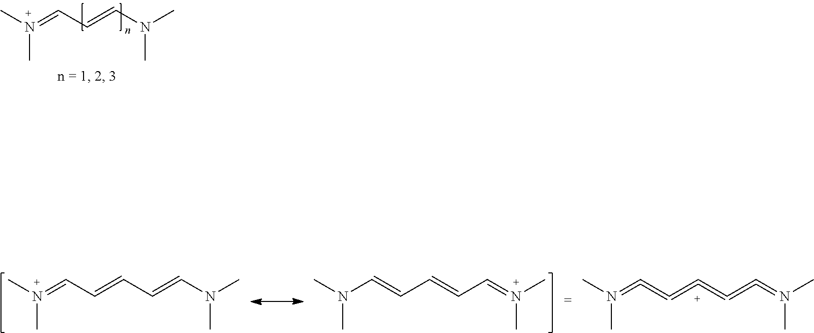

Due to their structure, cyanines have outstandingly high extinction coefficients often exceeding 100,000 Lmol⁻¹ cm⁻¹. Different substituents allow to control properties of the chromophore, such as absorbance wavelength, photostability, and fluorescence. For example, absorbance and fluorescence wavelength can be controlled by a choice of polymethine bridge length: longer cyanines possess higher absorbance and emission wavelengths up to near infrared region. Non-limiting examples of cyanine dyes include non-sulfonated cyanines, and sulfonated cyanines.

Available non-sulfonated dyes include, e.g., Cy3, Cy3.5, Cy5, Cy5.5, Cy7, and Cy7.5. Cy® stands for ‘cyanine’, and the first digit identifies the number of carbon atoms between the indolenine groups. Cy2 which is an oxazole derivative rather than indolenin, is an exception from this rule. The suffix 0.5 is added for benzo-fused cyanines. In certain embodiments, variation of the structures allows to change fluorescence properties of the molecules, and to cover most important part of visible and NIR spectrum with several fluorophores.

The structures of Cy3, Cy3.5, Cy5, Cy5.5, Cy7, and Cy7.5 are as follows:

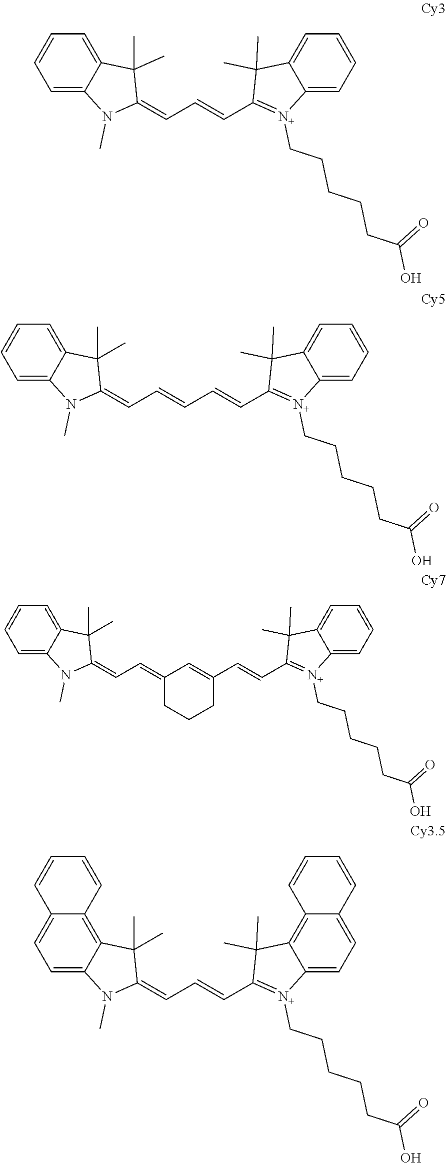 [SMILES: CN1/C(=C/C=C/C2=[N+](CCCCCC(=O)O)c3ccccc3C2(C)C)C(C)(C)c2ccccc21 ; CN1/C(=C/C=C/C=C/C2=[N+](CCCCCC(=O)O)c3ccccc3C2(C)C)C(C)(C)c2ccccc21 ; CN1/C(=C/C=C2C=C(/C=C/C3=[N+](CCCCCC(=O)O)c4ccccc4C3(C)C)CCC/2)C(C)(C)c2ccccc21 ; CN1/C(=C/C=C/C2=[N+](CCCCCC(=O)O)c3ccc4ccccc4c3C2(C)C)C(C)(C)c2c1ccc1ccccc21]

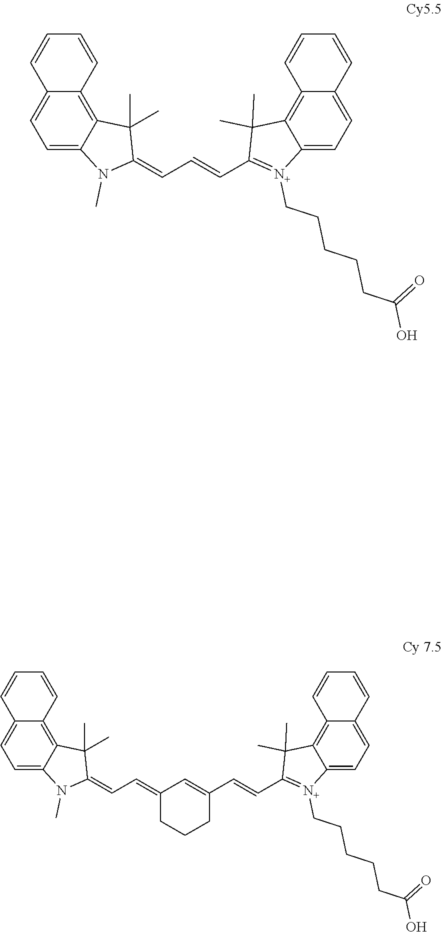 [SMILES: CN1/C(=C/C=C/C2=[N+](CCCCCC(=O)O)c3ccc4ccccc4c3C2(C)C)C(C)(C)c2c1ccc1ccccc21 ; CN1/C(=C/C=C2C=C(/C=C/C3=[N+](CCCCCC(=O)O)c4ccc5ccccc5c4C3(C)C)CCC/2)C(C)(C)c2c1ccc1ccccc21]

Sulfonated cyanines include additional sulfo-groups which, in some embodiments, facilitate dissolution of dye molecules in aqueous phase. In various embodiments, charged sulfonate groups decrease aggregation of dye molecules and heavily labeled conjugates.

Non-limiting examples of sulfonated cyanines include sulfo-Cy3, sulfo-Cy5, and sulfo-Cy7.

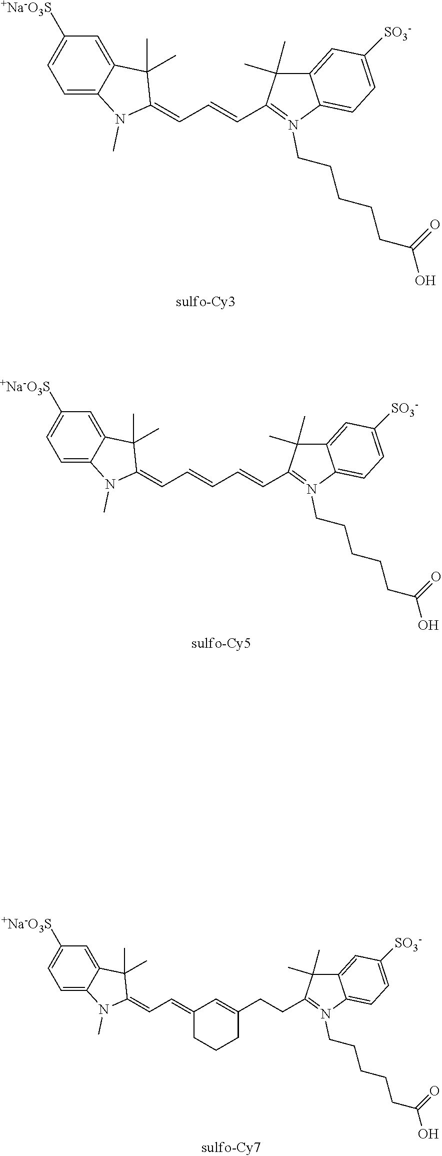

### IR800

The structure of IR800 maleimide is as follows:

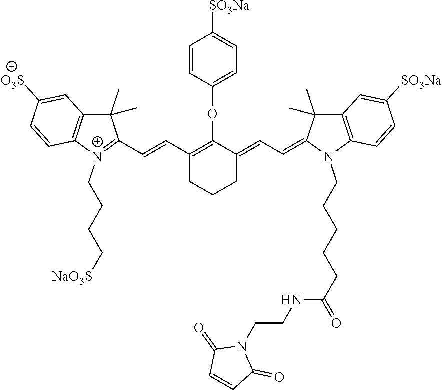

IR800 is also known as IRDye® 800CW Infrared Dye, and is available from LI-COR Biosciences (Nebraska, United States).

### Additional Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials, compounds, or steps “and those that do not materially affect the basic and novel characteristic(s)” of the claimed invention.

The compositions and elements of the compositions (e.g., peptides, moieties, and other components of the compositions) described herein may be purified. For example, purified naturally-occurring, synthetically produced, or recombinant compounds, e.g., polypeptides, nucleic acids, small molecules, or other agents, are separated from compounds with which they exist in nature. Purified compounds are at least 60% by weight (dry weight) the compound of interest. Preferably, the preparation is at least 75%, more preferably at least 90%, and most preferably at least 99% or 100%, by weight the compound of interest. Purity is measured by any appropriate standard method, for example, by column chromatography, polyacrylamide gel electrophoresis, or HPLC analysis.

Various embodiments of the invention relate to pH-triggered compounds (e.g., pH-triggered peptides) comprising “cargo” or a “moiety.” Depending on context, the cargo/moiety or may be referred to by a name or characteristic of an unconjugated form of the cargo/moiety regardless of whether the cargo/moiety is conjugated to a pH-triggered compound. For example, a small molecule known as “Small Molecule X” when in an unconjugated form may also be referred to herein as “Small Molecule X” when in a form that is bound to a pH-triggered compound (e.g., a pHLIP compound). Similarly, a “toxin” that is toxic only when free and unconjugated may still be referred to as a “toxin” when it is in a form that is bound to a pH-triggered compound (e.g., a pHLIP compound). In some embodiments, a cargo molecule is functional when free from a pH-triggered compound (e.g., after release from a pH-triggered compound, e.g., within a cell). In some embodiments, a cargo molecule is functional while still covalently linked to a pH-triggered compound.

As used herein, the singular forms “a,” “an,” and “the” include the plural reference unless the context clearly dictates otherwise. Thus, for example, a reference to “a pHLIP peptide,” “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

### Click Reactions

Compounds described herein (e.g., pHLIP peptides and compounds comprising multiple pHLIP peptides) can include a covalent bond between the compound and a cargo compound, between a linker and a cargo compound, between a pHLIP peptide and a linker, and between two pHLIP peptides. In some embodiments, a covalent bond has been formed by a bio-orthogonal reaction such as a cycloaddition reaction (e.g., a “click” reaction). Exemplary bio-orthogonal reactions suitable for the preparation for such compounds are described in, e.g., Zheng et al., “Development of Bioorthogonal Reactions and Their Applications in Bioconjugation,” Molecules, 2015, 20, 3190-3205. The diversity and commercial availability of peptide precursors are attractive for constructing the multifunctional entities described herein. Described herein are exemplary, non-limiting click reactions suitable for, e.g., the preparation of pH-triggered peptide compounds that include a covalent bond between the peptide and a cargo compound.

### Huisgen Cycloadditions

A category of click reactions includes Huisgen 1,3-dipolar additions of acetylenes to azides. See, e.g., Scheme 1.

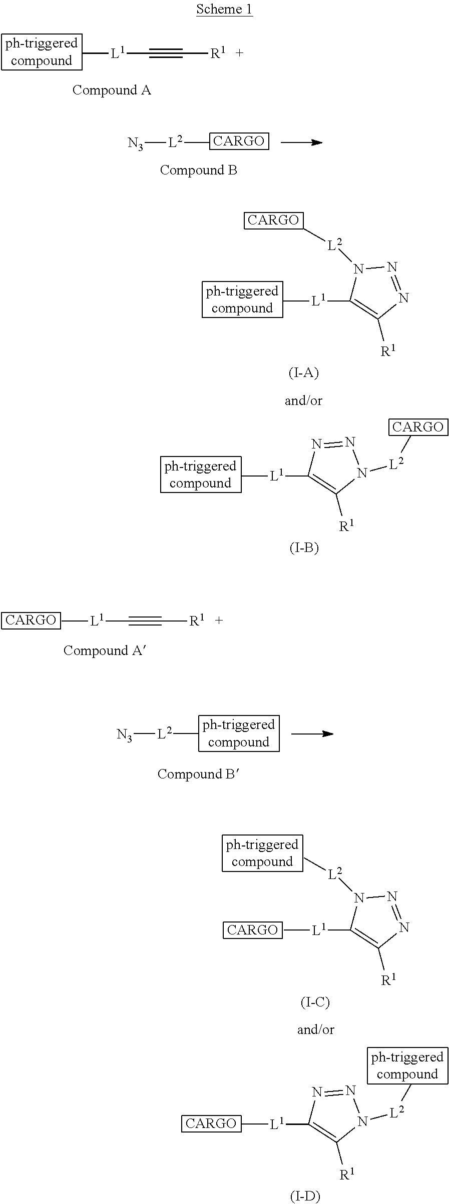

In embodiments, pH-triggered compound corresponds to any peptide or compound comprising multiple peptides disclosed herein. In certain embodiments, CARGO corresponds to any cargo compound described herein.

In embodiments, L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L¹ combines with R¹ to form a substituted or unsubstituted 8-membered cycloalkynylene ring, or L¹ comprises one or more amino acids as described herein.

In embodiments, R¹ is hydrogen, substituted or unsubstituted alkyl, or R¹ combines with L¹ to form a substituted or unsubstituted 8-membered cycloalkynylene ring, or L¹ comprises one or more amino acids as described herein.

In embodiments, L¹ combines with R¹ to form a substituted or unsubstituted 8-membered cycloalkynylene ring. In various embodiments, the 8-membered cycloalkynylene ring is unsubstituted. In some embodiments, the 8-membered cycloalkynylene ring comprises two fluoro substitutents (e.g., α to the alkynyl).

In embodiments, L² is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L² comprises one or more amino acids as described herein.

In embodiments, each R^{A} and R^{B} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the Huisgen cycloaddition is that described in Scheme 2 and Scheme 3.

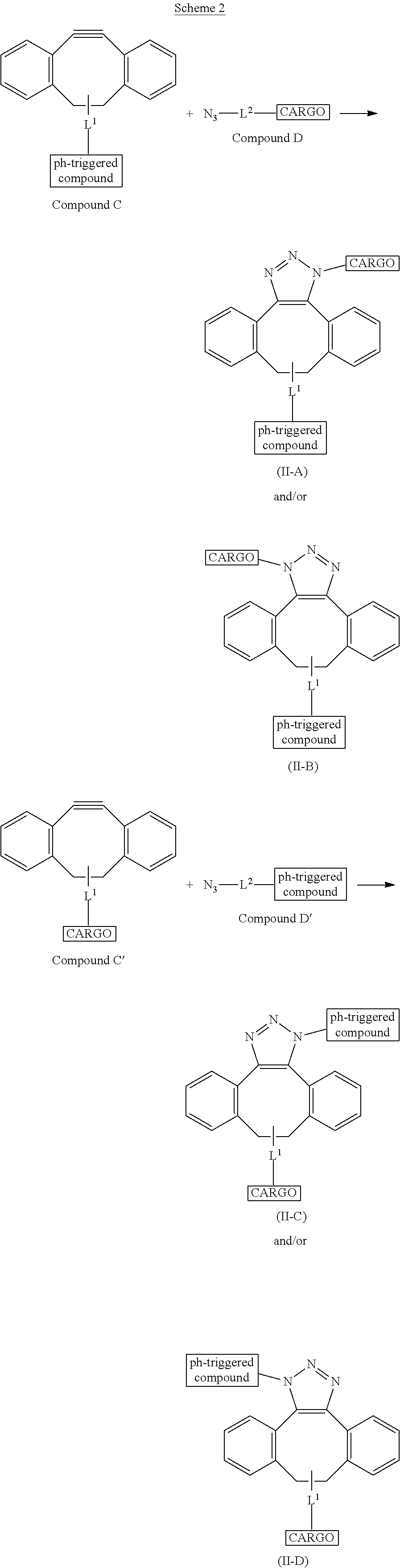

In embodiments, pH-triggered compound corresponds to any peptide or compound comprising multiple peptides disclosed herein. In certain embodiments, CARGO corresponds to any cargo compound described herein.

In embodiments, L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L¹ comprises one or more amino acids as described herein.

In embodiments, L² is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L² comprises one or more amino acids as described herein.

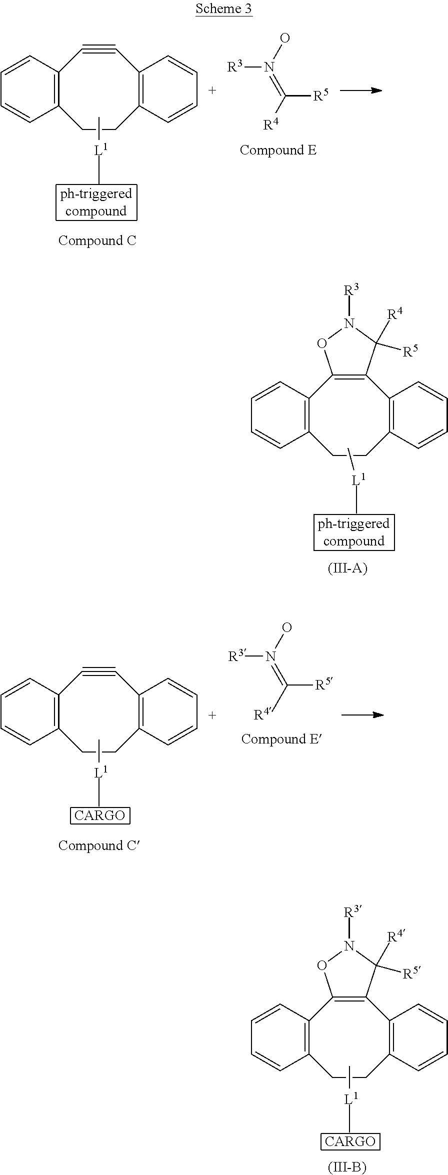

In embodiments, pH-triggered compound corresponds to any peptide or compound comprising multiple peptides disclosed herein. In various embodiments, CARGO corresponds to any cargo compound described herein.

In embodiments, L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L¹ comprises one or more amino acids as described herein.

In embodiments, one of R³, R⁴, and R⁵ is a cargo compound, and the other two variables are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, one of R^{3′}, R^{4′}, and R^{5′} is a pH-triggered peptide compound, the other two variables are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

Cycloadditions with Alkenes

In embodiments, certain activated alkenes (e.g., a strained alkene such as cis- or trans-cyclooctene or oxanorbornadiene), which may be represented as compound F or compound F′, can undergo cycloaddition reactions with, e.g., an azide (Scheme 4), a tetrazine (Scheme 5), or a tetrazole (Scheme 6).

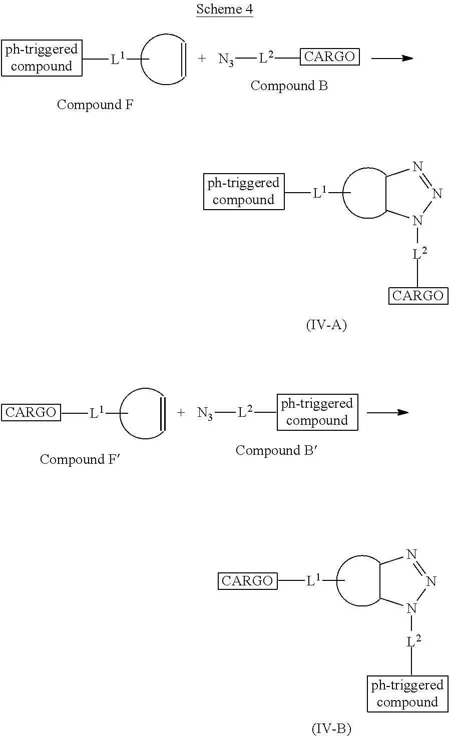

In embodiments, pH-triggered compound corresponds to any peptide or compound comprising multiple peptides disclosed herein. In some embodiments, CARGO corresponds to any cargo compound described herein.

In embodiments, L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L¹ comprises one or more amino acids as described herein.

In embodiments, L² is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L² comprises one or more amino acids as described herein.

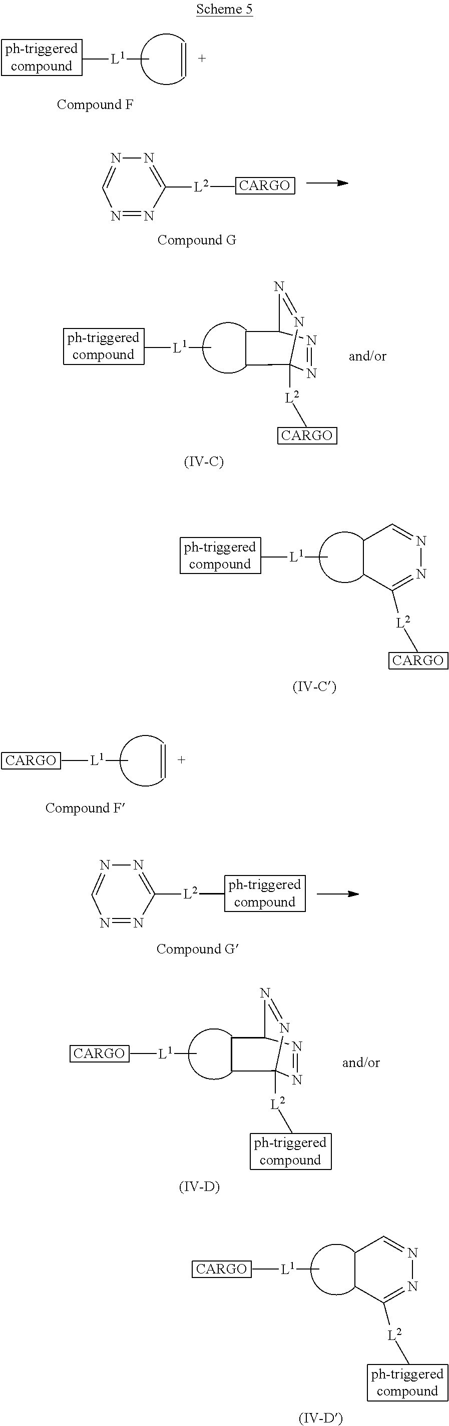

In embodiments, pH-triggered compound corresponds to any peptide or compound comprising multiple peptides disclosed herein. In certain embodiments, CARGO corresponds to any cargo compound described herein.

In embodiments, L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L¹ comprises one or more amino acids as described herein.

In embodiments, L² is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L² comprises one or more amino acids as described herein.

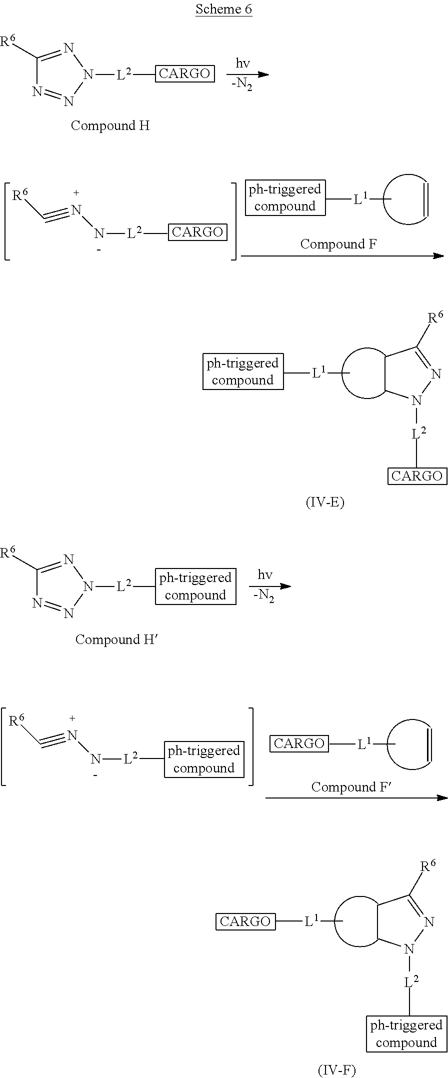

In embodiments, pH-triggered compound corresponds to any peptide or compound comprising multiple peptides disclosed herein. In various embodiments, CARGO corresponds to any cargo compound described herein.

In embodiments, L¹ is independently a bond, —NR^{A}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L¹ comprises one or more amino acids as described herein.

In embodiments, L² is independently a bond, —NR^{B}—, O, S, substituted or unsubstituted alkylene, substituted or unsubstituted alkenylene, substituted or unsubstituted alkynylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, or L² comprises one or more amino acids as described herein.

In embodiments, R⁶ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, the invention features any of the compounds described herein (e.g., any of Compounds A, A′, B, B′; C, C′, D, D′, E, E′, F, F′, G, G′H, or H′; a compound according to any one of formulas (I-A), (I-B), (I-C), (I-D), (II-A), (II-B), (II-C), (II-D), (III-A), (III-B), (IV-A), (IV-B), (IV-C), (IV-C′), (IV-D), (IV-D′), (IV-E), or (IV-F); a compound according to Formula (A) such as any one of Formulas (A4)-(A20); or a compound according to any of SEQ ID NOS: 1-4); or a pharmaceutically acceptable salt thereof.

In embodiments, the invention features a composition (e.g., a pharmaceutical composition) comprising any of the compounds described herein (e.g., any of Compounds A, A′, B, B′; C, C′, D, D′, E, E′, F, F′, G, G′H, or H′; a compound according to any one of formulas (I-A), (I-B), (I-C), (I-D), (II-A), (II-B3), (II-C), (II-D3), (III-A), (III-B), (IV-A), (IV-B), (IV-C), (IV-C′), (IV-D), (IV-D′), (IV-E), or (IV-F); a compound according to Formula (A) such as any one of Formulas (A4)-(A20); or a compound according to any of SEQ ID NOS: 1-4); or a pharmaceutically acceptable salt thereof.

The term “alkyl,” by itself or as part of another substituent, means, unless otherwise stated, a non-cyclic straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom (e.g. selected from the group consisting of O, N, P, S, Se and Si, and wherein the nitrogen, selenium, and sulfur atoms may optionally be oxidized, and the nitrogen heteroatom may optionally be quaternized). The heteroatom(s) 0, N, P, S, Se, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —C—H═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′- and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SeR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms (e.g. selected from N, O, and S, wherein the nitrogen and sulfur atoms are optionally oxidized, and the nitrogen atom(s) are optionally quaternized). Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one or more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

As used herein, the terms “heteroatom” or “ring heteroatom” are meant to include oxygen (O), nitrogen (N), sulfur (S), phosphorus (P), and silicon (Si).

Examples and embodiments are provided below to facilitate a more complete understanding of the invention. The following examples and embodiments illustrate the exemplary modes of making and practicing the invention. However, the scope of the invention is not limited to specific examples and embodiments disclosed, which are for purposes of illustration only, since alternative methods can be utilized to obtain similar results.

### EMBODIMENTS

Embodiments include the following embodiments P1 to P35.

Embodiment P1. A pH-triggered compound comprising a pH-triggered peptide (pHLIP peptide) that is covalently attached to at least one other pHLIP peptide via a linker or a covalent bond.

Embodiment P2. The compound of Embodiment P1 having the following structure:

[A]ₖ-linker
wherein
k is an integer from 2 to 32, and
each A is, individually, a pHLIP peptide comprising at least 8 consecutive amino acids, wherein
(i) at least 4 of the at least 8 consecutive amino acids are non-polar amino acids,
(ii) at least 1 of the at least 8 consecutive amino acids is protonatable, and
(iii) the pHLIP peptide has a higher affinity for a membrane lipid bilayer at pH 5.0 compared to the affinity at pH 8.0.

Embodiment P3. The compound of Embodiment P2, wherein each pHLIP peptide, individually, has the sequence: XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein,
(i) each Y is, individually, a non-polar amino acid with solvation energy, ΔG_{X}^{cor}>+0.50, or Gly;
(ii) each X is, individually, a protonatable amino acid,
(iii) n, m, i, j, l, h, g, f are each, individually, an integer from 1 to 8.

Embodiment P4. The compound of any one of Embodiments P1-P3, comprising at least two pHLIP peptides with different amino acid sequences or wherein each pHLIP peptide comprises the same amino acid sequence.

Embodiment P5. The compound of any one of Embodiments P1-P4, comprising the following structure:

A-L-B
wherein A is the first pHLIP peptide, B is the second pHLIP peptide, L is the linker, and each - is a covalent bond.

Embodiment P6. The compound of any one of Embodiments P1-P4, comprising the following structure:

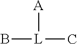

wherein A is the first pHLIP peptide, B is the second pHLIP peptide, C is the third pHLIP peptide, L is the linker, and each - is a covalent bond.

Embodiment P7. The compound of any one of Embodiments P1-P4, comprising the following structure:

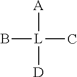

wherein A is the first pHLIP peptide, B is the second pHLIP peptide, C is the third pHLIP peptide, D is the fourth pHLIP peptide, L is the linker, and each - is a covalent bond.

Embodiment P8. The compound of any one of Embodiments P1-P7, comprising k pHLIP peptides, wherein (a) each pHLIP peptide has a unique amino acid sequence compared to each of the other pHLIP peptides in the compound, wherein k≥2; or (b) each of the k pHLIP peptides has an identical amino acid sequence, wherein each of the k pHLIP peptides is connected to each of the other k pHLIP peptides by a linker, wherein 1<k≤32.

Embodiment P9. The compound of any one of Embodiments P1-P8, wherein each pHLIP peptide has a net negative charge at a pH of about 7.25, 7.5, or 7.75 in water.

Embodiment P10. The compound of any one of Embodiments P1-P9, wherein each pHLIP peptide has an acid dissociation constant on abase 10 logarithmic scale (pKa) of less than about 4.0, 4.5, 5.0, 5.5, 6.0, 6.5, or 7.0.

Embodiment P11. The compound of any one of Embodiments P1-P10, wherein at least one of the pHLIP peptides comprises:
(a) 1 protonatable amino acid which is aspartic acid, glutamic acid, alpha-aminoadipic acid, or gamma-carboxyglutamic acid; or
(b) at least 2, 3, or 4 protonatable amino acids, wherein the protonatable amino acids comprise aspartic acid, glutamic acid, alpha-aminoadipic acid, gamma-carboxyglutamic acid, or any combination thereof.

Embodiment P12. The compound of any one of Embodiments P1-P11, wherein
(a) at least one of the pHLIP peptides comprises at least 1 non-native protonatable amino acid;
(b) at least one of the pHLIP peptides comprises at least 1 non-native protonatable amino acid, wherein the non-native protonatable amino acid comprises at least 1, 2, 3, or 4 carboxyl groups;
(c) at least one of the pHLIP peptides comprises at least 1, 2, 3, 4, 5, 6, 7, 8, 9, 10, 11, 12, 13, 14, 15, or 16 carboxyl groups;
(d) at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 coded amino acids;
(e) at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 non-coded amino acids;
(f) the amino acids of at least one of the pHLIP peptides are non-native amino acids;
(g) at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 D-amino acids;
(h) at least one of the pHLIP peptides comprises at least 1 non-coded amino acid, wherein the non-coded amino acid is an aspartic acid derivative, or a glutamic acid derivative;
(i) at least one of the pHLIP peptides comprises at least 8 consecutive amino acids, wherein, at least 2, 3, or 4 of the at least 8 consecutive amino acids are non-polar, and at least 1, 2, 3, or 4 of the at least 8 consecutive amino acids is protonatable;
(j) at least one of the pHLIP peptides comprises a functional group to which the linker is attached;
(k) the compound comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are linked together by the linker;
(l) the compound comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each directly linked to the linker by a covalent bond; or
(m) the pHLIP peptides are attached to the linker by covalent bonds.

Embodiment P13. The compound of any one of Embodiments P1-P12, comprising at least one pHLIP peptide that is attached to the linker by a covalent bond.

Embodiment P14. The compound of Embodiment P13, wherein
(a) the covalent bond is a peptide bond;
(b) the covalent bond is a disulfide bond, a bond between two selenium atoms, or a bond between a sulfur and a selenium atom;
(c) the covalent bond is a bond that has been formed by a click chemistry reaction; or
(d) the covalent bond is a bond that has been formed by a reaction between an azide and an alkyne, an alkyne and a strained difluorooctyne, a diaryl-strained-cyclooctyne and a 1,3-nitrone, a cyclooctene, trans-cycloalkene, or oxanorbornadiene and an azide, tetrazine, or tetrazole, an activated alkene or oxanorbornadiene and an azide, a strained cyclooctene or other activated alkene and a tetrazine, or a tetrazole that has been activated by ultraviolet light and an alkene.

Embodiment P15. The compound of any one of Embodiments P1-P14, wherein
(a) the covalent bond is a peptide bond;
(b) the covalent bond is not a peptide bond;
(c) the linker comprises an artificial polymer or a synthetically produced polymer that has the structure of a polymer that exists in nature;
(d) the linker comprises a polypeptide, a polylysine, a polyarginine, a polyglutamic acid, a polyaspartic acid, a polycysteine, or a polynucleic acid;
(e) the linker does not comprise an amino acid;
(f) the linker comprises a polysaccharide, a chitosan, or an alginate;
(g) the linker comprises a poly(ethylene glycol), a poly(lactic acid), a poly(glycolic acid), a poly(lactic-co-glycolic acid), a poly(malic acid), a polyorthoester, a poly(vinylalcohol), a poly(vinylpyrrolidone), a poly(methyl methacrylate), a poly(acrylic acid), a poly(acrylamide), a poly(methacrylic acid), a poly(amidoamine), a polyanhydrides, or a polycyanoacrylate;
(h) the linker comprises a linear polymer or a branched polymer;
(i) the linker comprises an organic compound structure;
(j) the linker comprises an organic compound structure, wherein the organic compound structure has a molecular weight less than about 10, 9, 8, 7, 6, 5, 4, 3, 2, 1, or 0.5 kilodaltons (kDa);
(k) the linker comprises poly(ethylene glycol); or
(l) the linker comprises poly(ethylene glycol), wherein the poly(ethylene glycol) has a molecular weight of 60 to 100,000 Daltons.

Embodiment P16. The compound of any one of Embodiments P1-P15, wherein the linker comprises a cell, a particle, a dendrimer, or a nanoparticle.

Embodiment P17. The compound of any one of Embodiments P1-P6,
(a) comprising at least one pHLIP peptide that comprises a functional group for cargo compound attachment;
(b) wherein the linker comprises a functional group for cargo compound attachment;
(c) comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each individually attached to a cargo compound via a linker;
(d) comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each individually directly attached to a cargo compound by a covalent bond;
(e) wherein at least one of the pHLIP peptides is attached to a cargo compound by a covalent bond, wherein the covalent bond is an ester bond, a disulfide bond, a bond between two selenium atoms, a bond between a sulfur and a selenium atom, or an acid-liable bond;
(f) wherein at least one of the pHLIP peptides is attached to a cargo compound by a covalent bond, wherein the covalent bond is a bond that has been formed by a click chemistry reaction; or
(g) wherein at least one of the pHLIP peptides is attached to a cargo compound by a covalent bond, wherein the covalent bond is a bond that has been formed by a click chemistry reaction.

Embodiment P18. The compound of Embodiment P17, wherein
(a) the functional group is a side chain of an amino acid of at least one pHLIP peptide;
(b) the functional group is a side chain of an amino acid of at least one pHLIP peptide, wherein the side chain is a side chain to which a cargo compound may be attached via a disulfide bond;
(c) the functional group comprises a free sulfhydryl (SH) or selenohydryl (SeH) group;
(d) the functional group comprises a cysteine, homocysteine, selenocysteine, or homoselenocysteine;
(e) the functional group comprises a primary amine;
(f) the functional group comprises an azido modified amino acid; or
(g) the functional group comprises an alkynyl modified amino acid.

Embodiment P19. The compound of any one of Embodiments P1-P18, wherein the linker is attached to a cargo compound via a covalent bond.

Embodiment P20. The compound of Embodiment P19, wherein
(a) the covalent bond is an ester bond, a disulfide bond, a bond between two selenium atoms, a bond between a sulfur and a selenium atom, or an acid-liable bond;
(b) the covalent bond is a bond that has been formed by a click chemistry reaction;
(c) the covalent bond is a bond that has been formed by a reaction between an azide and an alkyne, an alkyne and a strained difluorooctyne, a diaryl-strained-cyclooctyne and a 1,3-nitrone, a cyclooctene, trans-cycloalkene, or oxanorbornadiene and an azide, tetrazine, or tetrazole, an activated alkene or oxanorbornadiene and an azide, a strained cyclooctene or other activated alkene and a tetrazine, or a tetrazole that has been activated by ultraviolet light and an alkene.

Embodiment P21. The compound of any one of Embodiments P1-P20, further comprising a cargo compound.

Embodiment P22. The compound of Embodiment P21, wherein
(a) the cargo compound is polar or nonpolar;
(b) the cargo compound comprises a marker;
(c) the cargo compound comprises a prophylactic, therapeutic, diagnostic, radiation-enhancing, radiation-sensitizing, imaging, gene regulation, immune activation, cytotoxic, apoptotic, or research agent;
(d) the cargo compound comprises a dye, a fluorescent dye, a fluorescence quencher, or a fluorescent protein;
(e) the cargo compound comprises a magnetic resonance, positron emission tomography, single photon emission computed tomography, fluorescent, optoacoustic, ultrasound, or X-ray contrast imaging agent;
(f) the cargo compound comprises a peptide, a protein, an enzyme, or a polysaccharide;
(g) the cargo compound comprises an aptamer, an antigen, a protease, an amylase, a lipase, a Fc receptor, a tissue factor, or a complement component 3 (C3) protein;
(h) the cargo compound comprises a toxin, an inhibitor, a DNA intercalator, an alkylating agent, an antimetabolite, an anti-microtubule agents, a topoisomerase inhibitor, or an antibiotic compound;
(i) the cargo compound comprises an amanita toxin, a vinca alkaloid, a taxane, an anthracycline, a bleomycin, a nitrogen mustard, a nitrosourea, a tetrazine, an aziridine, a platinum-containing chemotherapeutic agent, cisplatin or a cisplatin derivative, a procarbazine, or a hexamethylmelamine;
(j) the cargo compound comprises a DNA, a DNA analog, a RNA, a RNA analog;
(k) the cargo compound comprises a peptide nucleic acid (PNA), a bis PNA, a gamma PNA, a locked nucleic acid (LNA), or a morpholino;
(l) the cargo compound comprises a chemotherapeutic compound;
(m) the cargo compound comprises an antimicrobial compound; or
(n) the cargo compound comprises a gene-regulation compound.

Embodiment P23. The compound of any one of Embodiments P1-P22, wherein at least one of the pHLIP peptides comprises an amino acid side chain that is radioactive or detectable by probing radiation.

Embodiment P24. The compound of any one of Embodiments P1-P23, wherein one or more atoms of the compound is a radioactive isotope or has been replaced with a stable isotope.

Embodiment P25. A formulation for a parenteral, a local, or a systemic administration comprising the compound of any one of Embodiments P1-P24.

Embodiment P26. A compound for the treatment of a superficial or muscle invasive bladder tumor comprising (i) a pHLIP peptide that is attached to at least one other pHLIP peptide via a peptide linker, and (ii) an amanitin toxic cargo.

Embodiment P27. A formulation for the ex vivo treatment of a biopsy specimen, a liquid biopsy specimen, surgically removed tissue, a surgically removed liquid, or blood, comprising the compound of any one of Embodiments P1-P24.

Embodiment P28. A pH-triggered peptide (pHLIP peptide) comprising the sequence of at least 8 to 25 consecutive amino acids that is present in any one of the following sequences:

(SEQ ID NO: 124)
X₂X₂RX₂X₂X₁X₂X₂X₂X₃X₃X₂X₂X₂X₂X₂X₁X₂X₂X₂X₂,
(SEQ ID NO: 125)
X₂X₂RX₂X₃X₁X₂X₂X₂X₃X₃X₂X₂X₂X₂X₂X₁X₂GX₂X₂,
(SEQ ID NO: 126)
X₂X₂RX₂X₃X₁X₂X₂X₃X₃X₂X₂X₂X₂X₂X₁X₂X₂X₂X₂X₂,
(SEQ ID NO: 127)
X₂X₂RX₂X₂X₁X₂X₂X₂X₃X₃X₂X₂X₂X₂X₂X₁X₂X₃X₂X₂,
(SEQ ID NO: 128)
X₂X₂X₂X₂X₁X₂X₂X₂X₂X₂X₃X₃X₂X₂X₂X₁X₂X₂RX₂X₂,
(SEQ ID NO: 129)
X₂X₂GX₂X₁X₂X₂X₂X₂X₂X₃X₃X₂X₂X₂X₁X₃X₂RX₂X₂,
(SEQ ID NO: 130)
X₂X₂X₂X₂X₂X₁X₂X₂X₂X₂X₂X₃X₃X₂X₂X₁X₃X₂RX₂X₂,
(SEQ ID NO: 131)
X₂X₂X₃X₂X₁X₂X₂X₂X₂X₂X₃X₃X₂X₂X₂X₁X₂X₂RX₂X₂,
(SEQ ID NO: 132)
GX₂X₂GX₂X₂GX₂X₁GX₂X₂GX₂X₂X₂GX₂X₂X₁GX₂X₂X₂GX₂,
(SEQ ID NO: 133)
X₂GX₂X₂X₂GX₁X₂X₂GX₂X₂X₂GX₂X₂GX₁X₂GX₂X₂GX₂X₂G,
(SEQ ID NO: 134)
X₂RX₂X₂X₂X₁X₂X₂X₂X₂X₃X₁X₃X₂X₂X₂X₁X₂X₂X₂,
(SEQ ID NO: 135)
X₂X₂X₂X₁X₂X₂X₂X₃X₁X₃X₂X₂X₂X₂X₁X₂X₂X₂RX₂,
(SEQ ID NO: 136)
X₂X₂RX₂X₂X₁X₂X₂X₂X₂X₃X₁X₃X₂X₂X₂XX₂,
(SEQ ID NO: 137)
X₂X₂QX₂X₂X₁X₂X₂X₂X₂X₃X₁X₃X₂X₂X₂X₁X₂,
(SEQ ID NO: 138)
X₂X₁X₂X₂X₂X₃X₁X₃X₂X₂X₂X₂X₁X₂X₂RX₂X₂,
(SEQ ID NO: 139)
X₂X₁X₂X₂X₂X₃X₁X₃X₂X₂X₂X₂X₁X₂X₂QX₂X₂,
(SEQ ID NO: 140)
X₂X₂X₂X₃X₃X₂X₂X₂X₂X₂NGX₂X₂X₂X₂X₁,
(SEQ ID NO: 141)
X₂X₂X₂X₃X₃X₂X₂X₂X₂X₂X₂GX₂X₂X₂X₂X₁,
(SEQ ID NO: 142)
X₂X₂RX₂X₂X₁X₂X₂X₂X₂X₃X₃X₂X₂X₂,
(SEQ ID NO: 143)
X₁X₂X₂X₂X₂GNX₂X₂X₂X₂X₂X₃X₃X₂X₂X₂,
(SEQ ID NO: 144)
X₁X₂X₂X₂X₂GX₂X₂X₂X₂X₂X₂X₃X₃X₂X₂X₂,
(SEQ ID NO: 145)
X₂X₂X₂X₃X₃X₂X₂X₂X₂X₁X₂X₂RX₂X₂,
(SEQ ID NO: 146)
GNX₂X₁GX₂X₂X₂X₃X₂GGX₁X₂X₂X₂X₂X₃X₂X₂X₂X₂X₂X₂X₁,
(SEQ ID NO: 147)
X₁GX₂X₂X₂X₃X₂GGX₁X₂X₂X₂X₂X₃X₁X₂X₂X₂X₂X₂X₁,
(SEQ ID NO: 147)
X₁GX₂X₂X₂X₃X₂GGX₁X₂X₂X₂X₂X₃X₁X₂X₂X₂X₂X₂X₁,
(SEQ ID NO: 149)
X₁X₂X₂X₂X₂X₂X₂X₃X₂X₂X₂X₂X₁GGX₂X₃X₂X₂X₂GX₁X₂NG,
(SEQ ID NO: 150)
X₁X₂X₂X₂X₂X₂X₁X₃X₂X₂X₂X₂X₁GGX₂X₃X₂X₂X₂GX₁,
(SEQ ID NO: 151)
X₁X₂X₂X₂X₂X₂X₁X₃X₂X₂X₂X₂X₁GGX₂X₃X₂X₂X₂GX₁,
(SEQ ID NO: 152)
X₂X₂X₁X₂X₂X₂GX₂X₂X₂X₂X₂X₃X₃X₂X₁X₂X₂X₂QX₂,
and
(SEQ ID NO: 153)
X₂QX₂X₂X₂X₁X₂X₃X₃X₂X₂X₂X₂X₂GX₂X₂X₂X₁X₂X₂,

wherein
   each X₁ is, individually, D, E, Gla, or Aad,
   each X₂ is, individually, A, I, L, M, F, P, W, Y, V, or G and
   each X₃ is, individually, S, T, or G.

Embodiment P29. A pHLIP peptide comprising at least 8 consecutive amino acids, wherein
(i) at least 4 of the 8 consecutive amino acids are non-polar amino acids,
(ii) at least 1 of the at least 8 consecutive amino acids is protonatable,
(iii) the pHLIP peptide has a higher affinity for a membrane lipid bilayer at pH 5.0 compared to the affinity at pH 8.0, and
(iv) the at least 8 consecutive amino acids comprise 8 consecutive amino acids in a sequence that is identical to a sequence of 8 consecutive amino acids that occurs in a naturally occurring human protein.

Embodiment P30. A pHLIP peptide having the sequence:
XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYₗXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein,
(i) each Y is, individually, a non-polar amino acid with solvation energy, ΔG_{X}^{cor}>+0.50, or Gly;
(ii) each X is, individually, a protonatable amino acid,
(iii) n, m, i, j, l, h, g, f are each, individually, an integer from 1 to 8.

Embodiment P31. A non-ocular cell comprising an exogenous nucleic acid encoding a pHLIP peptide comprising at least 8 consecutive amino acids with a sequence that is at least 85% identical to (i) a sequence of at least 8 consecutive amino acids that occurs in a naturally occurring human protein; or (ii) the reverse of a sequence of at least 8 consecutive amino acids that occurs in a naturally occurring human protein.

Embodiment P32. A non-ocular cell comprising a pHLIP peptide comprising at least 8 consecutive amino acids with a sequence that is at least 85% identical to (i) a sequence of at least 8 consecutive amino acids that occurs in a naturally occurring human protein; or (ii) the reverse of a sequence of at least 8 consecutive amino acids that occurs in a naturally occurring human protein expressed on the surface of said cell.

Embodiment P33. The non-ocular cell of Embodiment P32, wherein the at least 8 consecutive amino acids are located outside of the lipid bilayer of the cell membrane of said cell.

Embodiment P34. The non-ocular cell of Embodiment P32 or P33, wherein at least 85% of the expressed pHLIP peptide is presented on the exterior of said cell.

Embodiment P35. The non-ocular cell of any one of Embodiments P32-P34, which is a T-cell, a B-cell, a neutrophil, an eosinophil, a basophil, a lymphocyte, a monocyte, a dendritic cell, a natural killer cell, or a macrophage.

Further embodiments include the following embodiments 1 to 43.

Embodiment 1. A pH-triggered compound comprising a pH-triggered peptide (pHLIP peptide) that is covalently attached to at least one other pHLIP peptide via a linker or a covalent bond.

Embodiment 2. The compound of Embodiment 1, comprising the following structure:

A-L-B

wherein A is a first pHLIP peptide comprising the sequence DDQNPWRAYLDLLFPTDTLLLDLLW (SEQ ID NO: 1), B is a second pHLIP peptide comprising the sequence DDQNPWRAYLDLLFPTDTLLLDLLW (SEQ ID NO: 1), L is a polyethylene glycol linker, and each - is a covalent bond.

Embodiment 3. The compound of Embodiment 1 or 2, comprising at least one pHLIP peptide comprising one or more of the following sequences: AYLDLLFP (SEQ ID NO: 4), YLDLLFPT (SEQ ID NO: 5), LDLLFPTD (SEQ ID NO: 6), DLLFPTDT (SEQ ID NO: 7), LLFPTDT (SEQ ID NO: 8), LFPTDTLL (SEQ ID NO: 9), FPTDTLLL (SEQ ID NO: 10), PTDTLLLD (SEQ ID NO: 11), TDTLLLDL (SEQ ID NO: 12), DTLLLDLL (SEQ ID NO: 13), or TLLLDLLW (SEQ ID NO: 14).

Embodiment 4. The compound of any one of Embodiments 1-3, comprising at least one pHLIP peptide comprising the sequence DDQNPWRAYLDLLFPTDTLLLDLLW (SEQ ID NO: 1), ACDDQNPWRAYLDLLFPTDTLLLDLLWA (SEQ ID NO: 15), AKDDQNPWRAYLDLLFPTDTLLLDLLWA (SEQ ID NO: 16), ADDQNPWRAYLDLLFPTDTLLLDLLWCA (SEQ ID NO: 17), ADDQNPWRAYLDLLFPTDTLLLDLLWKA (SEQ ID NO: 18), ACDDQNPWRAYLDLLFPTDTLLLDLLWKA (SEQ ID NO: 19), or AKDDQNPWRAYLDLLFPTDTLLLDLLWCA (SEQ ID NO: 20).

Embodiment 5. The compound of Embodiment 4, comprising at least one pHLIP peptide comprising the sequence DDQNPWRAYLDLLFPTDTLLLDLLW (SEQ ID NO: 1).

Embodiment 6. The compound of Embodiment 1, comprising the following structure:

A-L-B

wherein A is a first pHLIP peptide comprising the sequence AEQNPIYWARYADWLFTTPLLLLDLALLVDADEGT (SEQ ID NO: 2), B is a second pHLIP peptide comprising the sequence AEQNPIYWARYADWLFTTPLLLLDLALLVDADEGT (SEQ ID NO: 2), L is a polyethylene glycol linker, and each - is a covalent bond.

Embodiment 7. The compound of Embodiment 1, comprising the following structure:

A-L-B

wherein A is a first pHLIP peptide comprising the sequence GLAGLAGLLGLEGLLGLPLGLLEGLWLGLELEGN (SEQ ID NO: 3), B is a second pHLIP peptide comprising the sequence GLAGLAGLLGLEGLLGLPLGLLEGLWLGLELEGN (SEQ ID NO: 3), L is a polyethylene glycol linker, and each - is a covalent bond.

Embodiment 8. The compound of any one of Embodiment 2 1-7 having the following structure:

[A]ₖ-linker
wherein
k is an integer from 2 to 32, and
each A is, individually, a pHLIP peptide comprising at least 8 consecutive amino acids, wherein
(i) at least 4 of the at least 8 consecutive amino acids are non-polar amino acids,
(ii) at least 1 of the at least 8 consecutive amino acids is protonatable, and
(iii) the pHLIP peptide has a higher affinity for a membrane lipid bilayer at pH 5.0 compared to the affinity at pH 8.0.

Embodiment 9. The compound of any one of Embodiments 1-8, wherein each pHLIP peptide, individually, has the sequence:
XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYₗXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein,
(i) each Y is, individually, a non-polar amino acid with solvation energy, G>+0.50, or Gly;
(ii) each X is, individually, a protonatable amino acid,
(iii) n, m, i, j, 1, h, g, f are each, individually, an integer from 1 to 8.

Embodiment 10. The compound of any one of Embodiments 1-9, comprising at least two pHLIP peptides with different amino acid sequences or wherein each pHLIP peptide comprises the same amino acid sequence.

Embodiment 11. The compound of any one of Embodiments 1-9, comprising the following structure:

A-L-B

wherein A is the first pHLIP peptide, B is the second pHLIP peptide, L is the linker, and each - is a covalent bond.

Embodiment 12. The compound of any one of Embodiments 1-9, comprising the following structure:

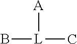

wherein A is the first pHLIP peptide, B is the second pHLIP peptide, C is the third pHLIP peptide, L is the linker, and each - is a covalent bond.

Embodiment 13. The compound of any one of Embodiments 1-9, comprising the following structure:

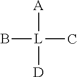

wherein A is the first pHLIP peptide, B is the second pHLIP peptide, C is the third pHLIP peptide, D is the fourth pHLIP peptide, L is the linker, and each - is a covalent bond.

Embodiment 14. The compound of any one of Embodiments 1-13, comprising k pHLIP peptides, wherein (a) each pHLIP peptide has a unique amino acid sequence compared to each of the other pHLIP peptides in the compound, wherein k≥2; or (b) each of the k pHLIP peptides has an identical amino acid sequence, wherein each of the k pHLIP peptides is connected to each of the other k pHLIP peptides by a linker, wherein 1<k≤32.

Embodiment 15. The compound of any one of Embodiments 1-14, wherein each pHLIP peptide has a net negative charge at a pH of about 7.25, 7.5, or 7.75 in water.

Embodiment 16. The compound of any one of Embodiments 1-15, wherein each pHLIP peptide has an acid dissociation constant on abase 10 logarithmic scale (pKa) of less than about 4.0, 4.5, 5.0, 5.5, 6.0, 6.5, or 7.0.

Embodiment 17. The compound of any one of Embodiments 1-16, wherein at least one of the pHLIP peptides comprises:
(a) 1 protonatable amino acid which is aspartic acid, glutamic acid, alpha-aminoadipic acid, or gamma-carboxyglutamic acid; or
(b) at least 2, 3, or 4 protonatable amino acids, wherein the protonatable amino acids comprise aspartic acid, glutamic acid, alpha-aminoadipic acid, gamma-carboxyglutamic acid, or any combination thereof.

Embodiment 18. The compound of any one of Embodiments 1-17, wherein
(a) at least one of the pHLIP peptides comprises at least 1 non-native protonatable amino acid;
(b) at least one of the pHLIP peptides comprises at least 1 non-native protonatable amino acid, wherein the non-native protonatable amino acid comprises at least 1, 2, 3, or 4 carboxyl groups;
(c) at least one of the pHLIP peptides comprises at least 1, 2, 3, 4, 5, 6, 7, 8, 9, 10, 11, 12, 13, 14, 15, or 16 carboxyl groups;
(d) at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 coded amino acids;
(e) at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 non-coded amino acids;
(f) the amino acids of at least one of the pHLIP peptides are non-native amino acids;
(g) at least one of the pHLIP peptides comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 40 D-amino acids;
(h) at least one of the pHLIP peptides comprises at least 1 non-coded amino acid, wherein the non-coded amino acid is an aspartic acid derivative, or a glutamic acid derivative;
(i) at least one of the pHLIP peptides comprises at least 8 consecutive amino acids, wherein, at least 2, 3, or 4 of the at least 8 consecutive amino acids are non-polar, and at least 1, 2, 3, or 4 of the at least 8 consecutive amino acids is protonatable;
(j) at least one of the pHLIP peptides comprises a functional group to which the linker is attached;
(k) the compound comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are linked together by the linker;
(l) the compound comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each directly linked to the linker by a covalent bond; or
(m) the pHLIP peptides are attached to the linker by covalent bonds.

Embodiment 19. The compound of any one of Embodiments 1-18, comprising at least one pHLIP peptide that is attached to the linker by a covalent bond.

Embodiment 20. The compound of Embodiment 19, wherein
(a) the covalent bond is a peptide bond;
(b) the covalent bond is a disulfide bond, a bond between two selenium atoms, or a bond between a sulfur and a selenium atom;
(c) the covalent bond is a bond that has been formed by a click chemistry reaction; or
(d) the covalent bond is a bond that has been formed by a reaction between an azide and an alkyne, an alkyne and a strained difluorooctyne, a diaryl-strained-cyclooctyne and a 1,3-nitrone, a cyclooctene, trans-cycloalkene, or oxanorbornadiene and an azide, tetrazine, or tetrazole, an activated alkene or oxanorbornadiene and an azide, a strained cyclooctene or other activated alkene and a tetrazine, or a tetrazole that has been activated by ultraviolet light and an alkene.

Embodiment 21. The compound any one of Embodiments 1-20, wherein
(a) the covalent bond is a peptide bond;
(b) the covalent bond is not a peptide bond;
(c) the linker comprises an artificial polymer or a synthetically produced polymer that has the structure of a polymer that exists in nature;
(d) the linker comprises a polypeptide, a polylysine, a polyarginine, a polyglutamic acid, a polyaspartic acid, a polycysteine, or a polynucleic acid;
(e) the linker does not comprise an amino acid;
(f) the linker comprises a polysaccharide, a chitosan, or an alginate;
(g) the linker comprises a poly(ethylene glycol), a poly(lactic acid), a poly(glycolic acid), a poly(lactic-co-glycolic acid), a poly(malic acid), a polyorthoester, a poly(vinylalcohol), a poly(vinylpyrrolidone), a poly(methyl methacrylate), a poly(acrylic acid), a poly(acrylamide), a poly(methacrylic acid), a poly(amidoamine), a polyanhydrides, or a polycyanoacrylate;
(h) the linker comprises a linear polymer or a branched polymer;
(i) the linker comprises an organic compound structure;
(j) the linker comprises an organic compound structure, wherein the organic compound structure has a molecular weight less than about 10, 9, 8, 7, 6, 5, 4, 3, 2, 1, or 0.5 kilodaltons (kDa);
(k) the linker comprises poly(ethylene glycol); or
(l) the linker comprises poly(ethylene glycol), wherein the poly(ethylene glycol) has a molecular weight of 60 to 100,000 Daltons.

Embodiment 22. The compound of any one of Embodiments 1-21, wherein the linker comprises a cell, a particle, a dendrimer, or a nanoparticle.

Embodiment 23. The compound of any one of Embodiments 1-22,
(a) comprising at least one pHLIP peptide that comprises a functional group for cargo compound attachment;
(b) wherein the linker comprises a functional group for cargo compound attachment;
(c) comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each individually attached to a cargo compound via a linker;
(d) comprising 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 pHLIP peptides that are each individually directly attached to a cargo compound by a covalent bond;
(e) wherein at least one of the pHLIP peptides is attached to a cargo compound by a covalent bond, wherein the covalent bond is an ester bond, a disulfide bond, a bond between two selenium atoms, a bond between a sulfur and a selenium atom, or an acid-liable bond;
(f) wherein at least one of the pHLIP peptides is attached to a cargo compound by a covalent bond, wherein the covalent bond is a bond that has been formed by a click chemistry reaction; or
(g) wherein at least one of the pHLIP peptides is attached to a cargo compound by a covalent bond, wherein the covalent bond is a bond that has been formed by a click chemistry reaction.

Embodiment 24. The compound of Embodiment 23, wherein
(a) the functional group is a side chain of an amino acid of at least one pHLIP peptide;
(b) the functional group is a side chain of an amino acid of at least one pHLIP peptide, wherein the side chain is a side chain to which a cargo compound may be attached via a disulfide bond;
(c) the functional group comprises a free sulfhydryl (SH) or selenohydryl (SeH) group;
(d) the functional group comprises a cysteine, homocysteine, selenocysteine, or homoselenocysteine;
(e) the functional group comprises a primary amine;
(f) the functional group comprises an azido modified amino acid; or
(g) the functional group comprises an alkynyl modified amino acid.

Embodiment 25. The compound of any one of Embodiments 1-24, wherein the linker is attached to a cargo compound via a covalent bond.

Embodiment 26. The compound of Embodiment 25, wherein
(a) the covalent bond is an ester bond, a disulfide bond, a bond between two selenium atoms, a bond between a sulfur and a selenium atom, or an acid-liable bond;
(b) the covalent bond is a bond that has been formed by a click chemistry reaction;
(c) the covalent bond is a bond that has been formed by a reaction between an azide and an alkyne, an alkyne and a strained difluorooctyne, a diaryl-strained-cyclooctyne and a 1,3-nitrone, a cyclooctene, trans-cycloalkene, or oxanorbornadiene and an azide, tetrazine, or tetrazole, an activated alkene or oxanorbornadiene and an azide, a strained cyclooctene or other activated alkene and a tetrazine, or a tetrazole that has been activated by ultraviolet light and an alkene.

Embodiment 27. The compound of any one of Embodiments 1-26, further comprising a cargo compound.

Embodiment 28. The compound of Embodiment 27, wherein
(a) the cargo compound is polar or nonpolar;
(b) the cargo compound comprises a marker;
(c) the cargo compound comprises a prophylactic, therapeutic, diagnostic, radiation-enhancing, radiation-sensitizing, imaging, gene regulation, immune activation, cytotoxic, apoptotic, or research agent;
(d) the cargo compound comprises a dye, a fluorescent dye, a fluorescence quencher, or a fluorescent protein;
(e) the cargo compound comprises a magnetic resonance, positron emission tomography, single photon emission computed tomography, fluorescent, optoacoustic, ultrasound, or X-ray contrast imaging agent;
(f) the cargo compound comprises a peptide, a protein, an enzyme, or a polysaccharide;
(g) the cargo compound comprises an aptamer, an antigen, a protease, an amylase, a lipase, a Fc receptor, a tissue factor, or a complement component 3 (C₃) protein;
(h) the cargo compound comprises a toxin, an inhibitor, a DNA intercalator, an alkylating agent, an antimetabolite, an anti-microtubule agents, a topoisomerase inhibitor, or an antibiotic compound;
(i) the cargo compound comprises an amanita toxin, a vinca alkaloid, a taxane, an anthracycline, a bleomycin, a nitrogen mustard, a nitrosourea, a tetrazine, an aziridine, a platinum-containing chemotherapeutic agent, cisplatin or a cisplatin derivative, a procarbazine, or a hexamethylmelamine;
(j) the cargo compound comprises a DNA, a DNA analog, a RNA, a RNA analog;
(k) the cargo compound comprises a peptide nucleic acid (PNA), a bis PNA, a gamma PNA, a locked nucleic acid (LNA), or a morpholino;
(l) the cargo compound comprises a chemotherapeutic compound;
(m) the cargo compound comprises an antimicrobial compound; or
(n) the cargo compound comprises a gene-regulation compound.

Embodiment 29. The compound of any one of Embodiments 1-28, wherein at least one of the pHLIP peptides comprises an amino acid side chain that is radioactive or detectable by probing radiation.

Embodiment 30. The compound of any one of Embodiments 1-29, wherein one or more atoms of the compound is a radioactive isotope or has been replaced with a stable isotope.

Embodiment 31. A formulation for a parenteral, a local, or a systemic administration comprising the compound of any one of Embodiments 1-30.

Embodiment 32. A compound for the treatment of a superficial or muscle invasive bladder tumor comprising (i) a pHLIP peptide that is attached to at least one other pHLIP peptide via a peptide linker, and (ii) an amanitin toxic cargo.

Embodiment 33. A formulation for the ex vivo treatment of a biopsy specimen, a liquid biopsy specimen, surgically removed tissue, a surgically removed liquid, or blood, comprising the compound of any one of Embodiments 1-30.

Embodiment 34. A pH-triggered peptide (pHLIP peptide) comprising the sequence of at least 8 to 25 consecutive amino acids that is present in any one of the following sequences:

(SEQ ID NO: 124)
X₂X₂RX₂X₂X₁X₂X₂X₂X₃X₃X₂X₂X₂X₂X₂X₁X₂X₂X₂X₂,
(SEQ ID NO: 125)
X₂X₂RX₂X₃X₁X₂X₂X₂X₃X₃X₂X₂X₂X₂X₂X₁X₂GX₂X₂,
(SEQ ID NO: 126)
X₂X₂RX₂X₃X₁X₂X₂X₃X₃X₂X₂X₂X₂X₂X₁X₂X₂X₂X₂X₂,
(SEQ ID NO: 127)
X₂X₂RX₂X₂X₁X₂X₂X₂X₃X₃X₂X₂X₂X₂X₂X₁X₂X₃X₂X₂,
(SEQ ID NO: 128)
X₂X₂X₂X₂X₁X₂X₂X₂X₂X₂X₃X₃X₂X₂X₂X₁X₂X₂RX₂X₂,
(SEQ ID NO: 129)
X₂X₂GX₂X₁X₂X₂X₂X₂X₂X₃X₃X₂X₂X₂X₁X₃X₂RX₂X₂,
(SEQ ID NO: 130)
X₂X₂X₂X₂X₂X₁X₂X₂X₂X₂X₂X₃X₃X₂X₂X₁X₃X₂RX₂X₂,
(SEQ ID NO: 131)
X₂X₂X₃X₂X₁X₂X₂X₂X₂X₂X₃X₃X₂X₂X₂X₁X₂X₂RX₂X₂,
(SEQ ID NO: 132)
GX₂X₂GX₂X₂GX₂X₁GX₂X₂GX₂X₂X₂GX₂X₂X₁GX₂X₂X₂GX₂,
(SEQ ID NO: 133)
X₂GX₂X₂X₂GX₁X₂X₂GX₂X₂X₂GX₂X₂GX₁X₂GX₂X₂GX₂X₂G,
(SEQ ID NO: 134)
X₂RX₂X₂X₂X₁X₂X₂X₂X₂X₃X₁X₃X₂X₂X₂X₁X₂X₂X₂,
(SEQ ID NO: 135)
X₂X₂X₂X₁X₂X₂X₂X₃X₁X₃X₂X₂X₂X₂X₁X₂X₂X₂RX₂,
(SEQ ID NO: 136)
X₂X₂RX₂X₂X₁X₂X₂X₂X₂X₃X₁X₃X₂X₂X₂XX₂,
(SEQ ID NO: 137)
X₂X₂QX₂X₂X₁X₂X₂X₂X₂X₃X₁X₃X₂X₂X₂X₁X₂,
(SEQ ID NO: 138)
X₂X₁X₂X₂X₂X₃X₁X₃X₂X₂X₂X₂X₁X₂X₂RX₂X₂,
(SEQ ID NO: 139)
X₂X₁X₂X₂X₂X₃X₁X₃X₂X₂X₂X₂X₁X₂X₂QX₂X₂,
(SEQ ID NO: 140)
X₂X₂X₂X₃X₃X₂X₂X₂X₂X₂NGX₂X₂X₂X₂X₁,
(SEQ ID NO: 141)
X₂X₂X₂X₃X₃X₂X₂X₂X₂X₂X₂GX₂X₂X₂X₂X₁,
(SEQ ID NO: 142)
X₂X₂RX₂X₂X₁X₂X₂X₂X₂X₃X₃X₂X₂X₂,
(SEQ ID NO: 143)
X₁X₂X₂X₂X₂GNX₂X₂X₂X₂X₂X₃X₃X₂X₂X₂,
(SEQ ID NO: 144)
X₁X₂X₂X₂X₂GX₂X₂X₂X₂X₂X₂X₃X₃X₂X₂X₂,
(SEQ ID NO: 145)
X₂X₂X₂X₃X₃X₂X₂X₂X₂X₁X₂X₂RX₂X₂,
(SEQ ID NO: 146)
GNX₂X₁GX₂X₂X₂X₃X₂GGX₁X₂X₂X₂X₂X₃X₂X₂X₂X₂X₂X₂X₁,
(SEQ ID NO: 147)
X₁GX₂X₂X₂X₃X₂GGX₁X₂X₂X₂X₂X₃X₁X₂X₂X₂X₂X₂X₁,
(SEQ ID NO: 147)
X₁GX₂X₂X₂X₃X₂GGX₁X₂X₂X₂X₂X₃X₁X₂X₂X₂X₂X₂X₁,
(SEQ ID NO: 149)
X₁X₂X₂X₂X₂X₂X₂X₃X₂X₂X₂X₂X₁GGX₂X₃X₂X₂X₂GX₁X₂NG,
(SEQ ID NO: 150)
X₁X₂X₂X₂X₂X₂X₁X₃X₂X₂X₂X₂X₁GGX₂X₃X₂X₂X₂GX₁,
(SEQ ID NO: 151)
X₁X₂X₂X₂X₂X₂X₁X₃X₂X₂X₂X₂X₁GGX₂X₃X₂X₂X₂GX₁,
(SEQ ID NO: 152)
X₂X₂X₁X₂X₂X₂GX₂X₂X₂X₂X₂X₃X₃X₂X₁X₂X₂X₂QX₂,
and
(SEQ ID NO: 153)
X₂QX₂X₂X₂X₁X₂X₃X₃X₂X₂X₂X₂X₂GX₂X₂X₂X₁X₂X₂,

wherein
   each X₁ is, individually, D, E, Gla, or Aad,
   each X₂ is, individually, A, I, L, M, F, P, W, Y, V, or G and
   each X₃ is, individually, S, T, or G.

Embodiment 35. A pHLIP peptide comprising at least 8 consecutive amino acids, wherein
(i) at least 4 of the 8 consecutive amino acids are non-polar amino acids,
(ii) at least 1 of the at least 8 consecutive amino acids is protonatable,
(iii) the pHLIP peptide has a higher affinity for a membrane lipid bilayer at pH 5.0 compared to the affinity at pH 8.0, and
(iv) the at least 8 consecutive amino acids comprise 8 consecutive amino acids in a sequence that is identical to a sequence of 8 consecutive amino acids that occurs in a naturally occurring human protein.

Embodiment 36. The pHLIP peptide of Embodiment 35, comprising the following sequence: LGGEIALW (SEQ ID NO: 322).

Embodiment 37. The pHLIP peptide of Embodiment 36, comprising the following sequence: NLEGFFATLGGEIALWSLVVLAIE (SEQ ID NO: 82).

Embodiment 38. A pHLIP peptide having the sequence:
XₙYₘ; YₘXₙ; XₙYₘXⱼ; YₘXₙYᵢ; YₘXₙYᵢXⱼ; XₙYₘXⱼYᵢ; YₘXₙYᵢXⱼYᵢ; XₙYₘXⱼYᵢXᵢ; YₘXₙYᵢXⱼYₗXₕ; XₙYₘXⱼYᵢXₕY_{g}; YₘXₙYᵢXⱼYₗXₕY_{g}; XₙYₘXⱼYᵢXₕY_{g}X_{f}; (XY)ₙ; (YX)ₙ; (XY)ₙYₘ; (YX)ₙYₘ; (XY)ₙXₘ; (YX)ₙXₘ; Yₘ(XY)ₙ; Yₘ(YX)ₙ; Xₙ(XY)ₘ; Xₙ(YX)ₘ; (XY)ₙYₘ(XY)ᵢ; (YX)ₙYₘ(YX)ᵢ; (XY)ₙXₘ(XY)ᵢ; (YX)ₙXₘ(YX)ᵢ; Yₘ(XY)ₙ; Yₘ(YX)ₙ; Xₙ(XY)ₘ; or Xₙ(YX)ₘ, wherein,
(i) each Y is, individually, a non-polar amino acid with solvation energy, ΔG_{X}^{cor}>+0.50, or Gly;
(ii) each X is, individually, a protonatable amino acid,
(iii) n, m, i, j, 1, h, g, f are each, individually, an integer from 1 to 8.

Embodiment 39. A non-ocular cell comprising an exogenous nucleic acid encoding a pHLIP peptide comprising at least 8 consecutive amino acids with a sequence that is at least 85% identical to (i) a sequence of at least 8 consecutive amino acids that occurs in a naturally occurring human protein; or (ii) the reverse of a sequence of at least 8 consecutive amino acids that occurs in a naturally occurring human protein.

Embodiment 40. A non-ocular cell comprising a pHLIP peptide comprising at least 8 consecutive amino acids with a sequence that is at least 85% identical to (i) a sequence of at least 8 consecutive amino acids that occurs in a naturally occurring human protein; or (ii) the reverse of a sequence of at least 8 consecutive amino acids that occurs in a naturally occurring human protein expressed on the surface of said cell.

Embodiment 41. The non-ocular cell of Embodiment 40, wherein the at least 8 consecutive amino acids are located outside of the lipid bilayer of the cell membrane of said cell.

Embodiment 42. The non-ocular cell of Embodiment 40 or 41, wherein at least 85% of the expressed pHLIP peptide is presented on the exterior of said cell.

Embodiment 43. The non-ocular cell of any one of Embodiments 40-42, which is a T-cell, a B-cell, a neutrophil, an eosinophil, a basophil, a lymphocyte, a monocyte, a dendritic cell, a natural killer cell, or a macrophage.

### EXAMPLES

### Example 1: pHLIP Compounds for Targeted Intracellular Delivery of Cargo Molecules to Tumors

pH (Low) Insertion Peptides (pHLIPs) target acidity at the surfaces of cancer cells and show utility in a wide range of applications, including optical and nuclear imaging, and the intracellular delivery of cell-impermeable and cell-permeable therapeutic agents. Here pHLIP constructs are introduced that improve the targeted delivery of agents into tumor cells. The constructs presented herein include pHLIP bundles, e.g., conjugates consisting of two or four pHLIP peptides linked together by polyethelyne glycol (PEG), and Var3 pHLIP variants containing either the non-standard amino acids γ-carboxyglutamic acid (Gla) or a GLL motif. The in vitro and in vivo performance of the constructs was compared with previous pHLIP variants. A wide range of experiments was performed on nine constructs including: i) biophysical measurements of steady-state and kinetic fluorescence, circular dichroism, and oriented circular dichroism, in order to study the pH-dependent insertion of pHLIP variants across the membrane lipid bilayer; ii) cell viability assays to gauge the pH-dependent potency of peptide-toxin constructs by assessing the intracellular delivery of the polar, cell-impermeable cargo molecule, amanitin, at physiological and low pH (pH 7.4 and 6.0, respectively); and iii) tumor targeting and biodistribution measurements using fluorophore-peptide conjugates in a breast cancer mouse model. The main principles of the design of pHLIP variants for various medical applications are discussed.

Targeting tumors based on the acidic environment at the surfaces of cancer cells presents several advantages to traditional biomarker targeting methods. Past studies have demonstrated the utility of the class of pH (Low) Insertion Peptides (pHLIPs) for targeting the acidity present in tumor tissue in applications such as fluorescence and nuclear imaging, and drug and gene therapy. Here, several pHLIPs are described, including pHLIP bundles, and these constructs are thoroughly evaluated alongside an improved generation of pHLIPs. Challenges relating to the design and accurate evaluation of pHLIPs are also discussed. The research elucidates the strengths and weaknesses of existing pHLIPs, proposes future peptide modifications that could further improve tumor targeting, and discusses the applicability of this improved generation of pHLIPs for drug delivery.

The targeted delivery of drugs to cancer cells maximizes their therapeutic effect while reducing side effects. Although many biomarkers exist that can be exploited to improve tumor targeting and treatment outcomes, such as various receptors overexpressed at the surfaces of cancer cells, the heterogeneity of the cancer cell population in an individual tumor and between tumors of various patients limits the effective use of biomarker targeting technologies. Additionally, rapid mutation increases the likelihood of the selection of cancer cell phenotypes that do not express high levels of the targeted biomarker. In both situations, biomarker targeting acts as a selection method that can lead to the development of drug resistance and poor patient outcomes (Marusyk A & Polyak K (2010) Biochim Biophys Acta 1805(1):105; Gillies et al. (2012) Nat Rev Cancer 12(7):487-493; Lloyd et al. (2016) Cancer Res 76(11):3136-3144). It is well known that acidosis is a characteristic ubiquitous to tumors, including both primary tumors and metastases (Estrella et al. (2013) Cancer Res 73(5):1524-1535). This acidic microenvironment is generated by the increased use of the glycolytic mechanism of energy production by cancer cells, and by the abundance of carbonic anhydrase proteins on the cancer cell surfaces. The extracellular pH is the lowest at the surface of cancer cells, and is significantly lower than normal physiological pH and the bulk extracellular pH in tumors (Zhang X, Lin Y, & Gillies R J (2010) J Nucl Med 51(8):1167-1170; Hashim et al. (2011) NMR in biomedicine 24(6):582-591; Anderson et al. (2016) Proc Natl Acad Sci USA 113(29):8177-8181). The low pH layer remains at the cancer cell surface even in well-perfused tumor areas. This layer of acidity on the surface of cancer cells is a targetable characteristic of tumor tissue, which is not subject of clonal selection, and the level of acidity is a predictor of tumor invasion and aggression. Rapidly growing tumor cells are more acidic.

The family of pH (low) insertion peptides (pHLIPs) comprises a variety of acidity-targeting peptides, each possessing different tumor-targeting characteristics. pHLIPs can be used in a wide variety of applications, so it is desirable to have a range of options for specific applications. The applications include i) fluorescence imaging (Reshetnyak et al. (2011) Mol Imaging Biol 13(6):1146-1156; Adochite et al. (2014) Mol Pharm 11(8):2896-2905; Tapmeier et al. (2015) Proc Natl Acad Sci USA 112(31):9710-9715) and fluorescence image-guided surgery (Golijanin et al. (2016) Proc Natl Acad Sci USA 113(42):11829-11834); ii) nuclear imaging including PET and SPECT (Macholl et al. (2012) Mol Imaging Biol 14(6):725-734; Demoin et al. (2016) Bioconjugate Chem 27(9):2014-2023); iii) therapeutic applications, such as the targeted delivery of polar toxins that cannot cross cell membranes (An et al. (2010) Proc Natl Acad Sci USA 107(47):20246-20250; Wijesinghe et al. (2011) Biochemistry-US 50(47):10215-10222), drug-like molecules that diffuse across cell membranes (Burns et al. (2015) Mol Pharm 12(4):1250-1258; Burns et al. (2017) Mol Pharm 14(2):415-422), and gene therapy (Cheng et al. (2015) Nature 518(7537):107-110); and iv) nanotechnology for enhancing the delivery of gold nanoparticles (Yao et al. (2013) Proc Natl Acad Sci USA 110(2):465-470; Daniels et al. (2017) Biochem Biophys Rep 10:62-69) or liposome-encapsulated payloads to cancer cells (Yao et al. (2013) J Control Release 167(3):228-237; Wijesinghe et al. (2013) Sci Rep 3:3560).

pHLIPs are triggered to insert across the membranes of cancer cells by the acidity at the cancer cell surface. The behavior of peptides in the pHLIP family is typically described in terms of three states: at physiological pH, peptides exist in equilibrium between a solvated state (State I) and a membrane-adsorbed state (State II); a decrease in pH shifts the equilibrium toward a membrane-inserted state (State III) (Reshetnyak et al. (2007) Biophys J 93(7):2363-2372). The mechanism of action of peptides in the pHLIP family is well understood: protonatable residues, which are interspersed throughout the hydrophobic middle region and the C-terminal, membrane-inserting region of the peptides, are negatively charged at physiological pH (e.g., pH 7.4) but become protonated and neutrally charged with a decrease in pH. The loss of charge and increase in overall hydrophobicity drives pHLIPs to partition into the hydrophobic core of the membrane bilayer, and triggers the formation of a transmembrane (TM) helix. This helix spans the lipid bilayer, leaving the N-terminus in the extracellular space and placing the C-terminus in the intracellular space, where, due to the more alkaline pH in the cytosol, the C-terminus can again become deprotonated and charged, stably anchoring the peptide in the cell membrane.

Following the extensive characterization of wild-type (WT) pHLIP, the first-generation of pHLIP variants was created to examine the effects on targeting due to fairly straightforward changes to the WT primary structure such as sequence truncation, the addition and replacement of some protonatable residues with others, and sequence reversal (Reshetnyak et al. (2008) Proc Natl Acad Sci USA 105(40):15340-15345; Karabadzhak et al. (2012) Biophys J 102(8):1846-1855; Weerakkody et al. (2013) Proc Natl Acad Sci USA 110(15):5834-5839). Of these first-generation variants, Variant 3 (Var3) appeared to have the most desirable insertion characteristics, and much research has been focused around the use of Var3 for various applications (Tapmeier et al. (2015) Proc Natl Acad Sci USA 112(31):9710-9715; Golijanin et al. (2016) Proc Natl Acad Sci USA 113(42):11829-11834; Cruz-Monserrate et al. (2014) Sci Rep 4:4410; Adochite et al. (2016) Mol Imaging Biol 113(42):11829-11834). Lately, additional variants have emerged that incorporate more exotic changes to the peptide primary structure; these changes include the use of the non-standard amino acids γ-carboxyglutamic acid (Gla), a residue with two protonatable carboxyl groups, and α-aminoadipic acid (Aad), a more hydrophobic version of the glutamic acid residue (Onyango et al. (2015) Angew Chem Int Edit 54(12):3658-3663), as well as the creation of a pHLIP peptide de novo, ATRAM (Nguyen et al. (2015) Biochemistry-US 54(43):6567-6575). Here, several additional members of the pHLIP family of peptides and pHLIP bundles are introduced, their biophysical properties are compared to some previously introduced variants, and the utility of nine pHLIPs in drug-delivery and tumor imaging is evaluated. Variants disclosed herein significantly expand the useful range of use in targeted cancer therapy.

pHLIP Constructs

Several pHLIP variants were investigated; data described herein shows results from nine variants, among them are Var3/Gla (with nonstandard amino acid Gla), Var3/GLL (with glycine-leucine-leucine motif), and pHLIP bundles. The pHLIP bundles include two- or four-armed polyethylene glycol (PEG) 2 kDa spacers conjugated with the Cys residue at the N-terminus of WT: PEG-2WT (FIG. 1A) and PEG-4WT (FIG. 1), respectively. The motivation was to increase affinity to membrane and enhance cooperativity of transition from the membrane-surface to the membrane-inserted peptide state, when C-terminal end of the peptide is translocated across bilayer, with main goal to enhance intracellular delivery of cargoes and targeting acidic tumors. Notwithstanding variation in peptide sequence due to the addition of a single N- or C-terminal cysteine or lysine residue for conjugation purposes (a list of all investigated peptides is provided in Table 9), the nine pHLIP variants studied were (molecular weights and retention times are provided in Table 10):

List of Main Groups of pHLIP Variants
List of Main Groups of pHLIP Variants Described in FIGS. 1-6

| | |
|---|---|
| WT: | AEQNPIYWARYADWLFTTPLLLLDLALLVDADEGT (SEQ ID NO: 2) |
| PEG-2WT: | Two-arm PEG conjugated to 2 WT |
| PEG-4WT: | Four-arm PEG conjugated to 4 WT |
| WT/Gla: | AEQNPIYWARYAGlaWLFTTPLLLLDLALLVDADEGT (SEQ ID NO: 44) |
| WT/Gla/Aad: | AEQNPIYWARYAGlaWLFTTPLLLLAadLALLVDADEGT (SEQ ID NO: 45) |
| Var3: | ADDQNPWRAYLDLLFPTDTLLLDLLW (SEQ ID NO: 46) |
| Var3/Gla: | ADDQNPWRAYLGlaLLFPTDTLLLDLLW (SEQ ID NO: 47) |
| Var3/GLL: | GEEQNPWLGAYLDLLFPLELLGLLELGLWG (SEQ ID NO: 48) |
| ATRAM: | GLAGLAGLLGLEGLLGLPLGLLEGLWLGLELEGN (SEQ ID NO: 3) |

Enhancement of affinity improves targeting, and higher cooperativity narrows the window of pH that produces TM drug delivery. The information about all pHLIP variants used in the study with additional variations from the addition of single N- or C-terminal cysteine or lysine residues for conjugation purposes is provided in Tables 9 and 10. Nine pHLIP variants are grouped together in various ways by shared characteristics. A WT-like group contains peptides with two protonatable residues (shown in bold in the list above) in the putative TM region, multiple protonatable residues in the membrane-inserting C-terminal region, and two tryptophan residues (residue W) both located at the beginning of the helix-forming TM region; this group includes WT, PEG-2WT and PEG-4WT, WT/Gla, and WT/Gla/Aad. A Var3-like group is based on Var3 from the first pHLIP series (Weerakkody et al. (2013) Proc Natl Acad Sci USA 110(15):5834-5839). This group includes Var3, Var3/Gla, and Var3/GLL, each of which have three protonatable residues in the TM region and tryptophan residues located at the beginning and end of the TM region. Considering this scheme, ATRAM, with its multiple glycine and leucine residues and single tryptophan located about two-thirds to the end of its TM part, is in a group of its own. Other subgroups could be considered as well: a subgroup of peptides that incorporate the non-standard Gla residue, shown in italics in the list above (i.e., WT/Gla, WT/Gla/Aad, and Var3/Gla), and another subgroup that includes peptides containing the GLL motif (Var3/GLL and ATRAM). When performing analysis of biophysical measurements, analyzing variants with respect to their group-mates becomes important: the very different characteristics of peptides from various groups make it difficult to accurately compare the behavior of all peptides at the same time.

Biophysical Steady-State and Kinetics Studies

A variety of spectroscopic techniques to probe pHLIP variants interactions with phospholipid bilayer of POPC liposomes including steady-state fluorescence spectroscopy, circular dichroism (CD), oriented circular dichroism (OCD), and stopped-flow fluorescence measurements were employed. Steady-state fluorescence and CD experiments were conducted in phosphate buffer titrated with hydrochloric acid to drop the pH from pH 8 to pH 4 to ensure consistency with previously published data (Weerakkody et al. (2013) Proc Natl Acad Sci USA 110(15):5834-5839; Nguyen et al. (2015) Biochemistry-US 54(43):6567-6575; Hunt et al. (1997) Biochemistry 36(49):15177-15192). At the same time, steady-state and kinetics fluorescence experiments measuring the pH-dependent transition from State II to State III were performed in phosphate buffer containing physiological concentrations of free calcium (1.25 mM) and magnesium (0.65 mM) ions found in blood.

It was established that in solution, PEG-2WT and PEG-4WT exists in compact coil conformations, where tryptophan and other aromatic residues can form stacking structures. As a result, exciton was reflected by the appearance of a minimum around 230 nm on CD spectra (FIGS. 1D and 1H). According to the changes of the tryptophan fluorescence, both constructs interacted with lipid bilayer of membrane and, most probably, PEG-4WT exhibits stronger binding than PEG-2WT in State II at pH 8. With a reduction of pH, both PEG-pHLIP variants inserted into membrane to form helices, and the transmembrane orientations of these helices were confirmed by OCD measurements (FIGS. 1E and 1I). In State III, the membrane-inserted state, the exciton signal generated by π-π stacking was no longer present. The pK of the transition from State II to State III was shifted to pH 6.6 and cooperativity of the transition was increased for PEG-4WT compared to PEG-2WT (FIGS. 1F and 1J).

Next, the study was extended and the groups consisting of WT, Var3, and ATRAM pHLIP variants were compared to the newly synthesized Var3/Gla and Var3/GLL pHLIP variants. The HPLC retention times of the peptides indicate increasing hydrophobicity within the groups in the following order, from less to more hydrophobic: WT, WT/Gla, WT/Gla/Aad and Var3, Var/Gla, Var3/GLL, and ATRAM, with ATRAM being the most hydrophobic (Table 10). Both Var3/Gla and Var3/GLL demonstrated a pH-dependent interaction with membrane (FIG. 5). Var3/GLL showed a higher percentage of membrane-inserted population at pH 8, which reflects a higher affinity of the peptide to the lipid bilayer both at physiological and high pH due to the increased hydrophobicity of the peptide.

As seen for previous pHLIP designs, the blue shift (or decrease in Stokes shift) in transition from State I to State II and State III was observed for all peptides (Table 11), indicating partitioning of the peptides into the lipid bilayer. However, the positions of fluorescence spectra maxima for peptides belonging to different groups cannot be compared directly, since the locations of the tryptophan residues within the peptides vary greatly. With this fact in mind, and without being bound by any theory, it can be concluded that peptides had very different conformations in State II at pH 8, and that the highest membrane affinity was exhibited by the PEG-pHLIPs, WT/Gla/Aad, Var3/GLL, and ATRAM peptides. PEG-pHLIPs have multiple binding sites due to the linking of multiple WT peptides within a single construct, which is expected to enhance binding affinity. At the same time, WT/Gla/Aad, Var3/GLL, and ATRAM were the most hydrophobic sequences, and thus exhibited stronger binding/insertion. It was also found that some peptides were especially sensitive to the presence of calcium and magnesium ions, namely WT, variants containing the Gla residue (WT/Gla, WT/Gla/Aad, and Var3/Gla) and ATRAM. This sensitivity was most obviously indicated by a decreased Stokes shift (usually 2-3 nm) in State I and/or State II (data not shown), and might reflect slight increases in the hydrophobicity of the peptides caused by the coordination of divalent cations resulting from the presence of closely spaced protonatable residues, such as those found in the C-terminal region of WT and, to some degree, in ATRAM, and to the presence of the Gla residue, with its two protonatable carboxyl groups, in the WT/Gla, WT/Gla/Aad, and Var3/Gla peptides. It was previously shown that the Gla residue possesses the ability to complex calcium ions (Cabaniss et al. (1991) Int J Pept Prot Res 37(1):33-38; Shikamoto et al. (2003) J Biol Chem 278(26):24090-24094; Huang et al. (2004) J Biol Chem 279(14):14338-14346). The decrease in Stokes shift in State II was likely due to the location of membrane-adsorbed peptides (especially more hydrophobic pHLIPs: WT/Gla/Aad, Var3/GLL, and ATRAM) deeper in the lipid membrane and/or a shift in peptide population from the solvated to the membrane-adsorbed state.

In contrast to tryptophan fluorescence, which is dependent on the location of tryptophan residues within the peptide sequence, the appearance of helicity is a more general parameter which can be compared between all peptides. FIG. 2A (and Table 11) present the ratio of ellipticity at 205 nm to 222 nm, an indicator of the degree of helicity (lower ratios indicate higher helicity), obtained for different peptides in different states. In State I, the lowest ratios were observed for pHLIP bundles, which correlate with the appearance of the exciton signal at 230 nm. In State II, the least unstructured peptides (ratios<1.5) were PEG-4WT, WT/Gla/Aad, Var3/GLL, ATRAM, and PEG-2WT, which exhibited a higher affinity to the membrane and an increase in the peptide-inserted population at pH 8. At low pH, all peptides exhibited similar helical content, which reflected insertion into the bilayer and the formation of TM helices.

The transitions from State II to State III investigated in steady-state and kinetics modes in the presence of physiological concentrations of calcium and magnesium ions demonstrated pK values in the range of pH 5.7 to 6.6, with highest cooperativity observed for PEG-4WT, and transition times varying from 0.1 to 37.5 s (Table 8). There are subtleties that affect the comparison and interpretation of the data such as: i) the peptides were in different starting conditions in State II at pH 8 due to greatly differing overall peptide hydrophobicity; ii) difference in peptides pK values, which reflect equilibrium between peptides' membrane-adsorbed and membrane-inserted populations; (iii) characteristic times, which report the movement of tryptophan residues into environments inside the membrane; however, sincethe tryptophan residues are located in different regions of each pHLIP, their movement into the membrane, as measured via changes in fluorescence parameters, should be expected to bedifferent; and (iv) the cooperativity of the transition is a somewhat unstable parameter in the fitting of experimental pHdependence data using the Henderson-Hasselbalch equation, especially if slopes are introduced at the initiation and completion of the transition (Barrera et al. (2011) J Mol Biol 413(2):359-371). Lower values of cooperativity (n<1) were observed for the peptides with tryptophan residues located at (Var3 group) or close (ATRAM) to the C-terminal end, which must be translocated across the cell membrane. ATRAM and Var3/GLL, which were the most hydrophobic pHLIPs and were therefore already located deeper in the membrane at pH 8, demonstrated the fastest times of insertion. As was shown previously, the removal of protonatable residues from the inserting C-terminus increases the rate of the transition from State II to State III (Karabadzhak et al. (2012) Biophys J 102(8):1846-1855; Weerakkody et al. (2013) Proc Natl Acad Sci USA 110(15):5834-5839). Thus, the group of Var3-like peptides exhibited fast insertion time (t<1 s). In the group of WT peptides, the time of insertion decreased as the hydrophobicity of the peptide increased, with insertion times listed in the following order (from longest to shortest time of insertion): WT, WT/Gla, WT/Gla/Aad, PEG-2WT, and PEG-4WT.

Intracellular Delivery of Polar Cargo

First, whether the pHLIP bundles could cause any cytotoxicity by themselves was evaluated. HeLa cells were treated with either PEG-2WT or PEG-4WT at physiological pH (pH 7.4) and low pH (pH 6.0) for two hours. No cytotoxic effect was observed at either pH, even when treating with concentrations up to 10 μM (construct concentration is presented as concentration of WT pHLIP).

Next, a proliferation assay was employed to evaluate the ability of pHLIPs to intracellularly delivery the toxin amanitin, a cell-impermeable polar cargo molecule (Moshnikova et al. (2013) Biochemistry-US 52(7):1171-1178; Weerakkody et al. (2016) Sci Rep 6:31322). For amanitin to induce cytotoxicity, it must be translocated across the cell membrane, be released from peptide carrier, and reach its target (RNA polymerase II) in nucleus. Amanitin was conjugated via a cleavable disulfide link to the inserting, C termi of the peptides. The translocation capabilities of the pHLIP-amanitin conjugates were probed by investigating the inhibition of proliferation of HeLa cells treated with increasing concentrations (up to 2 μM) of pHLIP-amanitin at either physiological pH (pH 7.4) or low pH (pH 6.0) for two hours, followed by removal of the constructs, transferring cells to normal cell culture media, and assessing cell death at 48 hours.

Each of the conjugates demonstrated pH-dependent cytotoxicity (FIG. 6). The calculated EC₂₀, EC₅₀, EC₅₀ at physiological and low pH are shown in Table 8. At low pH, the most potent were pHLIP bundles, which exhibited the highest cooperativity of transition from membrane-adsorbed to membrane-inserted states. The least toxic at normal pH among all constructs was Var3. FIG. 2B presents therapeutic index (TI), which is ratio of EC₅₀ at pH7.4 to EC₅₀ at pH6.0. The highest ratio of about 9 was obtained for WT/Gla and Var3, and TI was established to be around 5.5 for PEG-2WT, Var3/Gla and ATRAM. It is desirable to have high potency, which is the difference between cell viability at low and physiological pHs calculated at different concentrations of the construct (FIG. 3). All constructs exhibited a high potency (60 to 70%) at some particular concentrations; however, just a few constructs, namely Var3, Var3/Gla, and WT/Gla, demonstrated a high, stable potency over a wide range of concentrations. The pHLIP bundles displayed the highest potency at the lowest concentrations (0.1 μM to 0.2 μM). The potency of ATRAM peaked at concentrations around 0.5 μM, and declined sharply at higher concentrations; this decline is most likely associated with the increased hydrophobicity of ATRAM, which results in a high affinity for the cell membrane at normal and high pH and promotes the shift in equilibrium toward the membrane-inserted form that is associated with the translocation of cargo across the cell membrane.

Tumor Targeting

To investigate the tumor targeting and biodistribution characteristics of the pHLIP variants, the fluorescent dye Alexa Fluor 546 (AF546) was conjugated to the non-inserting, N-terminal ends of seven of the peptides. Previous data indicate excellent tumor targeting by AF546-pHLIPs (Adochite et al. (2014) Mol Pharm 11(8):2896-2905; Adochite et al. (2016) Mol Imaging Biol 18:686-696). In the case of pHLIP bundles, AF546 was conjugated to the inserting, C termini of the PEG-2WT and PEG-4WT pHLIPs, as the N termini were occupied by PEG polymers. A well-established model of acidic 4T1 murine breast tumors was used in the study; this model is targeted well by pHLIPs (Adochite et al. (2014) Mol Pharm 11(8):2896-2905; Adochite et al. (2016) Mol Imaging Biol 113(42):11829-11834). Following the development of breast tumors in the mouse flank, the fluorescent constructs were introduced by a single tail vein injection. Animals were euthanized four hours after the injection of the fluorescent conjugates, and the tumor and major organs (kidney, liver, lungs, spleen, and muscle) were collected and imaged. The four-hour post-injection time point was selected based on previous pharmacokinetics data which show that the highest tumor targeting with pHLIPs is observed four hours after the injection of construct (Adochite et al. (2014) Mol Pharm 11(8):2896-2905; Adochite et al. (2016) Mol Imaging Biol 113(42):11829-11834). The mean values of the surface fluorescence intensity of tumors, muscle, and organs are given in Table 12. The normalized tumor fluorescence intensity (normalized by tumor uptake of AF546-WT) for all constructs is shown in FIG. 4A. The highest tumor targeting was observed for the Var3 construct, as well as Var3/Gla and ATRAM. The tumor uptake of WT and Var3/GLL constructs were statistically significantly reduced in 1.6 and 2.6 times, respectively, compared to the uptake of Var3. The uptakes of WT/Gla and WT/Gla/Aad, as well as bundled pHLIPs were reduced even further compared to WT construct. It is possible that the decreased tumor targeting observed in the PEG-pHLIPs might be attributed to the fact that the AF546 dye was conjugated to the C-terminal, inserting end of these constructs. At the same time, the tumor-to-muscle ratio of WT-like group was in the range of 5.4 to 7.5. The highest tumor-to-muscle ratio was observed for Var3 (T/M=8.9) and PEG-2WT (T/M=7.5), and the lowest ratio was found for Var3/GLL (T/M=4.0) (FIG. 4B and Table 13). Only Var3/GLL among all variants demonstrated tumor to kidney ratio less than 1 (FIG. 4C and Table 13). The highest tumor to liver ratio was found for Var3 and Var3/Gla (FIG. 4D and Table 13). Without being bound by any theory, because PEG-2WT-AF546 and PEG-4WT-AF546 are several times larger than the other pHLIP variants, it was expected that they might have slower pharmacokinetics. Therefore, imaging was also performed at the 24-hour post-injection time point for the two PEG-pHLIP conjugates; however, significantly higher signal in tumors was not observed at 24 hours post-injection compared to 4 hours post-injection (see Table 12).

pHLIP Compounds for Targeted Intracellular Delivery of Cargo Molecules to Tumors

The study of pHLIPs was been extended by introducing additional variants and pHLIP bundles, and comparing their performance to the performance of recently introduced variants with non-standard amino acids (Gla and Aad) and the hydrophobic GLL motif A goal was to correlate the biophysical properties of the membrane interactions of different pHLIPs at physiological concentrations of free calcium and magnesium ions to the ability of these pHLIPs to move polar cargo across the cell membrane and to target acidic tumors.

The thermodynamic parameters of pK and cooperativity of pH-dependent transition from State II at pH 8 to State III at pH<5 can be taken as predictors of the performance of a pHLIP for drug delivery and tumor targeting (Burns et al. (2017) Mol Pharm 14(2):415-422; Onyango et al. (2015) Angew Chem Int Edit 54(12):3658-3663; Nguyen et al. (2015) Biochemistry-US 54(43):6567-6575). First, while pK is a rather stable fitting parameter, the cooperativity parameter (Hill coefficient) might vary over a wide range resulting from different fittings which are within the level of accuracy of the experimental measurements. Moreover, if different binding affinities are assumed, the Hill formulation loses validity. In general, highly cooperative transitions are hard to measure in biological systems with noise, especially when examining relatively short peptides like the class of pHLIP peptides (Onyango et al. (2015) Angew Chem Int Edit 54(12):3658-3663). Only if the biological system is approximated to be infinite can a phase transition occur (Sharma et al. (2015) Journal of Statistical Mechanics: Theory and Experiment P01034). Moreover, transition parameters for different peptides can only truly be compared when both peptides have precisely the same starting and ending states; although this condition is met for the membrane-inserted state (State III) of the peptides, which is very similar for all pHLIP variants, the condition that the initial state (State II) of the peptides be identical is not met. As hydrophobicity varies among peptides of the pHLIP family due to the difference in numbers of protonatable, polar, and hydrophobic residues and their location within the peptide sequences, the characteristics of the peptide population in the initial state of the transition also varies as these peptides position themselves at different interaction levels with the hydrophobic/hydrophilic boundary region of a bilayer.

The population percentages of inserted peptide presented in Table 14 were calculated from the pH-dependence transitions of pHLIP variants. The numbers represent the percentage of membrane-inserted peptides at varying pH assuming that at the beginning of the transition (State II) (i.e., at physiological pH and higher) the population of inserted peptides is about zero. In reality, close consideration of the interaction between a pHLIP variant and the membrane at pH 8, in conditions more alkaline than physiological conditions where the inserted peptide population should be even less than at physiological conditions, indicates that the most hydrophobic sequences, such as ATRAM and Var3/GLL, and bundled pHLIPs with multiple binding sites within a single construct, demonstrate a significant inserted peptide population. This is reflected by the loss of pH-dependent differences in translocation of the polar, cell-impermeable cargo amanitin with an increase in construct concentration (i.e., a decrease in potency at higher concentrations). Additionally, as previously shown using the pore-forming peptide melittin, helix formation, membrane binding, and insertion properties are very sensitive to primary structure changes involving glycine and leucine residues (Krauson et al. (2015) J Am Chem Soc 137:16144-16152). Ultimately, due to patient variability, it is crucial that potential therapeutic pHLIP constructs are able to discriminate between healthy and tumor tissue over a wide concentration range, meaning that a constant potency is necessary to avoid targeting normal tissue and the resulting significant side effects, suggesting that the properties of these variants may not be well suited for clinical development using agents that require tight targeting.

In addition to the steady-state experiments, it is important to probe tumor targeting and to examine the biodistribution of the constructs when injected into the high-flowrate blood stream, since targeted delivery is may be opposed by clearance from the blood. The best tumor targeting was shown by faster-inserting pHLIP constructs. Thus, in the design of new pHLIP variants, the biophysical kinetics parameters are considered in addition to the steady-state properties. These kinetics parameters are critical for the delivery and translocation of a cargo across membrane, since charges and the presence of cargo at the inserting end of a pHLIP slows down the process of insertion (Karabadzhak et al. (2012) Biophys J 102(8):1846-1855). Different cargoes linked to a pHLIP alter biodistribution and tumor targeting (Adochite et al. (2016) Mol Imaging Biol 113(42):11829-11834). Less polar pHLIP variants conjugated with hydrophobic cargoes might have a higher tendency toward targeting normal tissue and hepatic clearance. On other hand, the size of links in pHLIP bundles are used to tune biodistribution and re-direct clearance from renal to hepatic.

Among the pHLIP variants, Var3 demonstrated excellent performance in vitro, the most stable potency over a wide range of concentrations, and high tumor targeting. Variants containing the Gla residue, especially WT/Gla construct indeed showed an increase in the cooperativity of the membrane insertion transition as previously reported (Onyango et al. (2015) Angew Chem Int Edit 54(12):3658-3663), and improved therapeutic index. However the tumor targeting of WT/Gla was lower compared to the tumor targeting of WT. The γ-carboxyglutamic acid is not naturally encoded in the human genome, but is introduced into proteins through the post-translational carboxylation modification of glutamic acid and has two carboxyl groups. Several proteins are known to have Gla-rich domains, including many coagulation factors, which coordinate calcium ions, inducing conformational changes in the protein which enhance the hydrophobicity and affinity of the protein to the cell membrane bilayer (Kalafatis et al. (1996) Crit Rev Eukar Gene 6(1):87-101). Calcium complex formation by a pHLIP increases the hydrophobicity of the peptide and alters the interaction between peptide and membrane; however, despite the cost of synthesizing a peptide with Gla, such constructs are associated with significant advantages such as increased potency.

pHLIP peptides can be tailored to the specific medical application. For example, kidney clearance might be preferred to liver clearance for PET-pHLIP imaging constructs (Demoin et al. (2016) Bioconjugate Chem 27(9):2014-2023). High tumor-to-normal tissue fluorescence intensity ratios will be the key in fluorescence-guided surgical applications (Golijanin et al. (2016) Proc Natl Acad Sci USA 113(42):11829-11834). Delivery of highly toxic molecules, such as amanitin, are tailored for minimal off-targeting, thus achieving high potency and therapeutic index. However, for the delivery of polar peptide nucleic acids (PNAs) or other highly specific inhibitors of particular pathways in cancer cells, neither of which are associated with toxicity in normal cells, the requirement to reduce off-targeting is much lower, and the emphasis is shifted toward the efficiency of delivery, the goal being is to translocate as much cargo as possible (Cheng et al. (2015) Nature 518(7537):107-110; Reshetnyak et al. (2006) Proc Natl Acad Sci USA 103(17):6460-6465). The pHLIP bundles yield excellent results in these types of applications, supported by the observation that PEG-4WT is the most efficient at delivering the polar molecule amanitin to the intracellular space. Bundling multiple Var3 pHLIPs, in the same fashion in which two or four WT pHLIPs were linked, might be more advantageous. Var3 demonstrates membrane insertion rates orders of magnitude faster than the insertion rates of WT; with the knowledge that faster insertion rates observed in biophysical experiments correlate to better tumor targeting in vivo, it stands to reason that potential PEG-Var3 constructs might demonstrate better tumor targeting still.

In drug delivery applications, pHLIP peptides are best designed for the delivery of polar, cell-impermeable molecules (An et al. (2010) Proc Natl Acad Sci USA 107(47):20246-20250; Moshnikova et al. (2013) Biochemistry-US 52(7):1171-1178; Burns K E & Thevenin D (2015) Biochem J 472(3):287-295; Burns et al. (2016) Sci Rep 6:28465). The intracellular delivery of polar cargo could be further tuned by altering the link connecting the cargo to pHLIP, and/or by attaching modulator molecules to the inserting end of the peptide (An et al. (2010) Proc Natl Acad Sci USA 107(47):20246-20250; Wijesinghe et al. (2011) Biochemistry-US 50(47):10215-10222; Cheng et al. (2015) Nature 518(7537):107-110; Moshnikova et al. (2013) Biochemistry-US 52(7):1171-1178). Additionally, pHLIP are used for the tumor-targeted delivery of cell-permeable, drug-like molecules since it can significantly increase the time of retention in blood, positively alter the biodistribution of drugs that typically rely on passive diffusion, and enhance tumor targeting, leading to an increase in therapeutic index (Burns (2015) Mol Pharm 12(4):1250-1258). More polar pHLIP variants are expected to be better suited applications involving the intracellular delivery of cell-permeable cargoes.

The present disclosure establishes a set of properties for a number of pHLIPs, which can be selected for clinical development in different circumstances. This body of work, with the prior studies, opens pathways for targeted delivery using a range of imaging and therapeutic agents in the fight against cancer.

Materials and Methods

pHLIPs characterization and pHLIP bundle synthesis: All peptides were purchased from CS Bio Co. Peptides were characterized by reversed phase high-performance liquid chromatography (RP-HPLC) using Zorbax SB-C18 and Zorbax SB-C8, 4.6×250 mm 5 m columns (Agilent Technology). For biophysical measurements, PEG-2WT and PEG-4WT were made by conjugating either 2 kDa bifunctional maleimide-PEG-maleimide or 2 kDa 4-arm PEG-maleimide (Creative PEGWorks) to Cys-WT via an N-terminal cysteine residue. Purification of the PEG-pHLIP constructs was conducted using RP-HPLC. Peptide concentration was calculated by absorbance at 280 nm, where, for WT, WT/Gla, and WT/Gla/Aad, F280=13,940 M⁻¹ cm⁻¹; for Var3, Var3/Gla, and Var3/GLL, F280=12,660 M⁻¹ cm⁻¹; and for ATRAM, ε₂₈₀=5,690 M⁻¹ cm⁻¹. PEG construct concentration was presented in terms of peptide concentration, not molecular concentration.

Liposome preparation: Small unilamellar vesicles were used as model membranes and were prepared by extrusion. 1-palmitoyl-2-oleoyl-sn-glycero-3-phosphocholine (POPC; Avanti Polar Lipids) was dissolved in chloroform at a concentration of 12.5 mg/mL, then desolvated by rotary evaporation for two hours under high vacuum. The resulting POPC film was rehydrated in 10 mM phosphate buffer at pH 8, either with ions (1.25 mM calcium and 0.65 mM magnesium), or without ions, vortexed, and extruded fifteen times through a membrane with a pore size of 50 nm.

Steady-state fluorescence measurements: Steady-state fluorescence spectra were measured using a PC1 spectrofluorometer (ISS) with temperature control set to 25.0° C. The tryptophan fluorescence was excited using an excitation wavelength of 295 nm. Excitation and emission slits were set to 8 nm, and excitation and emission polarizers were set to 54.7° and 0.0°, respectively. Sample preparation was conducted 24 hours prior to experiments to allow for State II equilibration. A buffer-only sample was used as a baseline for State I, and a buffer-with-POPC-only sample was used as a baseline for States II and III.

pH dependence measurements: pH dependence measurements were taken with the PC1 spectrofluorometer by using the shift in the position of maximum of peptide fluorescence as an indication of changes of the peptide environment at varying pH. All pH dependence measurements were conducted at physiological concentrations of free calcium and magnesium ions (1.25 and 0.65 mM, respectively). After the addition of hydrochloric acid, the pH of solutions containing 5 μM peptide and 1 mM POPC were measured using an Orion PerHecT ROSS Combination pH Micro Electrode and an Orio Dual Star pH and ISE Benchtop Meter (Thermo Fisher Scientific) before and after spectrum measurement to ensure equilibration. The tryptophan fluorescence spectrum at each pH was recorded, and the spectra were analyzed using the Protein Fluorescence and Structural Toolkit (PFAST) (Shen et al. (2008) Proteins 71(4):1744-1754)(43) to determine the positions of spectral maxima (λₘₐₓ). The position of λₘₐₓ was plotted as a function of pH, and normalized, such as, λₘₐₓ^{initial}—the position of spectral maxima in the State II, was set to 1 and λₘₐₓ^{final}—the position of spectral maxima in the State III, was set to 0. The normalized pH-dependence was fit (using OriginLab software) with the Henderson-Hasselbach equation to determine the cooperativity (n) and transition mid-point (pK) of transition of the peptide population from State II to State III:

$$Normalized pH dependence = \frac{1}{1 + {10}^{n ( p H - pK )}} \qquad ( 1 )$$

Steady-state circular dichroism and oriented CD measurements: Steady-state CD was measured using an MOS-450 spectrometer (Bio-Logic Science Instruments) in the range of 190 to 260 nm with a step size of 1 nm, and with temperature control set to 25.0° C. Samples were prepared 24 hours prior to experiments to allow for State II equilibration. A buffer-only sample was used as baseline for State I, and a buffer-with-POPC-only sample was used as baseline for States II and III.

OCD was measured using supported planar POPC bilayers prepared using a Langmuir-Blodgett system (KSV Nima). Fourteen quartz slides with 0.2 mm spacers were used; after sonicating the slides in 5% cuvette cleaner (Contrad 70; Decon Labs) in deionized water (≥18.2 MΩ cm at 25° C.; Milli-Q Type 1 Ultrapure Water System, EMD Millipore) for fifteen minutes and rinsing with deionized water, the slides were immersed and sonicated for ten minutes in 2-propanol, sonicated again for ten minutes in acetone, sonicated a final time in 2-propanol for ten minutes, and rinsed thoroughly with deionized water. Lastly, the slides were immersed in a 3:1 solution of sulfuric acid to hydrogen peroxide for five minutes and rinsed three times in deionized water. The slides were stored in deionized water until they were used. POPC bilayers were deposited on the fourteen slides using the Langmuir-Blodgett minitrough: a 2.5 mg/mL solution of POPC in chloroform was spread on the subphase (deionized water) and the chloroform was allowed to evaporate for fifteen minutes, after which the POPC monolayer was compressed to 32 mN/m. A lipid monolayer was deposited on the slides by retrieving them from the subphase, after which a solution of 10 μM peptide and 500 μM of 50 nm POPC liposomes at pH 4 was added to the slides, resulting in the creation of the supported bilayer by fusion between the monolayer on the slides and the peptide-laden lipid vesicles. After incubation for six hours at 100% humidity, the slides were rinsed with buffer solution to remove excess liposomes, and the spaces between the cuvettes were filled with buffer at pH 4. Measurements were taken at three points during the experiment: immediately after the addition of the peptide/lipid solution (0 h), after the slides were rinsed to remove excess liposomes following the six-hour incubation time (6 h), and after an additional twelve-hour incubation time and rinse with buffer (18 h); these measurements were recorded on the MOS-450 spectrometer with sampling times of two seconds at each wavelength. Control measurements were conducted using a peptide solution between slides without supported bilayers and in the presence of POPC liposomes.

Kinetics measurements: Stopped-flow fluorescence measurements were made using an SFM-300 mixing system (Bio-Logic Science Instruments) in conjunction with the MOS-450 spectrometer. All solutions were degassed for fifteen minutes prior to loading into the stopped-flow system. pHLIP variants were incubated with POPC for 24 hours prior to the experiment to reach State II equilibrium, and insertion was induced by mixing equal volumes of pHLIP/POPC solutions with hydrochloric acid diluted to ensure a pH drop from pH 8 to pH 4. Kinetics data were fit by one-, two-, three-, or four-states exponential models in OriginLab.

Amanitin pHLIP conjugates: Alpha-amanitin (Sigma-Aldrich) was conjugated to succinimidyl 3-(2-piridyldithio)propionate) (SPDP; Thermo Fisher Scientific), followed by purification and conjugation of the SPDP-amanitin to the C-terminal cysteine residues of pHLIP peptides. For synthesis of PEG-2WT-amanitin and PEG-4WT-amanitin, Lys-WT-Cys with N-terminal lysine and C-terminal cysteine residues was used, and the Lys-WT-SPDP-amanitin was conjugated to dibenzocyclooctyne-sulfo-N-hydroxysuccinimidyl ester (DBCO-NHS ester; Sigma-Aldrich), resulting in DBCO-WT-SPDP-amanitin. Finally, 2-arm or 4-arm PEG-azide (Creative PEGWorks) was conjugated to DBCO-WT-SPDP-amanitin, resulting in PEG-DBCO-WT-SPDP-amanitin, with a cleavable disulfide bond present in SPDP, between the peptide and amanitin cargo. Construct concentration was calculated by absorbance at 310 nm, where, for α-amanitin, ε₃₁₀=13,000 M⁻¹ cm⁻¹. Construct concentration was presented in terms of peptide/amanitin concentration. Purification was conducted using reverse phase HPLC. Zorbax SB-C18 columns (9.4×250 mm, 5 μm; Agilent Technologies) were used for all peptide-amanitin conjugates other than ATRAM-amanitin, PEG-2WT-amanitin, and PEG-4WT-amanitin, for which Zorbax SB-C8 columns (9.4×250 mm, 5 μm; Agilent Technologies) were used.

Cell proliferation assay: Human cervix adenocarcinoma cells (HeLa; American Type Culture Collection) were authenticated, stored according to the supplier's instructions, and used within three months of frozen aliquot resuscitation. Cells were cultured in Dulbecco's modified Eagle's medium (DMEM; Sigma-Aldrich) at pH 7.4 with 4.5 g/L D-glucose, supplemented with 10% heat-inactivated fetal bovine serum (FBS; Sigma-Aldrich) and 10 μg/mL ciprofloxacin (Sigma-Aldrich), in a humidified atmosphere of 5% CO2 and 95% air at 37° C. The pH 6.0 medium was prepared by mixing 13.3 g of dry DMEM in 1 L of deionized water. HeLa cells were loaded in the wells of 96-well plates (5,000 cells/well) and incubated overnight. The standard growth medium was replaced with medium without FBS, at pH 6.0 or 7.4, containing increasing amounts of pHLIP-amanitin conjugates (from 0 up to 2.0 μM). Treatment with amanitin alone for two hours and at concentrations up to 2 μM does not induce cell death (Moshnikova et al. (2013) Biochemistry-US 52(7):1171-1178). After two-hour incubation with the pHLIP-amanitin conjugates, the constructs were removed and replaced with standard growth medium. Cell viability was assessed after 48 hours using the CellTiter 96 AQᵤₑₒᵤₛ One Solution Cell Proliferation Assay (Promega); the colorimetric reagent was added to cells for one hour, followed by absorption measurement at 490 nm. All samples were prepared in triplicate, and each experiment was repeated from 3 to 6 times for different constructs. All obtained cell proliferation data were normalized by corresponding controls (non-treated cells). There was no difference in viability of cells incubated with media (no constructs) at pH7.4 and pH6.0, therefore role of pH was excluded from the consideration. Normalized cell viability data obtained in different experiments were averaged, and presented as the logarithm of dose of pHLIP-amanitin constructs. The dose response function was used for fitting (using OriginLab software) of the obtained data (FIG. 6):

$$Cell viability = {A}_{b} + \frac{{A}_{t} - {A}_{b}}{1 + {10}^{p ( LOG x 0 - x )}} \qquad ( 2 )$$

where A_{b} and Aₜ are the bottom and the top asymptotes, respectively. The top asymptote was set as constant, 100%, while for bottom asymptote we allowed small variations in the range of 0 to 10%. p is the slope (cooperativity parameter) and LOG x0 is the center of the transition, the concentration for half response, which is used to calculate the EC₂₀, EC₅₀, EC₅₀ values:

EC₂₀=10^{(LOG x0+Log(0.25)/p)} (3)

EC₅₀=10^{LOG x0} (4)

EC₈₀=10^{(LOG x0+Log(4)/p)} (5)

Therapeutic index (TI) was calculated according to the equation:

$$TI = \frac{{EC}_{50}^{p H 7.4}}{{EC}_{50}^{p H 6.}} \qquad ( 6 )$$

Additionally, the cytotoxicity of the PEG-2WT and PEG-4WT constructs without amanitin was tested: these experiments demonstrated no cytotoxicity at physiological or low pH at treatment concentrations up to 10 μM.

Fluorescent pHLIP conjugates: Alexa Fluor 546 (AF546) C₅ maleimide (Thermo Fisher Scientific) was conjugated to N-terminal cysteine residues of WT, Var3, Var3/Gla, and ATRAM. Alexa Fluor 546 NHS Ester (Thermo Fisher Scientific) was conjugated to the N-terminal lysine residues of WT/Gla, WT/Gla/Aad, and Var3/GLL. For PEG-2WT and PEG-4WT, Cys-WT-Lys, with N-terminal cysteine and C-terminal lysine residues, was used, and was first conjugated to 2-arm maleimide-PEG-maleimide or 4-arm PEG-maleimide resulting in PEG-WT-Lys. Then, Alexa Fluor 546 NHS Ester was conjugated to the C-terminal lysine residue, resulting in 2-arm and 4-arm PEG-pHLIP constructs with C-terminal AF546 fluorophores. Construct concentration was calculated by absorbance at 554 nm, where, for AF546, ε₅₅₄=93,000 M⁻¹ cm⁻¹. Construct concentration was presented in terms of AF546/peptide concentration, not molecular concentration. Purification was conducted using RP-HPLC for all peptides other than PEG-4WT-AF546, which was purified via Amicon Ultra MWCO 10 kDa centrifugal filter (Sigma-Aldrich). Zorbax SB-C18 columns (9.4×250 mm, 5 μm; Agilent Technologies) were used for all AF546-peptide conjugates except AF546-ATRAM and PEG-2WT-AF546, for which Zorbax SB-C8 columns (9.4×250 mm, 5 μm; Agilent Technologies) were used.

Ex vivo imaging: All animal studies were conducted according to the animal protocol AN04-12-011 approved by the Institutional Animal Care and Use Committee at the University of Rhode Island, in compliance with the principles and procedures outlined by the National Institutes of Health for the care and use of animals. Mouse mammary cells (4T1; American Type Culture Collection) were subcutaneously implanted in the right flank (8×10⁵ cells/0.1 mL/flank) of adult female BALB/cAnNHsd mice (Envigo). When tumors reached approximately 5-6 mm in diameter, single tail vein injections of 100 μL, 40 μM fluorophore-pHLIP solutions in PBS were performed. Mice were euthanized 4 hours (or 24 hours) after injection, and necropsy was immediately performed. Tumors and major organs were cut in half and imaged using FX Kodak in-vivo image station connected to the Andor CCD. Mean surface fluorescence intensity of tumor, tissue and organs was obtained via analysis of fluorescent images in ImageJ (NIH) (Schneider et al. (2012) Nat Methods 9(7):671-675). The corresponding autofluorescence signal was subtracted to obtained net fluorescence intensities used in the study. Autofluorescence was calculated after imaging of tumor, tissue and organs collected from mice with no injection of fluorescent pHLIP constructs.

Tables

TABLE 8
The parameters, midpoint (pK), cooperativity (n) and time (t), characterizing
the pH-dependent transition of pHLIP variants in the presence of POPC
liposomes, are presented. EC₂₀, EC₅₀, EC₈₀ values were calculated for each
pHLIP-amanitin construct at different pHs by analyzing pH- and
concentration-dependent cell viability data (FIG. 6).

| | | | | EC₂₀, μM | | EC₅₀, μM | | EC₈₀, μM | |
|---|---|---|---|---|---|---|---|---|---|
| Peptide | pK | n | t (s) | pH 7.4 | pH 6.0 | pH 7.4 | pH 6.0 | pH 7.4 | pH 6.0 |
| WT | 6.5 | 1.8 ± 0.1 | 36.8 | 1.95 | 1.22 | 1.37 | 0.56 | 0.96 | 0.26 |
| WT/Gla | 6.2 | 1.5 ± 0.0 | 37.5 | 6.20 | 0.93 | 2.73 | 0.30 | 1.20 | 0.10 |
| WT/Gla/Aad | 6.6 | 1.4 ± 0.1 | 34.8 | 3.01 | 0.66 | 1.39 | 0.37 | 0.64 | 0.21 |
| PEG-2WT | 6.6 | 1.8 ± 0.2 | 18.8 | 1.98 | 0.54 | 1.03 | 0.19 | 0.53 | 0.07 |
| PEG-4WT | 6.6 | 2.2 ± 0.4 | 13.1 | 0.473 | 0.19 | 0.33 | 0.11 | 0.23 | 0.06 |
| Var3 | 5.7 | 0.9 ± 0.0 | 0.9 | 10.63 | 1.30 | 3.95 | 0.43 | 1.47 | 0.14 |
| Var3/Gla | 6.3 | 0.7 ± 0.0 | 0.7 | 5.12 | 1.34 | 2.76 | 0.50 | 1.48 | 0.19 |
| Var3/GLL | 6.6 | 0.4 ± 0.0 | 0.1 | 1.75 | 0.47 | 0.91 | 0.23 | 0.47 | 0.11 |
| ATRAM | 6.4 | 0.9 ± 0.1 | 0.1 | 2.06 | 0.40 | 1.23 | 0.22 | 0.74 | 0.12 |

TABLE 9
List of pHLIP sequences used in the study.

| Peptide | Sequence |
|---|---|
| Cys-WT | ACEQNPIYWARYADWLFTTPLLLLDLALLVDADEGT (SEQ ID NO: 313) |
| WT-Cys | AEQNPIYWARYADWLFTTPLLLLDLALLVDADEGCT (SEQ ID NO: 314) |
| Lys-WT-Cys | Ac-AKEQNPIYWARYADWLFTTPLLLLDLALLVDADECT (SEQ ID NO: 315) |
| Lys-WT/Gla-Cys | Ac-AKEQNPIYWARYAGlaWLFTTPLLLLDLALLVDADECT (SEQ ID NO: 316) |
| Lys-WT/Gla/Aad-Cys | Ac-AKEQNPIYWARYAGlaWLFTTPLLLLAadLALLVDADECT (SEQ ID NO: 317) |
| Cys-Var3 | ACDDQNPWRAYLDLLFPTDTLLLDLLWA (SEQ ID NO: 15) |
| Var3-Cys | ADDQNPWRAYLDLLFPTDTLLLDLLWCA (SEQ ID NO: 17) |
| Cys-Var3/Gla | ACDDQNPWRAYLGlaLLFPTDTLLLDLLWG (SEQ ID NO: 318) |
| Var3/Gla-Cys | ADDQNPWRAYLGlaLLFPTDTLLLDLLWCG (SEQ ID NO: 327) |
| Lys-Var3/GLL-Cys | Ac-GKEEQNPWLGAYLDLLFPLELLGLLELGLWCG (SEQ ID NO: 319) |
| Cys-ATRAM | ACGLAGLAGLLGLEGLLGLPLGLLEGLWLGLELEGN (SEQ ID NO: 320) |
| ATRAM-Cys | GLAGLAGLLGLEGLLGLPLGLLEGLWLGLELEGNCA (SEQ ID NO: 324) |

TABLE 10
Molecular weights (MW), retention time and percentage of
acetonitrile presented for 30 min method of 25% to 80%
gradient of acetonitrile/0.05% TFA in water/0.05% TFA used
in C18 and C8 columns for the elution of peptides.

| | | Zorbax SB-C18 | | Zorbax SB-C8 | |
|---|---|---|---|---|---|
| | | (4.6 × 250 mm, 5 μm) | | (4.6 × 250 mm, 5 μm) | |
| | | Reten- | | Reten- | |
| | | tion | | tion | |
| | | Time | % Aceto- | Time | % Aceto- |
| Peptide | MW (Da) | (min) | nitrile | (min) | nitrile |
| Cys-WT | 4111.7 | 21.6 | 64.6% | 20.6 | 62.8% |
| Lys-WT- | 4224.9 | 21.6 | 64.6% | 20.4 | 62.4% |
| Cys | | | | | |
| Lys-WT/ | 4283.1 | 22.3 | 65.9% | 21.2 | 63.9% |
| Gla-Cys | | | | | |
| Lys-WT/ | 4310.9 | 22.8 | 66.8% | 21.7 | 64.8% |
| Gla/Aad- | | | | | |
| Cys | | | | | |
| Cys-Var3 | 3292.8 | 19.7 | 61.1% | 19.6 | 60.9% |
| Cys-Var3/ | 3333.8 | 19.9 | 61.5% | 19.7 | 61.1% |
| Gla | | | | | |
| Lys-Var3/ | 3643.2 | 28.0 | 76.2% | 26.0 | 72.7% |
| GLL-Cys | | | | | |
| Cys- | 3516.2 | 30.6* | 93.0% | 27.2 | 74.9% |
| ATRAM | | | | | |

*Peptide eluted during washing with acetonitrile, after the completion of the gradient.

TABLE 11
Positions of maxima of tryptophan fluorescence spectra
(λₘₐₓ) and ratios of ellipticity at 205 nm to 222
nm of pHLIP constructs in States I, II, and III.

| | λₘₐₓ (nm) | | | Ellipticity at 205/222 nm | | |
|---|---|---|---|---|---|---|
| Construct | State I | State II | State III | State I | State II | State III |
| WT | 351.6 | 347.4 | 340.0 | 1.61 | 2.16 | 0.68 |
| WT/Gla | 348.2 | 347.1 | 338.2 | 2.02 | 1.60 | 0.70 |
| WT/Gla/Aad | 347.5 | 346.7 | 338.3 | 1.60 | 1.31 | 0.68 |
| PEG-2WT | 341.8 | 341.2 | 336.6 | 1.12 | 1.48 | 0.71 |
| PEG-4WT | 344.0 | 343.4 | 338.9 | 1.18 | 1.07 | 0.71 |
| Var3 | 350.2 | 346.8 | 339.9 | 2.35 | 1.55 | 0.71 |
| Var3/Gla | 351.4 | 345.7 | 339.2 | 2.72 | 1.76 | 0.85 |
| Var3/GLL | 349.3 | 343.3 | 341.2 | 2.21 | 1.32 | 0.84 |
| ATRAM | 345.5 | 341.4 | 333.0 | 2.45 | 1.44 | 0.85 |

TABLE 12
Fluorescence intensity obtained by ex vivo imaging of tumor, tissue, and organs 4 hours after
a single IV administration of the Alexa Fluor 546-pHLIP constructs (also data obtained at 24
hours post-injection are shown for PEG-2WT and PEG-4WT). Values of tissue autofluorescence are
provided in the last row. Sample size (n) is given in the last column for each construct.

| | Total Fluorescence Intensity in tumor, muscle and organs (a.u.) | | | | | | |
|---|---|---|---|---|---|---|---|
| Construct | Tumor | Muscle | Kidney | Liver | Lungs | Spleen | n |
| WT | 2335.7 ± 447.1 | 726.8 ± 58.5 | 988.5 ± 91.0 | 998.7 ± 142.0 | 545.8 ± 32.6 | 447.0 ± 31.7 | 5 |
| WT/Gla | 1372.1 ± 331.7 | 591.2 ± 73.2 | 880.2 ± 126.5 | 1102.1 ± 239.6 | 456.2 ± 68.6 | 387.9 ± 63.3 | 13 |
| WT/Gla/Aad | 1336.6 ± 304.5 | 559.6 ± 53.5 | 726.6 ± 149.9 | 1156.4 ± 120.8 | 448.5 ± 79.9 | 374.1 ± 45.0 | 13 |
| PEG-2WT | 1443.4 ± 178.5 | 545.3 ± 42.2 | 602.7 ± 79.2 | 1267.1 ± 146.2 | 462.0 ± 28.5 | 450.7 ± 17.3 | 5 |
| PEG-4WT | 912.8 ± 159.5 | 495.5 ± 23.0 | 523.4 ± 52.5 | 1209.7 ± 100.7 | 431.0 ± 54.9 | 403.7 ± 30.0 | 7 |
| Var3 | 3474.3 ± 924.2 | 760.7 ± 68.3 | 1263.9 ± 136.5 | 904.8 ± 93.4 | 564.3 ± 130.1 | 335.6 ± 45.0 | 7 |
| Var3/Gla | 2948.9 ± 540.6 | 889.3 ± 144.7 | 1390.0 ± 247.5 | 702.9 ± 140.9 | 784.5 ± 248.7 | 379.2 ± 47.8 | 5 |
| Var3/GLL | 1577.9 ± 364.2 | 694.2 ± 27.5 | 1690.8 ± 431.8 | 1194.3 ± 147.1 | 520.2 ± 18.6 | 442.8 ± 37.4 | 5 |
| ATRAM | 3039.5 ± 620.9 | 813.3 ± 105.4 | 1009.8 ± 183.9 | 1073.2 ± 146.3 | 716.6 ± 109.0 | 421.3 ± 35.3 | 9 |
| PEG-2WT (24 h) | 894.9 ± 151.8 | 486.3 ± 19.8 | 525.1 ± 37.6 | 753.6 ± 54.0 | 547.2 ± 64.8 | 346.3 ± 11.0 | 5 |
| PEG-4WT (24 h) | 663.5 ± 121.1 | 440.1 ± 7.7 | 464.2 ± 35.1 | 726.7 ± 69.6 | 482.8 ± 48.5 | 331.8 ± 4.4 | 5 |
| Autofluorescence | 426.9 ± 15.7 | 402.7 ± 11.5 | 325.6 ± 30.8 | 354.7 ± 28.3 | 392.7 ± 32.7 | 296.1 ± 23.2 | 12 |

TABLE 13
Tumor-to-muscle (T/M), tumor-to-kidney (T/K) and tumor-to liver
(T/L) ratios presented on FIGS. 4B, 4C, and 4D, respectively.

| Constructs | T/M | T/K | T/L |
|---|---|---|---|
| WT | 5.87 ± 0.64 | 2.95 ± 0.95 | 3.03 ± 0.76 |
| WT/Gla | 5.39 ± 1.65 | 1.77 ± 0.68 | 1.40 ± 0.76 |
| WT/Gla/Aad | 6.54 ± 3.15 | 2.59 ± 1.33 | 1.14 ± 0.37 |
| PEG-2WT | 7.48 ± 1.86 | 3.80 ± 0.77 | 1.13 ± 0.19 |
| PEG-4WT | 5.34 ± 1.53 | 2.55 ± 0.78 | 0.57 ± 0.17 |
| Var3 | 8.86 ± 3.61 | 3.38 ± 1.34 | 5.62 ± 1.90 |
| Var3/Gla | 5.30 ± 0.89 | 2.46 ± 0.64 | 8.29 ± 4.19 |
| Var3/GLL | 4.03 ± 1.54 | 0.87 ± 0.23 | 1.42 ± 0.55 |
| ATRAM | 6.56 ± 1.55 | 3.96 ± 1.05 | 3.72 ± 1.01 |

TABLE 14
The membrane-inserted populations of different pHLIP variants were
calculated using the pH dependence parameters pK and n (Table 8).

| pH | WT | WT/Gla | WT/Gla/Aad | PEG-2WT | PEG-4WT | Var3 | Var3/Gla | Var3/GLL | ATRAM |
|---|---|---|---|---|---|---|---|---|---|
| 7.4 | 2% | 2% | 7% | 4% | 2% | 3% | 15% | 32% | 11% |
| 7.2 | 5% | 3% | 13% | 8% | 5% | 4% | 19% | 37% | 16% |
| 7.0 | 11% | 6% | 22% | 16% | 12% | 6% | 24% | 41% | 22% |
| 6.8 | 22% | 11% | 34% | 30% | 27% | 9% | 31% | 45% | 30% |
| 6.5 | 50% | 26% | 58% | 60% | 62% | 16% | 42% | 52% | 45% |
| 6.2 | 78% | 50% | 78% | 84% | 88% | 26% | 54% | 59% | 60% |
| 6.0 | 89% | 67% | 87% | 92% | 95% | 35% | 62% | 63% | 70% |

### Example 2: Hemolysis Assay Performed with WT, Var3 and Var7 pHLIP Peptide

Hemolysis assays show ability of a construct to lyse red blood cells (RBCs). Peptide or compounds with multiple positive charges are typically very lytic. This study shows that pHLIPs (which have negative charges) are not lytic for RBC.

Single donor human whole blood was purchased from Innovative Research. RBCs were collected by centrifugation of whole blood at 2000 rpm for 10 minutes followed by washing three times with Dulbecco's PBS (DPBS) and re-suspended in DPBS at a concentration of 7.5% (v:v). Varying concentrations of WT, Var3 and Var7 peptides (2.5 μM, 5 μM and 10 μM) in 10 mM HEPES buffer, pH 7.4 containing 137 mM NaCl, 2.7 mM KCl, 1 mM CaCl₂) were added to RBCs to form 5% RBC suspension. The resultant mixtures were incubated at 37° C. for 2 hours and then centrifuged at 2000 rpm for 10 min. The hemolysis was assessed by the release of hemoglobin, which was monitored by measuring of absorbance at 450 nm. 10 mM HEPES buffer, pH 7.4 containing 137 mM NaCl, 2.7 mM KCl, 1 mM CaCl₂) and DPBS were used as negative controls. As positive controls, which result in 100% lysis of RBCs, we used i) water and ii) 10% of Triton X-100. The percentage of hemolysis was calculated as follows:

$$\% Hemolysis = 100 \cdot \frac{{OD}_{Test} - {OD}_{NC}}{{OD}_{PC} - {OD}_{NC}}$$

where, OD_{Test}, OD_{NC}, and OD_{PC} are the optical density reading (absorbance) values of the test sample, negative control and positive control, respectively. The assay was performed in triplicate. The lysis of RBCs was less than 1% in the case of WT, Var3 and Var7 pHLIP peptides.

### Example 3: Use of pHLIP Compound Comprising of pHLIP Peptide (Var3 Group), Linker (SPDP Crosslinker), and Cargo (Amanitin)

Methods

pHLIP peptide (Var3: ADDQNPWRAYLDLLFPTDTLLLDLLWCA (SEQ ID NO: 17)) was purchased from CS Bio Co. Peptide concentration was calculated by absorbance at 280 nm, ε₂₈₀=12,660 M⁻¹ cm⁻¹. Alpha-amanitin (Sigma-Aldrich) was conjugated to succinimidyl 3-(2-pyridyldithio)propionate) (SPDP; Thermo Fisher Scientific), followed by purification and conjugation of the SPDP-amanitin to the C-terminal cysteine residues of Var3 pHLIP peptides. Purification was conducted using reverse phase HPLC (Zorbax SB-C18 columns 9.4×250 mm, 5 μm; Agilent Technologies). Construct concentration was calculated by absorbance at 310 nm, where, for α-amanitin, ε₃₁₀=13,000 M⁻¹ cm⁻¹.

Ten different bladder cancer cell lines from ATCC (American Type Culture Collection) were authenticated, stored according to the supplier's instructions, and used within three months of frozen aliquot resuscitation. Cells were loaded in the wells of 96-well plates (5,000 cells/well) and incubated overnight. The standard growth medium was replaced with medium without FBS, at pH 6.0 or 7.4, containing increasing amounts of pHLIP-SPDP-amanitin composition. Treatment with amanitin alone for two hours and at concentrations up to 2 μM does not induce cell death as it was shown previously. After two-hour incubation with the pHLIP-SPDP-amanitin composition, the construct was removed and replaced with standard growth medium. Cell viability was assessed after 72 hours using the CellTiter 96 AQᵤₑₒᵤₛ One Solution Cell Proliferation Assay (Promega); the colorimetric reagent was added to cells for one hour, followed by absorption measurement at 490 nm. All samples were prepared in triplicate, and each experiment was repeated from 3-4 times for different cell lines. All obtained cell proliferation data were normalized by corresponding controls (non-treated cells at pH7.4). Normalized cell viability data obtained in different experiments were averaged, and presented as the logarithm of dose of pHLIP-SPDP-amanitin composition. The dose response function was used for global fitting (using OriginLab software) of the obtained data at both pH7.4 and pH6.0:

$$Cell viability = {A}_{b} + \frac{{A}_{t} - {A}_{b}}{1 + {10}^{p ( LOG x 0 - x )}}$$

where A_{b} and Aₜ are the bottom and the top asymptotes, respectively. The top asymptote was set as constant, 100%, while for bottom asymptote we allowed small variations in the range of 0 to 10%. p is the slope (cooperativity parameter) and LOG x0 is the center of the transition, the concentration for half response, which is used to calculate the EC₂₀, EC₅₀, EC₅₀ values:

EC₂₀=10^{(LOG x0+Log(0.25)/p)}

EC₅₀=10^{LOG x0}

EC₈₀=10^{(LOG x0+Log(4)/p)}

Therapeutic index (TI) was calculated according to the equation:

$$TI = \frac{{EC}_{50}^{p H 7.4}}{{EC}_{50}^{p H 6.}}$$

Use of pHLIP Compound Comprising of pHLIP Peptide (Var3 Group), Linker (SPDP Crosslinker), and Cargo (Amanitin)

Bladder cancer is the fifth most common cancer, comprising 5% of all new cancer cases in the United States, with 79,030 new cases of bladder cancer (about 60,490 in men and 18,540 in women) and about 16,870 deaths from bladder cancer (about 12,240 in men and 4,630 in women) estimated for 2017 in the US and over 450,000 cases worldwide. Almost all of these patients require continuous surveillance and treatments. The first treatment option of bladder cancer is a surgery, transurethral resection of bladder tumors (TURBT)—for the removal of cancerous lesions. TURBT is accompanied with perioperative or postoperative intravesical therapy. Immunotherapy includes the use of Bacillus Calmette-Guérin (BCG), a vaccine for prevention of tuberculosis, and interferons. Typically, immunotherapy might provide a good first outcome, but does not lead to cure and became ineffective at next steps, when chemotherapy is employed. Chemotherapy includes use of mitomycin, thiotepa, gemcitabine, doxorubicin and its derivatives. However, these drugs do not possess ability of targeting of cancer cells. Thus, high concentration of the drug is used for bladder instillation, which leads to the toxicity, since small drug molecules (<500 Da) are readily adsorbed by the bladder and reach the blood stream to induce systemic toxicity. At the same time the efficacy of the treatment is very moderate and the recurrence rate is very significant due to the lack of the ability to target and kill all cancer cells in the bladder.

A tumor targeting pHLIP compound comprising a pHLIP peptide, SPDP linker and amanitin cargo is proposed. Amanitin is a polar, cell-impermeable molecule, which cannot cross the plasma membrane of cells. A toxic effect after IV administration of amanitin or consumption of amanitin with food is associated with liver poisoning, since the liver has a special transporting system to take up cyclic compounds, like amanitin. Significant liver toxicity is not expected in the result of intravesical instillation. The tested pHLIP compound has been tested on the following 10 human bladder cancer cell lines:
5637, grade II carcinoma
J82, transitional cell carcinoma
UMUC3, transitional cell carcinoma
SCaBER, squamous cell carcinoma
T24, transitional cell carcinoma
TCCSUR, IV transitional cell carcinoma
RT4, transitional cell papilloma
HT-1197, urinary bladder carcinoma
HT-1376, grade III carcinoma
SW780, transitional cell carcinoma

FIGS. 7A-C represent normalized cell viability data vs. the logarithm of concentration of pHLIP-SPDP-amanitin composition (Var3-SPDP-Am) fitted by the dose response function (curves) to calculate the EC₂₀, EC₅₀, EC₈₀ values, which are presented in Table 15. FIG. 8 demonstrates therapeutic index (TI). The toxic effect was higher at low pH compared to normal pH in the case of all bladder cancer cell lines. The therapeutic index varied in the range from 3.6 to 11.3 with mean at 6.7±2.6. The pHLIP-SPDP-Amanitin composition could be used for the treatment of bladder cancer by intravesical instillation.

TABLE 15
The EC₂₀, EC₅₀, EC₈₀ values were calculated for each pHLIP-SPDP-amanitin composition at different
pHs by analyzing pH- and concentration-dependent cell viability data shown in FIG. 7.

| | EC₂₀, μM | | EC₅₀, μM | | EC₈₀, μM | | |
|---|---|---|---|---|---|---|---|
| Bladder cancer cell line | pH 7.4 | pH 6.0 | pH 7.4 | pH 6.0 | pH 7.4 | pH 6.0 | TI |
| HT-1197, urinary bladder carcinoma | 240.4 | 78.5 | 25.7 | 876.6 | 284.7 | 92.5 | 3.6 |
| 5637, grade II carcinoma | 148.7 | 45.1 | 13.7 | 858.3 | 408.6 | 194.5 | 9.1 |
| HT-1376, grade III carcinoma | 3625.7 | 429.8 | 51.0 | 7067.1 | 2128.8 | 641.2 | 5.0 |
| SCaBER, squamous cell carcinoma | 157.9 | 67.7 | 29.0 | 1753.8 | 768.2 | 336.5 | 11.3 |
| J82, transitional cell carcinoma | 249.3 | 79.3 | 25.2 | 877.7 | 339.5 | 131.3 | 4.3 |
| UM-UC-3, transitional cell carcinoma | 631.7 | 79.5 | 10.0 | 2078.8 | 390.0 | 73.2 | 4.9 |
| SW780, transitional cell carcinoma | 693.1 | 84.0 | 10.2 | 2191.7 | 405.5 | 75.0 | 4.8 |
| T-24, transitional cell carcinoma | 255.7 | 77.1 | 23.2 | 1204.0 | 527.4 | 231.0 | 6.8 |
| TCCSUR, IV transitional cell carcinoma | 1060.3 | 293.9 | 81.4 | 5058.3 | 2027.3 | 812.5 | 6.9 |
| RT4, transitional cell papilloma | 1119.3 | 92.2 | 7.6 | 2643.4 | 900.2 | 306.6 | 9.8 |

### Example 4: Tumors Targeting, Biodistribution and Kinetics Studies with an Exemplary ICG-pHLIP

An ICG-Var3 pHLIP imaging agent that has been chosen for further study and evaluation (ICG-pHLIP) is shown in FIG. 11, where ICG is indocyanine green fluorescent dye and Var3 is a tumor targeting pHLIP variant with the following sequence: Ala-Cys-Asp-Asp-Gln-Asn-Pro-Trp-Arg-Ala-Tyr-Leu-Asp-Leu-Leu-Phe-Pro-Thr-Asp-Thr-Leu-Leu-Leu-Asp-Leu-Leu-Trp-Ala (SEQ ID NO: 15)

Conjugation of ICG with pHLIP Var3

pHLIP Var3 (synthesized and purified by CS Bio) and ICG-maleimide (Intrace Medical) was dissolved in DMSO. Peptide and ICG-maleimide concentrations were calculated by measuring absorbance in methanol at 280 nm and 800 nm, respectively, and using extinction coefficients ε₂₈₀=12,660 M⁻¹cm⁻¹ for peptide and ε₈₀₀=137,000 M⁻¹cm⁻¹ for ICG. ICG-maleimide was conjugated with the peptide at a 1:1 molar ratio. Reaction went in DMSO in presence of 100 mM sodium phosphate, 150 mM NaCl buffer, pH 7.4 (saturated with argon) at 9:1 v/v ratio. The reaction mixture was incubated at room temperature for 2-3 hours and the progress of the reaction was monitored by analytical reverse phase HPLC using a Zorbax SB-C18 column (4.6×250 mm, 5 μm; Agilent Technologies) and a 20-80% binary solvent gradient system of water and acetonitrile with 0.05% TFA over 30 min. If needed, additional amounts of ICG-maleimide were added to the reaction mix to react with the peptide. ICG pHLIP® was purified by reverse phase HPLC using 9.4×250 Zorbax SB-C18 columns. Purity of the product was accessed by SELDI-TOF mass spectrometry and analytical HPLC using a Zorbax SB-C18 column (4.6×250 mm, 5 μm) with a binary solvent system using a 15-85% water and acetonitrile gradient with 0.05% TFA over 25 min (FIGS. 12A-B). The purity of the construct was more than 95%.

A cGMP manufacturing protocol of ICG-pHLIP® is developed by. The GLP material is produced for toxicity study (see Certificate of Analysis in FIG. 29) according to the developed protocol, which is used for the production of cGMP material from clinical trials.

### Stability Study

Stability studies were performed with i) ICG-pHLIP formulation in PBS containing 5% DMSO used in some of the proof of concept (PoC) animal studies and ii) ICG-pHLIP in PBS containing 5% Ethanol formulation used in some PoC animal studies, toxicity studies on mice, rats and dogs and formulation developed for human dosing (in PBS containing 5% Ethanol).

For the PBS/5% DMSO formulation, 1 mg of the lyophilized powder of ICG-pHLIP (96.8% purity), synthesized according to the protocol described above, was dissolved in 75 LEI of DMSO (to make 3.2 mM solution), next 10 μl of 3.2 mM stock was mixed with 190 μl PBS to make 0.16 mM solution of ICG pHLIP® (5% DMSO).

For the PBS/5% Ethanol formulation, 16 mg of the lyophilized powder of ICG-pHLIP (98.7% purity, GLP material manufactured by Iris Biotech) was dissolved by 30 sec vortexing in PBS containing 5% Ethanol (formulation proposed for human dosing).

Both formulations were kept at room temperature protected from light. The aliquots were taken at 0.5 or 1 b, 3 h, 6 h, 24 h, 48 h and 72 hours for analytical HPLC analysis using a Zorbax SB-C18 column (4.6×250 mm, 5 μm) with a binary solvent system using a 15-85% water and acetonitrile gradient with 0.05% TFA over 25 min. Results of HPLC analysis are provided in Appendix N2 and N3). The stability was constant up to 72 hours (we did not looked longer time points) at room temperature and both constructs preserved their original purity (FIGS. 13A-B).

### Absorption and Fluorescence of ICG-pHLIP

The concentration of the ICG-pHLIP was determined by ICG absorption at 800 nm, ε₈₀₀=137,000 M⁻¹ cm⁻¹ in DMSO or Methanol. The absorption and fluorescence spectra of ICG-pHLIP in DMSO and emission of ICG-pHLIP in PBS in presence of POPC liposomes, which mimic cellular membrane, are shown in FIG. 14.

### Cytotoxicity

Human mammary epithelial cells (HMEpC) were acquired from Cell Applications Inc, and authenticated, and stored according to supplier's instructions. Cells were cultured in mammary epithelial cell growth medium provided by Cell Applications Inc. HMEpC cells were loaded in the wells of 96-well plates (˜6,000 cells per well) and incubated overnight. The increasing amounts of ICG-pHLIP dissolved in cell growth medium were added to cells to have the following final concentration of ICG-pHLIP with cells: 0.125, 0.25, 0.5, 1, 2, 4, 8 and 16 μM. After 48 and 72 hours of incubation, a colorimetric reagent (CellTiter 96 AQᵤₑₒᵤₛ One Solution Assay, Promega) was added for 1 hour followed by measuring absorbance at 490 nm to assess cell viability. All samples were prepared in triplicate and each experiment was repeated several times. ICG-pHLIP did not show any cytotoxic effect at any tested concentration.

### Hemolysis Assay

Single donor human whole blood was purchased from Innovative Research. Red blood cells (RBCs) were collected by centrifugation of whole blood at 2000 rpm for 10 minutes followed by washing three times with Dulbecco's PBS (DPBS) and re-suspended in DPBS at a concentration of 7.5% (v:v). Varying concentrations of ICG-pHLIP (0.075, 0.15, 0.3, 0.6, 1.2 nmol) in DPBS were added to RBCs to give a 5% RBC suspension (total volume of solution with RBC was 150 μL). The resultant mixtures were incubated at 37° C. for 2 hours and then centrifuged at 2000 rpm for 10 min. Hemolysis was assessed by the release of hemoglobin, which was monitored by measuring the absorbance at 450 nm of the supernatant hemoglobin. DPBS was used as negative controls. As positive controls, which result in 100% lysis of RBCs, we used i) water and ii) 10% of Triton X-100. The percentage of hemolysis was calculated as follows:

$$\% Hemolysis = 100 \cdot \frac{{OD}_{Test} - {OD}_{NC}}{{OD}_{PC} - {OD}_{NC}}$$

where, OD_{Test}, OD_{NC}, and OD_{PC} are the optical density reading (absorbance) values of the test sample, negative control and positive control, respectively. The assay was performed in triplicate. The amount of RBC lysis was less than 2% in all samples. For the reference, in mice study 2.5 nmol of ICG pHLIP® is injected per mouse (a 20 g mouse has about 1.2 mL of blood), or 2.08 nmol/ml (the dose will be much lower in humans), while in hemolysis assay the maximum tested concentration was 8 nmol/ml.

### Animal Experiments

All animal studies were conducted according to the animal protocol AN04-12-011 approved by the Institutional Animal Care and Use Committee at the University of Rhode Island, in compliance with the principles and procedures outlined by the National Institutes of Health for the care and use of animals.

### Biodistribution and Kinetics

BALB/cAcNHsd mice ranging in age from 5 to 6 weeks obtained from Envigo RMS Inc were used in the study. Mouse mammary 4T1 cancer cells were subcutaneously implanted in the right flank (8×10⁵ cells/0.1 mL/flank) of adult female mice. Triple negative 4T1 tumor model closely mimics stage IV of human breast cancer. When tumors reached 5-6 mm in diameter, single tail vein injections of 2.5 nmol (or 0.5 mg/kg) of ICG-pHLIP in sterile PBS with 5% DMSO or 5% Ethanol (volume of the injection was 100 l) were performed. The whole body and ex vivo imaging was performed using a Stryker 1588 AIM clinical imaging system with L10 AIM Light Source, 1588 AIM Camera using a 10 mm or 5 mm scope. Whole-body mouse images, magnified images of shaved mouse flank with 4T1 tumor and excised 4T1 tumors demonstrating ICG pHLIP NIRF imaging are shown in FIGS. 15B, 15D, and 15F.

Animals were euthanized at time points: 5 min, 1 hr, 2 hrs, 4 hrs, 6 hrs, 16 hrs, 26 hrs and 48 hrs. Five animals were used for each time point plus seven control animals (mice, who did not receive ICG-pHLIP imaging construct). 100 μl of blood was collected immediately after euthanasia and mixed with 12.5 μl of citrate-dextrose anticoagulant solution (kept at 4° C.), and necropsy was performed. Tumor, muscles, skin, heart, lungs, liver, spleen, kidneys, brain, pancreas, bone, stomach, small and large intestines were collected, imaged immediately after collection, weighed, and fast frozen in liquid nitrogen.

Blood samples mixed with of anti-coagulant solution were placed in 384 well plates (MatTek, glass bottom) (15 μL per well) and imaged on an Odyssey IR scanner (Li-Cor Biosciences). To establish a calibration curve, known amounts of ICG-pHLIP (different concentrations) were added to the blood of control mice (mice, who did not receive ICG-pHLIP imaging construct), mixed with anticoagulant solution. The same amounts (15 μL) of blood samples were placed in 384 well plate and imaged together with all other blood samples. The digital images were processed using the Image J program to calculate mean fluorescence intensity. The calibration curve (known concentration of ICG-pHLIP in blood samples vs intensity) was constructed to calculate the amount of ICG-pHLIP in blood samples collected from the mice at different times after construct administration.

TABLE 16
Concentration of ICG-Var3 (nmol) in blood collected from animals at different time points after
single tail vein injection of 2.5 nmol of ICG-Var3. 5 animals were used for each time point.

| Time p.i. | 5 min | 1 hour | 2 hours | 4 hours | 6 hours | 16 hours | 26 hours |
|---|---|---|---|---|---|---|---|
| Concentration | 2.09 | 1.31 | 0.40 | 0.49 | 0.38 | 0.13 | 0.03 |
| in blood, nmol | 2.47 | 1.73 | 0.67 | 0.57 | 0.32 | 0.10 | 0.02 |
| | 2.02 | 0.93 | 0.47 | 0.48 | 0.35 | 0.16 | 0.02 |
| | 2.24 | 0.88 | 0.76 | 0.57 | 0.46 | 0.08 | 0.03 |
| | 2.56 | 1.40 | 0.64 | 0.41 | 0.41 | 0.11 | 0.02 |
| Mean ± St. D. | 2.28 ± 0.23 | 1.25 ± 0.35 | 0.59 ± 0.15 | 0.51 ± 0.07 | 0.38 ± 0.06 | 0.12 ± 0.03 | 0.02 ± 0.01 |

The ex vivo imaging of organs was performed using a Stryker 1588 AIM clinical imaging system with L10 AIM Light Source, 1588 AIM Camera using a 10 mm scope. The lens was spaced 4.3 cm away from the surface of the organs within an enclosed (light protected) area. The NIRF imaging of each organ was performed at three different laser intensities set on a hexadecimal scale as OB-22 (low), 12-5C (medium), and 20-3A (high). Representative images of organs are shown in FIGS. 16A and 16B. The digital images of organs were processed using program written in Python to determine the average level of intensity recorded in the green channel. Since organs were imaged in the dark on black mats, the background signal was determined by introducing an intensity threshold. All the pixels with intensity from the green channel above the set threshold were counted and the average green intensity per pixel was calculated. Thus, the mean intensity per organ (Table 17a) and averaged over organs from 5 animals (Table 17b) was calculated.

TABLE 17a
Fluorescence intensity (a.u.) obtained by ex vivo imaging of
organs collected at different time points after IV administration
of ICG pHLIP ®, 5 animals were used for each time point.

| | Time p.i. | | | | | | |
|---|---|---|---|---|---|---|---|
| | 5 | 1 | 2 | 4 | 6 | 16 | 26 |
| | min | hour | hours | hours | hours | hours | hours |
| Tumor | 46.3 | 92.0 | 119.7 | 205.3 | 193.8 | 208.2 | 173.3 |
| | 37.2 | 117.6 | 145.3 | 219.5 | 169.3 | 226.8 | 208.2 |
| | 32.4 | 73.8 | 151.9 | 191.8 | 250.5 | 233.9 | 182.1 |
| | 33.4 | 61.1 | 121.8 | 206.4 | 172.1 | 214.5 | 174.8 |
| | 31.7 | 157.9 | 131.1 | 233.0 | 207.3 | 230.6 | 174.4 |
| Liver | 380.8 | 541.2 | 560.0 | 532.7 | 517.1 | 382.1 | 213.8 |
| | 393.6 | 558.6 | 568.2 | 569.4 | 560.7 | 386.3 | 196.9 |
| | 469.0 | 519.7 | 555.7 | 541.0 | 489.1 | 455.1 | 217.1 |
| | 450.4 | 520.5 | 523.6 | 539.5 | 483.9 | 368.5 | 214.9 |
| | 405.4 | 525.1 | 505.3 | 517.6 | 584.9 | 410.3 | 223.0 |
| Kidneys | 145.9 | 202.1 | 175.6 | 191.7 | 169.6 | 154.1 | 121.4 |
| | 180.2 | 202.8 | 161.0 | 188.5 | 169.3 | 175.2 | 100.2 |
| | 165.6 | 179.8 | 152.4 | 175.7 | 173.3 | 183.1 | 108.4 |
| | 195.7 | 199.0 | 154.4 | 183.6 | 180.9 | 160.3 | 115.2 |
| | 171.2 | 184.9 | 136.8 | 188.0 | 176.9 | 180.4 | 113.8 |
| Heart | 174.5 | 188.4 | 164.5 | 191.7 | 153.6 | 128.2 | 67.8 |
| | 213.0 | 194.5 | 154.9 | 191.6 | 166.7 | 115.2 | 50.1 |
| | 211.8 | 170.2 | 183.9 | 174.5 | 155.5 | 146.4 | 63.4 |
| | 191.5 | 155.5 | 176.2 | 182.8 | 166.5 | 112.3 | 61.5 |
| | 199.6 | 189.6 | 158.3 | 187.5 | 173.0 | 143.6 | 63.6 |
| Lungs | 214.0 | 194.4 | 176.4 | 187.1 | 150.6 | 132.0 | 54.4 |
| | 218.3 | 202.2 | 163.5 | 209.3 | 165.3 | 115.2 | 54.5 |
| | 220.4 | 192.7 | 172.4 | 163.7 | 141.9 | 169.5 | 81.1 |
| | 215.4 | 185.8 | 180.7 | 196.1 | 180.4 | 133.2 | 53.2 |
| | 214.8 | 198.5 | 191.7 | 179.0 | 145.7 | 156.4 | 77.6 |
| Brain | 137.0 | 126.7 | 95.4 | 73.2 | 50.6 | 36.1 | 0 |
| | 153.9 | 151.5 | 79.9 | 78.5 | 48.7 | 37.9 | 0 |
| | 196.2 | 138.1 | 96.9 | 60.0 | 47.9 | 35.6 | 0 |
| | 172.2 | 136.8 | 53.8 | 76.5 | 55.2 | 33.7 | 0 |
| | 175.6 | 135.4 | 84.8 | 81.0 | 74.3 | 33.3 | 0 |
| Spleen | 146.1 | 158.7 | 134.3 | 158.3 | 133.6 | 102.2 | 39.9 |
| | 154.8 | 162.3 | 144.2 | 167.2 | 160.9 | 101.4 | 40.6 |
| | 168.9 | 161.1 | 135.4 | 143.8 | 152.7 | 150.1 | 50.7 |
| | 147.9 | 151.4 | 144.6 | 140.0 | 134.0 | 92.5 | 50.8 |
| | 155.5 | 155.5 | 126.2 | 145.2 | 159.3 | 135.3 | 58.1 |
| Pancreas | 169.1 | 163.4 | 121.6 | 101.6 | 70.7 | 72.6 | 46.5 |
| | 176.4 | — | 96.2 | 80.2 | 62.0 | 49.6 | 38.4 |
| | 186.6 | 140.5 | 41.3 | 96.3 | 87.1 | 121.2 | 34.2 |
| | 129.0 | 119.1 | 111.3 | 77.4 | 96.3 | 49.5 | 35.4 |
| | 104.9 | 107.9 | 74.8 | 104.7 | 95.1 | 58.6 | 0 |
| Bone | 73.8 | 67.3 | 40.3 | 90.1 | 58.7 | 76.7 | 43.1 |
| | 73.1 | 92.8 | 62.5 | 35.6 | 73.6 | 73.4 | 45.4 |
| | 88.8 | 77.0 | 48.3 | 55.3 | 61.2 | 58.3 | 42.3 |
| | 73.4 | 75.5 | 96.1 | 57.5 | 49.2 | 64.5 | 50.2 |
| | 87.8 | 57.5 | 40.4 | 76.9 | 90.8 | 58.2 | 32.8 |
| Stomach | 72.8 | 125.1 | 49.9 | 98.5 | 126.8 | 103.5 | 42.2 |
| | 82.5 | 81.7 | 91.1 | 107.6 | 129.7 | 86.3 | 48.3 |
| | 104.2 | 71.8 | 79.9 | 109.5 | 141.6 | 161.0 | 71.0 |
| | 99.6 | 98.0 | 98.4 | 116.4 | 119.8 | 92.1 | 62.2 |
| | 99.3 | 118.8 | 84.3 | 110.3 | 116.1 | 143.6 | 72.0 |
| Small | 62.2 | 78.3 | 64.3 | 83.5 | 40.4 | 77.6 | 34.3 |
| intestine | 45.5 | 97.2 | 57.8 | 54.9 | 58.8 | 107.8 | 43.8 |
| | 38.4 | 68.8 | 31.5 | 42.1 | 44.2 | 78.6 | 35.8 |
| | 33.0 | 62.1 | 45.4 | 73.1 | 68.2 | 80.9 | 41.6 |
| | 33.0 | 53.0 | 36.3 | 70.8 | 59.5 | 74.2 | 33.2 |
| Large | 42.4 | 49.1 | 0 | 0 | 32.3 | 37.1 | 0 |
| intestine | 35.4 | 32.5 | 32.2 | 39.4 | 33.4 | 42.6 | 0 |
| | 30.0 | 33.3 | 0 | 0 | 30.0 | 37.8 | 0 |
| | 47.8 | 27.0 | 0 | 0 | 39.8 | 36.6 | 0 |
| | 29.8 | 0 | 0 | 0 | 0 | 45.2 | 0 |
| Skin | 40.6 | 32.0 | 40.9 | 66.9 | 34.5 | 91.1 | 0 |
| | 1.0 | 39.4 | 43.4 | 51.0 | 66.1 | 60.5 | 31.9 |
| | 38.0 | 54.1 | 0 | 38.5 | 56.3 | 82.5 | 0 |
| | 33.4 | 38.2 | 36.8 | 39.6 | 37.3 | 50.7 | 57.0 |
| | 27.6 | 32.1 | 39.0 | 45.1 | 41.2 | 98.9 | 36.4 |
| Muscle | 36.5 | 0 | 0 | 36.2 | 0 | 0 | 0 |
| | 1.0 | 40.8 | 0 | 0 | 0 | 0 | 0 |
| | 1.0 | 0 | 0 | 41.7 | 0 | 0 | 42.9 |
| | 38.6 | 0 | 0 | 0 | 0 | 0 | 0 |
| | 35.9 | 0 | 0 | 53.0 | 0 | 0 | 0 |

TABLE 17b
Mean (and St. D.) of fluorescence intensity values presented in Table 17a.

| Time p.i. | 5 min | 1 hour | 2 hours | 4 hours | 6 hours | 16 hours | 26 hours |
|---|---|---|---|---|---|---|---|
| Tumor | 36.2 ± 6.0 | 100.5 ± 38.5 | 133.9 ± 14.2 | 211.2 ± 15.7 | 198.6 ± 33.0 | 222.8 ± 11.0 | 182.6 ± 14.7 |
| Liver | 419.8 ± 37.9 | 533.0 ± 16.7 | 542.6 ± 26.8 | 540.1 ± 18.8 | 527.1 ± 44.4 | 400.5 ± 34.1 | 213.1 ± 9.7 |
| Kidneys | 171.7 ± 18.4 | 193.7 ± 10.6 | 156.0 ± 14.1 | 185.5 ± 6.2 | 174.0 ± 5.0 | 170.6 ± 12.8 | 111.8 ± 7.9 |
| Heart | 198.1 ± 15.9 | 179.6 ± 16.4 | 167.6 ± 12.2 | 185.6 ± 7.2 | 163.0 ± 8.2 | 129.2 ± 15.7 | 61.3 ± 6.6 |
| Lungs | 216.6 ± 2.7 | 194.7 ± 6.2 | 177.0 ± 10.4 | 187.0 ± 17.2 | 156.8 ± 15.9 | 141.3 ± 21.5 | 64.2 ± 13.9 |
| Brain | 167.0 ± 22.5 | 137.7 ± 8.9 | 82.2 ± 17.4 | 73.8 ± 8.3 | 55.3 ± 11.0 | 35.3 ± 1.8 | 0.0 ± 0.0 |
| Spleen | 154.6 ± 9.0 | 157.8 ± 4.4 | 136.9 ± 7.7 | 150.9 ± 11.4 | 148.1 ± 13.4 | 116.3 ± 25.0 | 48.0 ± 7.7 |
| Pancreas | 153.2 ± 34.7 | 132.7 ± 24.5 | 89.0 ± 32.0 | 92.0 ± 2.5 | 82.2 ± 15.3 | 70.3 ± 30.0 | 30.9 ± 17.9 |
| Bone | 79.4 ± 8.1 | 74.0 ± 13.1 | 57.5 ± 23.4 | 63.1 ± 21.0 | 66.7 ± 16.0 | 66.2 ± 8.5 | 42.8 ± 6.4 |
| Stomach | 91.7 ± 13.4 | 99.1 ± 23.0 | 80.7 ± 18.6 | 108.4 ± 6.5 | 126.8 ± 9.9 | 117.3 ± 33.1 | 59.1 ± 13.4 |
| S. Intest. | 42.4 ± 12.2 | 71.9 ± 16.9 | 47.1 ± 13.9 | 64.9 ± 16.3 | 54.2 ± 11.6 | 83.8 ± 13.6 | 37.8 ± 4.7 |
| L. Intest. | 37.1 ± 7.9 | 28.4 ± 17.9 | 6.4 ± 14.4 | 7.9 ± 17.6 | 27.1 ± 15.6 | 40.5 ± 4.0 | 0.0 ± 0.0 |
| Skin | 27.9 ± 16.4 | 39.1 ± 9.0 | 32.0 ± 18.1 | 48.2 ± 11.6 | 47.1 ± 13.6 | 76.7 ± 20.4 | 25.1 ± 24.8 |
| Muscle | 22.2 ± 20.3 | 8.2 ± 18.2 | 0.0 ± 0.0 | 26.2 ± 24.6 | 0.0 ± 0.0 | 0.0 ± 0.0 | 8.6 ± 19.2 |

The blood clearance, biodistribution and kinetics of signal changes in tissue and organs at different time points after single tail vein administration of ICG-pHLIP are shown in FIGS. 17 and 18.

The fluorescence signals in organs and tissue were also measured in the tissue/organ homogenates and compared with the signals from the control tissue/organ homogenates (collected from control mice) mixed with known amounts of ICG-pHLIP. About 100 mg of tissue (tumor, liver or kidney) were homogenized with 2.5× (about 250 μL) volumes of DMSO using BioMasher II disposable homogenizers (DiagnoCine LLC). 30 μl of homogenate was placed into 384 well plate and imaged using an Odyssey IR scanner. Tumor, liver or kidney homogenates of control mice, mixed with known concentrations of ICG-pHLIP were used to establish the calibration curve. First, note that the fluorescence signals obtained on the control tumor, liver and kidney mixed with the known amounts of ICG-pHLIP were the same. Second, the course of signal changes in tumor, liver and kidney (data not shown) was in excellent correlation with the course of the kinetics presented on FIGS. 18A-F. It is rather difficult to establish precisely the amount of ICG-pHLIP in the tissue and organ samples; however, rough estimations can be made using the calibration curve. It is estimated that the amount of ICG-pHLIP in tumors reaches about 10-15% ID/g at 4 hours post injection and stays constant up to 16 hours, decaying slightly at 26 hours. ICG-pHLIP clearance from the blood follows two-phase kinetics with characteristic half lifetime of 0.6 hours, when signal drops on 66%, and 7.5 hours for complete clearance of the blood. The clearance seems to be predominantly hepatic, the level of the signal increases in liver with time and reaches maximum level at 1 hour p.i., followed by the decay of the signal after 6 hours. Heart, kidney and lungs are organs with a significant amount of blood, and they were imaged as is (with blood). It is seen on FIG. 16A that a significant amount of the signal is coming from the blood associated with these organs, and that the level of the signal in these organs decays with blood clearance. Another group of organs, such as spleen, pancreas, stomach and brain has a lower level of the signal, which also decays with blood clearance. Small and large intestines, bone, skin and muscle have very low levels of the signal; we estimated it to be less than 5-2% ID/g at all time points. Only the tumor shows a steady increase of the signal over the first 4 hours, and the fluorescence stays within the tumor for up to 26 hours, clearly indicating the targeting of tumor by ICG-pHLIP. At 26 hours, when the blood is cleared and the fluorescence signal in all organs is minimal, the contrast between tumor and surrounding tissue is very significant.

### Targeting Tumors in Different Mouse Tumor Models

Targeting of murine and human tumors was shown in six different tumor models in athymic female nude mice (strain Hsd Athymic Nude-Foxn1nu) ranging in age from 5 to 6 weeks (obtained from Envigo RMS Inc). The following tumors were established by subcutaneous injection of 1×10⁶ cells/0.1 ml/flank in both flanks of athymic nude mice: HeLa (cervical adenocarcinoma), M4A4 (breast ductal carcinoma), A549 (lung carcinoma), UM-UC3 (urinary bladder cancer), 4T1 (murine breast tumor). Human MDA-MB-231 (breast adenocarcinoma) tumors were established by injections of 1×10⁶ cells/0.05 ml in the mammary fat pad. Tumors reached different sizes (from very small to large) and 100 μl of tail vein injections of 2.5 nmol (0.5 mg/kg) of ICG-pHLIP in sterile PBS containing either 5% of DMSO or 5% of Ethanol were performed. Imaging was carried out at 24 hours after construct administration using the Stryker 1588 AIM imaging system. White light and NIRF whole-body imaging was performed while the animal was under gas (isoflurane) anesthesia (FIGS. 19-21). Next, the skin was removed from the tumor side and whole-body imaging of live animals was performed with the skin removed from the tumor side.

A resected tumor and tumor bed are shown in FIGS. 22A-C. The surgical removal of tumor was not successful; the residual tumor was left behind and clearly visualized by ICG-pHLIP NIRF imaging.

Animals were euthanized immediately after whole-body imaging, and the tumor with surrounding muscle was collected and imaged (FIGS. 23A-L). Fluorescent signals from small MDA-MB-231 breast tumors are seen both through the skin and when the skin is open. Both types of tumors i) grown at the surface or ii) grown very deep within muscle and undetectable by eye are very well revealed by ICG-pHLIP NIRF signal. Both small and big tumors are targeted with high precision.

Excised tumors with surrounding muscle are shown in FIGS. 24 and 25. Excellent tumor to muscle contrast is observed. To correlate ICG-pHLIP NIRF signal with tumor location immediately following NIRF imaging, the tumor-muscle pieces were frozen in tissue-tek OCT compound using liquid nitrogen, and stored at −80° C. until sectioned using a cryostat at −25° C. (Thermo Scientific HM525 NX) at a 5 μm thickness. The tumor slides were fixed in 4% formaldehyde and stained with hematoxylin and eosin (H&E) (Thermo Fisher Scientific and Poly Scientific R & D Corp). Some sections were covered with a drop of mounting medium (Permount®, Fisher Scientific) and then a cover slide was placed over the medium.

An excellent correlation between ICG-pHLIP NIRF imaging and H&E histopathology indicating tumor location are shown in FIGS. 25 and 26. Also, fluorescent (non-processed) and H&E slides with sections were examined under an Odyssey IR scanner, Stryker imager and an inverted fluorescence microscope (IX71 Olympus) using 10× and 40× objectives. It is clearly demonstrated in FIGS. 25-28 that tumors exhibit the highest ICG-pHLIP NIRF signal. It is interesting to note, that high fluorescence was observed in tissue surrounding tumor, marked by star (*) on FIGS. 26A and 27A. Investigation of the different parts of H&E stained section under a microscope using 10× objective indicated on the presence of cancer cells in areas surrounding main tumor mass (FIGS. 26E and 27E). Thus, it is not surprising to see strong fluorescent signal coming from these (tumor surrounding) areas. The magnified images of tumor part of the section obtained on a fluorescent inverted microscope with 40× objective in fluorescence and bright field modes are shown in FIGS. 26G-J. In all places where tissue was present (see dark areas in the FIG. 26H), the fluorescent signal was observed.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1-43. (canceled)

44. A method of treating cancer in a subject, comprising administering to the subject an effective amount of a pH-triggered compound comprising a cargo compound that is covalently attached to a pH-triggered peptide that is covalently attached to at least one other pH-triggered peptide via a linker or a covalent bond.

45. The method of claim 44, wherein the pH-triggered compound comprises the following structure:
A-L-B
wherein A is a first pH-triggered peptide comprising the sequence DDQNPWRAYLDLLFPTDTLLLDLLW (SEQ ID NO: 1), B is a second pH-triggered peptide comprising the sequence DDQNPWRAYLDLLFPTDTLLLDLLW (SEQ ID NO: 1), L is a polyethylene glycol linker, and each - is a covalent bond.

46. The method of claim 44, wherein the pH-triggered compound comprises at least one pH-triggered peptide comprising one or more of the following sequences: AYLDLLFP (SEQ ID NO: 4), YLDLLFPT (SEQ ID NO: 5), LDLLFPTD (SEQ ID NO: 6), DLLFPTDT (SEQ ID NO: 7), LLFPTDT (SEQ ID NO: 8), LFPTDTLL (SEQ ID NO: 9), FPTDTLLL (SEQ ID NO: 10), PTDTLLLD (SEQ ID NO: 11), TDTLLLDL (SEQ ID NO: 12), DTLLLDLL (SEQ ID NO: 13), or TLLLDLLW (SEQ ID NO: 14).

47. The method of claim 44, wherein the pH-triggered compound comprises at least one pH-triggered peptide comprising the sequence DDQNPWRAYLDLLFPTDTLLLDLLW (SEQ ID NO: 1).

48. The method of claim 44, wherein the pH-triggered compound has the following structure:
[A]k-linker
wherein
k is an integer from 2 to 32, and
each A is, individually, a pH-triggered peptide comprising at least 8 consecutive amino acids, wherein
(i) at least 4 of the at least 8 consecutive amino acids are non-polar amino acids,
(ii) at least 1 of the at least 8 consecutive amino acids is protonatable, and
(iii) the pH-triggered peptide has a higher affinity for a membrane lipid bilayer at pH 5.0 compared to the affinity at pH 8.0.

49. The method of claim 44, wherein the pH-triggered compound comprises at least two pH-triggered peptides with different amino acid sequences or wherein each pH-triggered peptide comprises the same amino acid sequence.

50. The method of claim 44, wherein
(a) the cargo compound is polar or nonpolar;
(b) the cargo compound comprises a marker;
(c) the cargo compound comprises a prophylactic, therapeutic, diagnostic, radiation-enhancing, radiation-sensitizing, imaging, gene regulation, immune activation, cytotoxic, apoptotic, or research agent;
(d) the cargo compound comprises a dye, a fluorescent dye, a fluorescence quencher, or a fluorescent protein;
(e) the cargo compound comprises a magnetic resonance, positron emission tomography, single photon emission computed tomography, fluorescent, optoacoustic, ultrasound, or X-ray contrast imaging agent;
(f) the cargo compound comprises a peptide, a protein, an enzyme, or a polysaccharide;
(g) the cargo compound comprises an aptamer, an antigen, a protease, an amylase, a lipase, a Fc receptor, a tissue factor, or a complement component 3 (C3) protein;
(h) the cargo compound comprises a toxin, an inhibitor, a DNA intercalator, an alkylating agent, an antimetabolite, an anti-microtubule agents, a topoisomerase inhibitor, or an antibiotic compound;
(i) the cargo compound comprises an amanita toxin, a vinca alkaloid, a taxane, an anthracycline, a bleomycin, a nitrogen mustard, a nitrosourea, a tetrazine, an aziridine, a platinum-containing chemotherapeutic agent, cisplatin or a cisplatin derivative, a procarbazine, or a hexamethylmelamine;
(j) the cargo compound comprises a DNA, a DNA analog, a RNA, a RNA analog;
(k) the cargo compound comprises a peptide nucleic acid (PNA), a bis PNA, a gamma PNA, a locked nucleic acid (LNA), or a morpholino;
(l) the cargo compound comprises a chemotherapeutic compound;
(m) the cargo compound comprises an antimicrobial compound; or
(n) the cargo compound comprises a gene-regulation compound.

51. The method of claim 44, wherein the linker is attached to the cargo compound via a covalent bond, wherein
(a) the covalent bond is an ester bond, a disulfide bond, a bond between two selenium atoms, a bond between a sulfur and a selenium atom, or an acid-liable bond;
(b) the covalent bond is a bond that has been formed by a click chemistry reaction; or
(c) the covalent bond is a bond that has been formed by a reaction between an azide and an alkyne, an alkyne and a strained difluorooctyne, a diaryl-strained-cyclooctyne and a 1,3-nitrone, a cyclooctene, trans-cycloalkene, or oxanorbornadiene and an azide, tetrazine, or tetrazole, an activated alkene or oxanorbornadiene and an azide, a strained cyclooctene or other activated alkene and a tetrazine, or a tetrazole that has been activated by ultraviolet light and an alkene.

52. A method for ex vivo diagnostics, comprising a fluorescent pH-triggered compound comprising a pH-triggered peptide that is covalently attached to at least one other pH-triggered peptide via a linker or a covalent bond, and further comprising:
(a) contacting a biological sample from a subject with a fluorescent pH-triggered compound comprising a fluorophore;
(b) contacting the biological sample with electromagnetic radiation comprising an excitation wavelength of the fluorophore; and
(c) detecting electromagnetic radiation emitted from the fluorescent pH-triggered compound.

53. The method of claim 52, wherein the compound comprises the following structure:
A-L-B
wherein A is a first pH-triggered peptide comprising the sequence DDQNPWRAYLDLLFPTDTLLLDLLW (SEQ ID NO: 1), B is a second pH-triggered peptide comprising the sequence DDQNPWRAYLDLLFPTDTLLLDLLW (SEQ ID NO: 1), L is a polyethylene glycol linker, and each - is a covalent bond.

54. The method of claim 52, wherein the fluorescent pH-triggered compound comprises at least one pH-triggered peptide comprising one or more of the following sequences: AYLDLLFP (SEQ ID NO: 4), YLDLLFPT (SEQ ID NO: 5), LDLLFPTD (SEQ ID NO: 6), DLLFPTDT (SEQ ID NO: 7), LLFPTDT (SEQ ID NO: 8), LFPTDTLL (SEQ ID NO: 9), FPTDTLLL (SEQ ID NO: 10), PTDTLLLD (SEQ ID NO: 11), TDTLLLDL (SEQ ID NO: 12), DTLLLDLL (SEQ ID NO: 13), or TLLLDLLW (SEQ ID NO: 14).

55. The method of claim 52, wherein the fluorescent pH-triggered compound has the following structure:
[A]k-linker
wherein
k is an integer from 2 to 32, and
each A is, individually, a pH-triggered peptide comprising at least 8 consecutive amino acids, wherein
(i) at least 4 of the at least 8 consecutive amino acids are non-polar amino acids,
(ii) at least 1 of the at least 8 consecutive amino acids is protonatable, and
(iii) the pH-triggered peptide has a higher affinity for a membrane lipid bilayer at pH 5.0 compared to the affinity at pH 8.0.

56. The method of claim 52, wherein the pH-triggered compound comprises at least two pH-triggered peptides with different amino acid sequences or wherein each pH-triggered peptide comprises the same amino acid sequence.

57. The method of claim 52, further comprising a cargo compound, wherein
(a) the cargo compound is polar or nonpolar;
(b) the cargo compound comprises a marker;
(c) the cargo compound comprises a prophylactic, therapeutic, diagnostic, radiation-enhancing, radiation-sensitizing, imaging, gene regulation, immune activation, cytotoxic, apoptotic, or research agent;
(d) the cargo compound comprises a dye, a fluorescent dye, a fluorescence quencher, or a fluorescent protein;
(e) the cargo compound comprises a magnetic resonance, positron emission tomography, single photon emission computed tomography, fluorescent, optoacoustic, ultrasound, or X-ray contrast imaging agent;
(f) the cargo compound comprises a peptide, a protein, an enzyme, or a polysaccharide;
(g) the cargo compound comprises an aptamer, an antigen, a protease, an amylase, a lipase, a Fc receptor, a tissue factor, or a complement component 3 (C3) protein;
(h) the cargo compound comprises a toxin, an inhibitor, a DNA intercalator, an alkylating agent, an antimetabolite, an anti-microtubule agents, a topoisomerase inhibitor, or an antibiotic compound;
(i) the cargo compound comprises an amanita toxin, a vinca alkaloid, a taxane, an anthracycline, a bleomycin, a nitrogen mustard, a nitrosourea, a tetrazine, an aziridine, a platinum-containing chemotherapeutic agent, cisplatin or a cisplatin derivative, a procarbazine, or a hexamethylmelamine;
(j) the cargo compound comprises a DNA, a DNA analog, a RNA, a RNA analog;
(k) the cargo compound comprises a peptide nucleic acid (PNA), a bis PNA, a gamma PNA, a locked nucleic acid (LNA), or a morpholino;
(l) the cargo compound comprises a chemotherapeutic compound;
(m) the cargo compound comprises an antimicrobial compound; or
(n) the cargo compound comprises a gene-regulation compound.

58. The method of claim 57, wherein a linker is attached to the cargo compound via a covalent bond, wherein
(a) the covalent bond is an ester bond, a disulfide bond, a bond between two selenium atoms, a bond between a sulfur and a selenium atom, or an acid-liable bond;
(b) the covalent bond is a bond that has been formed by a click chemistry reaction; or
(c) the covalent bond is a bond that has been formed by a reaction between an azide and an alkyne, an alkyne and a strained difluorooctyne, a diaryl-strained-cyclooctyne and a 1,3-nitrone, a cyclooctene, trans-cycloalkene, or oxanorbornadiene and an azide, tetrazine, or tetrazole, an activated alkene or oxanorbornadiene and an azide, a strained cyclooctene or other activated alkene and a tetrazine, or a tetrazole that has been activated by ultraviolet light and an alkene.

59. A pH-triggered compound comprising a pH-triggered peptide that is covalently attached to at least one other pH-triggered peptide via a linker or a covalent bond.

60. The pH-triggered compound of claim 59, wherein pH-triggered compound comprises the following structure:
A-L-B
wherein A is a first pH-triggered peptide comprising the sequence DDQNPWRAYLDLLFPTDTLLLDLLW (SEQ ID NO: 1), B is a second pH-triggered peptide comprising the sequence DDQNPWRAYLDLLFPTDTLLLDLLW (SEQ ID NO: 1), L is a polyethylene glycol linker, and each - is a covalent bond.

61. The pH-triggered compound of claim 59, wherein the pH-triggered compound comprises at least one pH-triggered peptide comprising one or more of the following sequences:
(SEQ ID NO: 4)
AYLDLLFP,
(SEQ ID NO: 5)
YLDLLFPT,
(SEQ ID NO: 6)
LDLLFPTD,
(SEQ ID NO: 7)
DLLFPTDT,
(SEQ ID NO: 8)
LLFPTDT,
(SEQ ID NO: 9)
LFPTDTLL,
(SEQ ID NO: 10)
FPTDTLLL,
(SEQ ID NO: 11)
PTDTLLLD,
(SEQ ID NO: 12)
TDTLLLDL,
(SEQ ID NO: 13)
DTLLLDLL,
or
(SEQ ID NO: 14)
TLLLDLLW

62. The pH-triggered compound of claim 59, wherein the pH-triggered compound comprises at least one pH-triggered peptide comprising the sequence
(SEQ ID NO: 1)
DDQNPWRAYLDLLFPTDTLLLDLLW

63. The pH-triggered compound of claim 59, further comprising a cargo compound, wherein
(a) the cargo compound is polar or nonpolar;
(b) the cargo compound comprises a marker;
(c) the cargo compound comprises a prophylactic, therapeutic, diagnostic, radiation-enhancing, radiation-sensitizing, imaging, gene regulation, immune activation, cytotoxic, apoptotic, or research agent;
(d) the cargo compound comprises a dye, a fluorescent dye, a fluorescence quencher, or a fluorescent protein;
(e) the cargo compound comprises a magnetic resonance, positron emission tomography, single photon emission computed tomography, fluorescent, optoacoustic, ultrasound, or X-ray contrast imaging agent;
(f) the cargo compound comprises a peptide, a protein, an enzyme, or a polysaccharide;
(g) the cargo compound comprises an aptamer, an antigen, a protease, an amylase, a lipase, a Fc receptor, a tissue factor, or a complement component 3 (C3) protein;
(h) the cargo compound comprises a toxin, an inhibitor, a DNA intercalator, an alkylating agent, an antimetabolite, an anti-microtubule agents, a topoisomerase inhibitor, or an antibiotic compound;
(i) the cargo compound comprises an amanita toxin, a vinca alkaloid, a taxane, an anthracycline, a bleomycin, a nitrogen mustard, a nitrosourea, a tetrazine, an aziridine, a platinum-containing chemotherapeutic agent, cisplatin or a cisplatin derivative, a procarbazine, or a hexamethylmelamine;
(j) the cargo compound comprises a DNA, a DNA analog, a RNA, a RNA analog;
(k) the cargo compound comprises a peptide nucleic acid (PNA), a bis PNA, a gamma PNA, a locked nucleic acid (LNA), or a morpholino;
(l) the cargo compound comprises a chemotherapeutic compound;
(m) the cargo compound comprises an antimicrobial compound; or
(n) the cargo compound comprises a gene-regulation compound.


SEQUENCE LISTING (331 sequences)

SEQ ID NO: 1 - protein, 25 aa, synthetic construct
  source 1..25: note = Synthetic peptide
DDQNPWRAYLDLLFPTDTLLLDLLW

SEQ ID NO: 2 - protein, 35 aa, synthetic construct
  source 1..35: note = Synthetic polypeptide
AEQNPIYWARYADWLFTTPLLLLDLALLVDADEGT

SEQ ID NO: 3 - protein, 34 aa, synthetic construct
  source 1..34: note = Synthetic polypeptide
GLAGLAGLLGLEGLLGLPLGLLEGLWLGLELEGN

SEQ ID NO: 4 - protein, 8 aa, synthetic construct
  source 1..8: note = Synthetic peptide
AYLDLLFP

SEQ ID NO: 5 - protein, 8 aa, synthetic construct
  source 1..8: note = Synthetic peptide
YLDLLFPT

SEQ ID NO: 6 - protein, 8 aa, synthetic construct
  source 1..8: note = Synthetic peptide
LDLLFPTD

SEQ ID NO: 7 - protein, 8 aa, synthetic construct
  source 1..8: note = Synthetic peptide
DLLFPTDT

SEQ ID NO: 8 - protein, 7 aa, synthetic construct
  source 1..7: note = Synthetic peptide
LLFPTDT

SEQ ID NO: 9 - protein, 8 aa, synthetic construct
  source 1..8: note = Synthetic peptide
LFPTDTLL

SEQ ID NO: 10 - protein, 8 aa, synthetic construct
  source 1..8: note = Synthetic peptide
FPTDTLLL

SEQ ID NO: 11 - protein, 8 aa, synthetic construct
  source 1..8: note = Synthetic peptide
PTDTLLLD

SEQ ID NO: 12 - protein, 8 aa, synthetic construct
  source 1..8: note = Synthetic peptide
TDTLLLDL

SEQ ID NO: 13 - protein, 8 aa, synthetic construct
  source 1..8: note = Synthetic peptide
DTLLLDLL

SEQ ID NO: 14 - protein, 8 aa, synthetic construct
  source 1..8: note = Synthetic peptide
TLLLDLLW

SEQ ID NO: 15 - protein, 28 aa, synthetic construct
  source 1..28: note = Synthetic peptide
ACDDQNPWRAYLDLLFPTDTLLLDLLWA

SEQ ID NO: 16 - protein, 28 aa, synthetic construct
  source 1..28: note = Synthetic peptide
AKDDQNPWRAYLDLLFPTDTLLLDLLWA

SEQ ID NO: 17 - protein, 28 aa, synthetic construct
  source 1..28: note = Synthetic peptide
ADDQNPWRAYLDLLFPTDTLLLDLLWCA

SEQ ID NO: 18 - protein, 28 aa, synthetic construct
  source 1..28: note = Synthetic peptide
ADDQNPWRAYLDLLFPTDTLLLDLLWKA

SEQ ID NO: 19 - protein, 29 aa, synthetic construct
  source 1..29: note = Synthetic peptide
ACDDQNPWRAYLDLLFPTDTLLLDLLWKA

SEQ ID NO: 20 - protein, 29 aa, synthetic construct
  source 1..29: note = Synthetic peptide
AKDDQNPWRAYLDLLFPTDTLLLDLLWCA

SEQ ID NO: 21 - protein, 27 aa, synthetic construct
  source 1..27: note = Synthetic peptide
ADDQNPWRAYLDLLFPTDTLLLDLLWG

SEQ ID NO: 22 - protein, 28 aa, synthetic construct
  source 1..28: note = Synthetic peptide
AKDDQNPWRAYLDLLFPTDTLLLDLLWG

SEQ ID NO: 23 - protein, 27 aa, synthetic construct
  source 1..27: note = Synthetic peptide; note = Synthetic peptide
ADDQNPWRAYLDLLFPTDTLLLDLLWA

SEQ ID NO: 24 - protein, 28 aa, synthetic construct
  source 1..28: note = Synthetic peptide
ACDDQNPWRAYLDLLFPTDTLLLDLLWG

SEQ ID NO: 25 - protein, 28 aa, synthetic construct
  source 1..28: note = Synthetic peptide
ADDQNPWRAYLDLLFPTDTLLLDLLWCG

SEQ ID NO: 26 - protein, 28 aa, synthetic construct
  source 1..28: note = Synthetic peptide
ADDQNPWRAYLDLLFPTDTLLLDLLWKG

SEQ ID NO: 27 - protein, 29 aa, synthetic construct
  source 1..29: note = Synthetic peptide
ACDDQNPWRAYLDLLFPTDTLLLDLLWKG

SEQ ID NO: 28 - protein, 29 aa, synthetic construct
  source 1..29: note = Synthetic peptide
AKDDQNPWRAYLDLLFPTDTLLLDLLWCG

SEQ ID NO: 29 - protein, 29 aa, synthetic construct
  source 1..29: note = Synthetic peptide
ACKDDQNPWRAYLDLLFPTDTLLLDLLWG

SEQ ID NO: 30 - protein, 29 aa, synthetic construct
  source 1..29: note = Synthetic peptide
AKDDQNPWRAYLDLLFPTDTLLLDLLWKA

SEQ ID NO: 31 - protein, 29 aa, synthetic construct
  source 1..29: note = Synthetic peptide
ACDDQNPWRAYLDLLFPTDTLLLDLLWCA

SEQ ID NO: 32 - protein, 27 aa, synthetic construct
  source 1..27: note = Synthetic peptide
ADDQNPWRAYLDLLFPTDTLLLDLLWK

SEQ ID NO: 33 - protein, 28 aa, synthetic construct
  source 1..28: note = Synthetic peptide
AKDDQNPWRAYLDLLFPTDTLLLDLLWC

SEQ ID NO: 34 - protein, 28 aa, synthetic construct
  source 1..28: note = Synthetic peptide
AKDDQNPWRAYLDLLFPTDTLLLDLLWK

SEQ ID NO: 35 - protein, 28 aa, synthetic construct
  source 1..28: note = Synthetic peptide
ACDDQNPWRAYLDLLFPTDTLLLDLLWC

SEQ ID NO: 36 - protein, 28 aa, synthetic construct
  source 1..28: note = Synthetic peptide
ACDDQNPWRAYLDLLFPTDTLLLDLLWK

SEQ ID NO: 37 - protein, 22 aa, synthetic construct
  source 1..22: note = Synthetic peptide
WARYADWLFTTPLLLLDLALLV

SEQ ID NO: 38 - protein, 22 aa, synthetic construct
  MOD_RES 6: note = Gamma-carboxyglutamic acid
  source 1..22: note = Synthetic peptide
WARYAXWLFTTPLLLLDLALLV

SEQ ID NO: 39 - protein, 22 aa, synthetic construct
  MOD_RES 6: note = Gamma-carboxyglutamic acid
  MOD_RES 17: note = Aad
  source 1..22: note = Synthetic peptide
WARYAXWLFTTPLLLLXLALLV

SEQ ID NO: 40 - protein, 21 aa, synthetic construct
  source 1..21: note = Synthetic peptide
PWRAYLDLLFPTDTLLLDLLW

SEQ ID NO: 41 - protein, 21 aa, synthetic construct
  MOD_RES 7: note = Gamma-carboxyglutamic acid
  source 1..21: note = Synthetic peptide
PWRAYLXLLFPTDTLLLDLLW

SEQ ID NO: 42 - protein, 25 aa, synthetic construct
  source 1..25: note = Synthetic peptide
PWLGAYLDLLFPLELLGLLELGLWG

SEQ ID NO: 43 - protein, 24 aa, synthetic construct
  source 1..24: note = Synthetic peptide
LLGLEGLLGLPLGLLEGLWLGLEL

SEQ ID NO: 44 - protein, 35 aa, synthetic construct
  MOD_RES 13: note = Gamma-carboxyglutamic acid
  source 1..35: note = Synthetic polypeptide
AEQNPIYWARYAXWLFTTPLLLLDLALLVDADEGT

SEQ ID NO: 45 - protein, 35 aa, synthetic construct
  MOD_RES 13: note = Gamma-carboxyglutamic acid
  MOD_RES 24: note = Aad
  source 1..35: note = Synthetic polypeptide
AEQNPIYWARYAXWLFTTPLLLLXLALLVDADEGT

SEQ ID NO: 46 - protein, 26 aa, synthetic construct
  source 1..26: note = Synthetic peptide
ADDQNPWRAYLDLLFPTDTLLLDLLW

SEQ ID NO: 47 - protein, 26 aa, synthetic construct
  MOD_RES 12: note = Gamma-carboxyglutamic acid
  source 1..26: note = Synthetic peptide
ADDQNPWRAYLXLLFPTDTLLLDLLW

SEQ ID NO: 48 - protein, 30 aa, synthetic construct
  source 1..30: note = Synthetic polypeptide
GEEQNPWLGAYLDLLFPLELLGLLELGLWG

SEQ ID NO: 49 - DNA, 23 bp, synthetic construct
  source 1..23: mol_type = other DNA; note = Synthetic oligonucleotide; note = Peptide nucleic acid
acccctatcacaattagcattaa

SEQ ID NO: 50 - DNA, 22 bp, synthetic construct
  source 1..22: mol_type = other DNA; note = Synthetic oligonucleotide; note = Peptide nucleic acid
tcaacatcagtctgataagcta

SEQ ID NO: 51 - DNA, 25 bp, synthetic construct
  source 1..25: mol_type = other DNA; note = Synthetic oligonucleotide; note = Peptide nucleic acid
cggtgtgagttctaccattgccaaa

SEQ ID NO: 52 - DNA, 10 bp, synthetic construct
  source 1..10: mol_type = other DNA; note = Synthetic oligonucleotide; note = Peptide nucleic acid
tactgcaggc

SEQ ID NO: 53 - DNA, 11 bp, synthetic construct
  source 1..11: mol_type = other DNA; note = Synthetic oligonucleotide; note = Peptide nucleic acid
gtaactctggg

SEQ ID NO: 54 - DNA, 10 bp, synthetic construct
  source 1..10: mol_type = other DNA; note = Peptide nucleic acid; note = Synthetic oligonucleotide
tctgtaactc

SEQ ID NO: 55 - DNA, 10 bp, synthetic construct
  source 1..10: mol_type = other DNA; note = Synthetic oligonucleotide; note = Peptide nucleic acid
ccctctctcc

SEQ ID NO: 56 - protein, 9 aa, synthetic construct
  source 1..9: note = Synthetic peptide
RRRQRRKKR

SEQ ID NO: 57 - protein, 21 aa, synthetic construct
  source 1..21: note = Synthetic peptide
WARYADWLFTTPLLLLDLALL

SEQ ID NO: 58 - protein, 21 aa, synthetic construct
  source 1..21: note = Synthetic peptide
YARYADWLFTTPLLLLDLALL

SEQ ID NO: 59 - protein, 21 aa, synthetic construct
  source 1..21: note = Synthetic peptide
WARYSDWLFTTPLLLYDLGLL

SEQ ID NO: 60 - protein, 21 aa, synthetic construct
  source 1..21: note = Synthetic peptide
WARYTDWFTTPLLLYDLALLA

SEQ ID NO: 61 - protein, 21 aa, synthetic construct
  source 1..21: note = Synthetic peptide
WARYTDWLFTTPLLLYDLGLL

SEQ ID NO: 62 - protein, 21 aa, synthetic construct
  source 1..21: note = Synthetic peptide
WARYADWLFTTPLLLLDLSLL

SEQ ID NO: 63 - protein, 21 aa, synthetic construct
  source 1..21: note = Synthetic peptide
LLALDLLLLPTTFLWDAYRAW

SEQ ID NO: 64 - protein, 21 aa, synthetic construct
  source 1..21: note = Synthetic peptide
LLALDLLLLPTTFLWDAYRAY

SEQ ID NO: 65 - protein, 21 aa, synthetic construct
  source 1..21: note = Synthetic peptide
LLGLDYLLLPTTFLWDSYRAW

SEQ ID NO: 66 - protein, 21 aa, synthetic construct
  source 1..21: note = Synthetic peptide
ALLALDYLLLPTTFWDTYRAW

SEQ ID NO: 67 - protein, 21 aa, synthetic construct
  source 1..21: note = Synthetic peptide
LLGLDYLLLPTTFLWDTYRAW

SEQ ID NO: 68 - protein, 26 aa, synthetic construct
  source 1..26: note = Synthetic peptide
GLAGLLGLEGLLGLPLGLLEGLWLGL

SEQ ID NO: 69 - protein, 26 aa, synthetic construct
  source 1..26: note = Synthetic peptide
LGLWLGELLGLPLGLLGELGLLGALG

SEQ ID NO: 70 - protein, 20 aa, synthetic construct
  source 1..20: note = Synthetic peptide
WRAYLDLLFPTDTLLLDLLW

SEQ ID NO: 71 - protein, 20 aa, synthetic construct
  source 1..20: note = Synthetic peptide
WLLDLLLTDTPFLLDLYARW

SEQ ID NO: 72 - protein, 18 aa, synthetic construct
  source 1..18: note = Synthetic peptide
WARYLEWLFPTETLLLEL

SEQ ID NO: 73 - protein, 18 aa, synthetic construct
  source 1..18: note = Synthetic peptide
WAQYLELLFPTETLLLEW

SEQ ID NO: 74 - protein, 18 aa, synthetic construct
  source 1..18: note = Synthetic peptide
LELLLTETPFLWELYRAW

SEQ ID NO: 75 - protein, 18 aa, synthetic construct
  source 1..18: note = Synthetic peptide
WELLLTETPFLLELYQAW

SEQ ID NO: 76 - protein, 17 aa, synthetic construct
  source 1..17: note = Synthetic peptide
WLFTTPLLLLNGALLVE

SEQ ID NO: 77 - protein, 17 aa, synthetic construct
  source 1..17: note = Synthetic peptide
WLFTTPLLLLPGALLVE

SEQ ID NO: 78 - protein, 15 aa, synthetic construct
  source 1..15: note = Synthetic peptide
WARYADLLFPTTLAW

SEQ ID NO: 79 - protein, 17 aa, synthetic construct
  source 1..17: note = Synthetic peptide
EVLLAGNLLLLPTTFLW

SEQ ID NO: 80 - protein, 17 aa, synthetic construct
  source 1..17: note = Synthetic peptide
EVLLAGPLLLLPTTFLW

SEQ ID NO: 81 - protein, 15 aa, synthetic construct
  source 1..15: note = Synthetic peptide
WALTTPFLLDAYRAW

SEQ ID NO: 82 - protein, 24 aa, synthetic construct
  source 1..24: note = Synthetic peptide
NLEGFFATLGGEIALWSLVVLAIE

SEQ ID NO: 83 - protein, 22 aa, synthetic construct
  source 1..22: note = Synthetic peptide
EGFFATLGGEIALWSDVVLAIE

SEQ ID NO: 84 - protein, 22 aa, synthetic construct
  source 1..22: note = Synthetic peptide
EGFFATLGGEIPLWSDVVLAIE

SEQ ID NO: 85 - protein, 24 aa, synthetic construct
  source 1..24: note = Synthetic peptide
EIALVVLSWLAIEGGLTAFFGELN

SEQ ID NO: 86 - protein, 22 aa, synthetic construct
  source 1..22: note = Synthetic peptide
EIALVVDSWLAIEGGLTAFFGE

SEQ ID NO: 87 - protein, 22 aa, synthetic construct
  source 1..22: note = Synthetic peptide
EIALVVDSWLPIEGGLTAFFGE

SEQ ID NO: 88 - protein, 21 aa, synthetic construct
  source 1..21: note = Synthetic peptide
ILDLVFGLLFAVTSVDFLVQW

SEQ ID NO: 89 - protein, 21 aa, synthetic construct
  source 1..21: note = Synthetic peptide
WQVLFDVSTVAFLLGFVLDLI

SEQ ID NO: 90 - protein, 21 aa, synthetic construct
  MOD_RES 6: note = D, E, gamma-carboxyglutamic acid or Aad
  MOD_RES 17: note = D, E, gamma-carboxyglutamic acid or Aad
  source 1..21: note = Synthetic peptide
WARYAXWLFTTPLLLLXLALL

SEQ ID NO: 91 - protein, 21 aa, synthetic construct
  MOD_RES 6: note = D, E, gamma-carboxyglutamic acid or Aad
  MOD_RES 17: note = D, E, gamma-carboxyglutamic acid or Aad
  source 1..21: note = Synthetic peptide
YARYAXWLFTTPLLLLXLALL

SEQ ID NO: 92 - protein, 21 aa, synthetic construct
  MOD_RES 6: note = D, E, gamma-carboxyglutamic acid or Aad
  MOD_RES 17: note = D, E, gamma-carboxyglutamic acid or Aad
  source 1..21: note = Synthetic peptide
WARYSXWLFTTPLLLYXLGLL

SEQ ID NO: 93 - protein, 21 aa, synthetic construct
  MOD_RES 6: note = D, E, gamma-carboxyglutamic acid or Aad
  MOD_RES 16: note = D, E, gamma-carboxyglutamic acid or Aad
  source 1..21: note = Synthetic peptide
WARYTXWFTTPLLLYXLALLA

SEQ ID NO: 94 - protein, 21 aa, synthetic construct
  MOD_RES 6: note = D, E, gamma-carboxyglutamic acid or Aad
  MOD_RES 17: note = D, E, gamma-carboxyglutamic acid or Aad
  source 1..21: note = Synthetic peptide
WARYTXWLFTTPLLLYXLGLL

SEQ ID NO: 95 - protein, 21 aa, synthetic construct
  MOD_RES 6: note = D, E, gamma-carboxyglutamic acid or Aad
  MOD_RES 17: note = D, E, gamma-carboxyglutamic acid or Aad
  source 1..21: note = Synthetic peptide
WARYAXWLFTTPLLLLXLSLL

SEQ ID NO: 96 - protein, 21 aa, synthetic construct
  MOD_RES 5: note = D, E, gamma-carboxyglutamic acid or Aad
  MOD_RES 16: note = D, E, gamma-carboxyglutamic acid or Aad
  source 1..21: note = Synthetic peptide
LLALXLLLLPTTFLWXAYRAW

SEQ ID NO: 97 - protein, 21 aa, synthetic construct
  MOD_RES 5: note = D, E, gamma-carboxyglutamic acid or Aad
  MOD_RES 16: note = D, E, gamma-carboxyglutamic acid or Aad
  source 1..21: note = Synthetic peptide
LLALXLLLLPTTFLWXAYRAY

SEQ ID NO: 98 - protein, 21 aa, synthetic construct
  MOD_RES 5: note = D, E, gamma-carboxyglutamic acid or Aad
  MOD_RES 16: note = D, E, gamma-carboxyglutamic acid or Aad
  source 1..21: note = Synthetic peptide
LLGLXYLLLPTTFLWXSYRAW

SEQ ID NO: 99 - protein, 21 aa, synthetic construct
  MOD_RES 6: note = D, E, gamma-carboxyglutamic acid or Aad
  MOD_RES 16: note = D, E, gamma-carboxyglutamic acid or Aad
  source 1..21: note = Synthetic peptide
ALLALXYLLLPTTFWXTYRAW

SEQ ID NO: 100 - protein, 21 aa, synthetic construct
  MOD_RES 5: note = D, E, gamma-carboxyglutamic acid or Aad
  MOD_RES 16: note = D, E, gamma-carboxyglutamic acid or Aad
  source 1..21: note = Synthetic peptide
LLGLXYLLLPTTFLWXTYRAW

SEQ ID NO: 101 - protein, 21 aa, synthetic construct
  MOD_RES 5: note = D, E, gamma-carboxyglutamic acid or Aad
  MOD_RES 16: note = D, E, gamma-carboxyglutamic acid or Aad
  source 1..21: note = Synthetic peptide
LLSLXLLLLPTTFLWXAYRAW

SEQ ID NO: 102 - protein, 26 aa, synthetic construct
  MOD_RES 9: note = D, E, gamma-carboxyglutamic acid or Aad
  MOD_RES 20: note = D, E, gamma-carboxyglutamic acid or Aad
  source 1..26: note = Synthetic peptide
GLAGLLGLXGLLGLPLGLLXGLWLGL

SEQ ID NO: 103 - protein, 26 aa, synthetic construct
  MOD_RES 7: note = D, E, gamma-carboxyglutamic acid or Aad
  MOD_RES 18: note = D, E, gamma-carboxyglutamic acid or Aad
  source 1..26: note = Synthetic peptide
LGLWLGXLLGLPLGLLGXLGLLGALG

SEQ ID NO: 104 - protein, 20 aa, synthetic construct
  MOD_RES 6: note = D, E, gamma-carboxyglutamic acid or Aad
  MOD_RES 12: note = D, E, gamma-carboxyglutamic acid or Aad
  MOD_RES 17: note = D, E, gamma-carboxyglutamic acid or Aad
  source 1..20: note = Synthetic peptide
WRAYLXLLFPTXTLLLXLLW

SEQ ID NO: 105 - protein, 20 aa, synthetic construct
  MOD_RES 4: note = D, E, gamma-carboxyglutamic acid or Aad
  MOD_RES 9: note = D, E, gamma-carboxyglutamic acid or Aad
  MOD_RES 15: note = D, E, gamma-carboxyglutamic acid or Aad
  source 1..20: note = Synthetic peptide
WLLXLLLTXTPFLLXLYARW

SEQ ID NO: 106 - protein, 18 aa, synthetic construct
  MOD_RES 6: note = D, E, gamma-carboxyglutamic acid or Aad
  MOD_RES 12: note = D, E, gamma-carboxyglutamic acid or Aad
  MOD_RES 17: note = D, E, gamma-carboxyglutamic acid or Aad
  source 1..18: note = Synthetic peptide
WARYLXWLFPTXTLLLXL

SEQ ID NO: 107 - protein, 18 aa, synthetic construct
  MOD_RES 6: note = D, E, gamma-carboxyglutamic acid or Aad
  MOD_RES 12: note = D, E, gamma-carboxyglutamic acid or Aad
  MOD_RES 17: note = D, E, gamma-carboxyglutamic acid or Aad
  source 1..18: note = Synthetic peptide
WAQYLXLLFPTXTLLLXW

SEQ ID NO: 108 - protein, 18 aa, synthetic construct
  MOD_RES 2: note = D, E, gamma-carboxyglutamic acid or Aad
  MOD_RES 7: note = D, E, gamma-carboxyglutamic acid or Aad
  MOD_RES 13: note = D, E, gamma-carboxyglutamic acid or Aad
  source 1..18: note = Synthetic peptide
LXLLLTXTPFLWXLYRAW

SEQ ID NO: 109 - protein, 18 aa, synthetic construct
  MOD_RES 2: note = D, E, gamma-carboxyglutamic acid or Aad
  MOD_RES 7: note = D, E, gamma-carboxyglutamic acid or Aad
  MOD_RES 13: note = D, E, gamma-carboxyglutamic acid or Aad
  source 1..18: note = Synthetic peptide
WXLLLTXTPFLLXLYQAW

SEQ ID NO: 110 - protein, 17 aa, synthetic construct
  MOD_RES 17: note = D, E, gamma-carboxyglutamic acid or Aad
  source 1..17: note = Synthetic peptide
WLFTTPLLLLNGALLVX

SEQ ID NO: 111 - protein, 17 aa, synthetic construct
  MOD_RES 17: note = D, E, gamma-carboxyglutamic acid or Aad
  source 1..17: note = Synthetic peptide
WLFTTPLLLLPGALLVX

SEQ ID NO: 112 - protein, 15 aa, synthetic construct
  MOD_RES 6: note = D, E, gamma-carboxyglutamic acid or Aad
  source 1..15: note = Synthetic peptide
WARYAXLLFPTTLAW

SEQ ID NO: 113 - protein, 17 aa, synthetic construct
  MOD_RES 1: note = D, E, gamma-carboxyglutamic acid or Aad
  source 1..17: note = Synthetic peptide
XVLLAGNLLLLPTTFLW

SEQ ID NO: 114 - protein, 17 aa, synthetic construct
  MOD_RES 1: note = D, E, gamma-carboxyglutamic acid or Aad
  source 1..17: note = Synthetic peptide
XVLLAGPLLLLPTTFLW

SEQ ID NO: 115 - protein, 15 aa, synthetic construct
  MOD_RES 10: note = D, E, gamma-carboxyglutamic acid or Aad
  source 1..15: note = Synthetic peptide
WALTTPFLLXAYRAW

SEQ ID NO: 116 - protein, 24 aa, synthetic construct
  MOD_RES 3: note = D, E, gamma-carboxyglutamic acid or Aad
  MOD_RES 12: note = D, E, gamma-carboxyglutamic acid or Aad
  MOD_RES 24: note = D, E, gamma-carboxyglutamic acid or Aad
  source 1..24: note = Synthetic peptide
NLXGFFATLGGXIALWSLVVLAIX

SEQ ID NO: 117 - protein, 22 aa, synthetic construct
  MOD_RES 1: note = D, E, gamma-carboxyglutamic acid or Aad
  MOD_RES 10: note = D, E, gamma-carboxyglutamic acid or Aad
  MOD_RES 16: note = D, E, gamma-carboxyglutamic acid or Aad
  MOD_RES 22: note = D, E, gamma-carboxyglutamic acid or Aad
  source 1..22: note = Synthetic peptide
XGFFATLGGXIALWSXVVLAIX

SEQ ID NO: 118 - protein, 22 aa, synthetic construct
  MOD_RES 1: note = D, E, gamma-carboxyglutamic acid or Aad
  MOD_RES 10: note = D, E, gamma-carboxyglutamic acid or Aad
  MOD_RES 16: note = D, E, gamma-carboxyglutamic acid or Aad
  MOD_RES 22: note = D, E, gamma-carboxyglutamic acid or Aad
  source 1..22: note = Synthetic peptide
XGFFATLGGXIPLWSXVVLAIX

SEQ ID NO: 119 - protein, 24 aa, synthetic construct
  MOD_RES 1: note = D, E, gamma-carboxyglutamic acid or Aad
  MOD_RES 13: note = D, E, gamma-carboxyglutamic acid or Aad
  MOD_RES 22: note = D, E, gamma-carboxyglutamic acid or Aad
  source 1..24: note = Synthetic peptide
XIALVVLSWLAIXGGLTAFFGXLN

SEQ ID NO: 120 - protein, 22 aa, synthetic construct
  MOD_RES 1: note = D, E, gamma-carboxyglutamic acid or Aad
  MOD_RES 7: note = D, E, gamma-carboxyglutamic acid or Aad
  MOD_RES 13: note = D, E, gamma-carboxyglutamic acid or Aad
  MOD_RES 22: note = D, E, gamma-carboxyglutamic acid or Aad
  source 1..22: note = Synthetic peptide
XIALVVXSWLAIXGGLTAFFGX

SEQ ID NO: 121 - protein, 22 aa, synthetic construct
  MOD_RES 1: note = D, E, gamma-carboxyglutamic acid or Aad
  MOD_RES 7: note = D, E, gamma-carboxyglutamic acid or Aad
  MOD_RES 13: note = D, E, gamma-carboxyglutamic acid or Aad
  MOD_RES 22: note = D, E, gamma-carboxyglutamic acid or Aad
  source 1..22: note = Synthetic peptide
XIALVVXSWLPIXGGLTAFFGX

SEQ ID NO: 122 - protein, 21 aa, synthetic construct
  MOD_RES 3: note = D, E, gamma-carboxyglutamic acid or Aad
  MOD_RES 16: note = D, E, gamma-carboxyglutamic acid or Aad
  source 1..21: note = Synthetic peptide
ILXLVFGLLFAVTSVXFLVQW

SEQ ID NO: 123 - protein, 21 aa, synthetic construct
  MOD_RES 6: note = D, E, gamma-carboxyglutamic acid or Aad
  MOD_RES 19: note = D, E, gamma-carboxyglutamic acid or Aad
  source 1..21: note = Synthetic peptide
WQVLFXVSTVAFLLGFVLXLI

SEQ ID NO: 124-131 - skipped (8 entries)

SEQ ID NO: 132 - protein, 26 aa, synthetic construct
  MOD_RES 2..3: note = A, I, L, M, F, P, W, Y, V, or G
  MOD_RES 5..6: note = A, I, L, M, F, P, W, Y, V, or G
  MOD_RES 8: note = A, I, L, M, F, P, W, Y, V, or G
  MOD_RES 9: note = D, E, gamma-carboxyglutamic acid or Aad
  MOD_RES 11..12: note = A, I, L, M, F, P, W, Y, V, or G
  MOD_RES 14..16: note = A, I, L, M, F, P, W, Y, V, or G
  MOD_RES 18..19: note = A, I, L, M, F, P, W, Y, V, or G
  MOD_RES 20: note = D, E, gamma-carboxyglutamic acid or Aad
  MOD_RES 22..24: note = A, I, L, M, F, P, W, Y, V, or G
  MOD_RES 26: note = A, I, L, M, F, P, W, Y, V, or G
  source 1..26: note = Synthetic peptide
GXXGXXGXXGXXGXXXGXXXGXXXGX

SEQ ID NO: 133 - protein, 26 aa, synthetic construct
  MOD_RES 1: note = A, I, L, M, F, P, W, Y, V, or G
  MOD_RES 3..5: note = A, I, L, M, F, P, W, Y, V, or G
  MOD_RES 7: note = D, E, gamma-carboxyglutamic acid or Aad
  MOD_RES 8..9: note = A, I, L, M, F, P, W, Y, V, or G
  MOD_RES 11..13: note = A, I, L, M, F, P, W, Y, V, or G
  MOD_RES 15..16: note = A, I, L, M, F, P, W, Y, V, or G
  MOD_RES 18: note = D, E, gamma-carboxyglutamic acid or Aad
  MOD_RES 19: note = A, I, L, M, F, P, W, Y, V, or G
  MOD_RES 21..22: note = A, I, L, M, F, P, W, Y, V, or G
  MOD_RES 24: note = A, I, L, M, F, P, W, Y, V, or G
  source 1..26: note = Synthetic peptide
XGXXXGXXXGXXXGXXGXXGXXGXXG

SEQ ID NO: 134-145 - skipped (12 entries)

SEQ ID NO: 146 - protein, 25 aa, synthetic construct
  MOD_RES 3: note = A, I, L, M, F, P, W, Y, V, or G
  MOD_RES 4: note = D, E, gamma-carboxyglutamic acid or Aad
  MOD_RES 6..8: note = A, I, L, M, F, P, W, Y, V, or G
  MOD_RES 9: note = S, T or G
  MOD_RES 10: note = A, I, L, M, F, P, W, Y, V, or G
  MOD_RES 13: note = D, E, gamma-carboxyglutamic acid or Aad
  MOD_RES 14..17: note = A, I, L, M, F, P, W, Y, V, or G
  MOD_RES 18: note = S, T, or G
  MOD_RES 19..24: note = A, I, L, M, F, P, W, Y, V, or G
  MOD_RES 25: note = D, E, gamma-carboxyglutamic acid or Aad
  source 1..25: note = Synthetic peptide
GNXXGXXXXXGGXXXXXXXXXXXXX

SEQ ID NO: 147-148 - skipped (2 entries)

SEQ ID NO: 149 - protein, 25 aa, synthetic construct
  MOD_RES 1: note = D, E, gamma-carboxyglutamic acid or Aad
  MOD_RES 2..7: note = A, I, L, M, F, P, W, Y, V, or G
  MOD_RES 8: note = S, T, or G
  MOD_RES 9..12: note = A, I, L, M, F, P, W, Y, V, or G
  MOD_RES 13: note = D, E, gamma-carboxyglutamic acid or Aad
  MOD_RES 16: note = A, I, L, M, F, P, W, Y, V, or G
  MOD_RES 17: note = S, T, or G
  MOD_RES 18..20: note = A, I, L, M, F, P, W, Y, V, or G
  MOD_RES 22: note = D, E, gamma-carboxyglutamic acid or Aad
  MOD_RES 23: note = A, I, L, M, F, P, W, Y, V, or G
  source 1..25: note = Synthetic Peptide
XXXXXXXXXXXXXGGXXXXXGXXNG

SEQ ID NO: 150-153 - skipped (4 entries)

SEQ ID NO: 154 - protein, 34 aa, synthetic construct
  source 1..34: note = Synthetic polypeptide
AEQNPIYWARYAEWLFTTPLLLLELALLVEAEET

SEQ ID NO: 155 - protein, 33 aa, synthetic construct
  source 1..33: note = Synthetic polypeptide
ADDQNPWRAYLDLLFPDTTDLLLLDLLWDADET

SEQ ID NO: 156 - protein, 33 aa, synthetic construct
  source 1..33: note = Synthetic polypeptide
AEEQNPWRAYLELLFPETTELLLLELLWEAEET

SEQ ID NO: 157 - protein, 34 aa, synthetic construct
  MOD_RES 13: note = Gamma-carboxyglutamic acid
  MOD_RES 24: note = Gamma-carboxyglutamic acid
  source 1..34: note = Synthetic polypeptide
AEQNPIYWARYAXWLFTTPLLLLXLALLVDADET

SEQ ID NO: 158 - protein, 34 aa, synthetic construct; identical to SEQ ID NO: 157
  MOD_RES 13: note = Aad
  MOD_RES 24: note = Aad
  source 1..34: note = Synthetic polypeptide

SEQ ID NO: 159 - protein, 34 aa, synthetic construct; identical to SEQ ID NO: 157
  MOD_RES 13: note = Aad
  MOD_RES 24: note = Gamma-carboxyglutamic acid
  source 1..34: note = Synthetic polypeptide

SEQ ID NO: 160 - protein, 33 aa, synthetic construct
  source 1..33: note = Synthetic polypeptide
CEQNPIYWARYADWHFTTPLLLLDLALLVDADE

SEQ ID NO: 161 - protein, 32 aa, synthetic construct
  source 1..32: note = Synthetic polypeptide
ADNNPWIYARYADLTTFPLLLLDLALLVDFDD

SEQ ID NO: 162 - protein, 32 aa, synthetic construct
  source 1..32: note = Synthetic polypeptide
ADNNPFIYARYADLTTWPLLLLDLALLVDFDD

SEQ ID NO: 163 - protein, 32 aa, synthetic construct
  source 1..32: note = Synthetic polypeptide
ADNNPFIYARYADLTTFPLLLLDLALLVDWDD

SEQ ID NO: 164 - protein, 32 aa, synthetic construct
  source 1..32: note = Synthetic polypeptide
ADNNPFPYARYADLTTWILLLLDLALLVDFDD

SEQ ID NO: 165 - protein, 32 aa, synthetic construct
  source 1..32: note = Synthetic polypeptide
ADNNPFIYAYRADLTTFPLLLLDLALLVDWDD

SEQ ID NO: 166 - protein, 32 aa, synthetic construct
  source 1..32: note = Synthetic polypeptide
ADNNPFIYATYADLRTFPLLLLDLALLVDWDD

SEQ ID NO: 167 - protein, 30 aa, synthetic construct
  source 1..30: note = Synthetic polypeptide
ADDQNPWRAYLDLLFPTDTLLLDLLWDADE

SEQ ID NO: 168 - protein, 26 aa, synthetic construct
  MOD_RES 18: note = Gamma-carboxyglutamic acid
  source 1..26: note = Synthetic peptide
ADDQNPWRAYLDLLFPTXTLLLDLLW

SEQ ID NO: 169 - protein, 26 aa, synthetic construct
  MOD_RES 23: note = Gamma-carboxyglutamic acid
  source 1..26: note = Synthetic peptide
ADDQNPWRAYLDLLFPTDTLLLXLLW

SEQ ID NO: 170 - protein, 26 aa, synthetic construct
  MOD_RES 12: note = Gamma-carboxyglutamic acid
  MOD_RES 18: note = Gamma-carboxyglutamic acid
  source 1..26: note = Synthetic peptide
ADDQNPWRAYLXLLFPTXTLLLDLLW

SEQ ID NO: 171 - protein, 26 aa, synthetic construct
  MOD_RES 12: note = Gamma-carboxyglutamic acid
  MOD_RES 23: note = Gamma-carboxyglutamic acid
  source 1..26: note = Synthetic peptide
ADDQNPWRAYLXLLFPTDTLLLXLLW

SEQ ID NO: 172 - protein, 26 aa, synthetic construct
  MOD_RES 18: note = Gamma-carboxyglutamic acid
  MOD_RES 23: note = Gamma-carboxyglutamic acid
  source 1..26: note = Synthetic peptide
ADDQNPWRAYLDLLFPTXTLLLXLLW

SEQ ID NO: 173 - protein, 26 aa, synthetic construct
  MOD_RES 12: note = Gamma-carboxyglutamic acid
  MOD_RES 18: note = Gamma-carboxyglutamic acid
  MOD_RES 23: note = Gamma-carboxyglutamic acid
  source 1..26: note = Synthetic peptide
ADDQNPWRAYLXLLFPTXTLLLXLLW

SEQ ID NO: 174 - protein, 26 aa, synthetic construct
  MOD_RES 12: note = Aad
  source 1..26: note = Synthetic peptide
ADDQNPWRAYLXLLFPTDTLLLDLLW

SEQ ID NO: 175 - protein, 26 aa, synthetic construct
  MOD_RES 18: note = Aad
  source 1..26: note = Synthetic peptide
ADDQNPWRAYLDLLFPTXTLLLDLLW

SEQ ID NO: 176 - protein, 26 aa, synthetic construct
  MOD_RES 23: note = Aad
  source 1..26: note = Synthetic peptide
ADDQNPWRAYLDLLFPTDTLLLXLLW

SEQ ID NO: 177 - protein, 26 aa, synthetic construct
  MOD_RES 12: note = Aad
  MOD_RES 18: note = Aad
  source 1..26: note = Synthetic peptide
ADDQNPWRAYLXLLFPTXTLLLDLLW

SEQ ID NO: 178 - protein, 26 aa, synthetic construct
  MOD_RES 12: note = Aad
  MOD_RES 23: note = Aad
  source 1..26: note = Synthetic peptide
ADDQNPWRAYLXLLFPTDTLLLXLLW

SEQ ID NO: 179 - protein, 26 aa, synthetic construct
  MOD_RES 18: note = Aad
  MOD_RES 23: note = Aad
  source 1..26: note = Synthetic peptide
ADDQNPWRAYLDLLFPTXTLLLXLLW

SEQ ID NO: 180 - protein, 26 aa, synthetic construct
  MOD_RES 12: note = Aad
  MOD_RES 18: note = Aad
  MOD_RES 23: note = Aad
  source 1..26: note = Synthetic peptide
ADDQNPWRAYLXLLFPTXTLLLXLLW

SEQ ID NO: 181 - protein, 26 aa, synthetic construct
  MOD_RES 12: note = Gamma-carboxyglutamic acid
  MOD_RES 18: note = Aad
  source 1..26: note = Synthetic peptide
ADDQNPWRAYLXLLFPTXTLLLDLLW

SEQ ID NO: 182 - protein, 26 aa, synthetic construct
  MOD_RES 12: note = Gamma-carboxyglutamic acid
  MOD_RES 23: note = Aad
  source 1..26: note = Synthetic peptide
ADDQNPWRAYLXLLFPTDTLLLXLLW

SEQ ID NO: 183 - protein, 26 aa, synthetic construct
  MOD_RES 12: note = Gamma-carboxyglutamic acid
  MOD_RES 18: note = Gamma-carboxyglutamic acid
  MOD_RES 23: note = Aad
  source 1..26: note = Synthetic peptide
ADDQNPWRAYLXLLFPTXTLLLXLLW

SEQ ID NO: 184 - protein, 26 aa, synthetic construct
  MOD_RES 12: note = Aad
  MOD_RES 18: note = Gamma-carboxyglutamic acid
  source 1..26: note = Synthetic peptide
ADDQNPWRAYLXLLFPTXTLLLDLLW

SEQ ID NO: 185 - protein, 26 aa, synthetic construct
  MOD_RES 12: note = Aad
  MOD_RES 23: note = Gamma-carboxyglutamic acid
  source 1..26: note = Synthetic peptide
ADDQNPWRAYLXLLFPTDTLLLXLLW

SEQ ID NO: 186 - protein, 26 aa, synthetic construct
  MOD_RES 12: note = Gamma-carboxyglutamic acid
  MOD_RES 18: note = Aad
  MOD_RES 23: note = Gamma-carboxyglutamic acid
  source 1..26: note = Synthetic peptide
ADDQNPWRAYLXLLFPTXTLLLXLLW

SEQ ID NO: 187 - protein, 29 aa, synthetic construct
  REGION 1..29: note = Description of Artificial Sequence: Synthetic peptide
GEEQNPWLGAYLDLLFPLELLGLLELGLW

SEQ ID NO: 188 - protein, 32 aa, synthetic construct
  source 1..32: note = Synthetic polypeptide
EQNPIYILDLVFGLLFAVTSVDFLVQWDDAGD

SEQ ID NO: 189 - protein, 24 aa, synthetic construct
  source 1..24: note = Synthetic peptide
NNEGFFATLGGEIALWSDVVLAIE

SEQ ID NO: 190 - protein, 25 aa, synthetic construct
  source 1..25: note = Synthetic peptide
DNNEGFFATLGGEIPLWSDVVLAIE

SEQ ID NO: 191 - protein, 34 aa, synthetic construct
  MOD_RES 2: note = D, E, gamma-carboxyglutamic acid or Aad
  MOD_RES 13: note = D, E, gamma-carboxyglutamic acid or Aad
  MOD_RES 24: note = D, E, gamma-carboxyglutamic acid or Aad
  MOD_RES 30: note = D, E, gamma-carboxyglutamic acid or Aad
  MOD_RES 32: note = D, E, gamma-carboxyglutamic acid or Aad
  MOD_RES 33: note = D, E, gamma-carboxyglutamic acid or Aad
  source 1..34: note = Synthetic polypeptide
AXQNPIYWARYAXWLFTTPLLLLXLALLVXAXXT

SEQ ID NO: 192 - protein, 33 aa, synthetic construct
  REGION 1..33: note = Description of Artificial Sequence: Synthetic polypeptide
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 17: note = D, E, gamma-carboxyglutamic acid or Aad
  MOD_RES 20: note = D, E, gamma-carboxyglutamic acid or Aad
  MOD_RES 25: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
AXXQNPWRAYLXLLFPXTTXLLLLXLLWXAXXT

SEQ ID NO: 193 - protein, 33 aa, synthetic construct; identical to SEQ ID NO: 192
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 17: note = D, E, gamma-carboxyglutamic acid or Aad
  MOD_RES 20: note = D, E, gamma-carboxyglutamic acid or Aad
  MOD_RES 25: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..33: note = Synthetic polypeptide

SEQ ID NO: 194 - protein, 34 aa, synthetic construct; identical to SEQ ID NO: 191
  MOD_RES 2: note = D, E, gamma-carboxyglutamic acid or Aad
  MOD_RES 13: note = D, E, gamma-carboxyglutamic acid or Aad
  MOD_RES 24: note = D, E, gamma-carboxyglutamic acid or Aad
  MOD_RES 30: note = D, E, gamma-carboxyglutamic acid or Aad
  MOD_RES 32: note = D, E, gamma-carboxyglutamic acid or Aad
  MOD_RES 33: note = D, E, gamma-carboxyglutamic acid or Aad
  source 1..34: note = Synthetic polypeptide

SEQ ID NO: 195 - protein, 34 aa, synthetic construct; identical to SEQ ID NO: 191
  MOD_RES 2: note = D, E, gamma-carboxyglutamic acid or Aad
  MOD_RES 13: note = D, E, gamma-carboxyglutamic acid or Aad
  MOD_RES 24: note = D, E, gamma-carboxyglutamic acid or Aad
  MOD_RES 30: note = D, E, gamma-carboxyglutamic acid or Aad
  MOD_RES 32: note = D, E, gamma-carboxyglutamic acid or Aad
  MOD_RES 33: note = D, E, gamma-carboxyglutamic acid or Aad
  source 1..34: note = Synthetic polypeptide

SEQ ID NO: 196 - protein, 34 aa, synthetic construct; identical to SEQ ID NO: 191
  MOD_RES 2: note = D, E, gamma-carboxyglutamic acid or Aad
  MOD_RES 13: note = D, E, gamma-carboxyglutamic acid or Aad
  MOD_RES 24: note = D, E, gamma-carboxyglutamic acid or Aad
  MOD_RES 30: note = D, E, gamma-carboxyglutamic acid or Aad
  MOD_RES 32: note = D, E, gamma-carboxyglutamic acid or Aad
  MOD_RES 33: note = D, E, gamma-carboxyglutamic acid or Aad
  source 1..34: note = Synthetic polypeptide

SEQ ID NO: 197 - protein, 33 aa, synthetic construct
  MOD_RES 2: note = D, E, gamma-carboxyglutamic acid or Aad
  MOD_RES 13: note = D, E, gamma-carboxyglutamic acid or Aad
  MOD_RES 24: note = D, E, gamma-carboxyglutamic acid or Aad
  MOD_RES 30: note = D, E, gamma-carboxyglutamic acid or Aad
  MOD_RES 32: note = D, E, gamma-carboxyglutamic acid or Aad
  MOD_RES 33: note = D, E, gamma-carboxyglutamic acid or Aad
  source 1..33: note = Synthetic polypeptide
CXQNPIYWARYAXWHFTTPLLLLXLALLVXAXX

SEQ ID NO: 198 - protein, 32 aa, synthetic construct
  MOD_RES 2: note = D, E, gamma-carboxyglutamic acid or Aad
  MOD_RES 13: note = D, E, gamma-carboxyglutamic acid or Aad
  MOD_RES 23: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..32: note = Synthetic polypeptide
AXNNPWIYARYAXLTTFPLLLLXLALLVXFXX

SEQ ID NO: 199 - protein, 32 aa, synthetic construct
  MOD_RES 2: note = D, E, gamma-carboxyglutamic acid or Aad
  MOD_RES 13: note = D, E, gamma-carboxyglutamic acid or Aad
  MOD_RES 23: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..32: note = Synthetic polypeptide
AXNNPFIYARYAXLTTWPLLLLXLALLVXFXX

SEQ ID NO: 200 - protein, 32 aa, synthetic construct
  MOD_RES 2: note = D, E, gamma-carboxyglutamic acid or Aad
  MOD_RES 13: note = D, E, gamma-carboxyglutamic acid or Aad
  MOD_RES 23: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..32: note = Synthetic polypeptide
AXNNPFIYARYAXLTTFPLLLLXLALLVXWXX

SEQ ID NO: 201 - protein, 32 aa, synthetic construct
  MOD_RES 2: note = D, E, gamma-carboxyglutamic acid or Aad
  MOD_RES 13: note = D, E, gamma-carboxyglutamic acid or Aad
  MOD_RES 23: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..32: note = Synthetic polypeptide
AXNNPFPYARYAXLTTWILLLLXLALLVXFXX

SEQ ID NO: 202 - protein, 32 aa, synthetic construct
  MOD_RES 2: note = D, E, gamma-carboxyglutamic acid or Aad
  MOD_RES 13: note = D, E, gamma-carboxyglutamic acid or Aad
  MOD_RES 23: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..32: note = Synthetic polypeptide
AXNNPFIYAYRAXLTTFPLLLLXLALLVXWXX

SEQ ID NO: 203 - protein, 32 aa, synthetic construct
  MOD_RES 2: note = D, E, gamma-carboxyglutamic acid or Aad
  MOD_RES 13: note = D, E, gamma-carboxyglutamic acid or Aad
  MOD_RES 23: note = D, E, gamma-carboxyglutamic acid or Aad
  MOD_RES 29: note = D, E, gamma-carboxyglutamic acid or Aad
  MOD_RES 31: note = D, E, gamma-carboxyglutamic acid or Aad
  MOD_RES 32: note = D, E, gamma-carboxyglutamic acid or Aad
  source 1..32: note = Synthetic polypeptide
AXNNPFIYATYAXLRTFPLLLLXLALLVXWXX

SEQ ID NO: 204 - protein, 30 aa, synthetic construct
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 18: note = D, E, gamma-carboxyglutamic acid or Aad
  MOD_RES 23: note = D, E, gamma-carboxyglutamic acid or Aad
  MOD_RES 27: note = D, E, gamma-carboxyglutamic acid or Aad
  MOD_RES 29: note = D, E, gamma-carboxyglutamic acid or Aad
  MOD_RES 30: note = D, E, gamma-carboxyglutamic acid or Aad
  source 1..30: note = Synthetic polypeptide
AXXQNPWRAYLXLLFPTXTLLLXLLWXAXX

SEQ ID NO: 205 - protein, 26 aa, synthetic construct
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 18: note = D, E, gamma-carboxyglutamic acid or Aad
  MOD_RES 23: note = D, E, gamma-carboxyglutamic acid or Aad
  source 1..26: note = Synthetic peptide
AXXQNPWRAYLXLLFPTXTLLLXLLW

SEQ ID NO: 206 - protein, 25 aa, synthetic construct
  MOD_RES 1: note = D, E, gamma-carboxyglutamic acid or Aad
  MOD_RES 2: note = D, E, gamma-carboxyglutamic acid or Aad
  MOD_RES 11: note = D, E, gamma-carboxyglutamic acid or Aad
  MOD_RES 17: note = D, E, gamma-carboxyglutamic acid or Aad
  MOD_RES 22: note = D, E, gamma-carboxyglutamic acid or Aad
  source 1..25: note = Synthetic peptide
XXQNPWRAYLXLLFPTXTLLLXLLW

SEQ ID NO: 207 - protein, 26 aa, synthetic construct
  MOD_RES 12: note = D, E, gamma-carboxyglutamic acid or Aad
  MOD_RES 18: note = D, E, gamma-carboxyglutamic acid or Aad
  MOD_RES 23: note = D, E, gamma-carboxyglutamic acid or Aad
  source 1..26: note = Synthetic peptide
ANNPFIYATYAXLLFPTXTLLLXLLW

SEQ ID NO: 208 - protein, 25 aa, synthetic construct
  MOD_RES 11: note = D, E, gamma-carboxyglutamic acid or Aad
  MOD_RES 17: note = D, E, gamma-carboxyglutamic acid or Aad
  MOD_RES 22: note = D, E, gamma-carboxyglutamic acid or Aad
  source 1..25: note = Synthetic peptide
NNPFIYATYAXLLFPTXTLLLXLLW

SEQ ID NO: 209 - protein, 25 aa, synthetic construct
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 22: note = D, E, gamma-carboxyglutamic acid or Aad
  source 1..25: note = Synthetic peptide
AXXQNPWRAYLXLRTFPLLLLXLAW

SEQ ID NO: 210 - protein, 24 aa, synthetic construct
  MOD_RES 1: note = D, E, gamma-carboxyglutamic acid or Aad
  MOD_RES 2: note = D, E, gamma-carboxyglutamic acid or Aad
  MOD_RES 11: note = D, E, gamma-carboxyglutamic acid or Aad
  MOD_RES 21: note = D, E, gamma-carboxyglutamic acid or Aad
  source 1..24: note = Synthetic peptide
XXQNPWRAYLXLRTFPLLLLXLAW

SEQ ID NO: 211 - protein, 26 aa, synthetic construct
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 22: note = D, E, gamma-carboxyglutamic acid or Aad
  source 1..26: note = Synthetic peptide
AXXQNPWRAYLXLRTFPLLLLXLALL

SEQ ID NO: 212 - protein, 25 aa, synthetic construct
  MOD_RES 1: note = D, E, gamma-carboxyglutamic acid or Aad
  MOD_RES 2: note = D, E, gamma-carboxyglutamic acid or Aad
  MOD_RES 11: note = D, E, gamma-carboxyglutamic acid or Aad
  MOD_RES 21: note = D, E, gamma-carboxyglutamic acid or Aad
  source 1..25: note = Synthetic peptide
XXQNPWRAYLXLRTFPLLLLXLALL

SEQ ID NO: 213 - protein, 25 aa, synthetic construct
  MOD_RES 11: note = D, E, gamma-carboxyglutamic acid or Aad
  MOD_RES 17: note = D, E, gamma-carboxyglutamic acid or Aad
  MOD_RES 22: note = D, E, gamma-carboxyglutamic acid or Aad
  source 1..25: note = Synthetic peptide
ANPIYWARYAXLLFPTXTLLLXLLW

SEQ ID NO: 214 - protein, 24 aa, synthetic construct
  MOD_RES 10: note = D, E, gamma-carboxyglutamic acid or Aad
  MOD_RES 16: note = D, E, gamma-carboxyglutamic acid or Aad
  MOD_RES 21: note = D, E, gamma-carboxyglutamic acid or Aad
  source 1..24: note = Synthetic peptide
NPIYWARYAXLLFPTXTLLLXLLW

SEQ ID NO: 215 - protein, 28 aa, synthetic construct
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 23: note = D, E, gamma-carboxyglutamic acid or Aad
  source 1..28: note = Synthetic peptide
AXXQNPWRAYLXWLFTTPLLLLXLALLV

SEQ ID NO: 216 - protein, 27 aa, synthetic construct
  MOD_RES 1: note = D, E, gamma-carboxyglutamic acid or Aad
  MOD_RES 2: note = D, E, gamma-carboxyglutamic acid or Aad
  MOD_RES 11: note = D, E, gamma-carboxyglutamic acid or Aad
  MOD_RES 22: note = D, E, gamma-carboxyglutamic acid or Aad
  source 1..27: note = Synthetic peptide
XXQNPWRAYLXWLFTTPLLLLXLALLV

SEQ ID NO: 217 - protein, 29 aa, synthetic construct
  MOD_RES 2: note = D, E, gamma-carboxyglutamic acid or Aad
  MOD_RES 13: note = D, E, gamma-carboxyglutamic acid or Aad
  MOD_RES 24: note = D, E, gamma-carboxyglutamic acid or Aad
  source 1..29: note = Synthetic peptide
CXQNPIYWARYAXWHFTTPLLLLXLALLV

SEQ ID NO: 218 - protein, 28 aa, synthetic construct
  MOD_RES 1: note = D, E, gamma-carboxyglutamic acid or Aad
  MOD_RES 12: note = D, E, gamma-carboxyglutamic acid or Aad
  MOD_RES 23: note = D, E, gamma-carboxyglutamic acid or Aad
  source 1..28: note = Synthetic peptide
XQNPIYWARYAXWHFTTPLLLLXLALLV

SEQ ID NO: 219 - protein, 26 aa, synthetic construct
  MOD_RES 2: note = D, E, gamma-carboxyglutamic acid or Aad
  MOD_RES 3: note = D, E, gamma-carboxyglutamic acid or Aad
  MOD_RES 12: note = D, E, gamma-carboxyglutamic acid or Aad
  MOD_RES 18: note = D, E, gamma-carboxyglutamic acid or Aad
  MOD_RES 23: note = D, E, gamma-carboxyglutamic acid or Aad
  source 1..26: note = Synthetic peptide
AXXQNPWRAYLXLLFPTXTLLLXLLV

SEQ ID NO: 220 - protein, 25 aa, synthetic construct
  MOD_RES 1: note = D, E, gamma-carboxyglutamic acid or Aad
  MOD_RES 2: note = D, E, gamma-carboxyglutamic acid or Aad
  MOD_RES 11: note = D, E, gamma-carboxyglutamic acid or Aad
  MOD_RES 17: note = D, E, gamma-carboxyglutamic acid or Aad
  MOD_RES 22: note = D, E, gamma-carboxyglutamic acid or Aad
  source 1..25: note = Synthetic peptide
XXQNPWRAYLXLLFPTXTLLLXLLV

SEQ ID NO: 221 - protein, 27 aa, synthetic construct
  MOD_RES 2: note = D, E, gamma-carboxyglutamic acid or Aad
  MOD_RES 13: note = D, E, gamma-carboxyglutamic acid or Aad
  MOD_RES 19: note = D, E, gamma-carboxyglutamic acid or Aad
  MOD_RES 24: note = D, E, gamma-carboxyglutamic acid or Aad
  source 1..27: note = Synthetic peptide
AXQNPIYWARYAXLLFPTXTLLLXLLW

SEQ ID NO: 222 - protein, 26 aa, synthetic construct
  MOD_RES 1: note = D, E, gamma-carboxyglutamic acid or Aad
  MOD_RES 12: note = D, E, gamma-carboxyglutamic acid or Aad
  MOD_RES 18: note = D, E, gamma-carboxyglutamic acid or Aad
  MOD_RES 23: note = D, E, gamma-carboxyglutamic acid or Aad
  source 1..26: note = Synthetic peptide
XQNPIYWARYAXLLFPTXTLLLXLLW

SEQ ID NO: 223 - protein, 27 aa, synthetic construct
  MOD_RES 2: note = D, E, gamma-carboxyglutamic acid or Aad
  MOD_RES 13: note = D, E, gamma-carboxyglutamic acid or Aad
  MOD_RES 19: note = D, E, gamma-carboxyglutamic acid or Aad
  MOD_RES 24: note = D, E, gamma-carboxyglutamic acid or Aad
  source 1..27: note = Synthetic peptide
AXQNPIYWARYLXLLFPTXTLLLXLLW

SEQ ID NO: 224 - protein, 26 aa, synthetic construct
  MOD_RES 1: note = D, E, gamma-carboxyglutamic acid or Aad
  MOD_RES 12: note = D, E, gamma-carboxyglutamic acid or Aad
  MOD_RES 18: note = D, E, gamma-carboxyglutamic acid or Aad
  MOD_RES 23: note = D, E, gamma-carboxyglutamic acid or Aad
  source 1..26: note = Synthetic peptide
XQNPIYWARYLXLLFPTXTLLLXLLW

SEQ ID NO: 225 - protein, 29 aa, synthetic construct
  MOD_RES 2: note = D, E, gamma-carboxyglutamic acid or Aad
  MOD_RES 3: note = D, E, gamma-carboxyglutamic acid or Aad
  MOD_RES 13: note = D, E, gamma-carboxyglutamic acid or Aad
  MOD_RES 19: note = D, E, gamma-carboxyglutamic acid or Aad
  MOD_RES 25: note = D, E, gamma-carboxyglutamic acid or Aad
  source 1..29: note = Synthetic peptide
GXXQNPWLGAYLXLLFPLXLLGLLXLGLW

SEQ ID NO: 226 - protein, 32 aa, synthetic construct
  MOD_RES 1: note = D, E, gamma-carboxyglutamic acid or Aad
  MOD_RES 9: note = D, E, gamma-carboxyglutamic acid or Aad
  MOD_RES 22: note = D, E, gamma-carboxyglutamic acid or Aad
  MOD_RES 28: note = D, E, gamma-carboxyglutamic acid or Aad
  MOD_RES 29: note = D, E, gamma-carboxyglutamic acid or Aad
  MOD_RES 32: note = D, E, gamma-carboxyglutamic acid or Aad
  source 1..32: note = Synthetic polypeptide
XQNPIYILXLVFGLLFAVTSVXFLVQWXXAGX

SEQ ID NO: 227 - protein, 24 aa, synthetic construct
  MOD_RES 3: note = D, E, gamma-carboxyglutamic acid or Aad
  MOD_RES 12: note = D, E, gamma-carboxyglutamic acid or Aad
  MOD_RES 18: note = D, E, gamma-carboxyglutamic acid or Aad
  MOD_RES 24: note = D, E, gamma-carboxyglutamic acid or Aad
  source 1..24: note = Synthetic peptide
NNXGFFATLGGXIALWSXVVLAIX

SEQ ID NO: 228 - protein, 25 aa, synthetic construct
  MOD_RES 1: note = D, E, gamma-carboxyglutamic acid or Aad
  MOD_RES 4: note = D, E, gamma-carboxyglutamic acid or Aad
  MOD_RES 13: note = D, E, gamma-carboxyglutamic acid or Aad
  MOD_RES 19: note = D, E, gamma-carboxyglutamic acid or Aad
  MOD_RES 25: note = D, E, gamma-carboxyglutamic acid or Aad
  source 1..25: note = Synthetic peptide
XNNXGFFATLGGXIPLWSXVVLAIX

SEQ ID NO: 229-230 - skipped (2 entries)

SEQ ID NO: 231 - protein, 33 aa, synthetic construct
  MOD_RES 2: note = D, E, gamma-carboxyglutamic acid or Aad
  MOD_RES 5..9: note = A, I, L, M, F, P, W, Y, V, or G
  MOD_RES 11..12: note = A, I, L, M, F, P, W, Y, V, or G
  MOD_RES 13: note = D, E, gamma-carboxyglutamic acid or Aad
  MOD_RES 14: note = A, I, L, M, F, P, W, Y, V, or G
  MOD_RES 16: note = A, I, L, M, F, P, W, Y, V, or G
  MOD_RES 17..18: note = S, T, or G
  MOD_RES 19..23: note = A, I, L, M, F, P, W, Y, V, or G
  MOD_RES 24: note = D, E, gamma-carboxyglutamic acid or Aad
  MOD_RES 25..29: note = A, I, L, M, F, P, W, Y, V, or G
  MOD_RES 30: note = D, E, gamma-carboxyglutamic acid or Aad
  MOD_RES 31: note = A, I, L, M, F, P, W, Y, V, or G
  MOD_RES 32..33: note = D, E, gamma-carboxyglutamic acid or Aad
  source 1..33: note = Synthetic polypeptide
CXQNXXXXXRXXXXHXXXXXXXXXXXXXXXXXX

SEQ ID NO: 232-240 - skipped (9 entries)

SEQ ID NO: 241 - protein, 27 aa, synthetic construct
  source 1..27: note = Synthetic peptide
ACDDQNPWRAYLDLLFPTDTLLLDLLW

SEQ ID NO: 242 - protein, 25 aa, synthetic construct
  source 1..25: note = Synthetic peptide
ACDDQNPWARYLDWLFPTDTLLLDL

SEQ ID NO: 243 - protein, 24 aa, synthetic construct
  source 1..24: note = Synthetic peptide
CDNNNPWRAYLDLLFPTDTLLLDW

SEQ ID NO: 244 - protein, 25 aa, synthetic construct
  source 1..25: note = Synthetic peptide
ACEEQNPWARYLEWLFPTETLLLEL

SEQ ID NO: 245 - protein, 27 aa, synthetic construct
  source 1..27: note = Synthetic peptide
ACEEQNPWRAYLELLFPTETLLLELLW

SEQ ID NO: 246 - protein, 24 aa, synthetic construct
  source 1..24: note = Synthetic peptide
CEEQQPWAQYLELLFPTETLLLEW

SEQ ID NO: 247 - protein, 24 aa, synthetic construct
  source 1..24: note = Synthetic peptide
CEEQQPWRAYLELLFPTETLLLEW

SEQ ID NO: 248 - protein, 25 aa, synthetic construct
  source 1..25: note = Synthetic peptide
AAEEQNPWARYLEWLFPTETLLLEL

SEQ ID NO: 249 - DNA, 6706 bp, Homo sapiens
  source 1..6706: mol_type = other DNA
agagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtatgagccgggtgtgggtggggtgtgcaggagcccgggagcatggaggggtctgggagagtcccgggcttggcggtggtggctgagaggccttctcccttctcctgtcctgtcaatgttatccaaagccctcatatattcagtcaacaaacaccattcatggtgatagccgggctgctgtttgtgcagggctggcactgaacactgccttgatcttatttggagcaatatgcgcttgtctaatttcacagcaagaaaactgagctgaggctcaaagaagtcaagcgccctgctggggcgtcacacagggacgggtgcagagttgagttggaagcccgcatctatctcgggccatgtttgcagcaccaagcctctgtttcccttggagcagctgtgctgagtcagacccaggctgggcactgagggagagctgggcaagccagacccctcctctctgggggcccaagctcagggtgggaagtggattttccattctccagtcattgggtcttccctgtgctgggcaatgggctcggtcccctctggcatcctctgcctcccctctcagcccctgtcctcaggtgcccctccagcctccctgccgcgttccaagtctcctggtgttgagaaccgcaagcagccgctctgaagcagttcctttttgctttagaataatgtcttgcatttaacaggaaaacagatggggtgctgcagggataacagatcccacttaacagagaggaaaactgaggcagggagaggggaagagactcatttagggatgtggccaggcagcaacaagagcctaggtctcctggctgtgatccaggaatatctctgctgagatgcaggaggagacgctagaagcagccattgcaaagctgggtgacggggagagcttaccgccagccacaagcgtctctctgccagccttgccctgtctcccccatgtccaggctgctgcctcggtcccattctcagggaatctctggccattgttgggtgtttgttgcattcaataatcacagatcactcagttctggccagaaggtgggtgtgccacttacgggtggttgttctctgcagggtcagtcccagtttacaaatattgtccctttcactgttaggaatgtcccagtttggttgattaactatatggccactctccctatggaacttcatggggtggtgagcaggacagatgtctgaattccatcatttccttcttcttcctctgggcaaaacattgcacattgcttcatggctcctaggagaggcccccacatgtccgggttatttcatttcccgagaagggagagggaggaaggactgccaattctgggtttccaccacctctgcattccttcccaacaaggaactctgccccacattaggatgcattcttctgctaaacacacacacacacacacacacacacaacacacacacacacacacacacacacacacacacaaaactccctaccgggttcccagttcaatcctgaccccctgatctgattcgtgtcccttatgggcccagagcgctaagcaaataacttcccccattccctggaatttctttgcccagctctcctcagcgtgtggtccctctgccccttccccctcctcccagcaccaagctctctccttccccaaggcctcctcaaatccctctcccactcctggttgccttcctagctaccctctccctgtctaggggggagtgcaccctccttaggcagtggggtctgtgctgaccgcctgctgactgccttgcaggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtaatggcactgagcagaagggaagaagctccgggggctctttgtagggtcctccagtcaggactcaaacccagtagtgtctggttccaggcactgaccttgtatgtctcctggcccaaatgcccactcagggtaggggtgtagggcagaagaagaaacagactctaatgttgctacaagggctggtcccatctcctgagccccatgtcaaacagaatccaagacatcccaacccttcaccttggctgtgcccctaatcctcaactaagctaggcgcaaattccaatcctctttggtctagtaccccgggggcagccccctctaaccttgggcctcagcagcaggggaggccacaccttcctagtgcaggtggccatattgtggccccttggaactgggtcccactcagcctctaggcgattgtctcctaatggggctgagatgagacacagtggggacagtggtttggacaataggactggtgactctggtccccagaggcctcatgtccctctgtctccagaaaattcccactctcacttccctttcctcctcagtcttgctagggtccatttcttaccccttgctgaatttgagcccaccccctggactttttccccatcttctccaatctggcctagttctatcctctggaagcagagccgctggacgctctgggtttcctgaggcccgtccactgtcaccaatatcaggaaccattgccacgtcctaatgacgtgcgctggaagcctctagtttccagaagctgcacaaagatcccttagatactctgtgtgtccatctttggcctggaaaatactctcaccctggggctaggaagacctcggtttgtacaaacttcctcaaatgcagagcctgagggctctccccacctcctcaccaaccctctgcgtggcatagccctagcctcagcgggcagtggatgctggggctgggcatgcagggagaggctgggtggtgtcatctggtaacgcagccaccaaacaatgaagcgacactgattccacaaggtgcatctgcatccccatctgatccattccatcctgtcacccagccatgcagacgtttatgatccccttttccagggagggaatgtgaagccccagaaagggccagcgctcggcagccaccttggctgttcccaagtccctcacaggcagggtctccctacctgcctgtcctcaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggtacgggccggggggtgggcggcctcacggctctgagggtccagcccccagcatgcatctgcggctcctgctccctggaggagccatggtctggacccgggtcccgtgtcctgcaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcaggtgcctactgcgggtgggagggccccagtgccccaggccacaggcgctgcctgccaaggacaagctacttcccagggcaggggagggggctccatcagggttactggcagcagtcttgggtcagcagtcccaatggggagtgtgtgagaaatgcagattcctggccccactcagaactgctgaatctcagggtgggcccaggaacctgcatttccagcaagccctccacaggtggctcagatgctcactcaggtgggagaagctccagtcagctagttctggaagcccaatgtcaaagtcagaaggacccaagtcgggaatgggatgggccagtctccataaagctgaataaggagctaaaaagtcttattctgaggggtaaaggggtaaagggttcctcggagaggtacctccgaggggtaaacagttgggtaaacagtctctgaagtcagctctgccattttctagctgtatggccctgggcaagtcaatttccttctctgtgctttggtttcctcatccatagaaaggtagaaagggcaaaacaccaaactcttggattacaagagataatttacagaacacccttggcacacagagggcaccatgaaatgtcacgggtgacacagcccccttgtgctcagtccctggcatctctaggggtgaggagcgtctgcctagcaggttccctccaggaagctggatttgagtggatggggcgctggaatcgtgaggggcagaagcaggcaaagggtcggggcgaacctcactaacgtgccagttccaagcacactgtgggcagccctggccctgactcaagcctcttgccttccagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccaccaagacctactgatctggagtcccacgttccccaaggccagcgggatgtgtgcccctcctcctcccaactcatctttcaggaacacgaggattcttgctttctggaaaagtgtcccagcttagggataagtgtctagcacagaatggggcacacagtaggtgcttaataaatgctggatggatgcaggaaggaatggaggaatgaatgggaagggagaacatatctatcctctcagaccctcgcagcagcagcaactcatacttggctaatgatatggagcagttgtttttccctccctgggcctcactttcttctcctataaaatggaaatcccagatccctggtcctgccgacacgcagctactgagaagaccaaaagaggtgtgtgtgtgtctatgtgtgtgtttcagcactttgtaaatagcaagaagctgtacagattctagttaatgttgtgaataacatcaattaatgtaactagttaattactatgattatcacctcctgatagtgaacattttgagattgggcattcagatgatggggtttcacccaaccttggggcaggtttttaaaaattagctaggcatcaaggccagaccagggctgggggttgggctgtaggcagggacagtcacaggaatgcagaatgcagtcatcagacctgaaaaaacaacactgggggagggggacggtgaaggccaagttcccaatgagggtgagattgggcctggggtctcacccctagtgtggggccccaggtcccgtgcctccccttcccaatgtggcctatggagagacaggcctttctctcagcctctggaagccacctgctcttttgctctagcacctgggtcccagcatctagagcatggagcctctagaagccatgctcacccgcccacatttaattaacagctgagtccctgatgtcatccttatctcgaagagcttagaaacaaagagtgggaaattccactgggcctaccttccttggggatgttcatgggccccagtttccagtttcccttgccagacaagcccatcttcagcagttgctagtccattctccattctggagaatctgctccaaaaagctggccacatctctgaggtgtcagaattaagctgcctcagtaactgctcccccttctccatataagcaaagccagaagctctagctttacccagctctgcctggagactaaggcaaattgggccattaaaagctcagctcctatgttggtattaacggtggtgggttttgttgctttcacactctatccacaggatagattgaaactgccagcttccacctgatccctgaccctgggatggctggattgagcaatgagcagagccaagcagcacagagtcccctggggctagaggtggaggaggcagtcctgggaatgggaaaaacccca

SEQ ID NO: 250 - protein, 348 aa, Homo sapiens [sequence omitted]

SEQ ID NO: 251 - DNA, 2768 bp, Homo sapiens
  source 1..2768: mol_type = other DNA
agagtcatccagctggagccctgagtggctgagctcaggccttcgcagcattcttgggtgggagcagccacgggtcagccacaagggccacagccatgaatggcacagaaggccctaacttctacgtgcccttctccaatgcgacgggtgtggtacgcagccccttcgagtacccacagtactacctggctgagccatggcagttctccatgctggccgcctacatgtttctgctgatcgtgctgggcttccccatcaacttcctcacgctctacgtcaccgtccagcacaagaagctgcgcacgcctctcaactacatcctgctcaacctagccgtggctgacctcttcatggtcctaggtggcttcaccagcaccctctacacctctctgcatggatacttcgtcttcgggcccacaggatgcaatttggagggcttctttgccaccctgggcggtgaaattgccctgtggtccttggtggtcctggccatcgagcggtacgtggtggtgtgtaagcccatgagcaacttccgcttcggggagaaccatgccatcatgggcgttgccttcacctgggtcatggcgctggcctgcgccgcacccccactcgccggctggtccaggtacatccccgagggcctgcagtgctcgtgtggaatcgactactacacgctcaagccggaggtcaacaacgagtcttttgtcatctacatgttcgtggtccacttcaccatccccatgattatcatctttttctgctatgggcagctcgtcttcaccgtcaaggaggccgctgcccagcagcaggagtcagccaccacacagaaggcagagaaggaggtcacccgcatggtcatcatcatggtcatcgctttcctgatctgctgggtgccctacgccagcgtggcattctacatcttcacccaccagggctccaacttcggtcccatcttcatgaccatcccagcgttctttgccaagagcgccgccatctacaaccctgtcatctatatcatgatgaacaagcagttccggaactgcatgctcaccaccatctgctgcggcaagaacccactgggtgacgatgaggcctctgctaccgtgtccaagacggagacgagccaggtggccccggcctaagacctgcctaggactctgtggccgactataggcgtctcccatcccctacaccttcccccagccacagccatcccaccaggagcagcgcctgtgcagaatgaacgaagtcacataggctccttaattttttttttttttttaagaaataattaatgaggctcctcactcacctgggacagcctgagaagggacatccaccaagacctactgatctggagtcccacgttccccaaggccagcgggatgtgtgcccctcctcctcccaactcatctttcaggaacacgaggattcttgctttctggaaaagtgtcccagcttagggataagtgtctagcacagaatggggcacacagtaggtgcttaataaatgctggatggatgcaggaaggaatggaggaatgaatgggaagggagaacatatctatcctctcagaccctcgcagcagcagcaactcatacttggctaatgatatggagcagttgtttttccctccctgggcctcactttcttctcctataaaatggaaatcccagatccctggtcctgccgacacgcagctactgagaagaccaaaagaggtgtgtgtgtgtctatgtgtgtgtttcagcactttgtaaatagcaagaagctgtacagattctagttaatgttgtgaataacatcaattaatgtaactagttaattactatgattatcacctcctgatagtgaacattttgagattgggcattcagatgatggggtttcacccaaccttggggcaggtttttaaaaattagctaggcatcaaggccagaccagggctgggggttgggctgtaggcagggacagtcacaggaatgcagaatgcagtcatcagacctgaaaaaacaacactgggggagggggacggtgaaggccaagttcccaatgagggtgagattgggcctggggtctcacccctagtgtggggccccaggtcccgtgcctccccttcccaatgtggcctatggagagacaggcctttctctcagcctctggaagccacctgctcttttgctctagcacctgggtcccagcatctagagcatggagcctctagaagccatgctcacccgcccacatttaattaacagctgagtccctgatgtcatccttatctcgaagagcttagaaacaaagagtgggaaattccactgggcctaccttccttggggatgttcatgggccccagtttccagtttcccttgccagacaagcccatcttcagcagttgctagtccattctccattctggagaatctgctccaaaaagctggccacatctctgaggtgtcagaattaagctgcctcagtaactgctcccccttctccatataagcaaagccagaagctctagctttacccagctctgcctggagactaaggcaaattgggccattaaaagctcagctcctatgttggtattaacggtggtgggttttgttgctttcacactctatccacaggatagattgaaactgccagcttccacctgatccctgaccctgggatggctggattgagcaatgagcagagccaagcagcacagagtcccctggggctagaggtggaggaggcagtcctgggaatgggaaaaacccca

SEQ ID NO: 252 - DNA, 75 bp, synthetic construct
  source 1..75: mol_type = other DNA; note = Synthetic oligonucleotide
aayytngarggnttyttygcnacnytnggnggngarathgcnytntggwsnytngtngtnytngcnathgartrr

SEQ ID NO: 253 - DNA, 72 bp, synthetic construct
  source 1..72: mol_type = other DNA; note = Synthetic oligonucleotide
aatttggagggcttctttgccaccctgggcggtgaaattgccctgtggtccttggtggtcctggccatcgag

SEQ ID NO: 254 - DNA, 75 bp, synthetic construct
  source 1..75: mol_type = other DNA; note = Synthetic oligonucleotide
garathgcnytngtngtnytnwsntggytngcnathgarggnggnytnacngcnttyttyggngarytnaaytrr

SEQ ID NO: 255 - DNA, 72 bp, synthetic construct
  source 1..72: mol_type = other DNA; note = Synthetic oligonucleotide
gagatcgccctggtcgtgttgtcctggctggccattgaaggtggcctgaccgcctttttcggcgagttgaat

SEQ ID NO: 256 - protein, 8 aa, synthetic construct
  source 1..8: note = Synthetic oligonucleotide
WARYADWL

SEQ ID NO: 257 - protein, 8 aa, synthetic construct
  source 1..8: note = Synthetic peptide
LWDAYRAW

SEQ ID NO: 258 - protein, 34 aa, synthetic construct
  source 1..34: note = Synthetic polypeptide
AEQNPIYWARYADWLFTTPLLLLDLALLVDADET

SEQ ID NO: 259 - protein, 34 aa, synthetic construct
  MOD_RES 13: note = Gamma-carboxyglutamic acid
  source 1..34: note = Synthetic polypeptide
AEQNPIYWARYAXWLFTTPLLLLDLALLVDADET

SEQ ID NO: 260 - protein, 34 aa, synthetic construct
  MOD_RES 24: note = Gamma-carboxyglutamic acid
  source 1..34: note = Synthetic polypeptide
AEQNPIYWARYADWLFTTPLLLLXLALLVDADET

SEQ ID NO: 261 - protein, 34 aa, synthetic construct; identical to SEQ ID NO: 259
  MOD_RES 13: note = Aad
  source 1..34: note = Synthetic polypeptide

SEQ ID NO: 262 - protein, 34 aa, synthetic construct; identical to SEQ ID NO: 260
  MOD_RES 24: note = Aad
  source 1..34: note = Synthetic polypeptide

SEQ ID NO: 263 - protein, 34 aa, synthetic construct; identical to SEQ ID NO: 157
  MOD_RES 13: note = Gamma-carboxyglutamic acid
  MOD_RES 24: note = Aad
  source 1..34: note = Synthetic polypeptide

SEQ ID NO: 264 - protein, 36 aa, synthetic construct
  source 1..36: note = Synthetic polypeptide
GGEQNPIYWARYADWLFTTPLLLLDLALLVDADEGT

SEQ ID NO: 265 - protein, 36 aa, synthetic construct; identical to SEQ ID NO: 264
  source 1..36: note = Synthetic polypeptide

SEQ ID NO: 266 - protein, 36 aa, synthetic construct
  source 1..36: note = Synthetic polypeptide
AAEQNPIYWARYADWLFTTPLLLLDLALLVDADEGT

SEQ ID NO: 267 - protein, 35 aa, synthetic construct; identical to SEQ ID NO: 2
  source 1..35: note = Synthetic polypeptide

SEQ ID NO: 268 - protein, 35 aa, synthetic construct
  source 1..35: note = Synthetic polypeptide
AEQNPIYWARYAKWLFTTPLLLLKLALLVDADEGT

SEQ ID NO: 269 - protein, 36 aa, synthetic construct
  source 1..36: note = Synthetic polypeptide
GGEQNPIYWARYADWLFTTPLLLLDLALLVNANQGT

SEQ ID NO: 270 - protein, 36 aa, synthetic construct
  source 1..36: note = Synthetic polypeptide
AAEQNPIYWARYADWLFTTPLLLLALALLVDADEGT

SEQ ID NO: 271 - protein, 36 aa, synthetic construct
  source 1..36: note = Synthetic polypeptide
AAEQNPIYWARYAAWLFTTPLLLLDLALLVDADEGT

SEQ ID NO: 272 - protein, 36 aa, synthetic construct
  source 1..36: note = Synthetic polypeptide
AAEQNPIYWARYADWLFTTALLLLDLALLVDADEGT

SEQ ID NO: 273 - protein, 36 aa, synthetic construct
  source 1..36: note = Synthetic polypeptide
AAEQNPIYWARYADWLFTTPLLLLELALLVDADEGT

SEQ ID NO: 274 - protein, 36 aa, synthetic construct
  source 1..36: note = Synthetic polypeptide
AAEQNPIYWARYAEWLFTTPLLLLDLALLVDADEGT

SEQ ID NO: 275 - protein, 39 aa, synthetic construct
  source 1..39: note = Synthetic polypeptide
AAEQNPIIYWARYADWLFTDLPLLLLDLLALLVDADEGT

SEQ ID NO: 276 - protein, 34 aa, synthetic construct
  source 1..34: note = Synthetic polypeptide
GEQNPIYWAQYADWLFTTPLLLLDLALLVDADEG

SEQ ID NO: 277 - protein, 35 aa, synthetic construct
  source 1..35: note = Synthetic polypeptide
GGEQNPIYWARYADWLFTTPLLLDLLALLVDADEG

SEQ ID NO: 278 - protein, 35 aa, synthetic construct
  source 1..35: note = Synthetic polypeptide
GGEQNPIYWARYADWLFTTPLLLLLDALLVDADEG

SEQ ID NO: 279 - protein, 36 aa, synthetic construct
  source 1..36: note = Synthetic polypeptide
GGEQNPIYWARYDAWLFTTPLLLLDLALLVDADEGT

SEQ ID NO: 280 - protein, 35 aa, synthetic construct
  source 1..35: note = Synthetic polypeptide
GGEQNPIYWARYAWDLFTTPLLLLDLALLVDADEG

SEQ ID NO: 281 - protein, 36 aa, synthetic construct
  source 1..36: note = Synthetic polypeptide
AAEQNPIYWARYADWLFTTGLLLLDLALLVDADEGT

SEQ ID NO: 282 - protein, 34 aa, synthetic construct
  source 1..34: note = Synthetic polypeptide
DDDEDNPIYWARYADWLFTTPLLLLHGALLVDAD

SEQ ID NO: 283 - protein, 35 aa, synthetic construct
  source 1..35: note = Synthetic polypeptide
DDDEDNPIYWARYAHWLFTTPLLLLHGALLVDADE

SEQ ID NO: 284 - protein, 34 aa, synthetic construct
  source 1..34: note = Synthetic polypeptide
AEQNPIYWARYADFLFTTPLLLLDLALLVDADET

SEQ ID NO: 285 - protein, 33 aa, synthetic construct
  source 1..33: note = Synthetic polypeptide
AEQNPIYFARYADWLFTTPLLLLDLALLVDADE

SEQ ID NO: 286 - protein, 34 aa, synthetic construct
  source 1..34: note = Synthetic polypeptide
AEQNPIYFARYADFLFTTPLLLLDLALLWDADET

SEQ ID NO: 287 - protein, 33 aa, synthetic construct
  source 1..33: note = Synthetic polypeptide
TEDADVLLALDLLLLPTTFLWDAYRAWYPNQEA

SEQ ID NO: 288 - protein, 30 aa, synthetic construct
  source 1..30: note = Synthetic polypeptide
AEDQNPYWARYADWLFTTPLLLLDLALLVD

SEQ ID NO: 289 - protein, 30 aa, synthetic construct
  source 1..30: note = Synthetic polypeptide
AEDQNPYWARYADWLFTTPLLLLELALLVE

SEQ ID NO: 290 - protein, 28 aa, synthetic construct
  source 1..28: note = Synthetic peptide
AEDQNPYWRAYADLFTPLTLLDLLALWD

SEQ ID NO: 291 - protein, 28 aa, synthetic construct
  source 1..28: note = Synthetic peptide
ADDDDDDPWQAYLDLLFPTDTLLLDLLW

SEQ ID NO: 292 - protein, 26 aa, synthetic construct
  source 1..26: note = Synthetic peptide
AEEQNPWRAYLELLFPTETLLLELLW

SEQ ID NO: 293 - protein, 24 aa, synthetic construct
  source 1..24: note = Synthetic peptide
ADDQNPWARYLDWLFPTDTLLLDL

SEQ ID NO: 294 - protein, 23 aa, synthetic construct
  source 1..23: note = Synthetic peptide
DNNNPWRAYLDLLFPTDTLLLDW

SEQ ID NO: 295 - protein, 24 aa, synthetic construct
  source 1..24: note = Synthetic peptide
AEEQNPWARYLEWLFPTETLLLEL

SEQ ID NO: 296 - protein, 25 aa, synthetic construct
  source 1..25: note = Synthetic peptide
DDDDDDPWQAYLDLFPTDTLALDLW

SEQ ID NO: 297 - protein, 23 aa, synthetic construct
  source 1..23: note = Synthetic peptide
EEQQPWAQYLELLFPTETLLLEW

SEQ ID NO: 298 - protein, 23 aa, synthetic construct
  source 1..23: note = Synthetic peptide
EEQQPWRAYLELLFPTETLLLEW

SEQ ID NO: 299 - protein, 23 aa, synthetic construct
  source 1..23: note = Synthetic peptide
AEDQNPWARYADWLFPTTLLLLD

SEQ ID NO: 300 - protein, 23 aa, synthetic construct
  source 1..23: note = Synthetic peptide
AEEQNPWARYAEWLFPTTLLLLE

SEQ ID NO: 301 - protein, 21 aa, synthetic construct
  source 1..21: note = Synthetic peptide
AEDQNPWARYADLLFPTTLAW

SEQ ID NO: 302 - protein, 21 aa, synthetic construct
  source 1..21: note = Synthetic peptide
AEEQNPWARYAELLFPTTLAW

SEQ ID NO: 303 - protein, 36 aa, synthetic construct
  source 1..36: note = Synthetic peptide
DDDDDNPNYWARYANWLFTTPLLLLNGALLVEAEET

SEQ ID NO: 304 - protein, 36 aa, synthetic construct
  source 1..36: note = Synthetic peptide
DDDDDNPNYWARYAPWLFTTPLLLLPGALLVEAEET

SEQ ID NO: 305 - protein, 34 aa, synthetic construct; identical to SEQ ID NO: 286
  source 1..34: note = Synthetic polypeptide

SEQ ID NO: 306 - protein, 34 aa, synthetic construct; identical to SEQ ID NO: 284
  source 1..34: note = Synthetic polypeptide

SEQ ID NO: 307 - protein, 20 aa, synthetic construct
  source 1..20: note = Synthetic peptide
AEQNPIYWARYADWLFTTPL

SEQ ID NO: 308 - protein, 22 aa, synthetic construct
  source 1..22: note = Synthetic peptide
AEQNPIYFARYADLLFPTTLAW

SEQ ID NO: 309 - protein, 22 aa, synthetic construct
  source 1..22: note = Synthetic peptide
AEQNPIYWARYADLLFPTTLAF

SEQ ID NO: 310 - protein, 22 aa, synthetic construct
  source 1..22: note = Synthetic peptide
AEQNPIYWARYADLLFPTTLAW

SEQ ID NO: 311 - protein, 20 aa, synthetic construct
  source 1..20: note = Synthetic peptide
AEQNPIYFARYADWLFTTPL

SEQ ID NO: 312 - protein, 20 aa, synthetic construct
  source 1..20: note = Synthetic peptide
EDQNPWARYADLLFPTTLAW

SEQ ID NO: 313 - protein, 36 aa, synthetic construct
  source 1..36: note = Synthetic polypeptide
ACEQNPIYWARYADWLFTTPLLLLDLALLVDADEGT

SEQ ID NO: 314 - protein, 36 aa, synthetic construct
  source 1..36: note = Synthetic polypeptide
AEQNPIYWARYADWLFTTPLLLLDLALLVDADEGCT

SEQ ID NO: 315 - protein, 36 aa, synthetic construct
  source 1..36: note = Synthetic polypeptide
AKEQNPIYWARYADWLFTTPLLLLDLALLVDADECT

SEQ ID NO: 316 - protein, 36 aa, synthetic construct
  MOD_RES 14: note = Gamma-carboxyglutamic acid
  source 1..36: note = Synthetic polypeptide
AKEQNPIYWARYAXWLFTTPLLLLDLALLVDADECT

SEQ ID NO: 317 - protein, 36 aa, synthetic construct
  MOD_RES 14: note = Gamma-carboxyglutamic acid
  MOD_RES 25: note = Aad
  source 1..36: note = Synthetic polypeptide
AKEQNPIYWARYAXWLFTTPLLLLXLALLVDADECT

SEQ ID NO: 318 - protein, 28 aa, synthetic construct
  MOD_RES 13: note = Gamma-carboxyglutamic acid
  source 1..28: note = Synthetic peptide
ACDDQNPWRAYLXLLFPTDTLLLDLLWG

SEQ ID NO: 319 - protein, 32 aa, synthetic construct
  source 1..32: note = Synthetic polypeptide
GKEEQNPWLGAYLDLLFPLELLGLLELGLWCG

SEQ ID NO: 320 - protein, 36 aa, synthetic construct
  source 1..36: note = Synthetic polypeptide
ACGLAGLAGLLGLEGLLGLPLGLLEGLWLGLELEGN

SEQ ID NO: 321 - protein, 25 aa, synthetic construct
  source 1..25: note = Synthetic peptide
AKEEQNPWARYLEWLFPTETLLLEL

SEQ ID NO: 322 - protein, 8 aa, synthetic construct
  source 1..8: note = Synthetic peptide
LGGEIALW

SEQ ID NO: 323 - protein, 35 aa, synthetic construct
  source 1..35: note = Synthetic polypeptide
AEQNPIYWARYANWLFTTPLLLLNLALLVDADEGT

SEQ ID NO: 324 - protein, 36 aa, synthetic construct
  source 1..36: note = Synthetic polypeptide
GLAGLAGLLGLEGLLGLPLGLLEGLWLGLELEGNCA

SEQ ID NO: 325 - protein, 8 aa, synthetic construct
  source 1..8: note = Synthetic peptide
LLFPTDTL

SEQ ID NO: 326 - protein, 27 aa, synthetic construct
  source 1..27: note = Synthetic peptide
ADDQNPWRAYLDLLFPTDTLLLDLLWC

SEQ ID NO: 327 - protein, 28 aa, synthetic construct
  MOD_RES 12: note = Gamma-carboxyglutamic acid
  source 1..28: note = Synthetic peptide
ADDQNPWRAYLXLLFPTDTLLLDLLWCG

SEQ ID NO: 328 - protein, 21 aa, synthetic construct
  source 1..21: note = Synthetic peptide
LLSLDLLLLPTTFLWDAYRAW

SEQ ID NO: 329 - protein, 5 aa, synthetic construct
  source 1..5: note = Synthetic peptide
WLXLL

SEQ ID NO: 330 - protein, 5 aa, synthetic construct
  source 1..5: note = Synthetic peptide
WLGLL

SEQ ID NO: 331 - DNA, 12 bp, synthetic construct
  source 1..12: mol_type = other DNA; note = Synthetic oligonucleotide; note = Peptide nucleic acid
catagtataagt